(19) United States
(12) Patent Application Publication
(10) Pub. No.: US 2021/0040464 A1
(43) Pub. Date: Feb. 11, 2021

(54) METHODS AND COMPOSITIONS FOR TREATMENT OF POLYGLUCOSAN DISORDERS

(71) Applicant: Valerion Therapeutics, LLC, Concord, MA (US)

(72) Inventors: Dustin D. Armstrong, Quincy, MA (US); Tracy McKnight, Oakland, CA (US)

(21) Appl. No.: 16/981,268

(22) PCT Filed: Mar. 15, 2019
(86) PCT No.: PCT/US2019/022566

§ 371 Date: Sep. 15, 2020

Related U.S. Application Data
(60) Provisional application No. 62/682,928, filed on Jun. 9, 2018.
(60) Provisional application No. 62/643,592, filed on Mar. 15, 2018.

(51) Int. Cl. C12N 9/26; C07K 16/00

(52) U.S. Cl. CPC C12N 9/2414; C07K 16/00; C07K 2319/00; C07K 2317/77; C07K 2317/622; C07K 2317/56; C12Y 302/0102; C12Y 302/01001; C07K 2317/55; C07K 2317/52

(57) ABSTRACT

In certain embodiments, the present disclosure provides compositions and methods for treating polyglucosan disorders.

### RELATED APPLICATION

This application claims the benefit of U.S. Provisional Application No. 62/643,592, filed Mar. 15, 2018, and U.S. Provisional Application No. 62/682,928, filed Jun. 9, 2018. The entire teachings of the above applications are incorporated herein by reference.

### BACKGROUND OF THE DISCLOSURE

Glycogen storage diseases and glycogen metabolism disorders are a series of diseases that are caused by defects in basic metabolizing enzymes, thereby resulting in defects in glycogen synthesis or breakdown within muscles, liver, neurons and other cell types. Glycogen storage diseases may be either genetic (usually as autosomal recessive disorders) or acquired (e.g., by intoxication with alkaloids) (Monga et al.,). There are a number of different types of glycogen storage diseases, including GSDs Types I-XI, GSD Type 0, as well as Lafora disease which is often termed a glycogen metabolism disorder. These diseases differ with regard to the enzyme that is mutated and/or primary tissue affected (Monga et al., 2011, Molecular Pathology of Liver Diseases, Molecular Pathology Library 5, Chapter 45; and Gentry, et al., 2013, FEBS J, 280(2):525-37).

### SUMMARY OF THE DISCLOSURE

There is a need in the art for methods and compositions for clearing glycogen build-up, particularly cytoplasmic glycogen build-up, or for treating the cytotoxic effects associated with glycogen build-up, in patients with glycogen storage diseases and glycogen metabolism disorders (e.g., Forbes-Cori and/or Andersen Disease and/or von Gierke Disease and/or Pompe Disease and/or Lafora Disease and/or Danon Disease and/or Alzheimer's Disease) as well as a need for alternative therapies for treating these diseases or disorders. The present disclosure provides such methods and compositions. For example, there exists a need for decreasing glycogen accumulation in, for example, cytoplasm of cells, such as muscle (e.g. cardiac and/or diaphragm) and/or liver and/or neuronal cells (e.g., brain cells). By way of further example, such methods and compositions may decrease cytoplasmic glycogen accumulation.

Accordingly, throughout the application, references to clearing glycogen build-up or decreasing glycogen accumulation (or like terms) encompass, unless otherwise specified, clearing or decreasing excess (e.g., beyond normal physiological level) glycogen, including clearing or decreasing excess glycogen present in an abnormal form (e.g., polyglucosan). In certain embodiments, the disclosure provides methods of clearing or decreasing excess polyglucosan (e.g., clearing or decreasing polyglucosan accumulation), such as in cytoplasm, such as in one or more of muscle cells (skeletal and/or cardiac), diaphragm, or neurons. In certain embodiments, clearing glycogen build-up or decreasing glycogen accumulation (or like terms) refers to doing so in, at least, cytoplasm of one or more affected cells. In certain embodiments, clearing glycogen build-up or decreasing glycogen accumulation, such as in, at least, cytoplasm, is or comprises clearing polyglucosan build-up or decreasing polyglucosan accumulation, such as in, at least, cytoplasm. Such methods and compositions would improve treatment of diseases or disorders, particularly in patients whose disease is severe enough and/or advanced enough to have significant abnormal cytoplasmic glycogen accumulation (e.g., of normal and/or abnormal glycogen). The present disclosure provides such methods and compositions.

In certain embodiments, the methods and compositions provided herein decrease glycogen build-up (e.g., such as clear glycogen build-up or decrease glycogen accumulation) in, at least, the cytoplasm. In certain embodiments, the methods and compositions of the present disclosure decrease polyglucosan build-up (e.g., build-up in, at least, the cytoplasm of cell(s), such as muscle and/or liver and/or diaphragm, and/or neuronal cell(s)). In certain embodiments, the methods and compositions of the present disclosure decrease glycogen, such as polyglucosan, build-up in, at least, cytoplasm of, at least muscle and/or neuronal cells.

In some embodiments, the disclosure provides for a method for treating a subject having Danon Disease, comprising administering to the subject a therapeutically effective amount of any of the chimeric polypeptides disclosed herein.

In some embodiments, the disclosure provides for a method for treating a subject having Alzheimer's Disease, comprising administering to the subject a therapeutically effective amount of any of the chimeric polypeptides disclosed herein

In some embodiments, the disclosure provides for a chimeric polypeptide comprising: (i) an alpha-amylase polypeptide, and (ii) an internalizing moiety; wherein the alpha-amylase polypeptide comprises the amino acid sequence of SEQ ID NO: 1; and wherein the internalizing moiety is an antibody or antigen binding fragment, wherein the antibody or antigen binding fragment comprises a heavy chain variable domain and a light chain variable domain; wherein the heavy chain variable domain comprises the amino acid sequence of SEQ ID NO: 2; and wherein the light chain variable domain comprises the amino acid sequence of SEQ ID NO: 3.

In some embodiments, the alpha-amylase polypeptide consists of the amino acid sequence of SEQ ID NO: 1. In some embodiments, the heavy chain comprises the leader sequence of SEQ ID NO: 4. In some embodiments, the light chain comprises the leader sequence of SEQ ID NO: 5. In some embodiments, the chimeric polypeptide has alpha-1,4-glucosidic bonds hydrolytic activity. In some embodiments, the chimeric polypeptide is capable of hydrolyzing alpha-1,4-glucosidic bonds in a cell-free system. In some embodiments, the chimeric polypeptide is capable of hydrolyzing alpha-1,4-glucosidic bonds in a cell from a subject having the disease. In some embodiments, the subject is a non-human animal. In some embodiments, the non-human animal is a mouse. In some embodiments, the subject is a human. In some embodiments, the cell is in vitro. In some embodiments, the cell is a muscle cell. In some embodiments, the cell is a cardiac muscle cell. In some embodiments, the cell is a brain cell. In some embodiments, the cell is a neuron. In some embodiments, the alpha-amylase polypeptide is chemically conjugated to the internalizing moiety. In some embodiments, the chimeric polypeptide comprises a fusion protein comprising the alpha-amylase polypeptide and all or a portion of the internalizing moiety. In some embodiments, the chimeric polypeptide does not include a linker interconnecting the alpha-amylase polypeptide to the internalizing moiety. In some embodiments, the fusion protein comprises a linker. In some embodiments, the linker conjugates or joins the alpha-amylase polypeptide to the internalizing moiety. In some embodiments, the linker is a cleavable linker. In some embodiments, the linker comprises the amino acid sequence of SEQ ID NO: 6. In some embodiments, all or a portion of the internalizing moiety is conjugated or joined, directly or via a linker, to the N-terminal amino acid of the alpha-amylase polypeptide. In some embodiments, wherein all or a portion of the internalizing moiety is conjugated or joined, directly or via a linker, to the C-terminal amino acid of the alpha-amylase polypeptide. In some embodiments, all or a portion of the internalizing moiety is conjugated or joined, directly or indirectly to an internal amino acid of the alpha-amylase polypeptide. In some embodiments, the internalizing moiety promotes delivery of the chimeric polypeptide into cells via an equilibrative nucleoside transporter (ENT) transporter. In some embodiments, the internalizing moiety promotes delivery of the chimeric polypeptide into cells via ENT2. In some embodiments, the internalizing moiety promotes delivery of the chimeric polypeptide into a muscle cell. In some embodiments, the muscle cell is a diaphragm muscle cell. In some embodiments, the internalizing moiety promotes delivery of the chimeric polypeptide into a neuronal cell. In some embodiments, the neuronal cell is a brain neuronal cell. In some embodiments, the internalizing moiety comprises an antibody. In some embodiments, the antibody is a monoclonal antibody. In some embodiments, the internalizing moiety comprises an antigen-binding fragment. In some embodiments, the antigen-binding fragment is a Fab. In some embodiments, the antigen-binding fragment is a Fab′. In some embodiments, the antigen-binding fragment is an scFv. In some embodiments, the chimeric polypeptide is produced recombinantly. In some embodiments, the chimeric polypeptide is produced in a prokaryotic or eukaryotic cell. In some embodiments, the eukaryotic cell is selected from a yeast cell, an avian cell, an insect cell, or a mammalian cell. In some embodiments, one or more glycosylation groups are conjugated to the chimeric polypeptide. In some embodiments, the chimeric polypeptide comprises the amino acid sequence of SEQ ID NO: 7. In some embodiments, the chimeric polypeptide comprises the amino acid sequence of SEQ ID NO: 8. In some embodiments, the chimeric polypeptide comprises the amino acid sequence of SEQ ID NOs: 7 and 8. In some embodiments, the chimeric polypeptide comprises the amino acid sequence of SEQ ID NO: 9. In some embodiments, the chimeric polypeptide comprises the amino acid sequence of SEQ ID NO: 10. In some embodiments, the chimeric polypeptide comprises the amino acid sequence of SEQ ID NOs: 9 and 10. In some embodiments, the chimeric polypeptide comprises the amino acid sequence of SEQ ID NO: 43. In some embodiments, the chimeric polypeptide comprises the amino acid sequence of SEQ ID NO: 8. In some embodiments, the chimeric polypeptide comprises the amino acid sequences of SEQ ID NOs: 8 and 43.

In some embodiments, the disclosure provides for a method for treating a subject having Lafora Disease, comprising administering to the subject a therapeutically effective amount of a chimeric polypeptide comprising: (i) a mature acid alpha-glucosidase (GAA) polypeptide, and (ii) an internalizing moiety.

In some embodiments, the disclosure provides for a method for delivering acid alpha-glucosidase activity into a cell from or of a subject having Lafora Disease, comprising contacting the cell with a chimeric polypeptide comprising: (i) a mature acid alpha-glucosidase polypeptide, and (ii) an internalizing moiety.

In some embodiments, the chimeric polypeptide has acid alpha-glucosidase activity. In some embodiments, the internalizing moiety is an antibody or antigen binding fragment, wherein the antibody or antigen binding fragment comprises a heavy chain variable domain and a light chain variable domain; wherein the heavy chain variable domain comprises the amino acid sequence of SEQ ID NO: 2; and wherein the light chain variable domain comprises the amino acid sequence of SEQ ID NO: 3. In some embodiments, the chimeric polypeptide or mature GAA polypeptide comprises the amino acid sequence of SEQ ID NO: 49, 50, or 51. In some embodiments, the mature GAA polypeptide has a molecular weight of approximately 70-76 kilodaltons, 70 kilodaltons, or 76 kilodaltons. In some embodiments, the subject is a non-human animal (e.g., a mouse). In some embodiments, the subject is a human. In some embodiments, the cell is in vitro. In some embodiments, the cell is a muscle cell. In some embodiments, the cell is a diaphragm muscle cell. In some embodiments, the cell is a brain cell. In some embodiments, the cell is a neuron. In some embodiments, the method results in clearance of glycogen. In some embodiments, the method results in degradation of Lafora bodies.

In some embodiments, the disclosure provides for a method for treating a subject having Danon Disease, comprising administering to the subject a therapeutically effective amount of a chimeric polypeptide comprising: (i) a mature acid alpha-glucosidase (GAA) polypeptide, and (ii) an internalizing moiety.

In some embodiments, the disclosure provides for a method for delivering acid alpha-glucosidase activity into a cell from or of a subject having Danon Disease, comprising contacting the cell with a chimeric polypeptide comprising: (i) a mature acid alpha-glucosidase polypeptide, and (ii) an internalizing moiety.

In some embodiments, the chimeric polypeptide has acid alpha-glucosidase activity. In some embodiments, the internalizing moiety is an antibody or antigen binding fragment, wherein the antibody or antigen binding fragment comprises a heavy chain variable domain and a light chain variable domain; wherein the heavy chain variable domain comprises the amino acid sequence of SEQ ID NO: 2; and wherein the light chain variable domain comprises the amino acid sequence of SEQ ID NO: 3. In some embodiments, the chimeric polypeptide or mature GAA polypeptide comprises the amino acid sequence of SEQ ID NO: 49, 50, or 51. In some embodiments, the mature GAA polypeptide has a molecular weight of approximately 70-76 kilodaltons, 70 kilodaltons, or 76 kilodaltons. In some embodiments, the subject is a non-human animal (e.g., a mouse). In some embodiments, the subject is a human. In some embodiments, the cell is in vitro. In some embodiments, the cell is a muscle cell. In some embodiments, the cell is a diaphragm muscle cell. In some embodiments, the cell is a brain cell. In some embodiments, the cell is a neuron. In some embodiments, the method results in clearance of glycogen.

In some embodiments, the disclosure provides for a method for treating a subject having a polyglucosan accumulation disease, comprising administering to the subject a therapeutically effective amount of a chimeric polypeptide comprising: (i) a mature acid alpha-glucosidase (GAA) polypeptide, and (ii) an internalizing moiety.

In some embodiments, the disclosure provides for a method for delivering acid alpha-glucosidase activity into a cell from or of a subject having a polyglucosan disease, comprising contacting the cell with a chimeric polypeptide comprising: (i) a mature acid alpha-glucosidase polypeptide, and (ii) an internalizing moiety.

In some embodiments, the chimeric polypeptide has acid alpha-glucosidase activity. In some embodiments, the internalizing moiety is an antibody or antigen binding fragment, wherein the antibody or antigen binding fragment comprises a heavy chain variable domain and a light chain variable domain; wherein the heavy chain variable domain comprises the amino acid sequence of SEQ ID NO: 2; and wherein the light chain variable domain comprises the amino acid sequence of SEQ ID NO: 3. In some embodiments, the chimeric polypeptide or mature GAA polypeptide comprises the amino acid sequence of SEQ ID NO: 49, 50, or 51. In some embodiments, the mature GAA polypeptide has a molecular weight of approximately 70-76 kilodaltons, 70 kilodaltons, or 76 kilodaltons. In some embodiments, the subject is a non-human animal (e.g., a mouse). In some embodiments, the subject is a human. In some embodiments, the cell is in vitro. In some embodiments, the cell is a muscle cell. In some embodiments, the cell is a diaphragm muscle cell. In some embodiments, the cell is a brain cell. In some embodiments, the cell is a neuron. In some embodiments, the method results in clearance of glycogen. In some embodiments, the polyglucosan accumulation disease is a glycogen storage disorder IV (GSD IV), glycogen storage disorder VII (GSD VII), glycogen storage disorder XV (GSD XV), RBCK1 deficiency, and/or PRKAG2 associated cardiomyopathy (PAC).

### BRIEF DESCRIPTION OF THE FIGURES

The patent or application file contains at least one drawing executed in color. Copies of this patent or patent application publication with color drawings will be provided by the Office upon request and payment of the necessary fee.

FIG. 1 demonstrates dose dependent uptake of Fab-amylase in ENT2+C2C12 myotubes. A comparison of −Fab-amylase and +Fab-amylase at 0.01 mg/ml and 0.1 mg/ml is provided. (Notes: Anti-H3L2, Rabbit pAb, 1:100; Donkey Anti-Rabbit-HRP, 1:20000).

FIG. 2 is a graph demonstrating glycogen reduction in ENT2+C2C12 myotubes.

FIG. 3 demonstrates the use of Fab-GAA as a therapy option for Danon Disease. Cardiac tissue from Danon patients was processed and it was shown that Fab-GAA resulted in a decrease in relative glucose concentration as compared to PBS treated samples.

FIGS. 4A-4B show purified Lafora bodies can be degraded by Fab-amylase but not by Fab-glucosidase. FIG. 4A is a graph showing the percent of degradation of Lafora bodies from the brain, heart, and skeletal muscle when treated with Fab-amylase, Fab-glucosidase or control. FIG. 4B is a graph showing the Lafora body content (μg per mL extract) of WT and KO mice treated with −Fab-amylase and +Fab-amylse.

FIGS. 5A-5B demonstrate injected Fab-amylase is active in the muscle and brain. FIG. 5A is a graph showing amylase activity in the muscle 1 hr post-injection, 2 hrs post-injection, 4 hrs post-injection, and 24 hrs post-injection. FIG. 5B shows amylase activity (lower panel) for samples of the brain identified (upper panel) immediately post-injection and 1 hour post-injection.

FIG. 6 shows Periodic acid-Schiff (PAS) staining of the Tibialis anterior (TA) muscle of an 8.5 month old female mouse injected with a vehicle (PBS) in the left leg (left panel) and Fab-Amylase in the right leg (right panel).

FIG. 7 shows Periodic acid-Schiff (PAS) staining of the Tibialis anterior (TA) muscle of an 8.5 month old female mouse injected with a vehicle (PBS) in the left leg (left panel) and Fab-Amylase in the right leg (right panel).

FIG. 8 shows Periodic acid-Schiff (PAS) staining of the Tibialis anterior (TA) muscle of an 8.5 month old female mouse injected with a vehicle (PBS) in the left leg (left panel) and a vehicle (PBS) in the right leg (right panel).

FIG. 9 shows Periodic acid-Schiff (PAS) staining of the Tibialis anterior (TA) muscle of a 4 month old female mouse injected with a vehicle (PBS) in the left leg (left panel) and Fab-Amylase in the right leg (right panel).

FIGS. 10A-10K demonstrate clearance of glycogen from the brain of mice treated with ICV pump administration of Fab-Amylase or Fab-GAA for 28 days. FIG. 10A is a graph showing glucose levels in the brain of mice treated with PBS. FIG. 10B is a graph showing glucose levels in the brain of mice treated with Fab-Amylase. FIGS. 10C-10F are graphs showing a comparison of glucose levels in the brain of mice treated with PBS and mice treated with Fab-Amylase (Fab-Amy). FIG. 10G is a graph showing glucose levels in the brain of mice treated with Fab-GAA. FIGS. 10H-10K are graphs showing a comparison of glucose levels in the brain of mice treated with PBS and mice treated with Fab-GAA.

FIGS. 11A-11C show Fab-Amylase distribution through the brain and uptake into neurons visualized using anti-amylase immunohistochemistry (IHC). Ab21156 was used at 1:5000. FIG. 11A shows pancreas and salivary glands as a positive control. Positive control tissue stains very dark (left and middle tissue) or dark enough to identify (right); islet cells and stroma cells are negative. FIG. 11B shows L4 brain as a negative control. The choroid plexus (bottom) and neurons (top panel), both at 20×, are negative. FIG. 11C shows anti-amylase staining of mice treated with ICV with Fab-Amylase for three days. The choroid plexus (bottom) and neurons (top panel), both at 20×, are positive.

FIGS. 12A-12B show glycogen content in gastrocnemius muscle. Glycogen content was measured in the gastroc muscle in untreated WT mice, untreated laforin knock out mice, and in Fab-Amylase treated laforin knock out mice. The right gastroc was injected with 30 mg/ml Fab-Amylase (Fab-Amy) three times over 7 days. The mice were sacrificed 24 hours after the last injection and glycogen was measured in the right and left gastrocs. WT: N=4 mice; untreated KO mice: N=4 mice; Fab-Amylase treated KO mice: N=4 mice. FIG. 12A is a graph showing glycogen content levels in the gastroc muscle of untreated WT mice, untreated laforin KO mice, and in Fab-Amylase treated laforin KO mice. FIG. 12B is a graph showing glycogen content in uninjected gastrocenemius muscle and injected gastrocenemius muscle of Fab-Amylase treated laforin knock mice.

FIG. 13 provides a schematic of various GAA construct designs. The fusions include 1) 3E10 Fab with GAA 70-952 fused to the C-terminus of the heavy chain Fab segment; 2) 3E10 Fab with GAA 61-952 fused to the C-terminus of the heavy chain Fab segment; 3) 3E10 Fab with a 5-amino acid linker and GAA 57-952 fused to the C-terminus of the heavy chain Fab segment; 4) 3E10 Fab with a 13-amino acid linker and GAA 67-952 fused to the C-terminus of the heavy chain Fab segment; 5) GAA with point mutations designed to enhance C-terminal fusion, a 13-amino acid linker, and a 3E10 Fab fused at the N-terminus of the light chain; 6) a 3E10 whole antibody fused to GAA at the C-terminus of the heavy chain, with a junction similar to that of construct 4 above; and 7) a 3E10 whole antibody fused to GAA at the C-terminus of the heavy chain, with a bovine GAA pro-sequence upstream of the mature GAA sequence.

FIG. 14 provides a graph demonstrating pH dependent specific activity using a glycogen substrate with a Citrate-P04 Buffer. The specific activity is measured in μmol/min/mg again pH, with activity peaking at a pH of around 5.0.

FIG. 15 summarizes the specific activity of Fab-GAA over a range of pH values from a pH of 3.5 to a pH of 7.0.

FIGS. 16A-16B provide a glucose standard curve (FIG. 16B) and the corresponding data (FIG. 16A).

FIG. 17 provides a Fab-GAA glycogen standard curve, with the corresponding data found in Table 7.

FIG. 18 provides images showing PAS staining of skeletal muscle for wild-type mice treated with PBS. Notes: 89LG; no PAS positive fibers.

FIG. 19 provides images showing PAS staining of skeletal muscle for wild-type mice treated with Fab-GAA. Notes: 98LG; no PAS positive fibers.

FIG. 20 provides images showing PAS staining of skeletal muscle for Lafora knock-out mice treated with PBS. Notes: 85LG; 38/44 and 42/26 PAS positive fibers.

FIG. 21 provides images showing PAS staining of skeletal muscle for Lafora knock-out mice treated with Fab-GAA. Notes: 93LG; 2/33 and 1/35 PAS positive fibers.

FIG. 22 provides images showing PAS staining of skeletal muscle for Lafora knock-out mice treated with Myozyme. Notes: 102LG; 2/59 and 3/56 PAS positive fibers.

FIG. 23 provides a graphical representation of a quantitative biochemical comparison of cardiac glycogen load in PBS, Fab-GAA, and Myozyme treated Lafora knock-out mice.

FIG. 24 provides an image showing RDR13 PAS staining of cardiac muscle for Lafora knock-out mouse treated with PBS. Notes: 85LG; >70% PAS positive fibers.

FIG. 25 provides images showing RDR13 PAS staining of cardiac muscle for Lafora knock-out mouse treated with Fab-GAA. Notes: 93LG; about 10% PAS positive fibers.

FIG. 26 provides images showing RDR13 PAS staining of cardiac muscle for Lafora knock-out mouse treated with Myozyme. Notes: 102LG; >50% PAS positive fibers.

FIG. 27 provides examples of key sequences utilized. With respect to the various fusions, note that the signal sequence for secretion is simply indicated as an amino acid sequence, but it is recommended that the intron within this sequence be used.

FIG. 28 provides a graph showing the concentration of Fab-Amylase in rat cells and in HEK293 cells.

FIG. 29 provides a diagram of Fab-Amylase.

FIGS. 30A-30B provides a Fab-AMY immunoblot, silverstain, and ELISA. FIG. 30A provides a Fab-AMY immunoblot (5 & 50 ng, Lanes 1 & 2 resp.) and silverstain (500 & 250 ng, Lanes 3 & 4 resp.), detect 100 kDa Fab-AMY. FIG. 30B provides an ELISA using an anti-Fab capture Ab followed by anti-amylase detection Ab (ab21156).

FIG. 31 shows Fab-AMY degrades Lafora bodies in vitro. Untreated Lafora bodies and Fab-AMY treated Lafora bodies are shown from the brain, heart, and skeletal muscle.

FIG. 32 shows Fab-AMY penetrates cells in vitro. A cell penetration assay was performed. Fab-AMY (1.3 uM) or human Amylase (1.3 uM) was applied to T47D cells overnight, washed and fixed in ethanol. Cell penetration was detected with goat anti-human F(ab′)2-alkaline phosphatase conjugate (anti-Human F(ab′)2-AP), or rabbit anti-human AMY2A-alkaline phosphatase conjugate (anti-AMY2A-AP).

FIG. 33 shows Fab-AMY delivered ICV penetrates all brain regions. Stains are provided showing Fab-AMY treated brain and untreated control brain.

FIG. 34 shows Lafora bodies are large glycogen aggregates visible by Periodic Acid Schiff (PAS). Lafora bodies are present in the brain (panels A, B, C), skeletal muscle (panel D), and heart (panel E, low mag; panel F, high mag).

FIGS. 35A-35E demonstrate continuous ICV infusion of Fab-AMY. Fab-AMY was delivered to WT brain by continuous ICV using Alzet pumps (FIG. 35A). Three days post injection six brain slices were collected (FIG. 35B). Fab-AMY was strongly detected in all slices by immunoblot (FIG. 35C), ELISA (FIG. 35D), and amylase activity (FIG. 35E). Levels of Fab-AMY measured by immunoblot, ELISA, and amylase activity were consistent. Note: *=Alzet catheter placement.

FIG. 36 shows Fab-AMY delivered via ICV to Lafora knock out mouse brain reduces glycogen load. Fab-AMY administration to Lafora knock out mice by ICV administration reduces glycogen load across all brain sections. Glycogen levels were normalized to protein content of brain homogenate for each individual slice. The average value for each mouse was used to calculate the mean and SD for each treatment group. # of animals (n values) are shown. Note: PBS; n=4, Fab-AMY; n=5. **P≤0.01; ***P≤0.001.

FIGS. 37A-37B demonstrate effect of Fab-GAA treatment on total polyglucosan content in skeletal muscle and heart from GBE1 neo/neo mouse models of adult polyglucosan body disease (APBD). Heart and skeletal muscle were harvested from GBE1 neo/neo mice and homogenized and then treated with Fab-GAA. The ability of Fab-GAA to breakdown polyglucosan was determined by measuring the residual glucose in the homogenate. In response to Fab-GAA, there was a 50% reduction in the glucose derived from both heart (FIG. 37B) and muscle (FIG. 37A), indicating effective degradation of polyglucosan by Fab-GAA.

FIGS. 38A-38B demonstrate effect of Fab-GAA on polyglucosan inclusions in tissue specimens from APBD patients with different gene mutations. Human tissue specimens from patients with a variety of polyglucosan accumulation diseases were treated with Fab-GAA. Frozen sections were incubated in either 10 mg/ml Fab-GAA or vehicle at 37° C. for 12 hours. Specimens were then PAS stained to compare the glycogen content in the two specimen groups. FIG. 38A shows a heart specimen from a patient with a GYG1 missense mutation (c.304G>C, p.(Asp102His) that had severe glycogenin-1 deficiency resulting in dilated cardiomyopathy that required a cardiac transplant. FIG. 38B shows a skeletal muscle specimen from a patient with multiple RBCK1 mutations (c.817dupC, p.(Leu273Profs*27)) and c.1465delA, p.(Thr489Profs*9) resulting in severe RBCK1 deficiency. Fab-GAA reduced polyglucosan in both tissue types despite the difference in the etiologies of the two glycogen storage abnormalities.

FIGS. 39A-39B demonstrate effect of Fab-GAA on polyglucosan Lafora body load following intramuscular injection into the gastrocnemius muscle and heart in Epm2a−/− mice. Three serial injections of 20 μL of 10 mg/mL Fab-GAA (N=3) or PBS (N=4) were administered into the right gastrocnemius of 10 month old Epm2a−/− mice over the course of one week, on days 1, 4, and 7. Age-matched wild type C57BL/6 mice were treated with PBS (N=3) using the same regimen. On day 8 the mice were euthanized and muscles, including hearts, were collected for polyglucosan determination. FIG. 39A shows that Fab-GAA treatment reduced polyglucosan levels by 42% relative to the PBS treated muscle. FIG. 39B shows that Fab-GAA treatment also reduced polyglucosan levels in the heart.

FIGS. 40A-40B demonstrate polyglucosan content in Epm2a−/− mouse hear t (FIG. 40A) and quadriceps (FIG. 40B) muscle after IV injection of 120 mg/kg Fab-GAA. Four serial tail vein injections of 0.90 mg Fab-GAA or PBS were administered to 6 month old Epm2a−/− mice (N=5 each treatment). Age-matched wild type C57BL/6 mice (N=4) were injected with PBS as a control cohort. Hearts and quadriceps muscle were collected and quantified for polyglucosan content. Fab-GAA treatment reduced polyglucosan LB loads in Epm2a−/− (KO) mice to wild type (WT) levels.

FIG. 41 shows periodic acid-Schiff stains of glycogen-rich regions in Epm2a−/− (KO) and wild type C57BL/6 (WT) mouse heart and quadriceps muscle. A reduction in the number of polyglucosan bodies in both tissues after treatment with Fab-GAA can be seen.

### DETAILED DESCRIPTION OF THE DISCLOSURE

Glycogen is a complex polysaccharide that provides a ready store of glucose to cells in the human body. Glycogen is found principally in the liver, where it is hydrolyzed and released into the bloodstream to provide glucose to other cells, and in muscle, where the glucose resulting from glycogen hydrolysis provides energy for muscle cells. The proteins laforin, malin and alpha-amylase are believed to play a role in glycogen clearance.

In some embodiments, the disclosure provides for a polypeptide comprising any of the amino acid sequences disclosed herein. In some embodiments, the disclosure provides for a polypeptide comprising an amino acid sequence that is at least 85%, 90%, 91%, 92%, 93%, 94%, 95%, 96%, 97%, 98%, 99% or 100% identical to any of the amino acid sequences disclosed herein.

### I. Polypeptides

Alpha-Amylase Polypeptides

In certain embodiments, the non-internalizing moiety polypeptide portion of a chimeric polypeptide of the disclosure (or a chimeric polypeptide for use in the methods of the disclosure) is an alpha-amylase polypeptide (e.g., a salivary or pancreatic alpha-amylase). In other words, in certain embodiments, alpha-amylase-containing chimeric polypeptides are provided. Exemplary alpha-amylase (e.g., a mature alpha-amylase) polypeptides for use in the methods and compositions of the disclosure are provided herein. In some embodiments, the alpha-amylase (e.g., a mature alpha-amylase) polypeptides have utility in clearing excess glycogen in diseased cells. In some embodiments, the diseased cells are the cells of a subject having a polyglucosan accumulation disease (e.g., a non-central nervous system (CNS) polyglucosan accumulation disease). In some embodiments, the diseased cells are the cells of a subject having a glycogen storage disease or a glycogen metabolic disorder. In some embodiments, the diseased cells are from a subject having Pompe Disease, Andersen Disease, von Gierke Disease, Lafora Disease, Forbes-Cori Disease, Danon Disease, and/or Alzheimer's Disease. In some embodiments, the diseased cells are from a subject having Danon Disease. In other embodiments, the diseased cells are from a subject having Alzheimer's Disease or dementia.

In certain embodiments, any of the alpha-amylase polypeptides referred to herein may be substituted with a gamma-amylase. In certain embodiments, the gamma-amylase is capable of catalyzing the hydrolysis of terminal 1,4-linked alpha-D-glucose residues successively from non-reducing ends of polysaccharide chains with the release of beta-glucose. In some embodiments, the gamma-amylase is also able to hydrolyze 1,6-alpha-glucosidic bonds when the next bond in sequence is 1,4 in a glycogen molecule.

In some embodiments, the alpha-amylase (e.g., a mature alpha-amylase) is a monomer. In some embodiments, the alpha-amylase is a dimer or a trimer. In some embodiments, the alpha-amylase has been mutated such that it is incapable of multimerizing (e.g., the alpha-amylase has been mutated such that it is incapable of dimerizing or trimerizing). In some embodiments, the alpha-amylase has been treated with an agent that inhibits multimerization (e.g., dimerization or trimerization) of the alpha-amylase. In some embodiments, the agent is a small molecule.

As used herein, the alpha-amylase polypeptides include various functional fragments and variants, fusion proteins, and modified forms of the wildtype alpha-amylase polypeptide. In particular embodiments, the alpha-amylase is a mature alpha-amylase. In certain embodiments, the alpha-amylase or fragment or variant thereof is a salivary alpha-amylase or fragment or variant thereof. In certain embodiments, the alpha-amylase or fragment or variant thereof is a pancreatic alpha-amylase or fragment or variant thereof. In certain embodiments, the alpha-amylase or fragment or variant thereof is a mammalian alpha-amylase or fragment or variant thereof. In particular embodiments, the alpha-amylase or fragment or variant thereof is a human alpha-amylase or fragment or variant thereof. Such functional fragments or variants, fusion proteins, and modified forms of the alpha-amylase polypeptides have at least a portion of the amino acid sequence of substantial sequence identity to the native alpha-amylase polypeptide, and retain the function of the native alpha-amylase polypeptide (e.g., ability to hydrolyze alpha-1,4-glucosidic bonds). It should be noted that “retain the function” does not mean that the activity of a particular fragment must be identical or substantially identical to that of the native protein although, in some embodiments, it may be. However, to retain the native activity, that native activity should be at least 50%, at least 60%, at least 70%, at least 75%, at least 80%, at least 85%, at least 90%, at least 95% that of the native protein to which such activity is being compared, with the comparison being made under the same or similar conditions. In some embodiments, retaining the native activity may include scenarios in which a fragment or variant has improved activity versus the native protein to which such activity is being compared, e.g., at least 105%, at least 110%, at least 120%, or at least 125%, with the comparison being bade under the same or similar conditions.

In certain embodiments, a functional fragment, variant, or fusion protein of an alpha-amylase polypeptide comprises an amino acid sequence that is at least 80%, 85%, 90%, 95%, 97%, 98%, 99% or 100% identical to an alpha-amylase polypeptide, such as a mature alpha-amylase polypeptide (e.g., at least 80%, 85%, 90%, 95%, 97%, 98%, 99% or 100% identical to SEQ ID NO: 1), or fragments thereof.

In certain embodiments, the alpha-amylase polypeptide for use in the chimeric polypeptides and methods of the disclosure is a full length or substantially full length alpha-amylase polypeptide, or a mature form of a full-length alpha-amylase. In certain embodiments, the alpha-amylase polypeptide for use in the chimeric polypeptide and methods of the disclosure is a functional fragment that has alpha-1,4-glucosidic bond hydrolytic activity.

In certain embodiments of any of the foregoing, the alpha-amylase portion of the chimeric polypeptide of the disclosure comprises an alpha-amylase polypeptide (e.g., a mature form), which in certain embodiments may be a functional fragment of an alpha-amylase polypeptide or may be a substantially full length alpha-amylase polypeptide.

In some embodiments, the alpha-amylase is the mature form of an alpha-amylase. In particular embodiments, the mature form of the alpha-amylase corresponds to amino acids 16-511 of SEQ ID NO: 36 (Genbank accession number NP_000690). In some embodiments, the mature form of the alpha-amylase corresponds to an amino acid sequence that is at least 80%, 85%, 90%, 91%, 92%, 93%, 94%, 95%, 96%, 97%, 98%, 99% or 100% identical to the amino acid sequence of SEQ ID NO: 1, or functional fragments thereof.

Suitable alpha-amylase polypeptides or functional fragments thereof for use in the chimeric polypeptides and methods of the disclosure have alpha-1,4-glucosidic bond hydrolytic activity, as evaluated in vitro or in vivo. Exemplary functional fragments comprise, at least 100, 125, 150, 175, 200, 225, 250, 275, 300, 325, 350, 375, 400, 425, 450, 475, 500, or 511 consecutive amino acid residues of a full length alpha-amylase polypeptide (e.g., SEQ ID NO: 36). In some embodiments, the functional fragment comprises 100-150, 100-200, 100-250, 100-300, 100-400, 100-450, 100-495, 200-495, 300-495, 400-495, 450-495, 475-495 consecutive amino acids of a mature alpha-amylase polypeptide (e.g., SEQ ID NO: 1). Similarly, in certain embodiments, the disclosure contemplates chimeric proteins where the alpha-amylase portion is a variant of any of the foregoing alpha-amylase polypeptides or bioactive fragments. Exemplary variants have an amino acid sequence at least 90%, 92%, 95%, 96%, 97%, 98%, or at least 99% identical to the amino acid sequence of a native (e.g. mature) alpha-amylase polypeptide or functional fragment thereof, and such variants retain the alpha-amylase variant's alpha-1,4-glucosidic bond hydrolytic activity. The disclosure contemplates chimeric polypeptides and the use of such polypeptides wherein the alpha-amylase portion comprises any of the alpha-amylase polypeptides, fragments, or variants described herein in combination with any internalizing moiety described herein. Moreover, in certain embodiments, the alpha-amylase portion of any of the foregoing chimeric polypeptides may, in certain embodiments, be a fusion protein. Any such chimeric polypeptides comprising any combination of alpha-amylase portions and internalizing moiety portions, and optionally including one or more linkers, one or more tags, etc., may be used in any of the methods of the disclosure.

In certain embodiments, fragments or variants of the alpha-amylase polypeptides can be obtained by screening polypeptides recombinantly produced from the corresponding fragment of the nucleic acid encoding an alpha-amylase polypeptide. In addition, fragments or variants can be chemically synthesized using techniques known in the art such as conventional Merrifield solid phase f-Moc or t-Boc chemistry. The fragments or variants can be produced (recombinantly or by chemical synthesis) and tested to identify those fragments or variants that can function as a native alpha-amylase polypeptide, for example, by testing their ability to treat Danon Disease and/or Alzheimer's Disease in vivo and/or by confirming in vitro (e.g., in a cell free or cell based assay) that the fragment or variant has alpha-1,4-glucosidic bond hydrolytic activity. An example of an in vitro assay for testing for activity of the alpha-amylase polypeptides disclosed herein would be to treat disease cells with or without the alpha-amylase-containing chimeric polypeptides and then, after a period of incubation, examining levels of polyglucosan.

In certain embodiments, the present disclosure contemplates modifying the structure of an alpha-amylase polypeptide for such purposes as enhancing therapeutic or prophylactic efficacy, or stability (e.g., ex vivo shelf life and resistance to proteolytic degradation in vivo). Modified polypeptides can be produced, for instance, by amino acid substitution, deletion, or addition. For instance, it is reasonable to expect, for example, that an isolated replacement of a leucine with an isoleucine or valine, an aspartate with a glutamate, a threonine with a serine, or a similar replacement of an amino acid with a structurally related amino acid (e.g., conservative mutations) will not have a major effect on the alpha-amylase biological activity of the resulting molecule. Conservative replacements are those that take place within a family of amino acids that are related in their side chains.

This disclosure further contemplates generating sets of combinatorial mutants of an alpha-amylase polypeptide, as well as truncation mutants, and is especially useful for identifying functional variant sequences. Combinatorially-derived variants can be generated which have a selective potency relative to a naturally occurring alpha-amylase polypeptide. Likewise, mutagenesis can give rise to variants which have intracellular half-lives dramatically different than the corresponding wild-type alpha-amylase polypeptide. For example, the altered protein can be rendered either more stable or less stable to proteolytic degradation or other cellular process which result in destruction of, or otherwise inactivation of alpha-amylase. Such variants can be utilized to alter the alpha-amylase polypeptide level by modulating their half-life. There are many ways by which the library of potential alpha-amylase variants sequences can be generated, for example, from a degenerate oligonucleotide sequence. Chemical synthesis of a degenerate gene sequence can be carried out in an automatic DNA synthesizer, and the synthetic genes then can be ligated into an appropriate gene for expression. The purpose of a degenerate set of genes is to provide, in one mixture, all of the sequences encoding the desired set of potential polypeptide sequences. The synthesis of degenerate oligonucleotides is well known in the art (see for example, Narang, S A (1983) Tetrahedron 39:3; Itakura et al., (1981) Recombinant DNA, Proc. 3rd Cleveland Sympos. Macromolecules, ed. AG Walton, Amsterdam: Elsevier pp 273-289; Itakura et al., (1984) Annu. Rev. Biochem. 53:323; Itakura et al., (1984) Science 198:1056; Ike et al., (1983) Nucleic Acid Res. 11:477). Such techniques have been employed in the directed evolution of other proteins (see, for example, Scott et al., (1990) Science 249:386-390; Roberts et al., (1992) PNAS USA 89:2429-2433; Devlin et al., (1990) Science 249: 404-406; Cwirla et al., (1990) PNAS USA 87: 6378-6382; as well as U.S. Pat. Nos. 5,223,409, 5,198,346, and 5,096,815).

Alternatively, other forms of mutagenesis can be utilized to generate a combinatorial library. For example, alpha-amylase polypeptide variants can be generated and isolated from a library by screening using, for example, alanine scanning mutagenesis and the like (Ruf et al., (1994) Biochemistry 33:1565-1572; Wang et al., (1994) J. Biol. Chem. 269:3095-3099; Balint et al., (1993) Gene 137:109-118; Grodberg et al., (1993) Eur. J. Biochem. 218:597-601; Nagashima et al., (1993) J. Biol. Chem. 268:2888-2892; Lowman et al., (1991) Biochemistry 30:10832-10838; and Cunningham et al., (1989) Science 244:1081-1085), by linker scanning mutagenesis (Gustin et al., (1993) Virology 193:653-660; Brown et al., (1992) Mol. Cell Biol. 12:2644-2652; McKnight et al., (1982) Science 232:316); by saturation mutagenesis (Meyers et al., (1986) Science 232:613); by PCR mutagenesis (Leung et al., (1989) Method Cell Mol Biol 1:11-19); or by random mutagenesis, including chemical mutagenesis, etc. (Miller et al., (1992) A Short Course in Bacterial Genetics, CSHL Press, Cold Spring Harbor, N.Y.; and Greener et al., (1994) Strategies in Mol Biol 7:32-34). Linker scanning mutagenesis, particularly in a combinatorial setting, is an attractive method for identifying truncated (bioactive) forms of the alpha-amylase polypeptide.

A wide range of techniques are known in the art for screening gene products of combinatorial libraries made by point mutations and truncations, and, for that matter, for screening cDNA libraries for gene products having a certain property. Such techniques will be generally adaptable for rapid screening of the gene libraries generated by the combinatorial mutagenesis of the alpha-amylase polypeptides. The most widely used techniques for screening large gene libraries typically comprises cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected. Each of the illustrative assays described below are amenable to high through-put analysis as necessary to screen large numbers of degenerate sequences created by combinatorial mutagenesis techniques.

In certain embodiments, an alpha-amylase polypeptide may include a peptidomimetic. As used herein, the term “peptidomimetic” includes chemically modified peptides and peptide-like molecules that contain non-naturally occurring amino acids, peptoids, and the like. Peptidomimetics provide various advantages over a peptide, including enhanced stability when administered to a subject. Methods for identifying a peptidomimetic are well known in the art and include the screening of databases that contain libraries of potential peptidomimetics. For example, the Cambridge Structural Database contains a collection of greater than 300,000 compounds that have known crystal structures (Allen et al., Acta Crystallogr. Section B, 35:2331 (1979)). Where no crystal structure of a target molecule is available, a structure can be generated using, for example, the program CONCORD (Rusinko et al., J. Chem. Inf. Comput. Sci. 29:251 (1989)). Another database, the Available Chemicals Directory (Molecular Design Limited, Informations Systems; San Leandro Calif.), contains about 100,000 compounds that are commercially available and also can be searched to identify potential peptidomimetics of the alpha-amylase polypeptides.

In certain embodiments, an alpha-amylase polypeptide may further comprise post-translational modifications. Exemplary post-translational protein modifications include phosphorylation, acetylation, methylation, ADP-ribosylation, ubiquitination, glycosylation, carbonylation, sumoylation, biotinylation or addition of a polypeptide side chain or of a hydrophobic group. As a result, the modified alpha-amylase polypeptides may contain non-amino acid elements, such as lipids, poly- or mono-saccharides, and phosphates. Effects of such non-amino acid elements on the functionality of an alpha-amylase polypeptide may be tested for its biological activity, for example, alpha-1,4-glucosidic bonds hydrolytic activity and/or its ability to treat Danon Disease and/or Alzheimer's Disease. In certain embodiments, the alpha-amylase polypeptide may further comprise one or more polypeptide portions that enhance one or more of in vivo stability, in vivo half-life, uptake/administration, and/or purification. In other embodiments, the internalizing moiety comprises an antibody or an antigen-binding fragment thereof.

In some embodiments, an alpha-amylase polypeptide is not N-glycosylated or lacks one or more of the N-glycosylation groups present in a wildtype alpha-amylase polypeptide. For example, the alpha-amylase polypeptide for use in the present disclosure may lack all N-glycosylation sites, relative to native alpha-amylase, or the alpha-amylase polypeptide for use in the present disclosure may be under-glycosylated, relative to native alpha-amylase. In some embodiments, the alpha-amylase polypeptide comprises a modified amino acid sequence that is unable to be N-glycosylated at one or more N-glycosylation sites. In some embodiments, asparagine (Asn) of at least one predicted N-glycosylation site (i.e., a consensus sequence represented by the amino acid sequence Asn-Xaa-Ser or Asn-Xaa-Thr) in the alpha-amylase polypeptide is substituted by another amino acid. In some embodiments, the asparagine at the amino acid position corresponding to residue 412 and/or 461 of SEQ ID NO: 1 is substitute by another amino acid acid. The disclosure contemplates that any one or more of the foregoing examples can be combined so that an alpha-amylase polypeptide of the present disclosure lacks one or more N-glycosylation sites, and thus is either not glycosylated or is under glycosylated relative to native alpha-amylase.

In some embodiments, an alpha-amylase polypeptide is not O-glycosylated or lacks one or more of the O-glycosylation groups present in a wildtype alpha-amylase polypeptide. In some embodiments, the alpha-amylase polypeptide comprises a modified amino acid sequence that is unable to be O-glycosylated at one or more O-glycosylation sites. In some embodiments, serine or threonine at any one or more predicted O-glycosylation site in the alpha-amylase polypeptide sequence is substituted or deleted. The disclosure contemplates that any one or more of the foregoing examples can be combined so that an alpha-amylase polypeptide of the present disclosure lacks one or more N-glycosylation and/or O-glycosylation sites, and thus is either not glycosylated or is under glycosylated relative to native alpha-amylase.

In one specific embodiment of the present disclosure, an alpha-amylase polypeptide may be modified with nonproteinaceous polymers. In one specific embodiment, the polymer is polyethylene glycol (“PEG”), polypropylene glycol, or polyoxyalkylenes, in the manner as set forth in U.S. Pat. Nos. 4,640,835; 4,496,689; 4,301,144; 4,670,417; 4,791,192 or 4,179,337. PEG is a well-known, water soluble polymer that is commercially available or can be prepared by ring-opening polymerization of ethylene glycol according to methods well known in the art (Sandler and Karo, Polymer Synthesis, Academic Press, New York, Vol. 3, pages 138-161).

By the terms “biological activity”, “bioactivity” or “functional” is meant the ability of the alpha-amylase polypeptide to carry out the functions associated with wildtype mature alpha-amylase polypeptides, for example, alpha-1,4-glucosidic bond hydrolytic activity or ability to hydrolyze polyglucosan. The terms “biological activity”, “bioactivity”, and “functional” are used interchangeably herein. As used herein, “fragments” are understood to include bioactive fragments (also referred to as functional fragments) or bioactive variants that exhibit “bioactivity” as described herein. That is, bioactive fragments or variants of alpha-amylase exhibit bioactivity that can be measured and tested. For example, bioactive fragments/functional fragments or variants exhibit the same or substantially the same bioactivity as native (i.e., wild-type, or normal) alpha-amylase polypeptide, and such bioactivity can be assessed by the ability of the fragment or variant to, e.g., hydrolyze alpha-1,4-glucosidic bonds in a carbohydrate. As used herein, “substantially the same” refers to any parameter (e.g., activity) that is at least 70% of a control against which the parameter is measured. In certain embodiments, “substantially the same” also refers to any parameter (e.g., activity) that is at least 75%, 80%, 85%, 90%, 92%, 95%, 97%, 98%, 99%, 100%, 102%, 105%, or 110% of a control against which the parameter is measured. In certain embodiments, fragments or variants of the mature alpha-amylase polypeptide will preferably retain at least 50%, 60%, 70%, 80%, 85%, 90%, 95% or 100% of the alpha-amylase biological activity associated with the native mature alpha-amylase polypeptide, when assessed under the same or substantially the same conditions.

In certain embodiments, fragments or variants of the alpha-amylase polypeptide have a half-life (t_{1/2}) which is enhanced relative to the half-life of the native protein. Preferably, the half-life of alpha-amylase fragments or variants is enhanced by at least 10%, 20%, 30%, 40%, 50%, 60%, 70%, 80%, 90%, 100%, 125%, 150%, 175%, 200%, 250%, 300%, 400% or 500%, or even by 1000% relative to the half-life of the native alpha-amylase polypeptide. In some embodiments, the protein half-life is determined in vitro, such as in a buffered saline solution or in serum. In other embodiments, the protein half-life is an in vivo half-life, such as the half-life of the protein in the serum or other bodily fluid of an animal. In addition, fragments or variants can be chemically synthesized using techniques known in the art such as conventional Merrifield solid phase f-Moc or t-Boc chemistry. The fragments or variants can be produced (recombinantly or by chemical synthesis) and tested to identify those fragments or variants that can function as well as or substantially similarly to a native alpha-amylase polypeptide.

With respect to methods of increasing alpha-amylase bioactivity in cells, the disclosure contemplates all combinations of any of the foregoing aspects and embodiments, as well as combinations with any of the embodiments set forth in the detailed description and examples. The described methods based on administering chimeric polypeptides or contacting cells with chimeric polypeptides can be performed in vitro (e.g., in cells or culture) or in vivo (e.g., in a patient or animal model). In certain embodiments, the method is an in vitro method. In certain embodiments, the method is an in vivo method.

In some aspects, the present disclosure also provides a method of producing any of the foregoing chimeric polypeptides as described herein. Further, the present disclosure contemplates any number of combinations of the foregoing methods and compositions.

In certain aspects, an alpha-amylase polypeptide may be a fusion protein which further comprises one or more fusion domains. Well known examples of such fusion domains include, but are not limited to, polyhistidine, Glu-Glu, glutathione S transferase (GST), thioredoxin, protein A, protein G, and an immunoglobulin heavy chain constant region (Fc), maltose binding protein (MBP), which are particularly useful for isolation of the fusion proteins by affinity chromatography. For the purpose of affinity purification, relevant matrices for affinity chromatography, such as glutathione-, amylase-, and nickel- or cobalt-conjugated resins are used. Fusion domains also include “epitope tags,” which are usually short peptide sequences for which a specific antibody is available. Well known epitope tags for which specific monoclonal antibodies are readily available include FLAG, influenza virus haemagglutinin (HA), His and c-myc tags. An exemplary His tag has the sequence HHHHHH (SEQ ID NO: 15), and an exemplary c-myc tag has the sequence EQKLISEEDL (SEQ ID NO: 16). In some cases, the fusion domains have a protease cleavage site, such as for Factor Xa or Thrombin, which allows the relevant protease to partially digest the fusion proteins and thereby liberate the recombinant proteins therefrom. The liberated proteins can then be isolated from the fusion domain by subsequent chromatographic separation. In certain embodiments, the alpha-amylase polypeptides may contain one or more modifications that are capable of stabilizing the polypeptides. For example, such modifications enhance the in vitro half-life of the polypeptides, enhance circulatory half-life of the polypeptides or reduce proteolytic degradation of the polypeptides.

In certain embodiments of any of the foregoing, the alpha-amylase portion of the chimeric polypeptide of the disclosure comprises an alpha-amylase polypeptide, which in certain embodiments may be a functional fragment of an alpha-amylase polypeptide or may be a substantially full length alpha-amylase polypeptide. In some embodiments, the alpha-amylase polypeptide lacks the methionine at the N-terminal-most amino acid position (e.g., lacks the methionine at the first amino acid of any one of SEQ ID NOs: 36 or 44). Suitable alpha-amylase polypeptides for use in the chimeric polypeptides and methods of the disclosure have alpha-1,4-glucosidic bond hydrolytic activity, as evaluated in vitro or in vivo. Exemplary functional fragments comprise, at least 100, 125, 150, 175, 200, 225, 250, 275, 300, 325, 350, 375, 400, 425, 450, 475, 500, or 511 consecutive amino acid residues of a full length alpha-amylase polypeptide (e.g., SEQ ID NOs: 36 or 44). In some embodiments, the functional fragment comprises 100-150, 100-200, 100-250, 100-300, 100-400, 100-500, 100-511, 200-500, 300-500, 400-500, 450-500, 475-500 or 500-511 consecutive amino acids of a full-length alpha-amylase polypeptide (e.g., SEQ ID NO: 36 or 44). Similarly, in certain embodiments, the disclosure contemplates chimeric proteins where the alpha-amylase portion is a variant of any of the foregoing alpha-amylase polypeptides or bioactive fragments. Exemplary variants have an amino acid sequence at least 90%, 92%, 95%, 96%, 97%, 98%, or at least 99% identical to the amino acid sequence of a native alpha-amylase polypeptide or functional fragment thereof, and such variants retain the alpha-amylase variant's alpha-1,4-glucosidic bond hydrolytic activity. The disclosure contemplates chimeric polypeptides and the use of such polypeptides wherein the alpha-amylase portion comprises any of the alpha-amylase polypeptides, fragments, or variants described herein in combination with any internalizing moiety described herein. Moreover, in certain embodiments, the alpha-amylase portion of any of the foregoing chimeric polypeptides may, in certain embodiments, by a fusion protein. Any such chimeric polypeptides comprising any combination of alpha-amylase portions and internalizing moiety portions, and optionally including one or more linkers, one or more tags, etc., may be used in any of the methods of the disclosure.

Acid Alpha-Glucosidase (GAA) Polypeptide

In certain embodiments, the non-internalizing moiety polypeptide portion of a chimeric polypeptide of the disclosure (or a chimeric polypeptide for use in the methods of the disclosure) is an acid alpha-glucosidase (GAA) polypeptide. In other words, in certain embodiments, acid alpha-glucosidase-containing chimeric polypeptides are provided. Exemplary acid alpha-glucosidase (e.g., a mature acid alpha-glucosidase) polypeptides for use in the methods and compositions of the disclosure are provided herein. In some embodiments, the acid alpha-glucosidase (e.g., a mature acid alpha-glucosidase) polypeptides have utility in clearing excess glycogen in diseased cells. In some embodiments, the diseased cells are the cells of a subject having a polyglucosan accumulation disease (e.g., a non-central nervous system (CNS) polyglucosan accumulation disease). In some embodiments, the diseased cells are the cells of a subject having a glycogen storage disease or a glycogen metabolic disorder. In some embodiments, the diseased cells are from a subject having Pompe Disease, Andersen Disease, von Gierke Disease, Lafora Disease, Forbes-Cori Disease, Danon Disease, Alzheimer's Disease, PRKAG2 associated cardiomyopathy (PAC), GSD VII, GSD XV, or RBCK1 deficiency. In some embodiments, the diseased cells are from a subject having Danon Disease. In other embodiments, the diseased cells are from a subject having PAC. In still other embodiments, the diseased cells are from a subject having Lafora Disease.

It has been demonstrated that mature acid alpha-glucosidase polypeptides have enhanced glycogen clearance as compared to the full length, precursor GAA (Bijvoet, et al, 1998, Hum Mol Genet, 7(11): 1815-24), whether at low pH (i.e., the pH of the lysosome or autophagic vacuole) or neutral pH (i.e., the pH of the cytoplasm) conditions. In addition, while mature acid alpha-glucosidase is a lysosomal protein that has optimal activity at lower pHs, mature acid alpha-glucosidase retains approximately 40% activity at neutral pH (i.e., the pH of the cytoplasm) (Martin-Touaux et al, 2002, Hum Mol Genet, 11(14): 1637-45). Accordingly, an acid alpha-glucosidase polypeptide comprising mature acid alpha-glucosidase is suitable for cytoplasmic delivery, and thus, suitable to address cytoplasmic glycogen accumulation.

As used herein, the mature acid alpha-glucosidase polypeptides include variants, and in particular the mature, active forms of the protein (the active about 76 kDa or about 70 kDa forms or similar forms having an alternative starting and/or ending residue, collectively termed “mature acid alpha-glucosidase” or “mature GAA”). The term “mature GAA” refers to a polypeptide having an amino acid sequence corresponding to that portion of the immature GAA protein that, when processed endogenously, has an apparent molecular weight by SDS-PAGE of about 70 kDa to about 76 kDa, as well as similar polypeptides having alternative starting and/or ending residues. Conjugates of the disclosure comprise a GAA polypeptide comprising mature GAA and, in certain embodiments, the GAA polypeptide lacks the signal sequence (amino acids 1-27 of SEQ ID NOs: 45 or 46 or the sequence designated by amino acids 1-56 of SEQ ID NO: 45 or 46). Exemplary mature GAA polypeptides include polypeptides having residues 122-782 of SEQ ID NOs: 45 or 46; residues 123-782 of SEQ ID NOs: 45 or 46; or residues 204-782 of SEQ ID NOs: 45 or 46. The term “mature GAA” includes polypeptides that are glycosylated in the same or substantially the same way as the endogenous, mature proteins, and thus have a molecular weight that is the same or similar to the predicted molecular weight. The term also includes polypeptides that are not glycosylated or are hyper-glycosylated, such that their apparent molecular weight differ despite including the same primary amino acid sequence. Any such variants or iso forms, functional fragments or variants, fusion proteins, and modified forms of the mature GAA polypeptides have at least a portion of the amino acid sequence of substantial sequence identity to the native mature GAA protein, and retain enzymatic activity.

In certain embodiments, a functional fragment, variant, or fusion protein of a mature GAA polypeptide comprises an amino acid sequence that is at least 80%, 85%, 90%, 95%, 97%, 98%, 99% or 100% identical to mature GAA polypeptides set forth in one or both of SEQ ID NOs: 47 and 48, or is at least 80%, 85%, 90%, 95%, 97%, 98%, 99% or 100% identical to mature GAA polypeptides corresponding to one or more of: residues 122-782 of SEQ ID NOs: 45 or 46; residues 123-782 of SEQ ID NOs: 45 or 46; or residues 204-782 of SEQ ID NOs: 45 or 46. In certain embodiments, a functional fragment, variant, or fusion protein of a GAA polypeptide comprises an amino acid sequence that is at least 80%, 85%, 90%, 95%, 97%, 98%, 99% or 100% identical to GAA polypeptides set forth in any one of SEQ ID NOs: 49, 50 and 51. In some embodiments, the GAA polypeptide is a GAA polypeptide from a non-human species, e.g., mouse, rat, dog, zebrafish, pig, goat, cow, horse, monkey or ape. In some embodiments, the GAA protein comprises the mature form, but not the full-length form, of a bovine GAA protein having the amino acid sequence of SEQ ID NO: 52.

In certain specific embodiments, the conjugate comprises a GAA polypeptide comprising mature GAA (e.g., the heterologous agent is a GAA polypeptide comprising mature GAA). The mature GAA polypeptide may be the 76 kDa or the 70 kDa form of GAA, or similar forms that use alternative starting and/or ending residues. As noted in Moreland et al. (Lysosomal Acid a-Glucosidase Consists of Four Different Peptides Processed from a Single Chain Precursor, Journal of Biological Chemistry, 280(8): 6780-6791, 2005), the nomenclature used for the processed forms of GAA is based on an apparent molecular mass as determined by SDS-PAGE. In some embodiments, mature GAA may lack the N-terminal sites that are normally glycosylated in the endoplasmic reticulum. An exemplary mature GAA polypeptide comprises SEQ ID NO: 47 or SEQ ID NO: 48. Further exemplary mature GAA polypeptide may comprise or consist of an amino acid sequence corresponding to about: residues 122-782 of SEQ ID NOs: 45 or 46; residues 123-782 of SEQ ID NOs: 45 or 46, such as shown in SEQ ID NO: 47; residues 204-782 of SEQ ID NOs: 45 or 46; residues 206-782 of SEQ ID NOs: 45 or 46; residues 288-782 of SEQ ID NOs: 45 or 46, as shown in SEQ ID NO: 48. Mature GAA polypeptides may also have the N-terminal and or C-terminal residues described above.

In certain embodiments, the conjugate does not comprise a full-length GAA polypeptide, but comprises a mature GAA polypeptide and at least a portion of the full-length GAA polypeptide. In certain embodiments, the conjugate comprises a GAA polypeptide but does not include residues 1-56 of SEQ ID NO: 45 or 46. In certain embodiments, the conjugate comprises a GAA polypeptide but does not include residues 1-56 of SEQ ID NO: 45 or 46. In certain embodiments the GAA polypeptide does not comprise the 110 kilodalton GAA precursor. All of these are examples of the heterologous agents of the disclosure, specifically examples of embodiments wherein the heterologous agent is a GAA polypeptide comprising mature GAA.

In certain embodiments, the GAA polypeptide portion of the conjugates described herein comprise a mature form of GAA that does not comprise a GAA translation product set forth in SEQ ID NO: 45. In some embodiments, neither the GAA polypeptide nor the conjugate comprise a contiguous amino acid sequence corresponding to the amino acids 1-27 or 1-56 of SEQ ID NO: 45 or 46. In some embodiments, the GAA polypeptide lacks at least a portion of the GAA full linker region (SEQ ID NO: 53), wherein the full linker region corresponds to amino acids 57-78 of SEQ ID NOs: 45 or 46. In particular embodiments, the GAA polypeptide does not comprise a contiguous amino acid sequence corresponding to the amino acids 1-60 of SEQ ID NOs: 45 or 46 (e.g., the GAA polypeptide comprises the amino acid sequence of SEQ ID NO: 49). In other embodiments, the GAA portion does not comprise a contiguous amino acid sequence corresponding to the amino acids 1-66 of SEQ ID NO: 45 or 46 (e.g., the GAA polypeptide comprises the amino acid sequence of SEQ ID NO: 50). In some embodiments, the GAA portion does not comprise a contiguous amino acid sequence corresponding to the amino acids 1-69 of SEQ ID NO: 45 or 46 (e.g., the GAA polypeptide comprises the amino acid sequence of SEQ ID NO: 51). In other embodiments, the mature GAA polypeptides may be glycosylated, or may not be glycosylated. For those GAA polypeptides that are glycosylated, the glycosylation pattern may be the same as that of naturally-occurring human GAA or may be different. One or more of the glycosylation sites on the precursor GAA protein may be removed in the final mature GAA construct. Further exemplary GAA polypeptides may comprise or consist of an amino acid sequence corresponding to any one of SEQ ID NOs: 49, 50 and 51.

In certain embodiments, a GAA polypeptide comprising mature GAA is human.

By the terms “biological activity”, “bioactivity” or “functional” is meant the ability of a conjugate comprising a GAA polypeptide to carry out the functions associated with wildtype GAA proteins, for example, the hydrolysis of a-1,4- and a-1,6-glycosidic linkages of glycogen, for example lysosomal glycogen. The terms “biological activity”, “bioactivity”, and “functional” are used interchangeably herein. In certain embodiments, the biological activity comprises the ability to hydrolyze glycogen. In other embodiments, the biological activity is the ability to lower the concentration of lysosomal and/or cytoplasmic glycogen. In still other embodiments, the conjugate has the ability to treat symptoms associated with Danon disease, Lafora Disease and/or other polyglucosan accumulation diseases (e.g., PAC). As used herein, “fragments” are understood to include bioactive fragments (also referred to as functional fragments) or bioactive variants that exhibit “bioactivity” as described herein. That is, bioactive fragments or variants of mature GAA exhibit bioactivity that can be measured and tested. For example, bioactive fragments/functional fragments or variants exhibit the same or substantially the same bioactivity as native (i.e., wild-type, or normal) GAA protein, and such bioactivity can be assessed by the ability of the fragment or variant to, e.g., hydrolyze glycogen in vitro or in vivo. As used herein, “substantially the same” refers to any parameter (e.g., activity) that is at least 70% of a control against which the parameter is measured. In certain embodiments, “substantially the same” also refers to any parameter (e.g., activity) that is at least 75%, 80%, 85%, 90%, 92%, 95%, 97%, 98%, 99%, 100%, 102%, 105%, or 110% of a control against which the parameter is measured, when assessed under the same or substantially the same conditions. In certain embodiments, fragments or variants of the mature GAA polypeptide will preferably retain at least 50%, 60%, 70%, 80%, 85%, 90%, 95% or 100% of the GAA biological activity associated with the native GAA polypeptide, when assessed under the same or substantially the same conditions.

In certain embodiments, fragments or variants of the mature GAA polypeptide have a half-life (t_{1/2}) which is enhanced relative to the half-life of the native protein. Preferably, the half-life of mature GAA fragments or variants is enhanced by at least 10%, 20%, 30%, 40%, 50%, 60%, 70%, 80%, 90%, 100%, 125%, 150%, 175%, 200%, 250%, 300%, 400% or 500%, or even by 1000% relative to the half-life of the native GAA protein, when assessed under the same or substantially the same conditions. In some embodiments, the protein half-life is determined in vitro, such as in a buffered saline solution or in serum. In other embodiments, the protein half-life is an in vivo half-life, such as the half-life of the protein in the serum or other bodily fluid of an animal. In addition, fragments or variants can be chemically synthesized using techniques known in the art such as conventional Merrifield solid phase f-Moc or t-Boc chemistry. The fragments or variants can be produced (recombinantly or by chemical synthesis) and tested to identify those fragments or variants that can function as well as, or substantially similarly to, a native GAA protein.

In certain embodiments, a conjugate comprising a GAA polypeptide and an internalizing moiety can enter into a cell, such as into the cytoplasm, in the presence of an agent that blocks mannose-6-phophate receptors (MPRs).

With respect to methods of increasing GAA bioactivity in cells, the disclosure contemplates all combinations of any of the foregoing aspects and embodiments, as well as combinations with any of the embodiments set forth in the detailed description and examples. The described methods based on administering conjugates or contacting cells with conjugates can be performed in vitro (e.g., in cells or culture) or in vivo (e.g., in a patient or animal model). In certain embodiments, the method is an in vitro method. In certain embodiments, the method is an in vivo method.

In some aspects, the present disclosure also provides a method of producing any of the foregoing conjugates as described herein. Further, the present disclosure contemplates any number of combinations of the foregoing methods and compositions.

In certain aspects, a mature GAA polypeptide may be a fusion protein which further comprises one or more fusion domains. Well-known examples of such fusion domains include, but are not limited to, polyhistidine, Glu-Glu, glutathione S transferase (GST), thioredoxin, protein A, protein G, and an immunoglobulin heavy chain constant region (Fc), maltose binding protein (MBP), which are particularly useful for isolation of the fusion proteins by affinity chromatography. For the purpose of affinity purification, relevant matrices for affinity chromatography, such as glutathione-, amylase-, and nickel- or cobalt-conjugated resins are used. Fusion domains also include “epitope tags,” which are usually short peptide sequences for which a specific antibody is available. Well known epitope tags for which specific monoclonal antibodies are readily available include FLAG, influenza virus haemagglutinin (HA), His, and c-myc tags. An exemplary His tag has the sequence HHHHHH (SEQ ID NO: 15), and an exemplary c-myc tag has the sequence EQKLISEEDL (SEQ ID NO: 16). It is recognized that any such tags or fusions may be appended to the mature GAA portion of the conjugate or may be appended to the internalizing moiety portion of the conjugate, or both. In certain embodiments, the conjugates comprise a “AGIH” portion (SEQ ID NO: 25) on the N-terminus (or within 10 amino acid residues of the N-terminus) of the conjugate, and such conjugates may be provided in the presence or absence of one or more epitope tags. In further embodiments, the conjugate comprises a serine at the N-terminal most position of the polypeptide. In some embodiments, the conjugates comprise an “SAGIH” (SEQ ID NO: 26) portion at the N-terminus (or within 10 amino acid residues of the N-terminus) of the polypeptide, and such conjugates may be provided in the presence or absence of one or more epitope tags.

In some cases, the fusion domains have a protease cleavage site, such as for Factor Xa or Thrombin, which allows the relevant protease to partially digest the fusion proteins and thereby liberate the recombinant proteins therefrom. The liberated proteins can then be isolated from the fusion domain by subsequent chromatographic separation. In certain embodiments, the mature GAA polypeptides may contain one or more modifications that are capable of stabilizing the polypeptides. For example, such modifications enhance the in vitro half-life of the polypeptides, enhance circulatory half-life of the polypeptides or reducing proteolytic degradation of the polypeptides.

In some embodiments, a GAA polypeptide may be a fusion protein with an Fc region of an immunoglobulin.

In certain embodiments of any of the foregoing, the GAA portion of the conjugate comprises one of the mature forms of GAA, e.g., the 76 kDa fragment, the 70 kDa fragment, similar forms that use an alternative start and/or stop site, or a functional fragment thereof. In certain embodiments, such mature GAA polypeptide or functional fragment thereof retains the ability to hydrolyze glycogen, as evaluated in vitro or in vivo. Further, in certain embodiments, the conjugate that comprises such a mature GAA polypeptide or functional fragment thereof can hydrolyze glycogen. Exemplary bioactive fragments comprise at least 50, at least 60, at least 75, at least 100, at least 125, at least 150, at least 175, at least 200, at least 225, at least 230, at least 250, at least 260, at least 275, or at least 300 consecutive amino acid residues of a full length mature GAA polypeptide.

In certain embodiments, the GAA polypeptide portion of the conjugates described herein comprise a mature form of GAA that does not comprise a GAA polypeptide set forth in SEQ ID NO: 45. In some embodiments, the GAA polypeptide lacks at least a portion of the GAA full linker region (SEQ ID NO: 53), wherein the full linker region corresponds to amino acids 57-78 of SEQ ID NOs: 45 or 46. In particular embodiments, the GAA polypeptide does not comprise a contiguous amino acid sequence corresponding to the amino acids 1-60 of SEQ ID NOs: 45 or 46. In other embodiments, the GAA portion does not comprise a contiguous amino acid sequence corresponding to the amino acids 1-66 of SEQ ID NO: 45 or 46. In some embodiments, the GAA portion does not comprise a contiguous amino acid sequence corresponding to the amino acids 1-69 of SEQ ID NO: 45 or 46.

In particular embodiments, the GAA polypeptide does not comprise a contiguous amino acid sequence corresponding to the amino acids 1-60 of SEQ ID NOs: 45 or 46 (e.g., the conjugate does not comprise amino acids 1-60 of SEQ ID NO: 45 or 46). In other embodiments, the GAA portion does not comprise a contiguous amino acid sequence corresponding to the amino acids 1-66 of SEQ ID NO: 45 or 46 (e.g., the conjugate does not comprise a contiguous amino acid sequence corresponding to amino acids 1-60 or 1-66 of SEQ ID NO: 45 or 46). In some embodiments, the GAA portion does not comprise a contiguous amino acid sequence corresponding to the amino acids 1-69 of SEQ ID NO: 45 or 46 (e.g., the conjugate does not comprise a contiguous amino acid sequence corresponding to amino acids 1-60 or 1-66 or 1-69 of SEQ ID NO: 45 or 46). Suitable combinations, as set forth herein, are specifically contemplated.

In certain embodiments, the GAA polypeptide comprises an amino acid sequence corresponding to amino acids 61-952 of SEQ ID NO: 45. In some embodiments, the conjugate comprises amino acids 61-952 of SEQ ID NO: 45 and does not include a contiguous amino acid sequence corresponding to amino acids 1-60 of SEQ ID NO: 45. In certain embodiments, the GAA polypeptide comprises an amino acid sequence corresponding to amino acids 67-952 of SEQ ID NO: 45. In some embodiments, the conjugate comprises amino acids 67-952 of SEQ ID NO: 45 and does not include a contiguous amino acid sequence corresponding to amino acids 1-60 or, in certain embodiments, 1-66, of SEQ ID NO: 45. In certain embodiments, the GAA polypeptide comprises an amino acid sequence corresponding to amino acids 70-952 of SEQ ID NO: 45. In some embodiments, the conjugate comprises amino acids 70-952 of SEQ ID NO: 45 and does not include a contiguous amino acid sequence corresponding to amino acids 1-60 or, in certain embodiments, 1-66 or, in certain embodiments, 1-70, of SEQ ID NO: 45. Conjugates comprising any such GAA polypeptides comprising mature GAA may be used to deliver GAA activity into cells.

In certain embodiments, the disclosure contemplates conjugates where the mature GAA portion is a variant of any of the foregoing mature GAA polypeptides or functional fragments. Exemplary variants have an amino acid sequence at least 90%, 92%, 95%, 96%, 97%, 98%, or at least 99% identical to the amino acid sequence of a native GAA polypeptide or bioactive fragment thereof, and such variants retain the ability of native GAA to hydrolyze glycogen, as evaluated in vitro or in vivo. The disclosure contemplates conjugates and the use of such proteins wherein the GAA portion comprises any of the mature GAA polypeptides, forms, or variants described herein in combination with any internalizing moiety described herein. Moreover, in certain embodiments, the mature GAA portion of any of the foregoing conjugates may, in certain embodiments, be a fusion protein. Any such conjugates comprising any combination of GAA portions and internalizing moiety portions, and optionally including one or more linkers, one or more tags, etc., may be used in any of the methods of the disclosure.

### II. Internalizing Moieties

As used herein, the term “internalizing moiety” refers to a polypeptide/protein capable of interacting with a target tissue or a cell type such that the moiety is internalized into the target tissue or the cell type.

As used herein, “antibodies or antigen binding fragments of the disclosure” refer to any one or more of the antibodies and antigen binding fragments provided herein. Antibodies and antigen binding fragments of the disclosure comprise a heavy chain comprising a heavy chain variable domain and a light chain comprising a light chain variable domain. A V_{H} domain comprises three CDRs, such as any of the CDRs provided herein and as defined or identified by the Kabat and/or IMGT systems. These CDRs are typically interspersed with framework regions (FR), and together comprise the V_{H} domain. Similarly, a VL comprises three CDRs, such as any of the CDRs provided herein and as defined by the Kabat and/or IMGT systems. These CDRs are typically interspersed with framework regions (FR), and together comprise the V_{L} domain. The FR regions, such as FR1, FR2, FR3, and/or FR4 can similarly be defined or identified by the Kabat or IMGT systems. Throughout the application, when CDRs are indicated as being, as identified or defined by the Kabat or IMGT systems, what is meant is that the CDRs are in accordance with that system (e.g., the Kabat CDRs or the IMGT CDRs). Any of these terms can be used to indicate whether the Kabat or IMGT CDRs are being referred to.

The disclosure contemplates that an antibody or antigen binding fragment may comprise any combination of a V_{H} domain, as provided herein, and a V_{L} domain, as provided herein. In certain embodiments, at least one of the V_{H} and/or V_{L} domains are humanized (collectively, antibodies or antigen binding fragments of the disclosure). Chimeric antibodies are also included. Any antibody or antigen binding fragment of the disclosure may be provided alone. In other embodiments, any antibody or antigen binding fragment of the disclosure may be provided as a conjugate associated with a heterologous agent. Non-limiting examples of heterologous agents, which may include polypeptides, peptides, small molecules (e.g., a chemotherapeutic agent small molecule), or polynucleotides, are provided herein. Conjugates may refer to an antibody or antigen binding fragment associated with a heterologous agent.

In some embodiments, the antibody or antigen-binding fragment is isolated and/or purified. Any of the antibodies or antigen-binding fragments described herein, including those provided in an isolated or purified form, may be provided as a composition, such as a composition comprising an antibody or antigen-binding fragment formulated with one or more pharmaceutical and/or physiological acceptable carriers and/or excipients. Any of the antibodies or antigen-binding fragments described herein, including compositions (e.g., pharmaceutical compositions) may be used in any of the methods described herein and may be optionally provided conjugated (e.g., interconnected; associated) with a heterologous agent. In some embodiments, the internalizing moiety is capable of interacting with a target tissue or a cell type to effect delivery of the heterologous agent into a cell (i.e., penetrate desired cell; transport across a cellular membrane; deliver across cellular membranes to, at least, the cytoplasm). Such conjugates may similarly be provided as a composition and may be used in any of the methods described herein.

Internalizing moieties having limited cross-reactivity are generally preferred. In certain embodiments, this disclosure relates to an internalizing moiety which selectively, although not necessarily exclusively, targets and penetrates muscle, liver and/or neuronal cells. In certain embodiments, the internalizing moiety has limited cross-reactivity, and thus preferentially targets a particular cell or tissue type. However, it should be understood that internalizing moieties of the subject disclosure do not exclusively target specific cell types. Rather, the internalizing moieties promote delivery to one or more particular cell types, preferentially over other cell types, and thus provide for delivery that is not ubiquitous. In certain embodiments, suitable internalizing moieties include, for example, antibodies, monoclonal antibodies, or derivatives or analogs thereof. In certain embodiments, the internalizing moiety mediates transit across cellular membranes via an ENT2 transporter. In some embodiments, the internalizing moiety helps the chimeric polypeptide effectively and efficiently transit cellular membranes. In some embodiments, the internalizing moiety transits cellular membranes via an equilibrative nucleoside (ENT) transporter. In some embodiments, the internalizing moiety transits cellular membranes via an ENT1, ENT2, ENT3 or ENT4 transporter. In some embodiments, the internalizing moiety transits cellular membranes via an equilibrative nucleoside transporter 2 (ENT2) and/or ENT3 transporter. In some embodiments, the internalizing moiety promotes delivery into muscle (e.g., cardiac or diaphragm muscle), liver, skin or neuronal (e.g., brain) cells. For any of the foregoing, in certain embodiments, the internalizing moiety is internalized into the cytoplasm. In certain embodiments, the internalizing moiety is internalized into the nucleus or lysosomes.

In certain embodiments, the internalizing moiety is an antibody or antibody fragment that binds DNA. In certain embodiments, the internalizing moiety is any of the antibody or antibody fragments described herein. In other words, in certain embodiments, the antibody or antibody fragment (e.g., antibody fragment comprising an antigen binding fragment) binds DNA. In certain embodiments, DNA binding ability is measured versus a double stranded DNA substrate. In certain embodiments, the internalizing moiety is an antibody or antibody fragment that binds DNA and can transit cellular membranes via ENT2. In certain embodiments, the internalizing moiety binds a DNA bubble.

In certain embodiments, the internalizing moiety promotes delivery of a chimeric polypeptide into the cytoplasm. In certain embodiments, the internalizing moiety delivers alpha-amylase activity into cells. In certain embodiments, the chimeric polypeptide of the disclosure comprises an alpha-amylase-containing chimeric polypeptide (e.g., the non-internalizing moiety portion comprises or consists of an alpha-amylase polypeptide). Any of the internalizing moieties described herein may be combined with any of the non-internalizing moiety polypeptide portions, as described herein, to generate a chimeric polypeptide of the disclosure.

In certain embodiments, the internalizing moiety is capable of binding polynucleotides. In certain embodiments, the internalizing moiety is capable of binding DNA. In certain embodiments, the internalizing moiety is an antibody capable of binding DNA. In certain embodiments, the internalizing moiety is capable of binding DNA with a K_{D} of less than 1 μM. In certain embodiments, the internalizing moiety is capable of binding DNA with a K_{D} of less than 100 nM, less than 75 nM, less than 50 nM, or even less than 30 nM. K_{D} can be measured using Surface Plasmon Resonance (SPR) or Quartz Crystal Microbalance (QCM), in accordance with currently standard methods. By way of example, a 3E10 antibody or antibody fragment, including an antibody or antibody fragment comprising a VH having the amino acid sequence set forth in SEQ ID NO: 17 and a VL having an amino acid sequence set forth in SEQ ID NO: 18 is known to bind DNA with a K_{D} of less than 100 nM. Thus, in certain embodiments, an internalizing moiety for use in the chimeric polypeptides of the disclosure is an antibody or antibody fragment (e.g., an antigen binding fragment) that can transit cellular membranes into the cytoplasm and binds to DNA.

This is also exemplary of an anti-DNA antibody. In certain embodiments, an internalizing moiety for use herein is an anti-DNA antibody or antigen binding fragment thereof. In certain embodiments, an internalizing moiety of the disclosure, such as an antibody or antibody fragment described herein, binds a given DNA substrate with higher affinity as compared to an antibody or scFv or Fv having the VH and VL of the antibody produced by the hybridoma deposited with the ATCC under ATCC accession number PTA-2439. In certain embodiments, an internalizing moiety for use in the methods of the present disclosure is not an antibody or antibody fragment having the VH and VL of the antibody produced by the hybridoma deposited with the ATCC under ATCC accession number PTA-2439. In some embodiments, an internalizing moiety for use in the methods of the present disclosure is not a murine antibody or antibody fragment.

In certain aspects, an internalizing moiety may comprise an antibody, including a monoclonal antibody, a polyclonal antibody, and a humanized antibody. In some embodiments, the internalizing moiety is a full-length antibody. In some embodiments, internalizing moieties may comprise antibody fragments, derivatives or analogs thereof, including without limitation: antibody fragments comprising antigen binding fragments (e.g., Fv fragments, single chain Fv (scFv) fragments, Fab fragments, Fab′ fragments, F(ab′)2 fragments), single domain antibodies, camelized antibodies and antibody fragments, humanized antibodies and antibody fragments, human antibodies and antibody fragments, and multivalent versions of the foregoing; multivalent internalizing moieties including without limitation: Fv fragments, single chain Fv (scFv) fragments, Fab′ fragments, F(ab′)2 fragments, single domain antibodies, camelized antibodies and antibody fragments, humanized antibodies and antibody fragments, human antibodies and antibody fragments, and multivalent versions of the foregoing; multivalent internalizing moieties including without limitation: monospecific or bispecific antibodies, such as disulfide stabilized Fv fragments, scFv tandems ((scFv)₂ fragments), diabodies, tribodies or tetrabodies, which typically are covalently linked or otherwise stabilized (i.e., leucine zipper or helix stabilized) scFv fragments; receptor molecules which naturally interact with a desired target molecule. In some embodiments, the antibodies or variants thereof may be chimeric, e.g., they may include variable heavy or light regions from the murine 3E10 antibody, but may include constant regions from an antibody of another species (e.g., a human). In some embodiments, the antibodies or variants thereof may comprise a constant region that is a hybrid of several different antibody subclass constant domains (e.g., any combination of IgG1, IgG2a, IgG2b, IgG3 and IgG4, from any species or combination of species). In some embodiments, the antibodies or variants thereof (e.g., the internalizing moiety) comprise the following constant domain scheme: IgG2a CH1-IgG1 hinge-IgG1 CH2-CH3, for example, any of the foregoing may be human IgG or murine IgG. Other suitable combinations are also contemplated. In other embodiments, the antibody comprises a full length antibody and the CH1, hinge, CH2, and CH3 is from the same constant domain subclass (e.g., IgG1). In some embodiments, the antibodies or variants thereof are antibody fragments (e.g., the internalizing moiety is an antibody fragment comprising an antigen binding fragment; e.g., the internalizing moiety is an antigen binding fragment) comprising a portion of the constant domain of an immunoglobulin, for example, the following constant domain scheme: IgG2a CH1-IgG1 upper hinge. In some embodiments, the antibodies or variants thereof are antibody fragments that comprise a sequence that is at least 85%, 90%, 91%, 92%, 93%, 94%, 95%, 96%, 97%, 98%, 99% or 100% identical to the amino acid sequence of SEQ ID NO: 11.

In some embodiments, the antibodies or variants thereof comprise a kappa constant domain (e.g., of the Km3 allotype). In some embodiments, the antibodies or variants thereof are antibody fragments that comprise a sequence that is at least 85%, 90%, 91%, 92%, 93%, 94%, 95%, 96%, 97%, 98%, 99% or 100% SEQ ID NO: 12. Heavy chain constant domains (whether for a full length antibody or for an antibody fragment (e.g., an antigen binding fragment) comprising an amino acid substitution, relative to native IgG domains, to decrease effector function and/or facilitate production are included within the scope of antibodies and antigen binding fragments. For example, one, two, three, or four amino acid substitutions in a heavy chain, relative to a native murine or human immunoglobulin constant region, such as in the hinge or CH2 domain of a heavy chain constant region.

In certain embodiments, an internalizing moiety comprises an antibody, and the heavy chain comprises a VH region, and a constant domain comprising a CH1, hinge, CH2, and CH3 domain. In certain embodiments, a heavy chain comprises a VH region, and a constant domain comprising a CH1 domain and, optionally, the upper hinge. The upper hinge may include, for example, 1, 2, 3, or 4 amino acid residues of the hinge region. In certain embodiments, the upper hinge does not include a cysteine residue. In certain embodiments, the upper hinge includes one or more consecutive residues N-terminal to a cysteine that exists in the native hinge sequence. In certain embodiments, the heavy chain comprises a CH region, and a constant domain comprising a CH1 domain and a hinge. In certain embodiments, the hinge (whether present as part of a full length antibody or an antibody fragment) comprises a C to S substitution at a position corresponding to Kabat position 222 (e.g., a C222S in the hinge, where the variation is at a position corresponding to Kabat position 222). In other words, in certain embodiments, the internalizing moiety comprises a serine residue, rather than a cysteine residue, in a hinge domain at a position corresponding to Kabat 222. In certain embodiments, the heavy chain comprises a constant domain comprising a CH1, hinge, CH2 and, optionally CH3 domain. In certain embodiments, a CH2 domain comprises an N to Q substitution at a position corresponding to Kabat position 297 (e.g., a N297Q in a CH2 domain, wherein the variation is at a position corresponding to Kabat position 297). In other words, in certain embodiments, the internalizing moiety comprises a glutamine, rather than an asparagine, at a position corresponding to Kabat position 297.

In some embodiments, the internalizing moiety comprises all or a portion of the Fc region of an immunoglobulin. In other words, in addition to an antigen binding portion, in certain embodiments, the internalizing moiety comprises all or a portion of a heavy chain constant region of an immunoglobulin (e.g., one or two polypeptide chains of a heavy chain constant region. As is known, each immunoglobulin heavy chain constant region comprises four or five domains. The domains are named sequentially as follows: CH1-hinge-CH2-CH3(-CH4). The DNA sequences of the heavy chain domains have cross-homology among the immunoglobulin classes, e.g., the CH2 domain of IgG is homologous to the CH2 domain of IgA and IgD, and to the CH3 domain of IgM and IgE. As used herein, the term, “immunoglobulin Fc region” is understood to mean the carboxyl-terminal portion of an immunoglobulin chain constant region, preferably an immunoglobulin heavy chain constant region, or a portion thereof. For example, an immunoglobulin Fc region may comprise 1) a CH1 domain, a CH2 domain, and a CH3 domain, 2) a CH1 domain and a CH2 domain, 3) a CH1 domain and a CH3 domain, 4) a CH2 domain and a CH3 domain, or 5) a combination of two or more domains and an immunoglobulin hinge region, or a portion of a hinger (e.g., an upper hinge). In certain embodiments, an internalizing moiety further comprises a light chain constant region (CL).

In some embodiments, the Fc portion of any of the internalizing moieties described herein has been modified such that it does not induce antibody-dependent cell-mediated cytotoxicity (ADCC). In some embodiments, the Fc portion has been modified such that it does not bind complement. In certain embodiments, a CH2 domain of the Fc portion comprises an N to Q substitution at a position corresponding to Kabat position 297 (e.g., a N297Q in a CH2 domain, wherein the variation is at a position corresponding to Kabat position 297). In other words, in certain embodiments, the internalizing moiety comprises a glutamine, rather than an asparagine, at a position corresponding to Kabat position 297.

In one embodiment, the class of immunoglobulin from which the heavy chain constant region is derived is IgG (Igγ) (γ subclasses 1, 2, 3, or 4). Other classes of immunoglobulin, IgA (Igα), IgD (Igδ), IgE (Igε) and IgM (Igμ), may be used. The choice of particular immunoglobulin heavy chain constant region sequences from certain immunoglobulin classes and subclasses to achieve a particular result is considered to be within the level of skill in the art. The portion of the DNA construct encoding the immunoglobulin Fc region preferably comprises at least a portion of a hinge domain, and preferably at least a portion of a CH₃ domain of Fcγ or the homologous domains in any of IgA, IgD, IgE, or IgM. Furthermore, it is contemplated that substitution or deletion of amino acids within the immunoglobulin heavy chain constant regions may be useful in the practice of the disclosure. One example would be to introduce amino acid substitutions in the upper CH2 region to create a Fc variant with reduced affinity for Fc receptors (Cole et al. (1997) J. IMMUNOL. 159:3613). One of ordinary skill in the art can prepare such constructs using well known molecular biology techniques.

In some embodiments, any of the internalizing moieties disclosed herein comprise a signal sequence conjugated to the heavy chain and/or the light chain amino acid sequence. In some embodiments, the heavy chain comprises a signal sequence that is at least 85%, 90%, 91%, 92%, 93%, 94%, 95%, 96%, 97%, 98%, 99% or 100% identical to the amino acid sequence of SEQ ID NO: 4. In some embodiments, the light chain comprises a signal sequence that is at least 85%, 90%, 91%, 92%, 93%, 94%, 95%, 96%, 97%, 98%, 99% or 100% identical to the amino acid sequence of SEQ ID NO: 5. In some embodiments, the signal sequence lacks the N-terminal Methionine. In some embodiments, any of the polypeptides disclosed herein lacks the N-terminal Methionine.

In some embodiments, the internalizing moiety is any peptide or antibody-like protein having the complementarity determining regions (CDRs) of the 3E10 antibody sequence, or of an antibody that binds the same epitope (e.g., the same target, such as DNA) as 3E10. Also, transgenic mice, or other mammals, may be used to express humanized or human antibodies. Such humanization may be partial or complete.

In certain embodiments, the internalizing moiety comprises the monoclonal antibody 3E10 or an antigen binding fragment thereof. In other embodiments, the internalizing moiety comprises an antibody or an antigen binding fragment thereof, such as any of the antigen binding fragments described herein. For example, the antibody or antigen binding fragment thereof may be monoclonal antibody 3E10, or a variant thereof that retains cell penetrating activity, or an antigen binding fragment of 3E10 or said 3E10 variant. Additionally, the antibody or antigen binding fragment thereof may be an antibody that binds to the same epitope (e.g., target, such as DNA) as 3E10, or an antibody that has substantially the same cell penetrating activity as 3E10, or an antigen binding fragment thereof. These are exemplary of agents that can transit cells via ENT2. In certain embodiments, the internalizing moiety is capable of binding polynucleotides. In certain embodiments, the internalizing moiety is capable of binding DNA, such as double-stranded blunt DNA. In certain embodiments, the internalizing moiety is capable of binding DNA with a K_{D} of less than 100 nM. In certain embodiments, the internalizing moiety is capable of binding DNA with a K_{D} of less than 100 nM, less than 75 nM, less than 50 nM, or even less than 30 nM. K_{D} is determined using SPR or QCM or ELISA, according to manufacturer's instructions and current practice. In some embodiments, K_{D} is determined using a fluorescence polarization assay.

In certain embodiments, the antigen binding fragment is an Fv or scFv fragment thereof. Monoclonal antibody 3E10 can be produced by a hybridoma 3E10 placed permanently on deposit with the American Type Culture Collection (ATCC) under ATCC accession number PTA-2439 and is disclosed in U.S. Pat. No. 7,189,396. This antibody has been shown to bind DNA. Additionally or alternatively, the 3E10 antibody can be produced by expressing in a host cell nucleotide sequences encoding the heavy and light chains of the 3E10 antibody. The term “3E10 antibody” or “monoclonal antibody 3E10” are used to refer to the antibody, regardless of the method used to produce the antibody. Similarly, when referring to variants or antigen-binding fragments of 3E10, such terms are used without reference to the manner in which the antibody was produced. At this point, 3E10 is generally not produced by the hybridoma but is produced recombinantly. Thus, in the context of the present application, 3E10 antibody, unless otherwise specified, will refer to an antibody having the sequence of the hybridoma or comprising a variable heavy chain domain comprising the amino acid sequence set forth in SEQ ID NO: 17 (which has a one amino acid substitution relative to that of the 3E10 antibody deposited with the ATCC, as described herein) and the variable light chain domain comprising the amino acid sequence set forth in SEQ ID NO: 18, and antibody fragments thereof.

The internalizing moiety may also comprise variants of mAb 3E10, such as variants of 3E10 which retain the same cell penetration characteristics as mAb 3E10, as well as variants modified by mutation to improve the utility thereof (e.g., improved ability to target specific cell types, improved ability to penetrate the cell membrane, improved ability to localize to the cellular DNA, convenient site for conjugation, and the like). Such variants include variants wherein one or more conservative or non-conservative substitutions are introduced into the heavy chain, the light chain and/or the constant region(s) of the antibody. Such variants include humanized versions of 3E10 or a 3E10 variant, particularly those with improved activity or utility, as provided herein. In some embodiments, the light chain or heavy chain may be modified at the N-terminus or C-terminus. Similarly, the foregoing description of variants applies to antigen binding fragments. Any of these antibodies, variants, or fragments may be made recombinantly by expression of the nucleotide sequence(s) in a host cell.

Monoclonal antibody 3E10 has been shown to penetrate cells to deliver proteins and nucleic acids into the cytoplasmic or nuclear spaces of target tissues (Weisbart R H et al., J Autoimmun 1998 October; 11(5):539-46; Weisbart R H, et al. Mol Immunol. 2003 March; 39(13):783-9; Zack D J et al., J Immunol. 1996 Sep. 1; 157(5):2082-8.). Further, the VH and Vk sequences of 3E10 are highly homologous to human antibodies, with respective humanness z-scores of 0.943 and −0.880. Thus, Fv3E10 is expected to induce less of an anti-antibody response than many other approved humanized antibodies (Abhinandan K R et al., Mol. Biol. 2007 369, 852-862). A single chain Fv fragment of 3E10 possesses all the cell penetrating capabilities of the original monoclonal antibody, and proteins such as catalase, dystrophin, HSP70 and p53 retain their activity following conjugation to Fv3E10 (Hansen J E et al., Brain Res. 2006 May 9; 1088(1):187-96; Weisbart R H et al., Cancer Lett. 2003 Jun. 10; 195(2):211-9; Weisbart R H et al., J Drug Target. 2005 February; 13(2):81-7; Weisbart R H et al., J Immunol. 2000 Jun. 1; 164(11):6020-6; Hansen J E et al., J Biol Chem. 2007 Jul. 20; 282(29):20790-3). The 3E10 is built on the antibody scaffold present in all mammals; a mouse variable heavy chain and variable kappa light chain. 3E10 can gain entry to cells via the ENT2 nucleotide transporter that is particularly enriched in skeletal muscle and cancer cells, and in vitro studies have shown that 3E10 is nontoxic. (Weisbart R H et al., Mol Immunol. 2003 March; 39(13):783-9; Pennycooke M et al., Biochem Biophys Res Commun. 2001 Jan. 26; 280(3):951-9). 3E10 may also be capable of transiting membranes via ENT3.

The internalizing moiety may also include mutants of mAb 3E10, such as variants of 3E10 which retain the same or substantially the same cell penetration characteristics as mAb 3E10, as well as variants modified by mutation to improve the utility thereof (e.g., improved ability to target specific cell types, improved ability to penetrate the cell membrane, improved ability to localize to the cellular DNA, improved binding affinity, and the like). Such mutants include variants wherein one or more conservative substitutions are introduced into the heavy chain, the light chain and/or the constant region(s) of the antibody. Numerous variants of mAb 3E10 have been characterized in, e.g., U.S. Pat. No. 7,189,396 and WO 2008/091911, the teachings of which are incorporated by reference herein in their entirety.

In certain embodiments, the internalizing moiety comprises an antibody or antigen binding fragment comprising an VH domain comprising an amino acid sequence at least 80%, 85%, 90%, 95%, 96%, 97%, 99%, or 100% identical to SEQ ID NO: 17 and/or a VL domain comprising an amino acid sequence at least 85%, 90%, 95%, 96%, 97%, 99%, or 100% identical to SEQ ID NO: 18, or a humanized variant thereof. It is understood that, when a signal sequence is included for expression of an antibody or antibody fragment, that signal sequence is generally cleaved and not presented in the finished chimeric polypeptide (e.g., the signal sequence is generally cleaved and present only transiently during protein production). Such internalizing moieties transit, in certain embodiments, cells via ENT2 and/or bind DNA. In certain embodiments, an internalizing moiety for use in the methods of the present disclosure (or an antibody or antigen binding fragment for such use) is not an antibody or antibody fragment having the VH and VL of the antibody produced by the hybridoma deposited with the ATCC under ATCC accession number PTA-2439. In some embodiments, an internalizing moiety for use in the methods of the present disclosure (or an antibody or antigen binding fragment for such use) is not an antibody or antibody fragment having a VH comprising the amino acid sequence set forth in SEQ ID NO: 17 and a VL comprising the amino acid sequence set forth in SEQ ID NO: 18.

In certain embodiments, the internalizing moiety is capable of binding polynucleotides. In certain embodiments, the internalizing moiety is capable of binding (specifically binding) DNA. In certain embodiments, the internalizing moiety is capable of binding DNA with a K_{D} of less than 100 nM. In certain embodiments, the internalizing moiety is capable of binding DNA with a K_{D} of less than 50 nM. In certain embodiments, the internalizing moiety is an anti-DNA antibody, such as an antibody or antigen binding fragment that binds double-stranded blunt DNA. In certain embodiments, the internalizing moiety is an anti-DNA antibody or antigen binding fragment (thereof), where K_{D} is evaluated versus a double stranded DNA substrate, such as provided herein.

In certain embodiments, the internalizing moiety is an antigen binding fragment, such as a single chain Fv of 3E10 (scFv) comprising SEQ ID NOs: 17 and 18. In certain embodiments, the internalizing moiety comprises a single chain Fv of 3E10 (or another antigen binding fragment), and the amino acid sequence of the V_{H} domain is at least 90%, 95%, 96%, 97%, 98%, 99%, or 100% identical to SEQ ID NO: 17, and amino acid sequence of the V_{L} domain is at least 90%, 95%, 96%, 97%, 98%, 99%, or 100% identical to SEQ ID NO: 18. The variant 3E10 or fragment thereof retains the function of an internalizing moiety. When the internalizing moiety is an scFv, the VH and VL domains are typically connected via a linker, such as a gly/ser linker. The VH domain may be N-terminal to the VL domain or vice versa.

In certain embodiments, the internalizing moiety is an antigen binding fragment, such as a Fab comprising a VH and a VL. In certain embodiments, the internalizing moiety is a Fab (or another antigen binding fragment, such as a Fab′), and the amino acid sequence of the V_{H} domain is at least 90%, 95%, 96%, 97%, 98%, 99%, or 100% identical to SEQ ID NO: 17. In certain embodiments, the internalizing moiety is a Fab (or another antigen binding fragment, such as a Fab′), and the amino acid sequence of the V_{L} domain is at least 90%, 95%, 96%, 97%, 98%, 99%, or 100% identical to SEQ ID NO: 18. Our VH and VL domains, or combinations thereof, described herein are similarly contemplated. In certain embodiments, when the internalizing moiety is a Fab the heavy chain comprises a CH1 domain and an upper hinge of an immunoglobulin constant region. In certain embodiments, the upper hinge comprises a substitution, relative to a native immunoglobulin constant region, such as to decrease effector function and/or to eliminate a cysteine (e.g., a C to S). In certain embodiments, the upper hinge does not include a cysteine.

In certain embodiments, an internalizing moiety for use in the methods of the present disclosure (or an antibody or antigen binding fragment for such use) is not an antibody or antibody fragment having the VH and VL of the antibody produced by the hybridoma deposited with the ATCC under ATCC accession number PTA-2439. In some embodiments, an internalizing moiety for use in the methods of the present disclosure (or an antibody or antigen binding fragment for such use) is not an antibody or antibody fragment having a VH comprising the amino acid sequence set forth in SEQ ID NO: 17 and a VL comprising the amino acid sequence set forth in SEQ ID NO: 18.

In certain embodiments, the constant domain of the antibody or antibody fragment (e.g., antigen binding fragment) comprises all or a portion of a human Fc domain. In certain embodiments, the internalizing moiety is a full length antibody, and the constant domain of the antibody comprises a CH1, hinge, CH2 and CH3 domain. In certain embodiments, the constant domain comprises one or more substitutions, relative to a native immunoglobulin, that reduce effector function. Optionally, in certain embodiments, such a constant domain may include one or more (e.g., 1 substitution, 2 substitutions, 3 substitutions) substitutions in the heavy chain constant domain, such as in the hinge and/or CH2 domains, such as to reduce effector function. Such substitutions are known in the art.

In certain embodiments, the internalizing moiety is an antigen binding fragment—a fragment of an antibody comprising an antigen binding fragment. Suitable such fragments of antibodies, such as scFv, Fab, Fab′ and the like are described herein. In certain embodiments, the internalizing moiety is an antigen binding fragment or a full length antibody. In certain embodiments, the internalizing moiety comprises a light chain comprising a constant region (CL). In certain embodiments, the internalizing moiety comprises a heavy chain comprising a constant region, wherein the constant region comprises a CH1 domain. In certain embodiments, the internalizing moiety comprises a heavy chain comprising a constant region and a light chain comprising a constant region, wherein the heavy chain constant region comprises a CH1 domain. Optionally, the internalizing moiety may further comprise a heavy chain constant region comprising all or a portion of a hinge (e.g., an upper hinge or more than the upper hinge). Optionally, the internalizing moiety may further comprise a heavy chain comprising a CH2 and/or CH3 domain.

In some embodiments, the internalizing moiety comprises one or more of the CDRs of the 3E10 antibody. In certain embodiments, the internalizing moiety comprises one or more of the CDRs of a 3E10 antibody comprising the amino acid sequence of a V_{H} domain that is identical to SEQ ID NO: 17 and the amino acid sequence of a V_{L} domain that is identical to SEQ ID NO: 18. The CDRs of the 3E10 antibody may be determined using any of the CDR identification schemes available in the art. For example, in some embodiments, the CDRs of the 3E10 antibody are defined according to the Kabat definition as set forth in Kabat et al. Sequences of Proteins of Immunological Interest, 5th Ed. Public Health Service, National Institutes of Health, Bethesda, Md. (1991). In other embodiments, the CDRs of the 3E10 antibody are defined according to Chothia et al., 1987, J Mol Biol. 196: 901-917 and Chothia et al., 1989, Nature. 342:877-883. In other embodiments, the CDRs of the 3E10 antibody are defined according to the international ImMunoGeneTics database (IMGT) as set forth in LeFranc et al., 2003, Development and Comparative Immunology, 27: 55-77. In other embodiments, the CDRs of the 3E10 antibody are defined according to Honegger A, Pluckthun A., 2001, J Mol Biol., 309:657-670. In some embodiments, the CDRs of the 3E10 antibody are defined according to any of the CDR identification schemes discussed in Kunik et al., 2012, PLoS Comput Biol. 8(2): e1002388. In order to number residues of a 3E10 antibody for the purpose of identifying CDRs according to any of the CDR identification schemes known in the art, one may align the 3E10 antibody at regions of homology of the sequence of the antibody with a “standard” numbered sequence known in the art for the elected CDR identification scheme. Maximal alignment of framework residues frequently requires the insertion of “spacer” residues in the numbering system, to be used for the Fv region. In addition, the identity of certain individual residues at any given site number may vary from antibody chain to antibody chain due to interspecies or allelic divergence.

In certain embodiments, the internalizing moiety comprises at least 1, 2, 3, 4, or 5 of the CDRs of 3E10 as determined using the Kabat CDR identification scheme (e.g., the CDRs set forth in SEQ ID NOs: 19-24; the internalizing moiety is an antibody or antigen binding fragment thereof comprising a heavy chain comprising CDR1, CDR2, and CDR 3, as set forth in SEQ ID NOs: 19-21, respectively, and a light chain comprising CDR1, CDR2, and CDR3, as set forth in SEQ ID NOs: 22-24, respectively; e.g., and these CDRs in the internalizing moiety are as determined using the Kabat scheme). In other embodiments, the internalizing moiety comprises at least 1, 2, 3, 4 or 5 of the CDRs of 3E10 as determined using the IMGT identification scheme (e.g., the CDRs set forth in SEQ ID NOs: 27-32; the internalizing moiety is an antibody or antigen binding fragment thereof comprising a heavy chain comprising CDR1, CDR2, and CDR 3, as set forth in SEQ ID NOs: 27-29, respectively, and a light chain comprising CDR1, CDR2, and CDR3, as set forth in SEQ ID NOs: 30-32, respectively; e.g., and these CDRs in the internalizing moiety are as determined using the IMGT identification scheme). In certain embodiments, the internalizing moiety comprises all six CDRs of 3E10 as determined using the Kabat CDR identification scheme (e.g., comprises SEQ ID NOs 19-24). In other embodiments, the internalizing moiety comprises all six CDRS of 3E10 as determined using the IMGT identification scheme (e.g., which are set forth as SEQ ID NOs: 27-32). For any of the foregoing, in certain embodiments, the internalizing moiety is an antibody that binds the same epitope (e.g., the same target, such as DNA) as 3E10 and/or the internalizing moiety competes with 3E10 for binding to antigen. Exemplary internalizing moieties target and transit cells via ENT2. Exemplary internalizing moieties comprise antibodies or antigen binding fragments that bind DNA, such as double stranded blunt DNA.

In certain embodiments, the internalizing moiety comprising an antibody fragment, and the antibody fragment comprises an antigen binding fragment, such as an Fab or Fab′. In other words, in certain embodiments, the internalizing moiety comprises an Fab or Fab′.

In certain embodiments, the internalizing moiety competes with binding for a DNA substrate, such as double-stranded blunt DNA, with an antibody (or antigen-binding fragment) of the antibody produced by hybridoma 3E10 placed permanently on deposit with the American Type Culture Collection (ATCC) under ATCC accession number PTA-2439.

Preparation of antibodies or fragments thereof (e.g., a single chain Fv fragment encoded by V_{H}-linker-V_{L} or V_{L}-linker-V_{H} or a Fab) is well known in the art. In particular, methods of recombinant production of mAb 3E10 antibody fragments have been described in WO 2008/091911. Further, methods of generating scFv fragments of antibodies or Fabs are well known in the art. When recombinantly producing an antibody or antibody fragment, a linker may be used. For example, typical surface amino acids in flexible protein regions include Gly, Asn and Ser. One exemplary linker is provided in SEQ ID NO: 6, 13 or 14. Permutations of amino acid sequences containing Gly, Asn and Ser would be expected to satisfy the criteria (e.g., flexible with minimal hydrophobic or charged character) for a linker sequence. Another exemplary linker is of the formula (G₄S)n, wherein n is an integer from 1-10, such as 2, 3, or 4. Other near neutral amino acids, such as Thr and Ala, can also be used in the linker sequence.

In addition to linkers interconnecting portions of, for example, an scFv, the disclosure contemplates the use of additional linkers to, for example, interconnect the alpha-amylase portion to the antibody portion of the chimeric polypeptide.

Preparation of antibodies may be accomplished by any number of well-known methods for generating monoclonal antibodies. These methods typically include the step of immunization of animals, typically mice, with a desired immunogen (e.g., a desired target molecule or fragment thereof). Once the mice have been immunized, and preferably boosted one or more times with the desired immunogen(s), monoclonal antibody-producing hybridomas may be prepared and screened according to well-known methods (see, for example, Kuby, Janis, Immunology, Third Edition, pp. 131-139, W.H. Freeman & Co. (1997), for a general overview of monoclonal antibody production, that portion of which is incorporated herein by reference). Over the past several decades, antibody production has become extremely robust. In vitro methods that combine antibody recognition and phage display techniques allow one to amplify and select antibodies with very specific binding capabilities. See, for example, Holt, L. J. et al., “The Use of Recombinant Antibodies in Proteomics,” Current Opinion in Biotechnology, 2000, 11:445-449, incorporated herein by reference. These methods typically are much less cumbersome than preparation of hybridomas by traditional monoclonal antibody preparation methods. In one embodiment, phage display technology may be used to generate an internalizing moiety specific for a desired target molecule. An immune response to a selected immunogen is elicited in an animal (such as a mouse, rabbit, goat or other animal) and the response is boosted to expand the immunogen-specific B-cell population. Messenger RNA is isolated from those B-cells, or optionally a monoclonal or polyclonal hybridoma population. The mRNA is reverse-transcribed by known methods using either a poly-A primer or murine immunoglobulin-specific primer(s), typically specific to sequences adjacent to the desired V_{H} and V_{L} chains, to yield cDNA. The desired V_{H} and V_{L} chains are amplified by polymerase chain reaction (PCR) typically using V_{H} and V_{L} specific primer sets, and are ligated together, separated by a linker. V_{H} and V_{L} specific primer sets are commercially available, for instance from Stratagene, Inc. of La Jolla, Calif. Assembled V_{H}-linker-V_{L} product (encoding an scFv fragment) is selected for and amplified by PCR. Restriction sites are introduced into the ends of the V_{H}-linker-V_{L} product by PCR with primers including restriction sites and the scFv fragment is inserted into a suitable expression vector (typically a plasmid) for phage display. Other fragments, such as an Fab′ fragment, may be cloned into phage display vectors for surface expression on phage particles. The phage may be any phage, such as lambda, but typically is a filamentous phage, such as fd and M13, typically M13.

In certain embodiments, an antibody or antibody fragment is made recombinantly in a host cell. In other words, once the sequence of the antibody is known (for example, using the methods described above), the antibody can be made recombinantly using standard techniques.

In certain embodiments, the internalizing moieties may be modified to make them more resistant to cleavage by proteases. For example, the stability of an internalizing moiety comprising a polypeptide may be increased by substituting one or more of the naturally occurring amino acids in the (L) configuration with D-amino acids. In various embodiments, at least 1%, 5%, 10%, 20%, 50%, 80%, 90% or 100% of the amino acid residues of internalizing moiety may be of the D configuration. The switch from L to D amino acids neutralizes the digestion capabilities of many of the ubiquitous peptidases found in the digestive tract. Alternatively, enhanced stability of an internalizing moiety comprising a peptide bond may be achieved by the introduction of modifications of the traditional peptide linkages. For example, the introduction of a cyclic ring within the polypeptide backbone may confer enhanced stability in order to circumvent the effect of many proteolytic enzymes known to digest polypeptides in the stomach or other digestive organs and in serum. In still other embodiments, enhanced stability of an internalizing moiety may be achieved by intercalating one or more dextrorotatory amino acids (such as, dextrorotatory phenylalanine or dextrorotatory tryptophan) between the amino acids of internalizing moiety. In exemplary embodiments, such modifications increase the protease resistance of an internalizing moiety without affecting the activity or specificity of the interaction with a desired target molecule.

The disclosure contemplates the use of internalizing moieties (including antibodies or antigen binding fragments of the disclosure) described based on any combination of any of the foregoing or following structural and/or functional characteristics. Any such internalizing moieties, such as antibodies or antigen-binding fragments, are considered antibodies and antigen binding fragments of the disclosure and can be used for any of the uses or methods described herein, such as to treat Lafora Disease.

Further Examples of Antibodies or Antigen-Binding Fragments, Such as Humanized Antibodies or Antigen Binding Fragments

In some embodiments, the disclosure provides any of the antibodies or antigen-binding fragments disclosed herein, wherein the antibody or antigen-binding fragment is humanized. In other words, one class of internalizing moiety, such as antibody or antigen binding fragment, is a humanized antibody or antigen binding fragment. Such internalizing moiety may be humanized in whole or in part. Numerous examples of such humanized internalizing moieties are provided herein and are also described in WO 2015/106290, which is incorporated herein in its entirety.

In one embodiment, the disclosure provides an antibody or antigen-binding fragment comprising a humanized antibody or antigen-binding fragment, wherein the humanized antibody or antigen-binding fragment comprises a light chain variable (VL) domain and a heavy chain variable (VH) domain; wherein the V_{H} domain is humanized and comprises:

a VH CDR1 having the amino acid sequence of SEQ ID NO: 27;

a VH CDR2 having the amino acid sequence of SEQ ID NO: 28; and

a VH CDR3 having the amino acid sequence of SEQ ID NO: 29;

and the VL is humanized and comprises:

a VL CDR1 having the amino acid sequence of SEQ ID NO: 30;

a VL CDR2 having the amino acid sequence of SEQ ID NO: 31; and

a VL CDR3 having the amino acid sequence of SEQ ID NO: 32;

which CDRs are in accordance with the IMGT system, and wherein the humanized antibody or antigen-binding fragment has increased DNA binding and/or cell penetration, relative to that of a murine 3E10 antibody comprising a light chain variable (VL) domain having the amino acid sequence of SEQ ID NO: 18 and a heavy chain variable (VH) domain having the amino acid sequence of SEQ ID NO: 17. In certain embodiments, when comparing an antibody or antigen-binding fragment of the disclosure to a murine antibody or to another humanized antibody, the suitable comparison is between two proteins of the same structure (e.g., comparing a full length antibody to another full length antibody or comparing an Fab to another Fab). However, in other embodiments, the comparison is to an scFv or Fv of the murine antibody as a constant basis for comparison.

In some embodiments, an asparagine is mutated to another amino acid residue in the VH or VL domains in order to reduce N-linked glycosylation of the humanized antibody or antibody fragment. This humanized antibody or antibody fragment is based on a murine parent antibody—specifically a murine 3E10 antibody comprising a heavy chain and a light chain, wherein the light chain comprises a VL comprising the amino acid sequence of SEQ ID NO: 18 and the heavy chain comprises a VH comprising the amino acid sequence of SEQ ID NO: 17. In preferred embodiments, the internalizing moieties and fragments are associated with at least the cell-penetration properties associated with the murine 3E10 antibody (e.g., retain at least 75%, 80%, 85%, 90%, 95%, or greater than 95%) of the cell penetration properties. In certain embodiments, the humanized antibody or antibody fragment has one or more preferable cell penetration characteristics, such as improved penetration efficiency. In other embodiments, the humanized antibody or antibody fragment has improved DNA binding activity and/or a different range of DNA substrate affinity or specificity.

As used herein, the term “fragment” or “antigen-binding fragment” of a humanized antibody moiety or “antigen binding fragment” includes any fragment of a humanized internalizing moiety that retains at least the cell-penetration and/or DNA binding properties associated with the murine 3E10 antibody. In this application, the terms “fragment” and “antigen binding fragment” are used interchangeably. Exemplary antibody fragments include scFv fragments, Fab fragments (e.g., Fab′ or F(ab′)2), and the like.

In some embodiments, the humanized internalizing moiety (e.g., the humanized antibody and antigen binding fragments of the disclosure) is not directly fused to any heterologous agent or not fused or otherwise linked to a therapeutic or toxic heterologous agent. However, in such embodiments, and as described in greater detail below, the internalizing moiety may still be post-translationally modified (e.g., glycosylated or) and/or provided as part of a composition.

In other embodiments, the humanized internalizing moiety (e.g., the antibodies or antigen binding fragments of the disclosure, such as humanized antibodies or antibody binding fragments) is fused to a heterologous agent or a therapeutic or toxic heterologous agent. In some embodiments, the internalizing moiety effects delivery of a heterologous agent into a cell (i.e., penetrate desired cell; transport across a cellular membrane; deliver across cellular membranes to, at least, the cytoplasm). In certain embodiments, this disclosure relates to an internalizing moiety which promotes delivery of a heterologous agent into muscle, liver and/or neuronal cells, as well as certain other cell types. This portion promotes entry of the conjugate into cells. Like the murine, parental antibody, the humanized antibody and antigen binding fragments of the disclosure promote entry into cells via an ENT transporter, such as an ENT2 transporter and/or an ENT3 transporter. Without being bound by theory, ENT2 is expressed preferentially in certain cell types, including muscle (skeletal and cardiac), neuronal and/or liver cells. Accordingly, conjugates (e.g., conjugates in which a humanized antibody or antigen binding fragment of the disclosure is conjugated to a heterologous agent) are delivered into cells, but generally not ubiquitously. Rather, the conjugates may be delivered with some level of enrichment for particular tissues, including skeletal muscle, cardiac muscle, diaphragm, liver and neurons.

In certain embodiments, the internalizing moiety is capable of binding polynucleotides (e.g., a target/antigen for an antibody of the disclosure is DNA). This is consistent with the properties of the 3E10 antibody which is known to bind DNA (e.g., to specifically bind DNA). In certain embodiments, the internalizing moiety is capable of binding DNA. In certain embodiments, the internalizing moiety is capable of binding DNA with a K_{D} of less than 100 nM. In certain embodiments, the internalizing moiety is capable of binding DNA (e.g., single stranded DNA or blunt double stranded DNA) with a K_{D} of less than 500 nM, less than 100 nM, less than 75 nM, less than 50 nM, or even less than 30 nM, less than 20 nM, less than 10 nM, or even less than 1 nM. K_{D} can be measured using Surface Plasmon Resonance (SPR) or Quartz Crystal Microbalance (QCM), or by ELISA, in accordance with currently standard methods. By way of example, an antibody or antibody fragment comprising a VH having the amino acid sequence set forth in SEQ ID NO: 2 and a VL having an amino acid sequence set forth in SEQ ID NO: 3 specifically binds DNA with a K_{D} of less than 100 nM, and is an example of an anti-DNA antibody. In certain embodiments, the internalizing moiety binds double-stranded, blunt DNA, and DNA binding activity is or can be demonstrated in a binding assay using blunt DNA (see, for example, Xu et. Al. (2009) EMBO Journal 28: 568-577; Hansen et al., (2012) Sci Translation Med 4: DOI 10.1126/scitranslmed.3004385), such as by ELISA, QCM, or Biacore. In certain embodiments, the internalizing moiety is an anti-DNA antibody. Thus, in certain embodiments, an internalizing moiety (e.g., an antibody or antigen binding fragment) for use alone or associated with a heterologous agent comprises an antibody or antibody fragment that can transit cellular membranes into the cytoplasm and/or the nucleus and is capable of binding to DNA. In certain embodiments, the antibody and antigen binding fragments of the disclosure, such as humanized antibodies and antigen binding fragments, are based upon a murine, parental 3E10 antibody having VH and VL domains, as described above.

Preferably, the humanized antibody has the same, substantially the same, or even improved cell penetration and/or DNA binding characteristics in comparison to the murine, parental antibody, including a murine parental antibody comprising, when present, a murine constant domain.

In certain embodiments, the antibodies and antigen binding fragments of the disclosure have the same CDRs, as defined using the IMGT system, as the murine, parent antibody (e.g., the antibody comprising a heavy chain comprising a VH comprising the amino acid sequence set forth in SEQ ID NO: 17 and a light chain comprising a VL comprising the amino acid sequence set forth in SEQ ID NO: 18). In certain embodiments, the antibodies and antigen binding fragments of the disclosure have at least one CDR of the heavy chain and/or the light chain that differs from that of the murine, parent antibody (e.g., differ at VH CDR2 and/or VL CDR2 and/or VL CDR1, according to Kabat). In some embodiments, a humanized antibody or antigen binding fragment of the disclosure comprises a V_{H} domain and a V_{L} domain comprising:

a VH CDR1 having the amino acid sequence of SEQ ID NO: 27;

a VH CDR2 having the amino acid sequence of SEQ ID NO: 28;

a VH CDR3 having the amino acid sequence of SEQ ID NO: 29;

a VL CDR1 having the amino acid sequence of SEQ ID NO: 30;

a VL CDR2 having the amino acid sequence of SEQ ID NO: 31; and

a VL CDR3 having the amino acid sequence of SEQ ID NO: 32, which CDRs are in accordance with the IMGT system.

In some embodiments, a humanized antibody or antigen binding fragment of the disclosure comprises a V_{H} domain and a V_{L} domain comprising:

a VH CDR1 having the amino acid sequence of SEQ ID NO: 19;

a VH CDR2 having the amino acid sequence of SEQ ID NO: 20; and

a VH CDR3 having the amino acid sequence of SEQ ID NO: 21, which CDRs are according to Kabat; and

a VL CDR1 having the amino acid sequence of SEQ ID NO: 30;

a VL CDR2 having the amino acid sequence of SEQ ID NO: 31; and

a VL CDR3 having the amino acid sequence of SEQ ID NO: 32, which CDRs are according to the IMGT system.

In some embodiments, a humanized antibody or antigen binding fragment of the disclosure comprises a V_{H} domain and a V_{L} domain comprising:

a VH CDR1 having the amino acid sequence of SEQ ID NO: 27;

a VH CDR2 having the amino acid sequence of SEQ ID NO: 28; and

a VH CDR3 having the amino acid sequence of SEQ ID NO: 29, which CDRs are according to the IMGT system, and

a VL CDR1 having the amino acid sequence of SEQ ID NO: 22;

a VL CDR2 having the amino acid sequence of SEQ ID NO: 23; and

a VL CDR3 having the amino acid sequence of SEQ ID NO: 24, which CDRs are according to Kabat.

In certain embodiments, an antibody or antigen binding fragment of the disclosure comprises a V_{H} domain comprising:

a VH CDR1 having the amino acid sequence of SEQ ID NO: 19;

a VH CDR2 having the amino acid sequence of SEQ ID NO: 37; and

a VH CDR3 having the amino acid sequence of SEQ ID NO: 21, which CDRs are according to the Kabat system, and

a V_{L} domain comprising

a VL CDR1 having the amino acid sequence of SEQ ID NO: 22 or 38;

a VL CDR2 having the amino acid sequence of SEQ ID NO: 39; and

a VL CDR3 having the amino acid sequence of SEQ ID NO: 24, which CDRs are according to Kabat.

As detailed throughout the application, the antibody or antigen-binding fragments of the disclosure, such as humanized antibody or antigen binding fragments, can be compared to the murine, parent antibody or to the original 3E10 antibody or antigen binding fragment thereof. Additionally or alternatively, antibodies of the disclosure (or antigen binding fragments thereof) can be compared to alternate antibodies and fragments (e.g., other humanized antibodies based on the same murine parent). In such scenarios, the comparison could be to an alternate antibody or antigen binding fragment have the foregoing 6 IMGT or Kabat CDRs, but have one or more changes in the framework regions relative to the humanized antibody or antigen-binding fragment of the disclosure. Also contemplated are antibodies or antigen binding fragments having the CDRs disclosed herein, but with one, two, three, or four amino acid substitutions in one or more CDRs (e.g., with one substitution in one CDR, with two substitution—one in each of two CDRS, or with three substitutions—one in each of three CDRs). When comparing activity, the ability and efficiency to penetrate cells, such as muscle, liver and/or neuronal cells, via ENT2 and/or ENT3 may be assessed. Activity will be considered comparable or substantially the same if it is approximately 70%, 75%, 80%, 85%, 90%, 95%, or greater than about 95% the activity of the murine, parental antibody. Activity is considered improved, relative to the murine, parental antibody, if a characteristic is at least about 5%, preferably at least about 10% better (e.g., approximately 105%, 110%, 115%, 120%, 125%, 130%, 150%, or greater than 150% the activity of the murine, parental antibody or an alternate humanized antibody). In certain embodiments, an activity is considered improved, relative to another antibody, if a characteristic is at least 2-fold better. In other embodiments, an activity is considered improved if a characteristic is at least 3-, 4-, 5-, 6-, 8, or 10-fold better.

In some embodiments, antibodies or humanized antibodies may comprise antibody fragments, derivatives or analogs thereof, including without limitation: antibody fragments comprising an antigen binding fragments (e.g., Fv fragments, single chain Fv (scFv) fragments, Fab fragments, Fab′ fragments, F(ab′)2 fragments, single domain antibodies, and multivalent versions of the foregoing; multivalent internalizing moieties including without limitation: Fv fragments, single chain Fv (scFv) fragments, Fab fragments, Fab′ fragments, F(ab′)2 fragments, single domain antibodies, camelized antibodies and antibody fragments, humanized antibodies and antibody fragments, human antibodies and antibody fragments, and multivalent versions of the foregoing; multivalent internalizing moieties including without limitation: monospecific or bispecific antibodies, such as disulfide stabilized Fv fragments, scFv tandems ((scFv)₂ fragments), diabodies, tribodies or tetrabodies, which typically are covalently linked or otherwise stabilized (i.e., leucine zipper or helix stabilized) scFv fragments; receptor molecules which naturally interact with a desired target molecule. In certain embodiments, the antigen-binding fragment is an scFv and a peptide linker interconnects the VH domain and the VL domain. In some embodiments, the antibodies or variants thereof may comprise a constant region that is a hybrid of several different antibody subclass constant domains (e.g., any combination of IgG1, IgG2a, IgG2b, IgG3 and IgG4).

In certain embodiments, the internalizing moiety is an antibody fragment comprising an antigen binding fragment. In other words, in certain embodiments, the internalizing moiety is not a full length antibody but is a fragment thereof comprising an antigen binding fragment. In certain embodiments, the internalizing moiety is an scFv, Fab, Fab′, or Fab2′. In certain embodiments, the internalizing moiety is a full length antibody comprising a heavy chain comprising a CH1, hinge, CH2, and CH3 domains, optionally substituted to reduce effector function, such as in the hinge and/or CH2 domains, as described herein. In certain embodiments, the heavy chain comprises a VH domain, and a constant domain comprising a CH1, hinge, CH2, and CH3 domain. In certain embodiments, a heavy chain comprises a VH domain, and a constant domain comprising a CH1 domain and, optionally the upper hinge. The upper hinge may include, for example, 1, 2, 3, or 4 amino acid residues of the hinge region. In certain embodiments, the upper hinge does not include a cysteine residue. In certain embodiments, the upper hinge includes one or more consecutive residues N-terminal to a cysteine that exists in the native hinge sequence. In certain embodiments, the heavy chain comprises a CH region, and a constant domain comprising a CH1 domain and a hinge. In certain embodiments, the hinge (whether present as part of a full length antibody or an antibody fragment) comprises a C to S substitution at a position corresponding to Kabat position 222 (e.g., a C222S in the hinge, where the variation is at a position corresponding to Kabat position 222). In other words, in certain embodiments, the internalizing moiety comprises a serine residue, rather than a cysteine residue, in a hinge domain at a position corresponding to Kabat 222. In certain embodiments, the heavy chain comprises a constant domain comprising a CH1, hinge, CH2 and, optionally CH3 domain. In certain embodiments, a CH2 domain comprises an N to Q substitution at a position corresponding to Kabat position 297 (e.g., a N297Q in a CH2 domain, wherein the variation is at a position corresponding to Kabat position 297). In other words, in certain embodiments, the internalizing moiety comprises a glutamine, rather than an asparagine, at a position corresponding to Kabat position 297.

In certain embodiments, an antibody or antigen binding fragment as disclosed herein is a full length antibody comprising CH1, hinge, CH2, and CH3 of a heavy chain constant domain and a light chain constant domain. In certain embodiments the heavy chain constant region comprises one or more of a CH1, CH2, and CH3 domains, optionally with a hinge.

Monoclonal antibody 3E10 can be produced by hybridoma 3E10 placed permanently on deposit with the American Type Culture Collection (ATCC) under ATCC accession number PTA-2439 and is disclosed in U.S. Pat. No. 7,189,396. This antibody has been shown to bind DNA. Additionally or alternatively, the 3E10 antibody can be produced by expressing in a host cell nucleotide sequences encoding the heavy and light chains of the 3E10 antibody. The term “3E10 antibody” or “monoclonal antibody 3E10” are used also herein to refer to a murine antibody (or antigen binding fragment) comprising the a VL domain comprising the amino acid sequence of SEQ ID NO: 18 and a VH domain comprising the amino acid sequence of SEQ ID NO:17, regardless of the method used to produce the antibody. Thus, in the context of the present application, 3E10 antibody will refer, unless otherwise specified, to an antibody having the sequence of the hybridoma or comprising a variable heavy chain domain comprising the amino acid sequence set forth in SEQ ID NO: 17 (which has a one amino acid substitution relative to that of the 3E10 antibody deposited with the ATCC, as described herein and previously demonstrated as retaining cell penetration and DNA binding activity) and the variable light chain domain comprising the amino acid sequence set forth in SEQ ID NO: 18. However, in the context of the present disclosure, the parent murine antibody used as the basis for humanization was an antibody comprising the VL domain comprising the amino acid sequence of SEQ ID NO: 18 and a VH domain comprising the amino acid sequence of SEQ ID NO: 17. The disclosure provides, in certain embodiments, humanized antibodies based on murine 3E10.

Similarly, when referring to variants or antigen-binding fragments of 3E10, such terms are used without reference to the manner in which the antibody was produced. At this point, 3E10 is generally produced recombinantly.

The humanized internalizing moiety may also be derived from variants of mAb 3E10, such as variants of 3E10 which retain the same cell penetration characteristics as mAb 3E10, as well as variants modified by mutation to improve the utility thereof (e.g., improved ability to target specific cell types, improved ability to penetrate the cell membrane, improved ability to localize to the cellular DNA, convenient site for conjugation, and the like). Such variants include variants wherein one or more conservative substitutions are introduced into the heavy chain, the light chain and/or the constant region(s) of the antibody. In some embodiments, the light chain or heavy chain may be modified at the N-terminus or C-terminus. Moreover, the antibody or antibody fragment may be modified to facilitate conjugation to a heterologous agent. Similarly, the foregoing description of variants applies to antigen binding fragments. Any of these antibodies, variants, or fragments may be made recombinantly by expression of the nucleotide sequence(s) in a host cell. Such internalizing moieties can transit cells via an ENT transporter, such as ENT2 and/or ENT3 and/or bind the same epitope (e.g., target, such as DNA) as 3E10.

The humanized internalizing moiety may also be derived from mutants of mAb 3E10, such as variants of 3E10 which retain the same or substantially the same cell penetration characteristics as mAb 3E10, as well as variants modified by mutation to improve the utility thereof (e.g., improved ability to target specific cell types, improved ability to penetrate the cell membrane, improved ability to localize to the cellular DNA, improved binding affinity, and the like). Such mutants include variants wherein one or more conservative substitutions are introduced into the heavy chain or the light chain. Numerous variants of mAb 3E10 have been characterized in, e.g., U.S. Pat. No. 7,189,396 and WO 2008/091911, the teachings of which are incorporated by reference herein in their entirety. In the examples provided herein, the parent, murine 3E10 comprises a VH comprising the amino acid sequence set forth in SEQ ID NO: 17 and a VL comprising the amino acid sequence set forth in SEQ ID NO: 18.

In certain embodiments, the internalizing moiety is an antigen binding fragment, such as a humanized single chain Fv (scFv). In other embodiments, the humanized antibody is a Fab′ fragment.

In some embodiments, the internalizing moiety is an antibody or antibody fragment comprising an immunoglobulin heavy chain constant region or fragment thereof. As is known, each immunoglobulin heavy chain constant region comprises four or five domains. The domains are named sequentially as follows: C_{H}1-hinge-C_{H}2-C_{H}3(-C_{H}4). The DNA sequences of the heavy chain domains have cross-homology among the immunoglobulin classes, e.g., the C_{H}2 domain of IgG is homologous to the C_{H}2 domain of IgA and IgD, and to the C_{H}3 domain of IgM and IgE. As used herein, the term, “immunoglobulin Fc region” is understood to mean the carboxyl-terminal portion of an immunoglobulin heavy chain constant region, preferably an immunoglobulin heavy chain constant region, or a portion thereof. For example, an immunoglobulin Fc region may comprise 1) a CH1 domain, a CH2 domain, and a CH3 domain, 2) a CH1 domain and a CH2 domain, 3) a CH1 domain and a CH3 domain, 4) a CH2 domain and a CH3 domain, or 5) a combination of two or more domains and an immunoglobulin hinge region. In one embodiment, the immunoglobulin Fc region comprises at least an immunoglobulin hinge region a CH2 domain and a CH3 domain, and lacks the CH1 domain. In one embodiment, the class of immunoglobulin from which the heavy chain constant region is derived is IgG (Igγ) (γ subclasses 1, 2, 3, or 4). Other classes of immunoglobulin, IgA (Iga), IgD (Igδ), IgE and IgM (Igp), may be used. The choice of appropriate immunoglobulin heavy chain constant regions is discussed in detail in U.S. Pat. Nos. 5,541,087, and 5,726,044. The choice of particular immunoglobulin heavy chain constant region sequences from certain immunoglobulin classes and subclasses to achieve a particular result is considered to be within the level of skill in the art. The portion of the DNA construct encoding the immunoglobulin Fc region may comprise at least a portion of a hinge domain, and preferably at least a portion of a CH3 domain of Fc γ or the homologous domains in any of IgA, IgD, IgE, or IgM. Furthermore, it is contemplated that substitution or deletion of amino acids within the immunoglobulin heavy chain constant regions may be useful in the practice of the disclosure. In certain embodiments, the constant region domains are human. In some embodiments, the Fc portion of any of the internalizing moieties described herein has been modified such that it does not induce antibody-dependent cell-mediated cytotoxicity (ADCC). In some embodiments, the Fc portion has been modified such that it does not bind complement. In certain embodiments, a CH2 domain comprises an N to Q substitution at a position corresponding to Kabat position 297 (e.g., a N297Q in a CH2 domain, wherein the variation is at a position corresponding to Kabat position 297). In other words, in certain embodiments, the internalizing moiety comprises a glutamine, rather than an asparagine, at a position corresponding to Kabat position 297.

In some embodiments, the antibody or antigen binding fragment comprises hybrid heavy chain constant regions, i.e., the antibody or antigen binding fragment comprise multiple heavy chain constant region domains selected from: a CH1 domain, a CH2 domain, a CH3 domain, and a CH4 domain; wherein at least one of the constant region domains in the antibody or antigen binding fragment is of a class or subclass of immunoglobulin distinct from the class or subclass of another domain in the antibody or antigen binding fragment. In some embodiments, at least one of the constant region domains in the antibody or antigen binding fragment is an IgG constant region domain, and at least one of the constant region domains in the antibody or antigen binding fragment is of a different immunoglobulin class, i.e., an IgA, IgD, IgE, or IgM constant region domain. In some embodiments, at least one of the constant region domains in the antibody or antigen binding fragment is an IgG1 constant region domain, and at least one of the constant region domains in the antibody or antigen binding fragment is of a different IgG subclass, i.e., an IgG2A, IgG2B, IgG3 or IgG4. Suitable constant regions may be human or from another species (e.g., murine). Humanized antibodies and antigen binding fragments of the disclosure are consider humanized regardless of whether and constant region sequence (heavy or light chain), if present, corresponds to that of a human immunoglobulin or corresponds to that of another species.

The cell penetrating ability of the humanized internalizing moieties or fragments or variants may be utilized to promote delivery of a heterologous agent. Humanized moieties derived from 3E10 are particularly well suited for this because of their demonstrated ability to effectively promote delivery to muscle, liver and neuronal cells. Thus, humanized internalizing moieties are especially useful for promoting effective delivery into cells in subjects, such as human patients or model organisms. In certain embodiments, antibodies and antigen binding fragments of the disclosure are useful as intermediates for further conjugation to a heterologous agent, such as a heterologous protein, peptide, polynucleotide, or small molecule. However, in other embodiments, the humanized internalizing moieties or fragments or variants are not utilized to deliver any heterologous agent.

Preparation of antibodies or fragments thereof (e.g., a single chain Fv fragment encoded by V_{H}-linker-V_{L} or V_{L}-linker-V_{H}) is well known in the art. In particular, methods of recombinant production of mAb 3E10 antibody fragments have been described in WO 2008/091911. Further, methods of generating scFv fragments of antibodies are well known in the art. When recombinantly producing an antibody or antibody fragment, a linker may be used. For example, typical surface amino acids in flexible protein regions include Gly, Asn and Ser. One exemplary linker is provided in SEQ ID NO: 6, 13 or 14. Permutations of amino acid sequences containing Gly, Asn and Ser would be expected to satisfy the criteria (e.g., flexible with minimal hydrophobic or charged character) for a linker sequence. Another exemplary linker is of the formula (G₄S)n, wherein n is an integer from 1-10, such as 2, 3, or 4. Other near neutral amino acids, such as Thr and Ala, can also be used in the linker sequence.

In addition to linkers interconnecting portions of, for example, an scFv, the disclosure contemplates the use of additional linkers to, for example, interconnect the heterologous agent to the antibody portion of a conjugate or to interconnect the heterologous agent portion to the antibody portion of conjugate.

Preparation of antibodies may be accomplished by any number of well-known methods for generating monoclonal antibodies. These methods typically include the step of immunization of animals, typically mice, with a desired immunogen (e.g., a desired target molecule or fragment thereof). Once the mice have been immunized, and preferably boosted one or more times with the desired immunogen(s), monoclonal antibody-producing hybridomas may be prepared and screened according to well-known methods (see, for example, Kuby, Janis, Immunology, Third Edition, pp. 131-139, W.H. Freeman & Co. (1997), for a general overview of monoclonal antibody production, that portion of which is incorporated herein by reference). Over the past several decades, antibody production has become extremely robust. In vitro methods that combine antibody recognition and phage display techniques allow one to amplify and select antibodies with very specific binding capabilities. See, for example, Holt, L. J. et al., “The Use of Recombinant Antibodies in Proteomics,” Current Opinion in Biotechnology, 2000, 11:445-449, incorporated herein by reference. These methods typically are much less cumbersome than preparation of hybridomas by traditional monoclonal antibody preparation methods. In one embodiment, phage display technology may be used to generate an internalizing moiety specific for a desired target molecule. An immune response to a selected immunogen is elicited in an animal (such as a mouse, rabbit, goat or other animal) and the response is boosted to expand the immunogen-specific B-cell population. Messenger RNA is isolated from those B-cells, or optionally a monoclonal or polyclonal hybridoma population. The mRNA is reverse-transcribed by known methods using either a poly-A primer or murine immunoglobulin-specific primer(s), typically specific to sequences adjacent to the desired V_{H} and V_{L} chains, to yield cDNA. The desired V_{H} and V_{L} chains are amplified by polymerase chain reaction (PCR) typically using V_{H} and V_{L} specific primer sets, and are ligated together, separated by a linker. V_{H} and V_{L} specific primer sets are commercially available, for instance from Stratagene, Inc. of La Jolla, Calif. Assembled V_{H}-linker-V_{L} product (encoding an scFv fragment) is selected for and amplified by PCR. Restriction sites are introduced into the ends of the V_{H}-linker-V_{L} product by PCR with primers including restriction sites and the scFv fragment is inserted into a suitable expression vector (typically a plasmid) for phage display. Other fragments, such as an Fab′ fragment, may be cloned into phage display vectors for surface expression on phage particles. The phage may be any phage, such as lambda, but typically is a filamentous phage, such as fd and M13, typically M13.

In certain embodiments, an antibody or antibody fragment is made recombinantly in a host cell. In other words, once the sequence of the antibody is known (for example, using the methods described above), the antibody can be made recombinantly using standard techniques.

In certain embodiments, the humanized internalizing moieties may be modified to make them more resistant to cleavage by proteases. For example, the stability of an internalizing moiety comprising a polypeptide may be increased by substituting one or more of the naturally occurring amino acids in the (L) configuration with D-amino acids. In various embodiments, at least 1%, 5%, 10%, 20%, 50%, 80%, 90% or 100% of the amino acid residues of internalizing moiety may be of the D configuration. The switch from L to D amino acids neutralizes the digestion capabilities of many of the ubiquitous peptidases found in the digestive tract. Alternatively, enhanced stability of an internalizing moiety comprising an peptide bond may be achieved by the introduction of modifications of the traditional peptide linkages. For example, the introduction of a cyclic ring within the polypeptide backbone may confer enhanced stability in order to circumvent the effect of many proteolytic enzymes known to digest polypeptides in the stomach or other digestive organs and in serum. In still other embodiments, enhanced stability of an internalizing moiety may be achieved by intercalating one or more dextrorotatory amino acids (such as, dextrorotatory phenylalanine or dextrorotatory tryptophan) between the amino acids of internalizing moiety. In exemplary embodiments, such modifications increase the protease resistance of an internalizing moiety without affecting the activity or specificity of the interaction with a desired target molecule.

A “Fab fragment” is comprised of one light chain and the C_{H}1 and variable regions of one heavy chain. Generally, the heavy chain of a Fab molecule cannot form a disulfide bond with another heavy chain molecule. A Fab may optionally include a portion of the hinge, such as the upper hinge.

A “Fab′ fragment” contains one light chain and one heavy chain that contains more of the constant region, between the C_{H}1 and C_{H}2 domains, such that an interchain disulfide bond can be formed between two heavy chains to form a F(ab′)₂ molecule.

A “F(ab′)₂ fragment” contains two light chains and two heavy chains containing a portion of the constant region between the CH1 and CH2 domains, such that an interchain disulfide bond is formed between two heavy chains.

Native antibodies are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (VH) followed by a number of constant domains (CH). Each light chain has a variable domain at one end (VL) and a constant domain (CL) at its other end; the constant domain of the light chain is aligned with the first constant domain of the heavy chain, and the light chain variable domain is aligned with the variable domain of the heavy chain. Light chains are classified as either lambda chains or kappa chains based on the amino acid sequence of the light chain constant region. The variable domain of a kappa light chain may also be denoted herein as VK.

The antibodies of the disclosure include full length or intact antibody, antibody fragments, native sequence antibody or amino acid variants, human, humanized (a form of chimeric antibodies), post-translationally modified, chimeric antibodies, immunoconjugates, and functional fragments thereof. The antibodies can be modified in the Fc region to provide desired effector functions or serum half-life.

Preparation of Antibodies

Naturally occurring antibody structural units typically comprise a tetramer. Each such tetramer typically is composed of two identical pairs of polypeptide chains, each pair having one full-length “light” chain (typically having a molecular weight of about 25 kDa) and one full-length “heavy” chain (typically having a molecular weight of about 50-70 kDa). The amino-terminal portion of each chain typically includes a variable region of about 100 to 110 or more amino acids that typically is responsible for antigen recognition. The carboxy-terminal portion of each chain typically defines a constant region responsible for effector function. Human light chains are typically classified as kappa and lambda light chains. Heavy chains are typically classified as mu, delta, gamma, alpha, or epsilon, and define the antibody's isotype as IgM, IgD, IgG, IgA, and IgE, respectively. IgG has several subclasses, including, but not limited to, IgG1, IgG2, IgG3, and IgG4. IgM has subclasses including, but not limited to, IgM1 and IgM2. IgA is similarly subdivided into subclasses including, but not limited to, IgA1 and IgA2. See, e.g., Fundamental Immunology, Ch. 7, 2.sup.nd ed., (Paul, W., ed.), 1989, Raven Press, N.Y. (incorporated by reference in its entirety for all purposes). The combination of the variable regions of each light chain/heavy chain pair typically forms the antigen-binding site. In some embodiments, antibodies or antigen binding fragments of the disclosure comprise the following constant domain scheme: IgG2a CH1-IgG1 hinge-IgG1 CH2-CH3. Other suitable combinations are also contemplated. In other embodiments, the antibody comprises a full length antibody and the CH1, hinge, CH2, and CH3 is from the same constant domain subclass (e.g., IgG1). In some embodiments, the antibodies or antigen binding fragment comprises an antigen binding fragment comprising a portion of the constant domain of an immunoglobulin, for example, the following constant domain scheme: IgG2a CH1-IgG1 upper hinge. In some embodiments, the antibodies or antigen binding fragments of the disclosure comprise a kappa constant domain (e.g., SEQ ID NO: 12).

The variable regions of each of the heavy chains and light chains typically exhibit the same general structure comprising four relatively conserved framework regions (FR) joined by three hyper variable regions, also called complementarity determining regions or CDRs. The CDRs from the two chains of each pair typically are aligned by the framework regions, which alignment may enable binding to a specific target (e.g., antigen, DNA in the context of the present disclosure). From N-terminal to C-terminal, both light and heavy chain variable regions typically comprise the domains FR1, CDR1, FR2, CDR2, FR3, CDR3 and FR4. The assignment of amino acids to each domain (FR or CDR) is typically in accordance with the definitions of Kabat Sequences of Proteins of Immunological Interest (1987 and 1991, National Institutes of Health, Bethesda, Md.). In certain embodiments, the CDRs of a particular antibody, such as an antibody provided herein, are CDRs, as defined by this Kabat system (e.g., the CDRs being referred to for an antibody or antigen binding fragment are identified using the Kabat system). Similarly, in certain embodiments, particularly when the CDRs are defined or identified as by the Kabat system, the FR regions are also defined and/or identified using the Kabat system. However, alternative systems for identifying CDR and FR regions are also available, including the IMGT system (described herein). In certain embodiments, the CDRs of a particular antibody, such as an antibody provided herein, are CDRs as defined by the IMGT system (e.g., CDRs for an antibody or antigen binding fragment are identified using the IMGT system).

Antibodies became useful and of interest as pharmaceutical agents with the development of monoclonal antibodies. Monoclonal antibodies are produced using any method that produces antibody molecules by continuous cell lines in culture. Examples of suitable methods for preparing monoclonal antibodies include the hybridoma methods of Kohler et al. (1975, Nature 256:495-497) and the human B-cell hybridoma method (Kozbor, 1984, J. Immunol. 133:3001; and Brodeur et al., 1987, Monoclonal Antibody Production Techniques and Applications, (Marcel Dekker, Inc., New York), pp. 51-63). In many cases, hybridomas are used to generate an initial antibody of murine or rodent origin. That initial antibody may then be modified, such as using recombinant techniques to produce rodent variants, chimeric antibodies, humanized antibodies and the like. Other methods exist to produce an initial antibody, and such methods are known in the art. However, regardless of the method used to generate an initial antibody or even a variant of that initial antibody, any given antibody of non-human origin can then be modified to increase its humanness.

It can be advantageous to increase the humanness of a non-human antibody to make it more suitable for use in human subject and cells, whether for diagnostic, therapeutic, or research purposes. Antibodies may be modified for use as therapeutics. Examples of such antibodies (including antibody fragments) include chimeric, humanized, and fully human antibodies. Numerous methods exist in the art for the generation of chimeric, humanized and human antibodies. In the context of the present disclosure, an antibody is considered humanized if at least one of the VH domain or VL domain is humanized. Moreover, a VH or VL domain is humanized if the amino acid sequence of at least a portion of at least one FR regions has been modified, relative to a parent murine antibody, such that the amino acid sequence of that portion corresponds to that of a human antibody or a human consensus sequence. In certain embodiments, at least one, two, three, or four FR regions of the VH domain and/or at least one, two, three, or four FR regions of the VL domain have been modified (in whole or in part) so that their sequence is more closely related to a human sequence. For any of the foregoing in certain embodiments, a humanized antibody fragment may be provided in the context of a human or non-human light chain and/or heavy chain constant region (e.g., comprising a CL and one or more of a CH1, hinge, CH2, and/or CH3 domains). In certain embodiments, a humanized antibody or antigen binding fragment of the disclosure is provided in the context of human light and/or heavy chain constant domains, when present. Numerous examples of humanized light and heavy chain variable domains based on a 3E10 parent antibody are provided herein. Antibodies and antibody binding fragments combining any of the humanized light chain variable domains and/or heavy chain variable domains described herein are exemplary of antibodies and antigen binding fragments of the disclosure.

Once the nucleotide sequences encoding such antibodies have been determined, chimeric or humanized antibodies may be produced by recombinant methods. Nucleic acids encoding the antibodies are introduced into host cells and expressed using materials and procedures generally known in the art.

In certain embodiments, the antibodies or antigen binding fragments of the disclosure are of the IgG1, IgG2, or IgG4 isotype. In certain embodiments of the disclosure, the antibodies comprise a human kappa light chain and a human IgG1, IgG2, or IgG4 heavy chain. In certain embodiments, the antibodies of the disclosure have been cloned for expression in mammalian cells.

Regardless of when an antibody of the disclosure is a full length antibody or an antigen binding fragment, antibodies and antigen binding fragments of the disclosure can be recombinantly expressed in cell lines. In these embodiments, sequences encoding particular antibodies can be used for transformation of a suitable host cell, such as a mammalian host cell or yeast host cell. According to these embodiments, transformation can be achieved using any known method for introducing polynucleotides into a host cell, including, for example packaging the polynucleotide in a virus (or into a viral vector) and transducing a host cell with the virus (or vector) or by transfection procedures known in the art. Generally, the transformation procedure used may depend upon the host to be transformed. Methods for introducing heterologous polynucleotides into mammalian cells are well known in the art and include, but are not limited to, dextran-mediated transfection, calcium phosphate precipitation, polybrene mediated transfection, protoplast fusion, electroporation, encapsulation of the polynucleotide(s) in liposomes, and direct microinjection of the DNA into nuclei.

According to certain embodiments of the disclosure, a nucleic acid molecule encoding the amino acid sequence of a heavy chain constant region (all or a portion), a heavy chain variable region of the disclosure, a light chain constant region, or a light chain variable region of the disclosure is inserted into an appropriate expression vector using standard ligation techniques. In a preferred embodiment, the heavy or light chain constant region is appended to the C-terminus of the appropriate variable region and is ligated into an expression vector. The vector is typically selected to be functional in the particular host cell employed (i.e., the vector is compatible with the host cell machinery such that amplification of the gene and/or expression of the gene can occur). For a review of expression vectors, see, Goeddel (ed.), 1990, Meth. Enzymol. Vol. 185, Academic Press. N.Y. In the context of antibody expression, both the heavy and light chain may be expressed from the same vector (e.g., from the same or different promoters present on the same vector) or the heavy and light chains may be expressed from different vectors. In certain embodiments, the heavy and light chains are expressed from different vectors which are transfected into the same host cell and co-expressed. Regardless of when the heavy and light chains are expressed in the same host cell from the same or a different vector, the chains can then associate to form an antibody (or antibody fragment, depending on the portions of the heavy and light chain being expressed).

In some embodiments, an antibody or antigen binding fragment of the disclosure is not conjugated to a heterologous agent. In other embodiments, an antibody or antigen binding fragment of the disclosure is conjugated to a heterologous agent. In certain embodiments, the heterologous agent is a protein or peptide. That protein or peptide may be expressed as an inframe, co-translation fusion protein with, for example, the heavy chain, and expressed as described herein. Chemical conjugation is also possible. Conjugated as described in detail herein and unless otherwise specified, refers to scenarios where any of the antibody or antigen binding portions of the disclosure are associated with or interconnected with the heterologous agent, regardless of the interconnection (e.g., the interconnection/association may comprise a chemical conjugation, covalent bond, di-sulfide bond, etc. or combinations thereof). In certain embodiments, at least a portion of the interconnection is via a covalent bond, such as the forming of a fusion protein between a heavy chain of the antibody of the disclosure and the heterologous agent (which may further associate with a light chain of the antibody of the disclosure). Accordingly, the disclosure provides such conjugates and pharmaceutical compositions comprising such conjugates. A conjugate is a molecule comprising an antibody or antigen binding portion of the disclosure associate with a heterologous agent. Similarly, antibodies or antigen binding fragments of the disclosure may further comprise a heterologous agent. Conjugates along molecules where the two portions are associated or interconnected (e.g., the interconnection may comprise a chemical conjugation, covalent bond, di-sulfide bond, etc. or combinations thereof). In certain embodiments, at least a portion of the interconnection is via a covalent bond, such as the forming of a fusion protein between a heavy chain of an antibody of the disclosure and the heterologous agent (which may further associate with a light chain of the antibody or antibody fragment of the disclosure).

Typically, expression vectors used in any of the host cells will contain sequences for plasmid maintenance and for cloning and expression of exogenous nucleotide sequences. Such sequences, collectively referred to as “flanking sequences” in certain embodiments will typically include one or more of the following nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the polypeptide to be expressed, and a selectable marker element. These portions of vectors are well known, and there are numerous generally available vectors that can be selected and used for the expression of proteins. One can readily selected vectors based on the desired host cell and application.

An origin of replication is typically a part of those prokaryotic expression vectors purchased commercially, and the origin aids in the amplification of the vector in a host cell. If the vector of choice does not contain an origin of replication site, one may be chemically synthesized based on a known sequence, and ligated into the vector. For example, the origin of replication from the plasmid pBR322 (New England Biolabs, Beverly, Mass.) is suitable for most gram-negative bacteria and various viral origins (e.g., SV40, polyoma, adenovirus, vesicular stomatitus virus (VSV), or papillomaviruses such as HPV or BPV) are useful for cloning vectors in mammalian cells. Generally, the origin of replication component is not needed for mammalian expression vectors (for example, the SV40 origin is often used only because it also contains the virus early promoter).

The expression and cloning vectors of the disclosure will typically contain a promoter that is recognized by the host organism and operably linked to the molecule encoding heavy and/or light chain. Promoters are untranscribed sequences located upstream (i.e., 5′) to the start codon of a structural gene (generally within about 100 to 1000 bp) that control the transcription of the structural gene. Promoters are conventionally grouped into one of two classes: inducible promoters and constitutive promoters. Inducible promoters initiate increased levels of transcription from DNA under their control in response to some change in culture conditions, such as the presence or absence of a nutrient or a change in temperature. Constitutive promoters, on the other hand, initiate continual gene product production; that is, there is little or no control over gene expression. A large number of promoters, recognized by a variety of potential host cells, are well known. A suitable promoter is operably linked to the DNA encoding the heavy chain or light chain comprising an antibody or antigen binding fragment of the disclosure. In certain embodiments, the same promoter is used for both the heavy and light chain. In other embodiments, different promoters (present on the same or different vectors) are used for each.

Suitable promoters for use with yeast hosts are also well known in the art. Yeast enhancers are advantageously used with yeast promoters. Suitable promoters for use with mammalian host cells are well known and include, but are not limited to, those obtained from the genomes of viruses such as polyoma virus, fowlpox virus, adenovirus (such as Adenovirus 2), bovine papilloma virus, avian sarcoma virus, cytomegalovirus, retroviruses, hepatitis-B virus and most preferably Simian Virus 40 (SV40). Other suitable mammalian promoters include heterologous mammalian promoters, for example, heat-shock promoters and the actin promoter.

Additional promoters which may be of interest include, but are not limited to: the SV40 early promoter region (Bernoist and Chambon, 1981, Nature 290:304-10); the CMV promoter; the promoter contained in the 3′ long terminal repeat of Rous sarcoma virus (Yamamoto et al., 1980, Cell 22:787-97); the herpes thymidine kinase promoter (Wagner et al., 1981, Proc. Natl. Acad. Sci. USA 78:1444-45); the regulatory sequences of the metallothionine gene (Brinster et al., 1982, Nature 296:39-42); prokaryotic expression vectors such as the beta-lactamase promoter (Villa-Kamaroff et al., 1978, Proc. Natl. Acad. Sci. USA 75:3727-31); or the tac promoter (DeBoer et al., 1983, Proc. Natl. Acad. Sci. USA 80:21-25). Also of interest are the following animal transcriptional control regions, which exhibit tissue specificity and have been utilized in transgenic animals: the elastase I gene control region that is active in pancreatic acinar cells (Swift et al., 1984, Cell 38:639-46; Ornitz et al., 1986, Cold Spring Harbor Symp. Quant. Biol. 50:399-409 (1986); MacDonald, 1987, Hepatology 7:425-515); the insulin gene control region that is active in pancreatic beta cells (Hanahan, 1985, Nature 315:115-22); the immunoglobulin gene control region that is active in lymphoid cells (Grosschedl et al., 1984, Cell 38:647-58; Adames et al., 1985, Nature 318:533-38; Alexander et al., 1987, Mol. Cell. Biol. 7:1436-44); the mouse mammary tumor virus control region that is active in testicular, breast, lymphoid and mast cells (Leder et al., 1986, Cell 45:485-95); the albumin gene control region that is active in liver (Pinkert et al., 1987, Genes and Devel. 1:268-76); the alpha-feto-protein gene control region that is active in liver (Krumlauf et al., 1985, Mol. Cell. Biol. 5:1639-48; Hammer et al., 1987, Science 235:53-58); the alpha 1-antitrypsin gene control region that is active in liver (Kelsey et al., 1987, Genes and Devel. 1:161-71); the beta-globin gene control region that is active in myeloid cells (Mogram et al., 1985, Nature 315:338-40; Kollias et al., 1986, Cell 46:89-94); the myelin basic protein gene control region that is active in oligodendrocyte cells in the brain (Readhead et al., 1987, Cell 48:703-12); the myosin light chain-2 gene control region that is active in skeletal muscle (Sani, 1985, Nature 314:283-86); and the gonadotropic releasing hormone gene control region that is active in the hypothalamus (Mason et al., 1986, Science 234:1372-78).

The vector may also include an enhancer sequence to increase transcription of DNA encoding light chain or heavy chain.

Expression vectors of the disclosure may be constructed from a starting vector such as a commercially available vector. Such vectors may or may not contain all of the desired flanking sequences. Where one or more of the flanking sequences described herein are not already present in the vector, they may be individually obtained and ligated into the vector. Methods used for obtaining each of the flanking sequences are well known to one skilled in the art.

After the vector has been constructed and a nucleic acid molecule encoding light chain or heavy chain or light chain and heavy chain comprising an antibody or antigen binding fragment of the disclosure has been inserted into the proper site of the vector, the completed vector may be inserted into a suitable host cell for amplification and/or polypeptide expression. The transformation of an expression vector into a selected host cell may be accomplished by well-known methods including transfection, infection, calcium phosphate co-precipitation, electroporation, microinjection, lipofection, DEAE-dextran mediated transfection, or other known techniques. The method selected will in part be a function of the type of host cell to be used. These methods and other suitable methods are well known to the skilled artisan, and are set forth, for example, in Sambrook et al., supra.

The host cell, when cultured under appropriate conditions, synthesizes the antibody or antigen binding fragment of the disclosure that can subsequently be collected from the culture medium (if the host cell secretes it into the medium) or directly from the host cell producing it (if it is not secreted). The selection of an appropriate host cell will depend upon various factors, such as desired expression levels, polypeptide modifications that are desirable or necessary for activity (such as glycosylation or phosphorylation) and ease of folding into a biologically active molecule.

Mammalian cell lines available as hosts for expression are well known in the art and include, but are not limited to, many immortalized cell lines available from the American Type Culture Collection (A.T.C.C.), including but not limited to Chinese hamster ovary (CHO) cells, HeLa cells, baby hamster kidney (BHK) cells, monkey kidney cells (COS), human hepatocellular carcinoma cells (e.g., Hep G2), and a number of other cell lines. In another embodiment, one may select a cell line from the B cell lineage that does not make its own antibody but has a capacity to make and secrete a heterologous antibody (e.g., mouse myeloma cell lines NSO and SP2/0). In other embodiments, a cell other than a mammalian cell is used, such as a yeast cell line (e.g., Pichia).

In certain embodiments, the cell line stably expresses an antibody or antigen binding fragment of the disclosure. In other embodiments, the cells transiently express an antibody or antigen binding fragment of the disclosure.

In certain embodiments is provided antibodies of the disclosure (including antigen binding fragments) that are substantially purified/isolated. Numerous methods, filters, and devices for substantially purifying antibodies grown in recombinant cell culture are available.

Antibody fragments can also be made by enzymatic digestion of a full length antibody.

In certain embodiments, the antibodies or antigen binding fragments of the disclosure, whether provided alone or as conjugates with a heterologous agent, are detectably labeled. In certain embodiments, the detectable label is itself an example of a heterologous agent. Methods for conjugation to a substance, such as a detectable label, are well known in the art. In one embodiment, the attached substance is a detectable label (also referred to herein as a reporter molecule). Suitable substances for attachment to include, but are not limited to, a fluorophore, a chromophore, a dye, a radioisotope, and combinations thereof. Methods for conjugation or covalently attaching another substance to an antibody are well known in the art.

The terms “label” or “labeled” refers to incorporation of a detectable marker, e.g., by incorporation of a radiolabeled amino acid or attachment to a polypeptide of biotin moieties that can be detected by marked avidin (e.g., streptavidin preferably comprising a detectable marker such as a fluorescent marker, a chemiluminescent marker or an enzymatic activity that can be detected by optical or colorimetric methods). Various methods of labeling polypeptides and glycoproteins are known in the art and may be used advantageously in the methods disclosed herein. Examples of labels for polypeptides include, but are not limited to, the following: radioisotopes or radionuclides (e.g., ³H, ¹⁴C, ¹⁵N, ³⁵S, ⁹⁰Y, ^{99m}Tc, ¹¹¹In, ¹²⁵I, ¹³¹I). In certain embodiments, the label is a radioactive isotope. Examples of suitable radioactive materials include, but are not limited to, iodine (¹²¹I, ¹²³I, ¹²⁵I, ¹³¹I), carbon (¹⁴C) sulfur (³⁵S), tritium (³H), indium (¹¹¹In,′ ¹¹²In, ¹¹¹mIn, ¹¹⁵mIn,), technetium (⁹⁹Tc, ^{99m}Tc), thallium (²⁰¹Ti) gallium (⁶⁸Ga, ⁶⁷Ga), palladium (¹⁰³Pd), molybdenum (⁹⁹Mo), xenon (¹³⁵Xe), fluorine (¹⁸F), ¹⁵³SM, ¹⁷⁷Lu, ¹⁵⁹Gd, ¹⁴⁹Pm, ¹⁴⁰La, ¹⁷⁵Yb, ¹⁶⁶Ho, ⁹⁰Y, ⁴⁷Sc, ¹⁸⁶Re, ¹⁸⁸Re, ¹⁴²Pr, ¹⁰⁵Rh and ⁹⁷Ru).

Further examples of labels include fluorescent labels (e.g., fluoroscein isothiocyanate (FITC), rhodamine, or lanthanide phosphors), enzymatic labels (e.g., horseradish peroxidase, β-galactosidase, luciferase, alkaline phosphatase), chemiluminescent labels, hapten labels such as biotinyl groups, and predetermined polypeptide epitopes recognized by a secondary reporter (e.g., leucine zipper pair sequences, binding sites for secondary antibodies, metal binding domains, epitope tags). In certain embodiments, labels are attached by spacer arms of various lengths to reduce potential steric hindrance.

When present, regardless of the particular label, one of skill can select an appropriate label to facilitate purification, diagnostic, or research use. In other embodiments, the heterologous agent is a therapeutic molecule and either does not include a detectable label and/or epitope tag, or includes a therapeutic molecule in addition to the detectable label and/or epitope tag.

“Humanized” refers to an immunoglobulin such as an antibody, wherein the amino acids directly involved in antigen binding, the so-called complementary determining regions (CDR), of the heavy and light chains are not necessarily of human origin, while at least a portion of the rest of the variable domain (e.g., one or more of FR1, FR2, FR3, FR4) of one or both chains of the immunoglobulin molecule, the so-called framework regions of the variable heavy and/or light chains, and, if present, optionally the constant regions of the heavy and light chains are modified so that their amino acid sequence more closely correspond to human sequences.

A “humanized antibody” as used herein in the case of a two or greater chain antibody is one where at least one chain is humanized. A humanized antibody chain has a variable region where one or more of the framework regions are human or contain alterations, relative to a murine parent, so that one or more framework regions are more human than a murine parent. A humanized antibody which is a single chain is one where the chain has a variable region where one or more of the framework regions are human or contain alterations, relative to a murine parent, so that one or more framework regions are more human. The non-human portions of the variable region of the humanized antibody chain or antigen-binding fragment is derived from a non-human source, particularly a non-human antibody, typically of rodent origin. The non-human contribution to the humanized antibody is typically provided in the form of at least one CDR region which is interspersed among framework regions derived from one (or more) human immunoglobulin(s). In addition, framework support residues may be altered to preserve binding affinity. Thus, as is understood in the art, an entire framework region or all of the framework regions on a particular chain need not contain residues corresponding to a human antibody in order for the antibody to be considered humanized.

A “humanized antibody” may further comprise constant regions (e.g., at least one constant region or portion thereof, in the case of a light chain, and in some embodiments three constant regions in the case of a heavy chain). The constant regions of a humanized antibody, if present, typically are human in origin.

In some embodiments, a humanized antibody is generated by first subjecting a murine 3E10 light or heavy chain antibody sequence (e.g., the murine 3E10 antibody light and heavy chain amino acid sequences of SEQ ID NO: 18 and 17, respectively) to a sequence database search (e.g., BLAST) in order to identify the top closest human immunoglobulin kappa or heavy chain homologues in sequence similarity (e.g., the top 1, 2, 3, 4, 5, 6, 7, 8, 9 or 10 closest immunoglobulin kappa or heavy chain homologues). The top closest human immunoglobulin kappa or heavy chain homologues are considered candidates for kappa or heavy chain CDR grafting. In some embodiments, sequence alignment tools, such as Vector NTi sequence alignment tools, are then used to analyze the chimeric amino acid sequences consisting of the CDRs from the 3E10 kappa or heavy chain and the framework regions of any one of the top human immunoglobulin kappa or heavy chain homologues.

In general, as used herein, humanized antibodies comprise one or two variable domains in which all or part of the CDR regions correspond to parts derived from the non-human parent sequence and in which all or part of the FR regions are derived from a human immunoglobulin sequence. The humanized antibody can then, optionally, comprise at least one portion of a constant region of immunoglobulin (Fc), in particular that of a selected reference human immunoglobulin.

In some embodiments, the antibodies and antigen binding fragments of the disclosure (e.g., an antibody or antigen binding fragment, such as a humanized antibody or antigen binding fragment) comprises one or more of the CDRs of the 3E10 antibody. In certain embodiments, the antibodies and antigen binding fragments comprise one or more of the CDRs of a 3E10 antibody comprising a V_{H} domain comprising the amino acid sequence set forth in SEQ ID NO: 17 and a V_{L} domain comprising the amino acid sequence set forth in SEQ ID NO: 18. Either or both of the Kabat or IMGT CDRs may be used to refer to or describe an antibody. CDRs of the 3E10 antibody or an antibody of the disclosure may be determined using any of the CDR identification schemes available in the art, and such scheme may be used to describe the antibody. For example, in some embodiments, the CDRs are defined according to the Kabat definition as set forth in Kabat et al. Sequences of Proteins of Immunological Interest, 5th Ed. Public Health Service, National Institutes of Health, Bethesda, Md. (1991). In other embodiments, the CDRs are defined according to Chothia et al., 1987, J Mol Biol. 196: 901-917 and Chothia et al., 1989, Nature. 342:877-883. In other embodiments, the CDRs are defined according to the international ImMunoGeneTics database (IMGT) as set forth in LeFranc et al., 2003, Development and Comparative Immunology, 27: 55-77. In other embodiments, the CDRs of the 3E10 antibody are defined according to Honegger A, Pluckthun A., 2001, J Mol Biol., 309:657-670. In some embodiments, the CDRs are defined according to any of the CDR identification schemes discussed in Kunik et al., 2012, PLoS Comput Biol. 8(2): e1002388. In certain embodiments, antibodies and antigen binding fragments of the disclosure comprise one or more differences in the Kabat CDRs as compared to the murine, parent antibody. For example, in certain embodiments, the antibodies and antigen binding fragments of the disclosure differ at VH CDR2 and/or VL CDR2 and, optionally, at VL CDR1 in comparison to the murine, parent antibody. However, in certain embodiments, such antibodies share the IMGT CDRs of the murine, parent antibody.

Herein, the amino acid positions of residues in the VH and VL domains are referred to by linear sequence relative to, for example, SEQ ID NO: 17 or 18. Thus, the sequence of the VH and/or VL of an antibody or antigen binding fragment of the disclosure can be described relative to the corresponding amino acid position(s) of SEQ ID NO: 17 or 18. For example, a VH or VL domain may include an alteration at a particular amino acid position, and that position may correspond to a particular position in SEQ ID NO: 17 or 18.

However, the CDR identification scheme also provides numbering systems that may be used to facilitate comparisons between antibodies. Although not specifically used herein, one of skill in the art can readily use the available numbering scheme to refer to the CDRs described herein using a uniform numbering system, rather than by referring to the linear sequence. In certain embodiments, to number residues of an antibody for the purpose of identifying CDRs according to any of the CDR identification schemes known in the art, one may align the antibody at regions of homology of the sequence of the antibody with a “standard” numbered sequence known in the art for the elected CDR identification scheme. Maximal alignment of framework residues frequently requires the insertion of “spacer” residues in the numbering system, to be used for the Fv region. In addition, the identity of certain individual residues at any given site number may vary from antibody chain to antibody chain due to interspecies or allelic divergence. These uniform schemes for numbering residues are not expressly used herein, but can be readily used based on the disclosed sequences and identified CDRs.

In certain embodiments, the antibodies and antigen binding fragments of the disclosure (e.g., a humanized antibody or antigen binding fragment of the disclosure) comprises Kabat CDRs. In some embodiments, the antibodies and antigen binding fragments comprise a V_{H} CDR1 that corresponds to amino acid residues 31-35 of SEQ ID NO: 17, a V_{H} CDR2 that corresponds to amino acid residues 50-66 of SEQ ID NO: 17, and/or a V_{H} CDR3 that corresponds to amino acid residues 99-105 of SEQ ID NO: 17. We note that this numbering of amino acid residues is with reference to the linear amino acid sequence of SEQ ID NO: 17. One of skill in the art can readily use the Kabat system to identify these residues using Kabat numbering. In certain embodiments, the antibodies and antigen binding fragments comprise a V_{L} CDR1 that corresponds to amino acid residues 24-38 of SEQ ID NO: 18, a V_{L} CDR2 that corresponds to amino acid residues 54-60 of SEQ ID NO: 18, and/or a V_{L} CDR3 that corresponds to amino acid residues 93-101 of SEQ ID NO: 18. We note that this numbering of amino acid residues is with reference to the linear amino acid sequence of SEQ ID NO: 18. One of skill in the art can readily use the Kabat system to identify these residues using Kabat numbering.

In certain embodiments, the antibodies and antigen binding fragments of the disclosure comprise CDRs that are defined using the IMGT system. In some embodiments, the antibodies and antigen binding fragments comprise V_{H} CDR1 that corresponds to amino acid residues 26-33 of SEQ ID NO: 17, a V_{H} CDR2 that corresponds to amino acid residues 51-58 of SEQ ID NO: 17, and/or a V_{H} CDR3 that corresponds to amino acid residues 97-105 of SEQ ID NO: 17. We note that this numbering of amino acid residues is with reference to the linear amino acid sequence of SEQ ID NO: 17. In certain embodiments, the antibodies and antigen binding fragments comprise a V_{L} CDR1 that corresponds to amino acid residues 27-36 of SEQ ID NO: 18, a V_{L} CDR2 that corresponds to amino acid residues 54-56 of SEQ ID NO: 18, and/or a V_{L} CDR3 that corresponds to amino acid residues 93-101 of SEQ ID NO: 18. We note that this numbering of amino acid residues is with reference to the linear amino acid sequence of SEQ ID NO: 18. In certain embodiments, an antibody or antigen binding fragment of the disclosure comprises all 6 of the foregoing CDRs. In certain embodiments, the antibody or antigen binding fragment comprises 4 of the foregoing CDRs, and a VH CDR2 as set forth in SEQ ID NO: 37 and a VL CDR 2 as set forth in SEQ ID NO: 39.

In certain embodiments, the antibodies and antigen binding fragments of the disclosure comprise at least 1, 2, 3, 4, or 5 of the CDRs of 3E10 as determined using the Kabat CDR identification scheme (e.g., the CDRs set forth in SEQ ID NOs: 19-24). In certain embodiments, the antibody or antigen binding fragment further comprises a VH CDR2 as set forth in SEQ ID NO: 37 and/or a VL CDR2 as set forth in SEQ ID NO: 38 and/or a VL CDR1 as set forth in SEQ ID NO: 39. In certain embodiments, the antibodies and antigen binding fragments comprise at least 1, 2, 3, 4 or 5 of the CDRS of 3E10 as determined using the IMGT identification scheme (e.g., the CDRs set forth in SEQ ID NOs: 27-32). In certain embodiments, the antibodies and antigen binding fragments comprise all six CDRs of 3E10 as determined using the Kabat CDR identification scheme (e.g., comprises SEQ ID NOs 19-24). In other embodiments, the antibodies and antigen binding fragments comprise all six CDRS of 3E10 as determined using the IMGT identification scheme (e.g., which are set forth as SEQ ID NOs: 27-32). For any of the foregoing, in certain embodiments, the antibodies and antigen binding fragments is an antibody that binds the same epitope (e.g., the same target, such as DNA) as 3E10 and/or the internalizing moiety competes with 3E10 for binding to antigen (e.g., DNA). Exemplary antibodies and antigen binding fragments can transit cells via ENT2 and/or ENT3. In certain embodiments, antibodies or antigen binding fragments of the disclosure comprise 6 of the foregoing CDRs, but include 1, 2 3, or 4 amino acid substitutions in one or more CDRs. For example, the antibodies or antigen binding fragments comprise 3 CDR substitutions: one substitution in each of three CDRs.

In certain embodiments, antibodies or antigen binding fragments of the disclosure (e.g., a humanized antibody or antigen binding fragment of the disclosure) comprise an amino acid sequence having at least one, two, three, four, or five amino acid alterations in one or more CDRs using IMGT numbering (e.g., in one or more CDRs having the amino acid sequence of any one of SEQ ID NOs: 27-32, such as having 1-2, 1-3, 1-4, or 1-5 alternations) or Kabat numbering (e.g., in one or more CDRs having the amino acid sequence of any one of SEQ ID NOs: 19-24, such as having 1-2, 1-3, 1-4, or 1-5 alterations). In certain embodiments, antibodies or antigen binding fragments of the disclosure (e.g., a humanized antibody or antigen binding fragment of the disclosure) comprise an amino acid sequence having at least one, two, three, four, or five amino acid alterations in one or more CDRs using Kabat numbering (e.g., in one or more CDRs having the amino acid sequence of any one of SEQ ID NOs: 19-24, such as have 2, 3, 4, or 5 alterations) In some embodiments, antibodies or antigen binding fragments of the disclosure comprise a V_{L} domain comprising one or more of the following amino acid alterations: M37L, H38A or E59Q, as compared with and numbered with respect to the linear amino acid sequence of SEQ ID NO: 18. In some embodiments, any of the antibodies or antigen binding fragments disclosed herein comprise a V_{H} domain comprising a T63S alteration, as compared with and numbered with respect to the linear amino acid sequence of SEQ ID NO: 17. In some embodiments, antibodies or antigen binding fragments of the disclosure comprise a V_{L} domain comprising an E59Q alteration as compared with and numbered with respect to the linear amino acid sequence of SEQ ID NO: 18, and a V_{H} domain comprising a T63S alteration as compared with and numbered with respect to the linear amino acid sequence of SEQ ID NO: 17.

Without wishing to be bound by theory, one of the surprising findings of the present disclosure is the ability to generate antibodies and antigen-binding fragments that—have improved DNA binding activity versus murine 3E10, and further include an amino acid alteration (here, a substitution) in certain Kabat CDRs. Moreover, in certain embodiments, these improved antibodies having CDR substitutions are, in certain embodiments, also humanized.

In certain embodiments, an internalizing moiety of the disclosure, such as an antibody or antibody fragment described herein, binds a given DNA substrate with higher affinity as compared to an antibody or scFv or Fv having the VH and VL of the antibody produced by the hybridoma deposited with the ATCC under ATCC accession number PTA-2439. In certain embodiments, an internalizing moiety for use in the methods of the present disclosure is not an antibody or antibody fragment having the VH and VL of the antibody produced by the hybridoma deposited with the ATCC under ATCC accession number PTA-2439. In some embodiments, an internalizing moiety for use in the methods of the present disclosure is not a murine antibody or antibody fragment.

In certain embodiments, the antibodies and antigen binding fragments of the disclosure comprise a variable heavy chain domain comprising at least one CDR different from the corresponding CDR set forth in SEQ ID NO: 17, as determined using the Kabat CDR identification scheme. In some embodiments, the at least one different CDR is V_{H} CDR2 as set forth in SEQ ID NO: 37.

In certain embodiments, the antibodies and antigen binding fragments of the disclosure comprise a variable light chain domain comprising at least one CDR different from the corresponding CDR set forth in SEQ ID NO: 18, as determined using the Kabat CDR identification scheme. In some embodiments, the at least one different CDR is a V_{L} CDR1 as set forth in SEQ ID NO: 38. In some embodiments, the at least one different CDR is a V_{L} CDR2 as set forth in SEQ ID NO: 39.

Where the acceptor is derived from a human immunoglobulin, one may optionally select a human framework sequence that is selected based on its homology to the donor framework sequence by aligning the donor framework sequence with various human framework sequences in a collection of human framework sequences, and select the most homologous framework sequence as the acceptor. The acceptor human framework may be from or derived from human antibody germline sequences available in public databases. Regardless of the specific methodologies used to generate a humanized antibody or antibody fragment, the antibody must be evaluated to make sure that it (i) retains the desired function of the parent, murine antibody (or optionally has enhanced function); (ii) does not have deleterious properties that make it difficult to make or use; and preferably (iii) possesses one or more advantageous properties in comparison to the murine, parent antibody. Whether and to what extent any or all of these occur for any specific humanized antibody is unpredictable and uncertain. This is particularly true where substitutions are also introduced into the CDRs. Moreover, amongst a panel of humanized antibodies or antibody fragments, some may not have the required activity and one or more antibodies that do have the required activity may have advantageous properties in comparison to other humanized antibodies. This too is unpredictable and uncertain.

In certain embodiments, the antibodies or antigen-binding fragments of the disclosure comprise a light chain variable (VL) domain and a heavy chain variable (VH) domain; wherein the VL domain is humanized and comprises:

a VH CDR1 having the amino acid sequence of SEQ ID NO: 27;

a VH CDR2 having the amino acid sequence of SEQ ID NO: 28; and

a VH CDR3 having the amino acid sequence of SEQ ID NO: 29; which CDRs are in accordance with the IMGT system

and the VH domain is humanized and comprises:

a VL CDR1 having the amino acid sequence of SEQ ID NO: 30;

a VL CDR2 having the amino acid sequence of SEQ ID NO: 31; and

a VL CDR3 having the amino acid sequence of SEQ ID NO: 32; which CDRs are in accordance with the IMGT system, and wherein the antibody or antigen-binding fragment has increased DNA binding and/or cell penetration, relative to that of a murine 3E10 antibody comprising a light chain variable (VL) domain having the amino acid sequence of SEQ ID NO: 18 and a heavy chain variable (VH) domain having the amino acid sequence of SEQ ID NO: 17.

In certain embodiments the antibodies or antigen-binding fragments of the disclosure comprise a light chain variable (VL) domain and a heavy chain variable (VH) domain; wherein the VH domain comprises:

a VH CDR1 having the amino acid sequence of SEQ ID NO: 19;

a VH CDR2 having the amino acid sequence of SEQ ID NO: 37; and

a VH CDR3 having the amino acid sequence of SEQ ID NO: 21,

which CDRs are according to the Kabat system;
and the VL comprises:

a VL CDR1 having the amino acid sequence of SEQ ID NO: 38;

a VL CDR2 having the amino acid sequence of SEQ ID NO: 39; and

a VL CDR3 having the amino acid sequence of SEQ ID NO: 24,

which CDRs are according to the Kabat system;
wherein the antibody or antigen-binding fragment binds DNA.

In certain embodiments the antibodies or antigen-binding fragments of the disclosure comprise a light chain variable (VL) domain and a heavy chain variable (VH) domain; wherein the VH domain comprises:

a VH CDR1 having the amino acid sequence of SEQ ID NO: 19;

a VH CDR2 having the amino acid sequence of SEQ ID NO: 37; and

a VH CDR3 having the amino acid sequence of SEQ ID NO: 21,

which CDRs are according to Kabat;
and the VL comprises:

a VL CDR1 having the amino acid sequence of SEQ ID NO: 22;

a VL CDR2 having the amino acid sequence of SEQ ID NO: 39; and

a VL CDR3 having the amino acid sequence of SEQ ID NO: 24,

which CDRs are according to Kabat;
wherein the antibody or antigen-binding fragment binds DNA.

In certain embodiments, antibodies or antigen binding fragments of the disclosure penetrate cells (e.g., can transit the plasma membrane and enter into cells, such as cells expressing ENT2).

In some embodiments, the VH domain is humanized. In some embodiments, the VL domain is humanized

In certain embodiments the antibodies or antigen-binding fragments of the disclosure comprise a V_{L} domain that comprises the amino acid sequence set forth in SEQ ID NO: 35, or an amino acid sequence that differs from SEQ ID NO: 35 by the presence of a total of 1, 2, 3, 4, 5, or 6 amino acid substitutions, insertions and/or deletions in the framework regions, as defined by the IMGT system, relative to SEQ ID NO: 35. In other embodiments, the V_{L} domain comprises the amino acid sequence set forth in SEQ ID NO: 3, or an amino acid sequence that differs from SEQ ID NO: 3 by the presence of a total of 1, 2, 3, 4, 5, or 6 amino acid substitutions, insertions and/or deletions in the framework regions, as defined by the IMGT system, relative to SEQ ID NO: 3. In some embodiments, the VL domain comprises the amino acid sequence set forth in SEQ ID NO: 35. In some embodiments, the VL domain comprises the amino acid sequence set forth in SEQ ID NO: 3.

In certain embodiments the antibodies or antigen-binding fragments of the disclosure comprise a V_{H} domain that comprises the amino acid sequence set forth in SEQ ID NO: 33, or an amino acid sequence that differs from SEQ ID NO: 33 by the presence of a total of 1, 2, 3, 4, 5, or 6 amino acid substitutions, insertions and/or deletions in the framework regions, as defined by the IMGT system, relative to SEQ ID NO: 33. In some embodiments, the V_{H} domain comprises the amino acid sequence set forth in SEQ ID NO: 34, or an amino acid sequence that differs from SEQ ID NO: 34 by the presence of a total of 1, 2, 3, 4, 5, or 6 amino acid substitutions, insertions and/or deletions in the framework regions, as defined by the IMGT system, relative to SEQ ID NO: 34. In some embodiments, the V_{H} domain comprises the amino acid sequence set forth in SEQ ID NO: 2, or an amino acid sequence that differs from SEQ ID NO: 2 by the presence of a total of 1, 2, 3, 4, 5, or 6 amino acid substitutions, insertions and/or deletions in the framework regions, as defined by the IMGT system, relative to SEQ ID NO: 2. In some embodiments, the VH domain comprises the amino acid sequence set forth in SEQ ID NO: 33. In some embodiments, the VH domain comprises the amino acid sequence set forth in SEQ ID NO: 34. In some embodiments, the VH domain comprises the amino acid sequence set forth in SEQ ID NO: 2.

In certain embodiments the antibodies or antigen-binding fragments of the disclosure comprise a light chain variable (V_{L}) domain and a heavy chain variable (V_{H}) domain; wherein the V_{L} domain is humanized and comprises the amino acid sequence set forth in SEQ ID NO: 3; wherein the V_{H} domain comprises three CDRs of the amino acid sequence set forth in SEQ ID NO: 17, wherein the antibody or antigen-binding fragment binds DNA and penetrates cells.

In certain embodiments the antibodies or antigen-binding fragments of the disclosure comprise a light chain variable (V_{L}) domain and a heavy chain variable (V_{H}) domain; wherein the V_{L} domain is humanized and comprises the amino acid sequence set forth in SEQ ID NO: 35; wherein the V_{H} domain comprises three CDRs of the amino acid sequence set forth in SEQ ID NO: 17, wherein the antibody or antigen-binding fragment binds DNA and penetrates cells.

In certain embodiments the antibodies or antigen-binding fragments of the disclosure comprise a light chain variable (V_{L}) domain and a heavy chain variable (V_{H}) domain; wherein the V_{H} domain is humanized and comprises the amino acid sequence set forth in SEQ ID NO: 2; wherein the V_{L} domain comprises three CDRs of the amino acid sequence set forth in SEQ ID NO: 18, wherein the antibody or antigen-binding fragment binds DNA and penetrates cells.

In certain embodiments the antibodies or antigen-binding fragments of the disclosure comprise a light chain variable (V_{L}) domain and a heavy chain variable (V_{H}) domain; wherein the V_{H} domain is humanized and comprises the amino acid sequence set forth in SEQ ID NO: 33; wherein the V_{L} domain comprises three CDRs of the amino acid sequence set forth in SEQ ID NO: 18, wherein the antibody or antigen-binding fragment binds DNA and penetrates cells.

In certain embodiments the antibodies or antigen-binding fragments of the disclosure comprise a light chain variable (V_{L}) domain and a heavy chain variable (V_{H}) domain; wherein the V_{H} domain comprises the amino acid sequence set forth in SEQ ID NO: 34; wherein the V_{L} domain comprises three CDRs of the amino acid sequence set forth in SEQ ID NO: 18, wherein the antibody or antigen-binding fragment binds DNA and penetrates cells.

In certain embodiments the antibodies or antigen-binding fragments of the disclosure comprise a light chain variable (V_{L}) domain and a heavy chain variable (V_{H}) domain; wherein the V_{H} domain is humanized and comprises the amino acid sequence set forth in SEQ ID NO: 2; wherein the V_{L} domain comprises three CDRs of the amino acid sequence set forth in SEQ ID NO: 3, wherein the antibody or antigen-binding fragment binds DNA and penetrates cells.

In certain embodiments, the V_{H} domain of the antibodies or antigen-binding fragments described herein comprise:

a VH CDR1 having the amino acid sequence of SEQ ID NO: 27;

a VH CDR2 having the amino acid sequence of SEQ ID NO: 28; and

a VH CDR3 having the amino acid sequence of SEQ ID NO: 29.

In certain embodiments, the V_{L} domain of the antibodies or antigen-binding fragments described herein comprise:

a VL CDR1 having the amino acid sequence of SEQ ID NO: 30;

a VL CDR2 having the amino acid sequence of SEQ ID NO: 31; and

a VL CDR3 having the amino acid sequence of SEQ ID NO: 32.

In some embodiments, the antibodies or antigen-binding fragments disclosed herein comprise a light chain variable (V_{L}) domain and a heavy chain variable (V_{H}) domain; wherein the V_{L} domain comprises the amino acid sequence set forth in SEQ ID NO: 3; wherein the V_{H} domain comprises three CDRs of the amino acid sequence set forth in SEQ ID NO: 17, wherein the antibody or antigen-binding fragment binds DNA and penetrates cells. In some embodiments, the antibodies or antigen-binding fragments disclosed herein comprise a light chain variable (V_{L}) domain and a heavy chain variable (V_{H}) domain; wherein the V_{L} domain comprises the amino acid sequence set forth in SEQ ID NO: 35; wherein the V_{H} domain comprises three CDRs of the amino acid sequence set forth in SEQ ID NO: 17, wherein the antibody or antigen-binding fragment binds DNA and penetrates cells. In some embodiments, the antibodies or antigen-binding fragments disclosed herein comprise a light chain variable (V_{L}) domain and a heavy chain variable (V_{H}) domain; wherein the V_{H} domain comprises the amino acid sequence set forth in SEQ ID NO: 2; wherein the V_{L} domain comprises three CDRs of the amino acid sequence set forth in SEQ ID NO: 18, wherein the antibody or antigen-binding fragment binds DNA and penetrates cells. In some embodiments, the antibodies or antigen-binding fragments disclosed herein comprise a light chain variable (V_{L}) domain and a heavy chain variable (V_{H}) domain; wherein the V_{H} domain comprises the amino acid sequence set forth in SEQ ID NO: 33; wherein the V_{L} domain comprises three CDRs of the amino acid sequence set forth in SEQ ID NO: 18, wherein the antibody or antigen-binding fragment binds DNA and penetrates cells. In some embodiments, the antibodies or antigen-binding fragments disclosed herein comprise a light chain variable (V_{L}) domain and a heavy chain variable (V_{H}) domain; wherein the V_{H} domain comprises the amino acid sequence set forth in SEQ ID NO: 34; wherein the V_{L} domain comprises three CDRs of the amino acid sequence set forth in SEQ ID NO: 18, wherein the antibody or antigen-binding fragment binds DNA and penetrates cells.

In some embodiments, an antibody or antigen-binding fragment of the disclosure comprises: a) a humanized V_{H} domain that comprises the amino acid sequence of SEQ ID NO: 2, and b) a V_{L} domain that comprises the amino acid sequence of SEQ ID NO: 18. In some embodiments, an antibody or antigen-binding fragment of the disclosure comprises: a) a humanized V_{H} domain that comprises the amino acid sequence of SEQ ID NO: 2, and b) a humanized V_{L} domain that comprises the amino acid sequence of SEQ ID NO: 3. In some embodiments, an antibody or antigen-binding fragment of the disclosure comprises: a) a humanized V_{H} domain that comprises the amino acid sequence of SEQ ID NO: 2, and b) a humanized V_{L} domain that comprises the amino acid sequence of SEQ ID NO: 35. In some embodiments, an antibody or antigen-binding fragment of the disclosure comprises: a) a humanized V_{H} domain that comprises the amino acid sequence of SEQ ID NO: 33, and b) a V_{L} domain that comprises the amino acid sequence of SEQ ID NO: 18. In some embodiments, an antibody or antigen-binding fragment of the disclosure comprises: a) a humanized V_{H} domain that comprises the amino acid sequence of SEQ ID NO: 33, and b) a humanized V_{L} domain that comprises the amino acid sequence of SEQ ID NO: 3. In some embodiments, an antibody or antigen-binding fragment of the disclosure comprises: a) a humanized V_{H} domain that comprises the amino acid sequence of SEQ ID NO: 33, and b) a humanized V_{L} domain that comprises the amino acid sequence of SEQ ID NO: 35. In some embodiments, an antibody or antigen-binding fragment of the disclosure comprises: a) a humanized V_{H} domain that comprises the amino acid sequence of SEQ ID NO: 34, and b) a V_{L} domain that comprises the amino acid sequence of SEQ ID NO: 18. In some embodiments, an antibody or antigen-binding fragment of the disclosure comprises: a) a humanized V_{H} domain that comprises the amino acid sequence of SEQ ID NO: 34, and b) a humanized V_{L} domain that comprises the amino acid sequence of SEQ ID NO: 3. In some embodiments, an antibody or antigen-binding fragment of the disclosure comprises: a) a humanized V_{H} domain that comprises the amino acid sequence of SEQ ID NO: 34, and b) a humanized V_{L} domain that comprises the amino acid sequence of SEQ ID NO: 35. In some embodiments, an antibody or antigen-binding fragment of the disclosure comprises: a) a V_{H} domain that comprises the amino acid sequence of SEQ ID NO: 17, and b) a humanized V_{L} domain that comprises the amino acid sequence of SEQ ID NO: 3. In some embodiments, an antibody or antigen-binding fragment of the disclosure comprises: a) a V_{H} domain that comprises the amino acid sequence of SEQ ID NO: 17, and b) a humanized V_{L} domain that comprises the amino acid sequence of SEQ ID NO: 35.

In some embodiments, an antibody or antigen-binding fragment of the disclosure includes a signal sequence. In some embodiments, the signal sequence is conjugated to the N-terminal portion of any of the V_{L} sequences disclosed herein (e.g., SEQ ID NO: 3). In some embodiments, the signal sequence conjugated to the light chain is SEQ ID NO: 5. In some embodiments, the signal sequence is conjugated to the N-terminal portion of any of the V_{H} sequences disclosed herein (e.g., SEQ ID NO: 2). In some embodiments, the signal sequence conjugated to the heavy chain is SEQ ID NO: 4. It is understood that, when a signal sequence is included for expression of an antibody or antibody fragment, that signal sequence is generally cleaved and not present in the finished polypeptide (e.g., the signal sequence is generally cleaved and present only transiently during protein production).

In some embodiments, the V_{H} domain of any of the antibodies or antigen-binding fragments of the disclosure described herein comprise one or more of the following amino acid alterations: V5Q, E6Q, L11V, V12I, K13Q, R18L, K19R, V37I, E42G, A49S, T63S, A75S, F80Y, T84N, S88A, M93V, T111L or L112V, as compared with an numbered with reference to the amino acid sequence of SEQ ID NO: 17. In other words, in certain embodiments, an antibody or antigen-binding fragment comprises one or more amino acid alteration at a position corresponding to the foregoing, where the corresponding position is compared with SEQ ID NO: 17. In certain embodiments, the V_{H} domain comprises one or more of the following amino acid alterations: V5Q, L11V, K13Q, R18L, K19R, V37I, E42G, A49S, T63S, A75S, F80Y, T84N, M93V, T111L or L112V, as compared with and numbered with reference to the amino acid sequence of SEQ ID NO: 17. In certain embodiments, the V_{H} domain comprises at least 5, at least 6, at least 7, at least 8, at least 9, at least 10, at least 11, at least 12, at least 13, at least 14, at least 15, at least 16, or at least 17 of said alterations, as compared with and numbered with reference to the amino acid sequence of SEQ ID NO: 17. In certain embodiments, at least one of the alterations in the V_{H} domain is a V5Q alteration, as compared with and numbered with reference to the amino acid sequence of SEQ ID NO: 17. In certain embodiments, at least one of the alterations in the V_{H} domain is a E6Q alteration, as compared with and numbered with reference to the amino acid sequence of SEQ ID NO: 17. In certain embodiments, at least one of the alterations in the V_{H} domain is a L11V alteration, as compared with and numbered with reference to the amino acid sequence of SEQ ID NO: 17. In certain embodiments, at least one of the alterations in the V_{H} domain is a V37I alteration, as compared with and numbered with reference to the amino acid sequence of SEQ ID NO: 17. In certain embodiments, the V_{H} domain retains a serine at the amino acid position corresponding to amino acid position 88 of SEQ ID NO: 17. In certain embodiments, the V_{H} domain retains a valine at the amino acid position corresponding to amino acid position 12 of SEQ ID NO: 17. In certain embodiments, the V_{H} domain retains a tryptophan at the amino acid position corresponding to amino acid position 47 of SEQ ID NO: 17. All operable combinations of the foregoing are contemplated, as are combinations with any of the aspect and embodiments provided herein for the VL. The foregoing numbering of amino acid residues is with reference to linear amino acid sequence of a given VH and the disclosure contemplates humanized antibodies and antigen binding fragments having one or more of the recited substitutions at a position corresponding to the recited position in the murine, parent VH or VL.

In certain embodiments of any of the foregoing, or of any of the aspects and embodiments disclosed herein, the V_{L} domain of any of the humanized antibodies or antigen-binding fragments described herein comprise one or more of the following amino acid alterations: V3Q, L4M, A9S, A12S, V13A, L15V, Q17D, A19V, S22T, M37L, H38A, G45E, Q46K, P47A, E59Q, A64S, H76T, N78T, H80S, P81S, V82L, E83Q, E84P, A87V, A87F, or G104A, as compared with and numbered with reference to the amino acid sequence of SEQ ID NO: 18. In certain embodiments, the V_{L} domain comprises one or more of the following amino acid alterations: V3Q, L4M, A9S, A12S, V13A, L15V, Q17D, A19V, G45E, Q46K, P47A, E59Q, A64S, H76T, N78T, H805, P81S, V82L, E83Q, E84P, A87V, or G104A, as compared with and numbered with reference to the amino acid sequence of SEQ ID NO: 18. In certain embodiments, the V_{L} domain comprises at least 5, at least 6, at least 7, at least 8, at least 9, at least 10, at least 11, at least 12, at least 13, at least 14, at least 15, at least 16, at least 17, at least 18, at least 19, at least 20, at least 21, or at least 22 of said amino acid alterations, as compared with and numbered with reference to the amino acid sequence of SEQ ID NO: 18.

It should be understood that any of the foregoing variations at particular positions are referred to relative to the amino acid sequence set forth in SEQ ID NO: 18 or 17. An antibody or antigen binding fragment of the disclosure may comprise one or more of such amino acid alterations at the corresponding position, relative to the amino acid sequence of SEQ ID NO: 18 or 17. By way of example, in certain embodiments, the VH domain comprises an L to V alteration at a position corresponding to position 11 of SEQ ID NO: 17 (e.g., an L11V alteration). This is exemplary of how all of the foregoing alterations can also be described, and such description is expressly contemplated. By way of another example, in certain embodiments, the VL domain comprises a V to Q alteration at a position corresponding to position 3 of SEQ ID NO: 18 (e.g., a V3Q alteration).

In certain embodiments, the V_{L} domain comprises a serine at each of the amino acid positions corresponding to amino acid positions 80 and 81 of SEQ ID NO: 18. In certain embodiments, the V_{L} domain retains a lysine at the amino acid position corresponding to amino acid position 53 of SEQ ID NO: 18. In certain embodiments, the V_{L} domain does not have any one or more of the following amino acid combinations:

a) asparagine and serine at the amino acid positions corresponding to amino acid positions 80 and 81 of SEQ ID NO: 18, respectively; or

b) asparagine and glycine at the amino acid positions corresponding to amino acid positions 80 and 81 of SEQ ID NO: 18, respectively; or

c) asparagine and proline at the amino acid positions corresponding to amino acid positions 80 and 81 of SEQ ID NO: 18, respectively. All operable combinations of the foregoing are contemplated, as are combinations with any of the aspect and embodiments provided herein for the VH. The foregoing numbering of amino acid residues is with reference to linear amino acid sequence of a given VH and the disclosure contemplates humanized antibodies and antigen binding fragments having one or more of the recited substitutions at a position corresponding to the recited position in the murine, parent VH or VL.

In some embodiments, the humanized internalizing moiety (e.g., a humanized antibody or antigen-binding fragment comprising a light chain variable (V_{L}) domain comprising the amino acid sequence set forth in SEQ ID NO: 3 and a heavy chain variable (V_{H}) domain comprising the amino acid sequence set forth in SEQ ID NO: 2) is associated with at least one superior physiological or biological property as compared to a reference non-humanized internalizing moiety (e.g., the murine, parent 3E10 antibody). In other embodiments, the humanized internalizing moiety is associated with at least two superior physiological or biological properties as compared to a reference non-humanized internalizing moiety. In other embodiments, the humanized internalizing moiety is associated with at least three superior physiological or biological properties as compared to a reference non-humanized internalizing moiety (e.g., the murine, parent 3E10 antibody). In some embodiments, the reference non-humanized internalizing moiety comprises the murine parent antibody comprising a VH comprising the amino acid sequence of SEQ ID NO: 17 and a VL comprising the amino acid sequence of SEQ ID NO: 18. In some embodiments, the reference humanized internalizing moiety is an antibody comprising the amino acid sequence of SEQ ID NO: 42. In some embodiments, the reference internalizing moiety is a humanized antibody or antigen binding fragment comprising the V_{H} amino acid sequence of SEQ ID NO: 41 and the V_{L} amino acid sequence of SEQ ID NO: 40.

In certain embodiments, the antibodies or antigen-binding fragments described herein are humanized and are associated with at least one superior biological or physiological property as compared to a murine antibody, which murine antibody comprises a V_{L} domain comprising the amino acid sequence set forth in SEQ ID NO: 18 and a V_{H} domain comprising the amino acid sequence set forth in SEQ ID NO: 17, and/or as compared to an alternative antibody or antigen-binding fragment thereof, wherein said alternative antibody or antigen-binding fragment comprises a V_{L} domain comprising the CDRs of the amino acid sequence set forth in SEQ ID NO: 18 and a V_{H} domain comprising the CDRs of the amino acid sequence set forth in SEQ ID NO: 17; and wherein said alternative antibody or fragment does not comprise a V_{L} domain comprising the amino acid sequence of SEQ ID NO: 3 or 35, and/or wherein said alternative antibody or fragment does not comprise a V_{H} domain comprising the amino acid sequence of any of SEQ ID NOs: 2, 33 or 34; or, in some embodiments, wherein said alternative antibody or fragment does not comprise a V_{L} domain comprising the amino acid sequence of SEQ ID NO: 3, and/or wherein said alternative antibody or fragment does not comprise a V_{H} domain comprising the amino acid sequence of any of SEQ ID NOs: 2.

In some embodiments, a humanized internalizing moiety of the disclosure (e.g., a humanized antibody or antigen-binding fragment thereof comprises a light chain variable (V_{L}) domain comprising the amino acid sequence set forth in SEQ ID NO: 3 and a heavy chain variable (V_{H}) domain comprising the amino acid sequence set forth in SEQ ID NO: 2) is associated with at least one superior physiological or biological property as compared to an alternative internalizing moiety or fragment thereof (e.g., a different humanized antibody based on the same parent, murine antibody and, optionally, having the same CDRs). In other embodiments, a humanized internalizing moiety of the disclosure is associated with at least two superior physiological or biological properties as compared to the alternative internalizing moiety (e.g., a different humanized antibody based on the same parent, murine antibody and, optionally, having the same CDRs). In other embodiments, the humanized internalizing moiety of the disclosure is associated with at least three superior physiological or biological properties as compared to the alternative internalizing moiety (e.g., a different humanized antibody based on the same parent, murine antibody and, optionally, having the same CDRs). In some embodiments, the alternative antibody is the parent antibody from which the humanized antibody was derived (e.g., the parent, murine antibody). In some embodiments, the alternative antibody is another humanized antibody that is derived from the 3E10 antibody but that has a different amino acid sequence than the humanized internalizing moieties or antigen-binding fragments thereof of the present disclosure. In some embodiments, an antibody or antigen binding fragment of the disclosure has one or more improved characteristics in comparison to the murine parent antibody and/or an alternative humanized antibody. In some embodiments, the alternative humanized antibody has one, two, or three amino acid substitutions in the Kabat CDRs, as compared to an antibody of the disclosure. In some embodiments, the alternative internalizing moiety or fragment thereof comprises:

a VH CDR1 having the amino acid sequence of SEQ ID NO: 19;

a VH CDR2 having the amino acid sequence of SEQ ID NO: 20;

a VH CDR3 having the amino acid sequence of SEQ ID NO: 21;

a VL CDR1 having the amino acid sequence of SEQ ID NO: 22;

a VL CDR2 having the amino acid sequence of SEQ ID NO: 23; and

a VL CDR3 having the amino acid sequence of SEQ ID NO: 24, which CDRs are defined in accordance with Kabat, but does not comprise the same scaffold amino acid sequence present in the humanized internalizing moieties or fragments thereof of the present disclosure (e.g. a humanized internalizing moiety or fragment thereof comprising the amino acid sequence of any of SEQ ID NOs: 2, 3 or 38-40).

In some embodiments, the superior biological or physiological property associated with the humanized internalizing moieties or fragments of the disclosure described herein is that the humanized internalizing moiety or antigen-binding fragment is associated with reduced immunogenicity in a human patient as compared to the immunogenicity of the non-humanized or to the alternative antibody or antigen-binding fragment in a human patient. The skilled worker is familiar with numerous assays for determining the immunogenicity of the antibodies. In preferred embodiments, the humanized antibodies of the disclosure are associated with reduced immunogenicity in a human patient, but retain the cell penetration properties associated with the murine 3E10 antibody.

In some embodiments, the superior biological or physiological property associated with the humanized internalizing moieties or fragments of the disclosure described herein is that the humanized internalizing moiety or antigen-binding fragment is associated with increased solubility in a physiologically acceptable carrier as compared to the solubility of the non-humanized or to the alternative antibody or antigen-binding fragment in the same type of physiologically acceptable carrier. As used herein, a physiologically acceptable carrier includes include water, ethanol, polyols (such as glycerol, propylene glycol, polyethylene glycol, and the like), and suitable mixtures thereof, vegetable oils, such as olive oil, and injectable organic esters, such as ethyl oleate. In some embodiments, the humanized internalizing moiety or fragment is associated with at least 5%, 10%, 25%, 50%, 75%, 100%, 150%, 200% or 300% greater solubility in a physiologically acceptable carrier as compared to a non-humanized or alternative internalizing moiety or antigen-binding fragment in the same type of physiologically acceptable carrier. The skilled worker is aware of routine experiments that may be utilized for testing the solubility of the humanized internalizing moieties or fragments thereof. Examples of solubility assays include standard turbidity or light-scattering assays, commercial solubility assays, such as the OptiSol™ solubility assay kit (DiLyx, Seattle, Wash.), or the protein solubility assay screen described in Bondos et al., 2003, Analytical Biochemistry, 316:223-231 may be utilized.

In some embodiments, the superior biological or physiological property associated with the humanized internalizing moieties or fragments of the disclosure described herein is that the humanized internalizing moiety or antigen-binding fragment is associated with a higher expression level in a type of cell as compared to the expression level of the non-humanized or alternative antibody or antigen-binding fragment in the same type of cell. In some embodiments, the humanized internalizing moiety or fragment is associated with at least 5%, 10%, 25%, 50%, 75%, 100%, 150%, 200% or 300% higher expression level in a cell as compared to the expression level of a non-humanized or alternative internalizing moiety or antigen-binding fragment in the same type of cell. The skilled worker is aware of routine experiments that may be utilized for testing the expression level of the humanized internalizing moieties or fragments thereof.

In some embodiments, the superior biological or physiological property associated with the humanized internalizing moieties or fragments of the disclosure described herein is that the humanized internalizing moiety or antigen-binding fragment is associated with lower toxicity (e.g., cytotoxicity and/or genotoxicity) in a cell type as compared to the toxicity in the same type of cell that is associated with the non-humanized or alternative antibody or antigen-binding fragment. In some embodiments, the humanized internalizing moiety or fragment is associated with at least 5%, 10%, 25%, 50%, 75%, 100%, 150%, 200% or 300% lower toxicity as compared to the toxicity of a non-humanized or alternative internalizing moiety or antigen-binding fragment in the same type of cell. In some embodiments the cell is a mammalian cell. In some embodiments the cell is a human cell. In some embodiments, the cell is in an organism, such as a mammal. In some embodiments, the cell is a human cell in a human organism. The skilled worker is aware of routine experiments that may be utilized for testing the toxicity of the humanized internalizing moieties or fragments thereof. For example, the toxicity of the humanized internalizing moieties or fragments of the disclosure and of the non-humanized or alternative internalizing moieties or fragments thereof may be tested in an in vitro cell or cell culture, such as in a cell or cell culture derived from human cells, or may be tested in an in vitro animal model such as a mouse or rat.

In some embodiments, the superior biological or physiological property associated with the humanized internalizing moieties or fragments of the disclosure described herein is that the humanized internalizing moiety or antigen-binding fragment is associated with reduced aggregation in a physiologically acceptable carrier as compared to aggregation of the non-humanized or alternative antibody or antigen-binding fragment in the same type of physiologically acceptable carrier. In some embodiments, the humanized internalizing moiety or fragment is associated with at least 5%, 10%, 25%, 50%, 75%, 100%, 150%, 200% or 300% less aggregation in a physiologically acceptable carrier as compared to a non-humanized or alternative internalizing moiety or antigen-binding fragment in the same type of physiologically acceptable carrier. In some embodiments, the humanized antibody or antigen-binding fragment in a pharmaceutically acceptable carrier is associated with reduced aggregation after a period of at least 1 hour, 6 hours, 12 hours, 18 hours, 24 hours, 36 hours, 2 days, 5 days, one week, two weeks, four weeks, one month, two months, three months, six months or one year. The skilled worker is aware of routine experiments that may be utilized for testing the aggregation of the humanized internalizing moieties or fragments thereof. Examples of aggregation assays include standard turbidity or light-scattering assays (e.g., A600 nm assay), visual inspection, SDS-PAGE, commercial aggregation assays, such as the OptiSol™ aggregation assay kit (DiLyx, Seattle, Wash.), HP-SEC analysis, or the protein aggregation assay screen described in Bondos et al., 2003, Analytical Biochemistry, 316:223-231 may be utilized.

In some embodiments, the superior biological or physiological property associated with the humanized internalizing moieties or antigen-binding fragments of the disclosure described herein is that the humanized internalizing moiety or antigen-binding fragment is associated with increased stability in a physiologically acceptable carrier as compared to the stability of the non-humanized or alternative antibody or antigen-binding fragment in the same type of physiologically acceptable carrier. In some embodiments, the humanized internalizing moiety or fragment is associated with at least 5%, 10%, 25%, 50%, 75%, 100%, 150%, 200% or 300% greater stability in a physiologically acceptable carrier as compared to a non-humanized or alternative internalizing moiety or antigen-binding fragment in the same type of physiologically acceptable carrier. In some embodiments, the humanized antibody or antigen-binding antigen-binding fragment in a pharmaceutically acceptable carrier is associated with increased stability after a period of at least 1 hour, 6 hours, 12 hours, 18 hours, 24 hours, 36 hours, 2 days, 5 days, one week, two weeks, four weeks, one month, two months, three months, six months or one year as compared to a non-humanized or alternative internalizing moiety or antigen-binding fragment in the same type of physiologically acceptable carrier. The skilled worker is aware of routine experiments that may be utilized for testing the stability of the humanized internalizing moieties or fragments thereof. For example, the skilled worker could test the stability of the humanized and non-humanized or alternative internalizing moieties or fragments thereof after various intervals of being stored in a physiologically acceptable carrier. Commercial assays such as the ProteoStat™ Thermal shift stability assay (Enzo, Farmingdale, N.Y.) may be utilized in assessing the stability of the moieties or fragments thereof. Alternatively, the stability of the moieties or fragments thereof may be determined by HP-SEC or by SDS-PAGE analysis.

In some embodiments, the superior biological or physiological property associated with the humanized internalizing moieties or antigen-binding fragments of the disclosure described herein is that the humanized internalizing moiety or antigen-binding fragment is associated with improved cell penetration as compared to the cell penetration of the non-humanized or alternative antibody or antigen-binding fragment. In some embodiments, the improved penetration is due to the increased efficiency of the humanized internalizing moiety or antigen-binding fragment to be internalized by an ENT transporter (e.g., an ENT2 and/or ENT3 transporter). In some embodiments, the humanized internalizing moiety or fragment is associated with at least 5%, 10%, 25%, 50%, 75%, 100%, 150%, 200% or 300% greater cell penetration as compared to a non-humanized or alternative internalizing moiety or antigen-binding fragment in the same type of physiologically acceptable carrier. The skilled worker is aware of routine experiments that may be utilized for testing the cell penetration of the humanized internalizing moieties or fragments thereof. For example, the humanized internalizing moieties or fragments thereof may be labeled (e.g. fluorescently or radiolabeled) and administered to a cell or cell culture in order to determine the cell penetration of the humanized internalizing moieties or fragments thereof. Alternatively, the humanized internalizing moieties or fragments may be administered to a cell or cell culture and then detected with a secondary agent, e.g., a fluorescently labeled or radiolabeled secondary antibody, in order to determine the cell penetration of the humanized internalizing moieties or fragments thereof.

In some embodiments, the superior biological or physiological property associated with the humanized internalizing moieties or fragments of the disclosure described herein is that the humanized internalizing moiety or antigen-binding fragment is associated with reduced glycosylation in a cell type as compared to the glycosylation of the non-humanized or alternative antibody or antigen-binding fragment in the same cell type. In some embodiments, an asparagine is mutated to another amino acid residue in the VH or VL domains in order to reduce N-linked glycosylation of the humanized antibody or antibody fragment. In other embodiments, the superior biological or physiological property associated with the humanized internalizing moieties or fragments described herein is that the humanized internalizing moiety or antigen-binding fragment is associated with increased glycosylation in a cell type as compared to the glycosylation of the non-humanized or alternative antibody or antigen-binding fragment in the same cell type. In some embodiments, the superior biological or physiological property associated with the humanized internalizing moieties or fragments described herein is that the humanized internalizing moiety or antigen-binding fragment is associated with a specific pattern of glycosylation in a cell type that differs from the glycosylation pattern of the non-humanized or alternative internalizing moiety or antigen-binding fragment in the same type of cell. For example, the humanized internalizing moiety or antigen-binding fragment may be hemi-glycosylated in a cell type while the non-humanized or alternative internalizing moiety or antigen-binding fragment is not hemi-glycosylated in the same type of cell. In some embodiments, the superior biological or physiological property associated with the humanized internalizing moieties or fragments described herein is that the humanized internalizing moiety or antigen-binding fragment is post-translationally modified with a specific glycosylation group in a cell type that differs from the post-translational modification of the non-humanized or alternative internalizing moiety or antigen-binding fragment in the same type of cell. The skilled worker is aware of routine experiments that may be utilized for testing the glycosylation patterns of the humanized internalizing moieties or fragments thereof. Examples of experiments for testing the glycosylation levels and patterns of the internalizing moieties and fragments thereof include protocols described in Mohammad, 2002, Protein Protocols Handbook, pages 795-802; standard procedures involving mass spectrometry and/or HPLC; GLYCO-PRO™ (Sigma-Aldrich); and Qproteome Total Glycoprotein Kit™ (Qiagen, Valencia, Calif.). In order to identify the exact sites of glycosylation in a protein sequence, standard endoproteinase cleavage may be performed (e.g. tryptic digest) followed by analysis by LC/MS or HILIC-MS/MS, similar to the protocols described in Zauner G et al., 2010, J Sep Sci., 33:903-10.

In some embodiments, the superior biological or physiological property associated with the humanized internalizing moieties or fragments of the disclosure described herein is that the humanized internalizing moiety or antigen-binding fragment is associated with reduced deamidation in a physiologically acceptable carrier as compared to deamidation of the non-humanized or alternative antibody or antigen-binding fragment in the same type of physiologically acceptable carrier. In some embodiments, the humanized internalizing moiety or fragment is associated with at least 5%, 10%, 25%, 50%, 75%, 100%, 150%, 200% or 300% less deamidation in a physiologically acceptable carrier as compared to a non-humanized or alternative internalizing moiety or antigen-binding fragment in the same type of physiologically acceptable carrier. In some embodiments, the humanized antibody or antigen-binding fragment in a pharmaceutically acceptable carrier is associated with reduced deamidation after a period of at least 1 hour, 6 hours, 12 hours, 18 hours, 24 hours, 36 hours, 2 days, 5 days, one week, two weeks, four weeks, one month, two months, three months, six months or one year as compared to a non-humanized or alternative internalizing moiety or antigen-binding fragment in the same type of physiologically acceptable carrier. The skilled worker is aware of routine experiments that may be utilized for testing the deamidation of the humanized internalizing moieties or fragments thereof. Examples of assays for testing protein deamidation include commercially available deamidation assays such as the ISOQUANT® Isoaspartate Detection Kit (Promega, Madison Wis.) or Dionex UltiMate 3000 Titanium System (Dionex, Sunnyvale, Calif.). Other assays may include peptide mapping. See generally, Kalgahtgi, K., & Horvath, C. “Rapid Peptide Mapping by High Performance Liquid Chromatography”, J. Chromatography 443, 343-354 (1988).

In some embodiments, the superior biological or physiological property associated with the humanized internalizing moieties or fragments of the disclosure described herein is that the humanized internalizing moiety or antigen-binding fragment is associated with reduced oxidation in a physiologically acceptable carrier as compared to oxidation of the non-humanized or alternative antibody or antigen-binding fragment in the same type of physiologically acceptable carrier. In some embodiments, the humanized internalizing moiety or fragment is associated with at least 5%, 10%, 25%, 50%, 75%, 100%, 150%, 200% or 300% less oxidation in a physiologically acceptable carrier as compared to a non-humanized or alternative internalizing moiety or antigen-binding fragment in the same type of physiologically acceptable carrier. In some embodiments, the humanized antibody or antigen-binding antigen-binding fragment in a pharmaceutically acceptable carrier is associated with reduced oxidation after a period of at least 1 hour, 6 hours, 12 hours, 18 hours, 24 hours, 36 hours, 2 days, 5 days, one week, two weeks, four weeks, one month, two months, three months, six months or one year as compared to a non-humanized or alternative internalizing moiety or antigen-binding fragment in the same type of physiologically acceptable carrier. The skilled worker is aware of routine experiments that may be utilized for testing the oxidation of the humanized internalizing moieties or fragments thereof. For example, oxidation levels may be assessed by using any one of several commercially available oxidation assays, such as the Methionine Sulfoxide Immunoblotting Kit (Cayman Chemical, Ann Arbor, Mich.). Other assays may include peptide mapping. See generally, Kalgahtgi, K., & Horvath, C. “Rapid Peptide Mapping by High Performance Liquid Chromatography”, J. Chromatography 443, 343-354 (1988).

In some embodiments, the superior biological or physiological property associated with the humanized internalizing moieties or fragments of the disclosure described herein is that the humanized internalizing moiety or antigen-binding fragment is associated with reduced lipidation when produced in a cell type as compared to the lipidation of the non-humanized or alternative antibody or fragment when produced in the same type of cell. In other embodiments, the superior biological or physiological property associated with the humanized internalizing moieties or fragments described herein is that the humanized internalizing moiety or antigen-binding fragment is associated with increased lipidation when produced in a cell type as compared to the lipidation of the non-humanized or alternative antibody or antigen-binding fragment when produced in the same type of cell. In some embodiments, the superior biological or physiological property associated with the humanized internalizing moieties or fragments described herein is that the humanized internalizing moiety or antigen-binding fragment is associated with a specific pattern of lipidation when produced in a cell type that differs from the lipidation pattern of the non-humanized or alternative internalizing moiety or antigen-binding fragment when produced in the same type of cell. In some embodiments, the superior biological or physiological property associated with the humanized internalizing moieties or fragments described herein is that the humanized internalizing moiety or antigen-binding fragment is post-translationally modified with a specific lipidation group when produced in a cell type that differs from the post-translational modification of the non-humanized or alternative internalizing moiety or antigen-binding fragment when produced in the same type of cell. The skilled worker is aware of routine experiments that may be utilized for testing the lipidation patterns of the humanized internalizing moieties or fragments thereof. For example, the internalizing moieties or fragments thereof may be assessed by the protocols described in Gelb et al., 1999, Protein Lipidation Protocols, Humana Press, pages 1-256.

In some embodiments, the superior biological or physiological property associated with the humanized internalizing moieties or fragments of the disclosure described herein is that the humanized internalizing moiety or antigen-binding fragment is capable of binding a polynucleotide (e.g., DNA) with higher affinity (lower K_{D}) as compared to the binding affinity of the non-humanized, parent antibody or an alternative antibody or fragment, such as a different humanized antibody. In some embodiments, the humanized internalizing moiety or fragment is associated with at least 5%, 10%, 25%, 50%, 75%, 100%, 150%, 200% or 300% stronger binding affinity for a polynucleotide (e.g., DNA; double stranded blunt DNA) as compared to a non-humanized or alternative internalizing moiety or antigen-binding fragment in the same type of physiologically acceptable carrier. The skilled worker is aware of routine experiments that may be utilized for testing the binding affinity (K_{D}) of the humanized internalizing moieties or fragments thereof. Binding affinity can be measured using Surface Plasmon Resonance (SPR) or Quartz Crystal Microbalance (QCM), in accordance with currently standard methods and the manufacturer's protocols.

Homing Peptides

In certain aspects, an internalizing moiety may comprise a homing peptide which selectively directs the subject chimeric alpha-amylase polypeptide to a target tissue (e.g., muscle). For example, delivering a chimeric polypeptide to the muscle can be mediated by a homing peptide comprising an amino acid sequence of ASSLNIA. Further exemplary homing peptides are disclosed in WO 98/53804. Homing peptides for a target tissue (or organ) can be identified using various methods well known in the art. Additional examples of homing peptides include the HIV transactivator of transcription (TAT) which comprises the nuclear localization sequence Tat48-60; Drosophila antennapedia transcription factor homeodomain (e.g., Penetratin which comprises Antp43-58 homeodomain 3rd helix); Homo-arginine peptides (e.g., Arg7 peptide-PKC-E agonist protection of ischemic rat heart); alpha-helical peptides; cationic peptides (“superpositively” charged proteins). In some embodiments, the homing peptide transits cellular membranes via an equilibrative nucleoside (ENT) transporter. In some embodiments, the homing peptide transits cellular membranes via an ENT1, ENT2, ENT3 or ENT4 transporter. In some embodiments, the homing peptide targets ENT2. In other embodiments, the homing peptide targets muscle cells. The muscle cells targeted by the homing peptide may include skeletal, cardiac or smooth muscle cells. In other embodiments, the homing peptide targets neurons, epithelial cells, liver cells, kidney cells or Leydig cells.

In certain embodiments, the homing peptide is capable of binding polynucleotides. In certain embodiments, the homing peptide is capable of binding DNA. In certain embodiments, the homing peptide is capable of binding DNA with a K_{D} of less than 1 μM. In certain embodiments, the homing peptide is capable of binding DNA with a K_{D} of less than 100 nM.

Additionally, homing peptides for a target tissue (or organ) can be identified using various methods well known in the art. Once identified, a homing peptide that is selective for a particular target tissue can be used, in certain embodiments.

An exemplary method is the in vivo phage display method. Specifically, random peptide sequences are expressed as fusion peptides with the surface proteins of phage, and this library of random peptides are infused into the systemic circulation. After infusion into host mice, target tissues or organs are harvested, the phage is then isolated and expanded, and the injection procedure repeated two more times. Each round of injection includes, by default, a negative selection component, as the injected virus has the opportunity to either randomly bind to tissues, or to specifically bind to non-target tissues. Virus sequences that specifically bind to non-target tissues will be quickly eliminated by the selection process, while the number of non-specific binding phage diminishes with each round of selection. Many laboratories have identified the homing peptides that are selective for vasculature of brain, kidney, lung, skin, pancreas, intestine, uterus, adrenal gland, retina, muscle, prostate, or tumors. See, for example, Samoylova et al., 1999, Muscle Nerve, 22:460; Pasqualini et al., 1996, Nature, 380:364; Koivunen et al., 1995, Biotechnology, 13:265; Pasqualini et al., 1995, J. Cell Biol., 130:1189; Pasqualini et al., 1996, Mole. Psych., 1:421, 423; Rajotte et al., 1998, J. Clin. Invest., 102:430; Rajotte et al., 1999, J. Biol. Chem., 274:11593. See, also, U.S. Pat. Nos. 5,622,699; 6,068,829; 6,174,687; 6,180,084; 6,232,287; 6,296,832; 6,303,573; 6,306,365. Homing peptides that target any of the above tissues may be used for targeting an alpha-amylase protein to that tissue.

Additional Targeting to Lysosomes and Autophagic Vesicles

A traditional method of targeting a protein to lysosomes is modification of the protein with M6P residues, which directs their transport to lysosomes through interaction of M6P residues and M6PR molecules on the inner surface of structures such as the Golgi apparatus or late endosome. Transport of endogenous alpha-amylase to the lysosome depends on M6P and M6PR interaction. In certain embodiments, chimeric polypeptides of the present disclosure (e.g., polypeptides comprising alpha-amylase; and an internalizing moiety) may further include modification to facilitate additional targeting to the lysosome through M6PRs or in pathways independent of M6PRs. Such targeting moieties may be added, for example, at the N-terminus or C-terminus of a chimeric polypeptide, and via conjugation to 3E10 or alpha-amylase. In other embodiments, the alpha-amylase portion of a chimeric polypeptide comprises all or some of the endogenous sequences to facilitate M6P transport.

In some embodiments, the chimeric polypeptides of the present disclosure are transported to lysosomes via the cellular process of autophagy. Autophagy is a catabolic mechanism that involves cell degradation of unnecessary or dysfunctional cellular components through the lysosomal machinery. During this process, targeted cytoplasmic constituents are isolated from the rest of the cell within vesicles called autophagosomes, which are then fused with lysosomes and degraded or recycled. Uptake of proteins into autophagic vesicles is mediated by the formation of a membrane around the targeted region of a cell and subsequent fusion of the vesicle with a lysosome. Several mechanisms for autophagy are known, including macroautophagy in which organelles and proteins are sequestered within the cell in a vesicle called an autophagic vacuole. Upon fusion with the lysosome, the contents of the autophagic vacuole are degraded by acidic lysosomal hydrolases. In microautophagy, lysosomes engulf cytoplasm directly, and in chaperone-mediated autophagy, proteins with a consensus peptide sequence are bound by a hsc70-containing chaperone-cochaperone complex, which is recognized by a lysosomal protein and translocated across the lysosomal membrane. Autophagic vacuoles have a lysosomal environment (low pH), which is conducive for activity of enzymes.

Autophagy naturally occurs in muscle cells of mammals (Masiero et al, 2009, Cell Metabolism, 10(6): 507-15). As the autophagic vacuoles take up proteins from the cytoplasm, the chimeric polypeptides of the present disclosure are expected to be taken up by glycogen-containing autophagic vesicles, where the chimeric polypeptides would be free to degrade any glycogen present within those vacuoles. As such, in some embodiments, the chimeric polypeptides are capable of being taken up by autophagic vacuoles without addition of any autophagic vacuole-specific targeting motif.

In certain embodiments, the chimeric polypeptides of the present disclosure may further include modification to facilitate additional targeting to autophagic vesicles. One known chaperone-targeting motif is KFERQ-like motif. Accordingly, this motif can be added to chimeric polypeptides as described herein, in order to target the polypeptides for autophagy. Such targeting moieties may be added, for example, at the N-terminus or C-terminus of a chimeric polypeptide, and via conjugation to 3E10 or alpha-amylase.

M6P residues or chaperone-targeting motifs may be added to the alpha-amylase polypeptides.

### III. Chimeric Polypeptides

The disclosure provides chimeric polypeptides comprising an internalizing moiety portion and a non-internalizing moiety portion. As detailed above, a non-internalizing moiety polypeptide portion comprises or consists of an alpha-amylase polypeptide (e.g., a mature alpha-amylase). Alternatively, a non-internalizing moiety polypeptide portion comprises or consists of an acid alpha-glucosidase (e.g., a mature acid alpha-glucosidase). Numerous examples of internalizing moieties, and each of the potential non-internalizing moiety polypeptide portions are described above, and all suitable combinations of internalizing moiety portions and non-internalizing moiety polypeptide portions to generate chimeric polypeptides are contemplated.

Without being bound by theory, the association of the alpha-amylase polypeptide (e.g., a mature alpha-amylase polypeptide) or the acid alpha-glucosidase (e.g., a mature acid alpha-glucosidase) with the internalizing moiety portion facilitates delivery of the chimeric polypeptide, and thus, the non-internalizing moiety portion to the cytoplasm and, optionally, to the lysosome and/or autophagic vesicles. In certain embodiments, the internalizing moiety delivers alpha-amylase activity into cells. In certain embodiments, the internalizing moiety delivers acid alpha-glucosidase activity into cells. In certain embodiments, the chimeric polypeptide of the disclosure comprises an alpha-amylase-containing chimeric polypeptide (e.g., the non-internalizing moiety portion comprises or consists of an alpha-amylase polypeptide). In certain embodiments, the chimeric polypeptide of the disclosure comprises an acid alpha-glucosidase-containing chimeric polypeptide (e.g., the non-internalizing moiety portion comprises or consists of an acid alpha-glucosidase polypeptide). Any of the internalizing moieties described herein may be combined with any of the non-internalizing moiety polypeptide portions, as described herein, to generate a chimeric polypeptide of the disclosure.

The disclosure provides chimeric polypeptides (e.g., chimeric polypeptides of the disclosure). Chimeric polypeptides for use in the methods disclosed herein can be made in various manners. The chimeric polypeptides may comprise any of the internalizing moiety portions and the alpha-amylase polypeptide portions disclosed herein. In other aspects, the chimeric polypeptides may comprise any of the internalizing moiety portions and the acid alpha-glucosidase polypeptide portions disclosed herein. Chimeric polypeptides of the disclosure may comprise (i) an alpha-amylase polypeptide portion and (ii) an internalizing moiety portion. Alternatively, chimeric polypeptides of the disclosure may comprise (i) an acid alpha-glucosidase portion and (ii) an internalizing moiety portion. In addition, any of the chimeric polypeptides disclosed herein may be utilized in any of the methods or compositions disclosed herein. In some embodiments, an internalizing moiety (e.g. an antibody or antigen-binding fragment) is linked, directly or indirectly, to any of the polypeptides and/or fragments and/or variants disclosed herein.

In some embodiments, the alpha-amylase polypeptide is a mature alpha-amylase and comprises the amino acid sequence of SEQ ID NO: 1, or variants or fragments thereof, fused to the C-terminus of an internalizing moiety. In some embodiments, the alpha-amylase polypeptide comprises the amino acid sequence of SEQ ID NO: 1, or variants or fragments thereof, fused to the C-terminus of the heavy chain segment of a Fab internalizing moiety. In some embodiments, the alpha-amylase polypeptide comprises the amino acid sequence of SEQ ID NO: 1, or variants or fragments thereof, fused to the C-terminus of the heavy chain segment of a full-length antibody internalizing moiety.

In some embodiments, the acid alpha-glucosidase polypeptide is a mature acid alpha-glucosidase and comprises the amino acid sequence of SEQ ID NO: 49, 50, or 51, or variants or fragments thereof, fused to the C-terminus of an internalizing moiety. In some embodiments, the acid alpha-glucosidase polypeptide comprises the amino acid sequence of SEQ ID NO: 49, 50, or 51, or variants or fragments thereof, fused to the C-terminus of the heavy chain segment of a Fab internalizing moiety. In some embodiments, the acid alpha-glucosidase polypeptide comprises the amino acid sequence of SEQ ID NO: 49, 50, or 51, or variants or fragments thereof, fused to the C-terminus of the heavy chain segment of a full-length antibody internalizing moiety.

In some embodiments, the chimeric polypeptide comprises: (i) an acid alpha-glucosidase polypeptide, and (ii) an internalizing moiety; wherein the acid alpha-glucosidase polypeptide comprises an amino acid sequence that is at least 85%, 90%, 91%, 92%, 93%, 94%, 95%, 96%, 97%, 98%, 99% or 100% identical to the amino acid sequence of SEQ ID NO: 49; and wherein the internalizing moiety is an antibody or antigen binding fragment, wherein the antibody or antigen binding fragment comprises a heavy chain variable domain and a light chain variable domain; wherein the heavy chain variable domain comprises an amino acid sequence that is at least 85%, 90%, 91%, 92%, 93%, 94%, 95%, 96%, 97%, 98%, 99% or 100% identical to the amino acid sequence of SEQ ID NO: 2; and wherein the light chain variable domain comprises an amino acid sequence that is at least 85%, 90%, 91%, 92%, 93%, 94%, 95%, 96%, 97%, 98%, 99% or 100% identical to the amino acid sequence of SEQ ID NO: 3. In some embodiments, the chimeric polypeptide comprises: (i) an acid alpha-glucosidase polypeptide, and (ii) an internalizing moiety; wherein the acid alpha-glucosidase polypeptide comprises an amino acid sequence that is at least 85%, 90%, 91%, 92%, 93%, 94%, 95%, 96%, 97%, 98%, 99% or 100% identical to the amino acid sequence of SEQ ID NO: 50; and wherein the internalizing moiety is an antibody or antigen binding fragment, wherein the antibody or antigen binding fragment comprises a heavy chain variable domain and a light chain variable domain; wherein the heavy chain variable domain comprises an amino acid sequence that is at least 85%, 90%, 91%, 92%, 93%, 94%, 95%, 96%, 97%, 98%, 99% or 100% identical to the amino acid sequence of SEQ ID NO: 2; and wherein the light chain variable domain comprises an amino acid sequence that is at least 85%, 90%, 91%, 92%, 93%, 94%, 95%, 96%, 97%, 98%, 99% or 100% identical to the amino acid sequence of SEQ ID NO: 3. In some embodiments, the chimeric polypeptide comprises: (i) an acid alpha-glucosidase polypeptide, and (ii) an internalizing moiety; wherein the acid alpha-glucosidase polypeptide comprises an amino acid sequence that is at least 85%, 90%, 91%, 92%, 93%, 94%, 95%, 96%, 97%, 98%, 99% or 100% identical to the amino acid sequence of SEQ ID NO: 51; and wherein the internalizing moiety is an antibody or antigen binding fragment, wherein the antibody or antigen binding fragment comprises a heavy chain variable domain and a light chain variable domain; wherein the heavy chain variable domain comprises an amino acid sequence that is at least 85%, 90%, 91%, 92%, 93%, 94%, 95%, 96%, 97%, 98%, 99% or 100% identical to the amino acid sequence of SEQ ID NO: 2; and wherein the light chain variable domain comprises an amino acid sequence that is at least 85%, 90%, 91%, 92%, 93%, 94%, 95%, 96%, 97%, 98%, 99% or 100% identical to the amino acid sequence of SEQ ID NO: 3.

In some embodiments, the chimeric polypeptide comprises: (i) an alpha-amylase polypeptide, and (ii) an internalizing moiety; wherein the alpha-amylase polypeptide comprises an amino acid sequence that is at least 85%, 90%, 91%, 92%, 93%, 94%, 95%, 96%, 97%, 98%, 99% or 100% identical to the amino acid sequence of SEQ ID NO: 1; and wherein the internalizing moiety is an antibody or antigen binding fragment, wherein the antibody or antigen binding fragment comprises a heavy chain variable domain and a light chain variable domain; wherein the heavy chain variable domain comprises an amino acid sequence that is at least 85%, 90%, 91%, 92%, 93%, 94%, 95%, 96%, 97%, 98%, 99% or 100% identical to the amino acid sequence of SEQ ID NO: 2; and wherein the light chain variable domain comprises an amino acid sequence that is at least 85%, 90%, 91%, 92%, 93%, 94%, 95%, 96%, 97%, 98%, 99% or 100% identical to the amino acid sequence of SEQ ID NO: 3. In some embodiments, the chimeric polypeptide comprises: (i) an alpha-amylase polypeptide, and (ii) an internalizing moiety; wherein the alpha-amylase polypeptide comprises the amino acid sequence of SEQ ID NO: 1; and wherein the internalizing moiety is an antibody or antigen binding fragment, wherein the antibody or antigen binding fragment comprises a heavy chain variable domain and a light chain variable domain; wherein the heavy chain variable domain comprises the amino acid sequence of SEQ ID NO: 2; and wherein the light chain variable domain comprises the amino acid sequence of SEQ ID NO: 3. In some embodiments, the heavy chain comprises the leader sequence of SEQ ID NO: 4. In some embodiments, the light chain comprises the leader sequence of SEQ ID NO: 5. In some embodiments, the disclosure provides a chimeric polypeptide that does not include a leader sequence, for example, the leader sequence has been processed. In some embodiments, the chimeric polypeptide comprises a linker interconnecting the alpha-amylase polypeptide to the internalizing moiety. In some embodiments, the linker comprises the amino acid sequence of SEQ ID NO: 6. In some embodiments, the chimeric polypeptide comprises a heavy chain amino acid sequence lacking a leader sequence (e.g., lacking the leader sequence of SEQ ID NO: 4). In some embodiments, the chimeric polypeptide comprises the amino acid sequence of SEQ ID NO: 7. In some embodiments, the chimeric polypeptide comprises a light chain amino acid sequence lacking a leader sequence (e.g., lacking the leader sequence of SEQ ID NO: 5). In some embodiments, the chimeric polypeptide comprises the amino acid sequence of SEQ ID NO: 8. In some embodiments, the chimeric polypeptide comprises the amino acid sequence of both SEQ ID NOs: 7 and 8.

In some embodiments, the chimeric polypeptide comprises an amino acid sequence that is at least 85%, 90%, 91%, 92%, 93%, 94%, 95%, 96%, 97%, 98%, 99% or 100% identical to the amino acid sequence of SEQ ID NO: 9. In some embodiments, the chimeric polypeptide comprises an amino acid sequence that is at least 85%, 90%, 91%, 92%, 93%, 94%, 95%, 96%, 97%, 98%, 99% or 100% identical to the amino acid sequence of SEQ ID NO: 10. In some embodiments, the chimeric polypeptide comprises the amino acid sequences of both SEQ ID NOs: 9 and 10. In some embodiments, the chimeric polypeptide comprises an amino acid sequence that is at least 85%, 90%, 91%, 92%, 93%, 94%, 95%, 96%, 97%, 98%, 99% or 100% identical to the amino acid sequence of SEQ ID NO: 43. In some embodiments, the chimeric polypeptide comprises an amino acid sequence that is at least 85%, 90%, 91%, 92%, 93%, 94%, 95%, 96%, 97%, 98%, 99% or 100% identical to the amino acid sequence of SEQ ID NO: 8. In some embodiments, the chimeric polypeptide comprises the amino acid sequences of both SEQ ID NOs: 8 and 43.

In some embodiments, a chimeric polypeptide comprising any of the mature alpha-amylase polypeptide or fragments or variants thereof disclosed herein and any of the antibodies or antigen binding fragments disclosed herein (e.g., a protein comprising the amino acid sequences of SEQ ID NOs: 8 and 43), has a higher biological activity (e.g., at least 10%, 20%, 30%, 40%, 50%, 60%, 70%, 80%, 90%, 100%, 150%, or 200% higher biological activity) at a slightly acidic pH (e.g., pH 5.5) as compared to a reference wildtype mature alpha amylase (e.g., an alpha-amylase consisting of the amino acid sequence of SEQ ID NO: 1). In some embodiments, the chimeric polypeptide has a higher biological activity (e.g., at least 10%, 20%, 30%, 40%, 50%, 60%, 70%, 80%, 90%, 100%, 150%, or 200% higher biological activity) at a slightly acidic pH (e.g., pH 5.5) as compared to the biological activity of the same chimeric polypeptide at a neutral pH (e.g., pH 7.0). In some embodiments, the chimeric polypeptide has a higher biological activity (e.g., at least 10%, 20%, 30%, 40%, 50%, 60%, 70%, 80%, 90%, 100%, 150%, or 200% higher biological activity) at a slightly acidic pH (e.g., pH 5.5) as compared to the biological activity of the same chimeric polypeptide at a more acidic pH (e.g., pH 4.3). In some embodiments, the “slightly acidic pH” is selected from the group consisting of ranges 4.5 to 6.5; 4.8 to 6.3; 5.2 to 6.2; 5.3 to 6.3; 5.0 to 6.0; 5.2 to 5.8; 5.3 to 5.7; 5.4 to 5.6; or at 5.5. In some embodiments, the chimeric polypeptide has highest biological activity at a pH range of 4.5 to 6.5; 4.8 to 6.3; 5.2 to 6.2; 5.3 to 6.3; 5.0 to 6.0; 5.2 to 5.8; 5.3 to 5.7; 5.4 to 5.6; or at 5.5. In some embodiments, the biological activity is the ability of the alpha-amylase portion of the chimeric polypeptide to hydrolyze glycogen. In some embodiments, the biological activity may be measured using a glycogen digestion assay, similar to the assay described in the Exemplification section provided herein.

In certain embodiments, potential configurations include the use of truncated portions of an antibody's heavy and light chain sequences (e.g., mAB 3E10) as needed to maintain the functional integrity of the attached alpha-amylase. Further still, the internalizing moiety can be linked to an exposed internal (non-terminus) residue of alpha-amylase or a fragment and/or variant thereof. In some embodiments, any combination of the alpha-amylase-internalizing moiety configurations can be employed, thereby resulting in a alpha-amylase:internalizing moiety ratio that is greater than 1:1 (e.g., two alpha-amylase molecules to one internalizing moiety).

The polypeptide (e.g., the alpha-amylase polypeptide or the acid alpha-glucosidase polypeptide) and the internalizing moiety may be linked directly to each other. Alternatively, they may be linked to each other via a linker sequence, which separates alpha-amylase polypeptide and the internalizing moiety by a distance sufficient to ensure that each domain properly folds into its secondary and tertiary structures. Preferred linker sequences (1) should adopt a flexible extended conformation, (2) should not exhibit a propensity for developing an ordered secondary structure which could interact with the functional domains of the alpha-amylase polypeptide or the internalizing moiety, and (3) should have minimal hydrophobic or charged character, which could promote interaction with the functional protein domains. Typical surface amino acids in flexible protein regions include Gly, Asn and Ser. Permutations of amino acid sequences containing Gly, Asn and Ser would be expected to satisfy the above criteria for a linker sequence. Other near neutral amino acids, such as Thr and Ala, can also be used in the linker sequence. In a specific embodiment, a linker sequence length of about 20 amino acids can be used to provide a suitable separation of functional protein domains, although longer or shorter linker sequences may also be used. The length of the linker sequence separating the alpha-amylase polypeptide from the internalizing moiety can be from 5 to 500 amino acids in length, or more preferably from 5 to 100 amino acids in length. Preferably, the linker sequence is from about 5-30 amino acids in length. In preferred embodiments, the linker sequence is from about 5 to about 20 amino acids, and is advantageously from about 10 to about 20 amino acids. In other embodiments, the linker joining the alpha-amylase polypeptide to an internalizing moiety can be a constant domain of an antibody (e.g., constant domain of mAb 3E10 or all or a portion of an Fc region of another antibody). In certain embodiments, the linker is a cleavable linker. In certain embodiments, the linker sequence comprises the linker sequence of SEQ ID NO: 6. In certain embodiments, the internalizing moiety is an antibody or antibody fragment and the conjugation includes chemical or recombinant conjugation to a constant domain, such as the constant domain of a heavy chain of the antibody or antibody fragment. In such embodiments, it is appreciated that the alpha-amylase polypeptide and internalizing moiety may be further associated via the association between the heavy chain and light chain of the antibody or antibody fragment. This is also included within the scope of the conjugation.

In other embodiments, the polypeptide (e.g., alpha-amylase polypeptide or acid alpha-glucosidase polypeptide) or functional fragment thereof may be conjugated or joined directly to the internalizing moiety. For example, a recombinantly conjugated chimeric polypeptide can be produced as an in-frame fusion of the alpha-amylase portion and the internalizing moiety portion. In certain embodiments, the linker may be a cleavable linker. In any of the foregoing embodiments, the internalizing moiety may be conjugated (directly or via a linker) to the N-terminal or C-terminal amino acid of the alpha-amylase polypeptide. In other embodiments, the internalizing moiety may be conjugated (directly or indirectly) to an internal amino acid of the alpha-amylase polypeptide. Note that the two portions of the construct are conjugated/joined to each other. Unless otherwise specified, describing the chimeric polypeptide as a conjugation of the alpha-amylase portion to the internalizing moiety is used equivalently as a conjugation of the internalizing moiety to the alpha-amylase portion. Further, unless otherwise specified, conjugation and/or joining refers to either chemical or genetic conjugation.

In certain embodiments, the chimeric polypeptides of the present disclosure can be generated using well-known cross-linking reagents and protocols. For example, there are a large number of chemical cross-linking agents that are known to those skilled in the art and useful for cross-linking the alpha-amylase polypeptide with an internalizing moiety (e.g., an antibody). For example, the cross-linking agents are heterobifunctional cross-linkers, which can be used to link molecules in a stepwise manner Heterobifunctional cross-linkers provide the ability to design more specific coupling methods for conjugating proteins, thereby reducing the occurrences of unwanted side reactions such as homo-protein polymers. A wide variety of heterobifunctional cross-linkers are known in the art, including succinimidyl 4-(N-maleimidomethyl) cyclohexane-1-carboxylate (SMCC), m-Maleimidobenzoyl-N-hydroxysuccinimide ester (MBS); N-succinimidyl (4-iodoacetyl) aminobenzoate (SIAB), succinimidyl 4-(p-maleimidophenyl) butyrate (SMPB), 1-ethyl-3-β-dimethylaminopropyl) carbodiimide hydrochloride (EDC); 4-succinimidyloxycarbonyl-a-methyl-a-(2-pyridyldithio)-tolune (SMPT), N-succinimidyl 3-(2-pyridyldithio) propionate (SPDP), succinimidyl 6-[3-(2-pyridyldithio) propionate] hexanoate (LC-SPDP). Those cross-linking agents having N-hydroxysuccinimide moieties can be obtained as the N-hydroxysulfosuccinimide analogs, which generally have greater water solubility. In addition, those cross-linking agents having disulfide bridges within the linking chain can be synthesized instead as the alkyl derivatives so as to reduce the amount of linker cleavage in vivo. In addition to the heterobifunctional cross-linkers, there exists a number of other cross-linking agents including homobifunctional and photoreactive cross-linkers. Disuccinimidyl subcrate (DSS), bismaleimidohexane (BMH) and dimethylpimelimidate.2 HCl (DMP) are examples of useful homobifunctional cross-linking agents, and bis-[B-(4-azidosalicylamido)ethyl]disulfide (BASED) and N-succinimidyl-6(4′-azido-2′-nitrophenylamino)hexanoate (SANPAH) are examples of useful photoreactive cross-linkers for use in this disclosure. For a recent review of protein coupling techniques, see Means et al. (1990) Bioconjugate Chemistry. 1:2-12, incorporated by reference herein.

One particularly useful class of heterobifunctional cross-linkers, included above, contain the primary amine reactive group, N-hydroxysuccinimide (NHS), or its water soluble analog N-hydroxysulfosuccinimide (sulfo-NHS). Primary amines (lysine epsilon groups) at alkaline pH's are unprotonated and react by nucleophilic attack on NHS or sulfo-NHS esters. This reaction results in the formation of an amide bond, and release of NHS or sulfo-NHS as a by-product. Another reactive group useful as part of a heterobifunctional cross-linker is a thiol reactive group. Common thiol reactive groups include maleimides, halogens, and pyridyl disulfides. Maleimides react specifically with free sulfhydryls (cysteine residues) in minutes, under slightly acidic to neutral (pH 6.5-7.5) conditions. Halogens (iodoacetyl functions) react with —SH groups at physiological pH's. Both of these reactive groups result in the formation of stable thioether bonds. The third component of the heterobifunctional cross-linker is the spacer arm or bridge. The bridge is the structure that connects the two reactive ends. The most apparent attribute of the bridge is its effect on steric hindrance. In some instances, a longer bridge can more easily span the distance necessary to link two complex biomolecules.

In some embodiments, the chimeric polypeptide comprises multiple linkers. For example, if the chimeric polypeptide comprises an scFv internalizing moiety, the chimeric polypeptide may comprise a first linker conjugating the alpha-amylase to the internalizing moiety, and a second linker in the scFv conjugating the V_{H} domain (e.g., SEQ ID NO: 2) to the V_{L} domain (e.g., SEQ ID NO: 3).

Preparing protein-conjugates using heterobifunctional reagents is a two-step process involving the amine reaction and the sulfhydryl reaction. For the first step, the amine reaction, the protein chosen should contain a primary amine. This can be lysine epsilon amines or a primary alpha amine found at the N-terminus of most proteins. The protein should not contain free sulfhydryl groups. In cases where both proteins to be conjugated contain free sulfhydryl groups, one protein can be modified so that all sulfhydryls are blocked using for instance, N-ethylmaleimide (see Partis et al. (1983) J. Pro. Chem. 2:263, incorporated by reference herein). Ellman's Reagent can be used to calculate the quantity of sulfhydryls in a particular protein (see for example Ellman et al. (1958) Arch. Biochem. Biophys. 74:443 and Riddles et al. (1979) Anal. Biochem. 94:75, incorporated by reference herein).

In certain specific embodiments, chimeric polypeptides of the disclosure can be produced by using a universal carrier system. For example, a alpha-amylase polypeptide can be conjugated to a common carrier such as protein A, poly-L-lysine, hex-histidine, and the like. The conjugated carrier will then form a complex with an antibody which acts as an internalizing moiety. A small portion of the carrier molecule that is responsible for binding immunoglobulin could be used as the carrier.

In certain embodiments, chimeric polypeptides of the disclosure can be produced by using standard protein chemistry techniques such as those described in Bodansky, M. Principles of Peptide Synthesis, Springer Verlag, Berlin (1993) and Grant G. A. (ed.), Synthetic Peptides: A User's Guide, W. H. Freeman and Company, New York (1992). In addition, automated peptide synthesizers are commercially available (e.g., Advanced ChemTech Model 396; Milligen/Biosearch 9600). In any of the foregoing methods of cross-linking for chemical conjugation of alpha-amylase to an internalizing moiety, a cleavable domain or cleavable linker can be used. Cleavage will allow separation of the internalizing moiety and the alpha-amylase polypeptide. For example, following penetration of a cell by a chimeric polypeptide, cleavage of the cleavable linker would allow separation of alpha-amylase from the internalizing moiety.

In certain embodiments, the chimeric polypeptides comprising a alpha-amylase polypeptide and an internalizing moiety portion can be generated as a fusion protein containing the alpha-amylase polypeptide and the internalizing moiety. In certain embodiments, the chimeric polypeptides of the present disclosure can be generated as a fusion protein containing a alpha-amylase polypeptide and an internalizing moiety (e.g., an antibody or a homing peptide), expressed as one contiguous polypeptide chain. In certain embodiments, the chimeric polypeptide is generated as a fusion protein that comprises an alpha-amylase polypeptide portion and internalizing moiety portion. In preparing such fusion protein, a fusion gene is constructed comprising nucleic acids which encode a alpha-amylase polypeptide and an internalizing moiety, and optionally, a peptide linker sequence to span the alpha-amylase polypeptide and the internalizing moiety. The use of recombinant DNA techniques to create a fusion gene, with the translational product being the desired fusion protein, is well known in the art. Both the coding sequence of a gene and its regulatory regions can be redesigned to change the functional properties of the protein product, the amount of protein made, or the cell type in which the protein is produced. The coding sequence of a gene can be extensively altered—for example, by fusing part of it to the coding sequence of a different gene to produce a novel hybrid gene that encodes a fusion protein. Examples of methods for producing fusion proteins are described in PCT applications PCT/US87/02968, PCT/US89/03587 and PCT/US90/07335, as well as Traunecker et al. (1989) Nature 339:68, incorporated by reference herein. Essentially, the joining of various DNA fragments coding for different polypeptide sequences is performed in accordance with conventional techniques, employing blunt-ended or stagger-ended termini for ligation, restriction enzyme digestion to provide for appropriate termini, filling in of cohesive ends as appropriate, alkaline phosphatase treatment to avoid undesirable joining, and enzymatic ligation. Alternatively, the fusion gene can be synthesized by conventional techniques including automated DNA synthesizers. In another method, PCR amplification of gene fragments can be carried out using anchor primers which give rise to complementary overhangs between two consecutive gene fragments which can subsequently be annealed to generate a chimeric gene sequence (see, for example, Current Protocols in Molecular Biology, Eds. Ausubel et al. John Wiley & Sons: 1992). The chimeric polypeptides encoded by the fusion gene may be recombinantly produced using various expression systems as is well known in the art (also see below).

Recombinantly conjugated chimeric polypeptides include embodiments in which the alpha-amylase polypeptide is conjugated to the N-terminus or C-terminus of the internalizing moiety. Exemplary chimeric polypeptides in which alpha-amylase are conjugated to variant light and heavy chains of Fv3E10 are indicated in SEQ ID NOs: 3 and 2, respectively.

Recombinantly conjugated chimeric polypeptides include embodiments in which the internalizing moiety is N-terminal to the alpha-amylase polypeptide and embodiments in which the internalizing moiety is C-terminal to the alpha-amylase polypeptide portion. We note that methods of making fusion proteins recombinantly are well known in the art. Any of the chimeric proteins described herein can readily be made recombinantly. This includes proteins having one or more tags and/or one or more linkers. For example, if the chimeric polypeptide comprises an scFv internalizing moiety, the chimeric polypeptide may comprise a first linker interconnection the internalizing moiety to the alpha-amylase polypeptide portion, and a second linker in the scFv conjugating the V_{H} domain. Moreover, in certain embodiments, the chimeric polypeptides comprise a “AGIH” portion (SEQ ID NO: 25) on the N-terminus of the chimeric polypeptide (or within 10 amino acid residues of the N-terminus), and such chimeric polypeptides may be provided in the presence or absence of one or more epitope tags. In further embodiments, the chimeric polypeptide comprises a serine at the N-terminal most position of the polypeptide. In some embodiments, the chimeric polypeptides comprise an “SAGIH” (SEQ ID NO: 26) portion at the N-terminus of the polypeptide (or within 10 amino acid residues of the N-terminus), and such chimeric polypeptides may be provided in the presence or absence of one or more epitope tags.

In some embodiments, the chimeric polypeptides comprise a signal sequence (e.g., SEQ ID NO: 4 or 5). In some embodiments, the signal sequence (e.g., SEQ ID NO: 5) is at the N-terminus of the light chain sequence of any of the antibodies or antigen binding fragments disclosed herein. In some embodiments, the signal sequence (e.g., SEQ ID NO: 5) is at the N-terminus of the amino acid sequence SEQ ID NO: 3, or fragments or variants thereof. In some embodiments, the signal sequence (e.g., SEQ ID NO: 4) is at the N-terminus of the heavy chain sequence of any of the antibodies or antigen binding fragments disclosed herein. In some embodiments, the signal sequence (e.g., SEQ ID NO: 4) is at the N-terminus of the amino acid sequence SEQ ID NO: 2, or fragments or variants thereof.

In some embodiments, the chimeric polypeptides are produced recombinantly in cells. In some embodiments, the cells are bacteria (e.g., E. coli), yeast (e.g., Picchia), insect cells (e.g., Sf9 cells) or mammalian cells (e.g., CHO or HEK-293 cells). Chimeric polypeptides of the disclosure are, in certain embodiments, made in any of the foregoing cells in culture using art recognized techniques for making and purifying protein from cells or cell supernatant.

The presence in the chimeric polypeptide of all or a portion of an immunoglobulin or an epitope tag, such as an HA or myc tag, provides a region for purification of chimeric polypeptide.

In some embodiments, the immunogenicity of the chimeric polypeptide may be reduced by identifying a candidate T-cell epitope within a junction region spanning the chimeric polypeptide and changing an amino acid within the junction region as described in U.S. Patent Publication No. 2003/0166877.

Chimeric polypeptides according to the disclosure can be used for numerous purposes. We note that any of the chimeric polypeptides described herein can be used in any of the methods described herein, and such suitable combinations are specifically contemplated.

Chimeric polypeptides described herein can be used to deliver alpha-amylase polypeptide to cells. In certain embodiments, chimeric polypeptides deliver alpha-amylase to neuronal cells. In certain embodiments, chimeric polypeptides deliver alpha-amylase to cardiac cells. Thus, the chimeric polypeptides can be used to facilitate transport of alpha-amylase to cells in vitro or in vivo. By facilitating transport to cells, the chimeric polypeptides improve delivery efficiency, thus facilitating working with alpha-amylase polypeptide in vitro or in vivo. Further, by increasing the efficiency of transport, the chimeric polypeptides may help decrease the amount of alpha-amylase needed for in vitro or in vivo experimentation. Moreover, by facilitating delivery to the cytoplasm, the chimeric polypeptides and methods of the disclosure can address the problems associated with cytoplasmic accumulation of glycogen in, for example, Forbes-Cori and/or Andersen Disease and/or Pompe Disease and/or von Gierke Disease and/or Lafora Disease and/or Danon Disease and/or Alzheimer's Disease.

The chimeric polypeptides can be used to study the function of alpha-amylase in cells in culture, as well as to study transport of alpha-amylase. The chimeric polypeptides can be used to identify binding partners for alpha-amylase in cells, such as transport between cytoplasm and lysosome. The chimeric polypeptides can be used in screens to identify modifiers (e.g., small organic molecules or polypeptide modifiers) of alpha-amylase activity in a cell. The chimeric polypeptides can be used to help treat or alleviate the symptoms of Forbes-Cori and/or Andersen Disease and/or Pompe Disease and/or von Gierke Disease and/or Lafora Disease and/or Danon Disease and/or Alzheimer's Disease in humans or in an animal model. The foregoing are merely exemplary of the uses for the subject chimeric polypeptides.

Any of the chimeric polypeptides described herein, including chimeric polypeptides combining any of the features of the alpha-amylase polypeptides, internalizing moieties, and linkers, may be used in any of the methods of the disclosure.

### IV. Nucleic Acids and Expression

In certain embodiments, the present disclosure makes use of nucleic acids for producing an alpha-amylase polypeptide (including a mature alpha-amylase polypeptide and functional fragments, variants, and fusions thereof). In certain embodiments, the present disclosure makes use of nucleic acids for producing an acid alpha-glucosidase polypeptide (including a mature acid alpha-glucosidase polypeptide and functional fragments, variants, and fusions thereof). In certain specific embodiments, the nucleic acids may further comprise DNA which encodes an internalizing moiety for making a recombinant chimeric protein of the disclosure.

In certain embodiments, the disclosure relates to isolated or recombinant nucleic acid sequences that are at least 80%, 85%, 90%, 95%, 97%, 98%, 99% or 100% identical to a region of an alpha-amylase nucleotide sequence (e.g., GenBank Accession No. AH002672.1 or AH002671.1). In some embodiments, the nucleotide sequence encodes a mature alpha-amylase polypeptide sequence. In particular embodiments, the alpha-amylase nucleotide sequence encodes an alpha-amylase polypeptide that lacks the amino acids corresponding to amino acids 1-15 of SEQ ID NO: 1. In further embodiments, the alpha-amylase nucleic acid sequences can be isolated, recombinant, and/or fused with a heterologous nucleotide sequence, or in a DNA library.

In certain embodiments, alpha-amylase nucleic acids also include nucleotide sequences that hybridize under highly stringent conditions to any of the above-mentioned nucleotide sequences, or complement sequences thereof. One of ordinary skill in the art will understand readily that appropriate stringency conditions which promote DNA hybridization can be varied. For example, one could perform the hybridization at 6.0× sodium chloride/sodium citrate (SSC) at about 45° C., followed by a wash of 2.0×SSC at 50° C. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or temperature or salt concentration may be held constant while the other variable is changed. In one embodiment, the disclosure provides nucleic acids which hybridize under low stringency conditions of 6×SSC at room temperature followed by a wash at 2×SSC at room temperature.

Isolated nucleic acids which differ from the native alpha-amylase nucleic acids due to degeneracy in the genetic code are also within the scope of the disclosure. For example, a number of amino acids are designated by more than one triplet. Codons that specify the same amino acid, or synonyms (for example, CAU and CAC are synonyms for histidine) may result in “silent” mutations which do not affect the amino acid sequence of the protein. However, it is expected that DNA sequence polymorphisms that do lead to changes in the amino acid sequences of the subject proteins will exist among mammalian cells. One skilled in the art will appreciate that these variations in one or more nucleotides (up to about 3-5% of the nucleotides) of the nucleic acids encoding a particular protein may exist among individuals of a given species due to natural allelic variation. Any and all such nucleotide variations and resulting amino acid polymorphisms are within the scope of this disclosure.

In some embodiments, any of the nucleic acids disclosed herein are codon optimized for expression in a particular cell expression system, e.g., a mammalian cell, a yeast cell, a bacterial cell, a plant cell or an insect cell. In some embodiments, the nucleic acids are codon optimized for expression in a mammalian cell, such as a CHO or HEK-293 cell.

In certain embodiments, the recombinant alpha-amylase nucleic acids may be operably linked to one or more regulatory nucleotide sequences in an expression construct. Regulatory nucleotide sequences will generally be appropriate for a host cell used for expression. Numerous types of appropriate expression vectors and suitable regulatory sequences are known in the art for a variety of host cells. Typically, said one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are contemplated by the disclosure. The promoters may be either naturally occurring promoters, or hybrid promoters that combine elements of more than one promoter. An expression construct may be present in a cell on an episome, such as a plasmid, or the expression construct may be inserted in a chromosome. In a preferred embodiment, the expression vector contains a selectable marker gene to allow the selection of transformed host cells. Selectable marker genes are well known in the art and will vary with the host cell used. In certain aspects, this disclosure relates to an expression vector comprising a nucleotide sequence encoding a alpha-amylase polypeptide, such as any of the alpha-amylase polypeptides described herein, and operably linked to at least one regulatory sequence. Regulatory sequences are art-recognized and are selected to direct expression of the encoded polypeptide. Accordingly, the term regulatory sequence includes promoters, enhancers, and other expression control elements. Exemplary regulatory sequences are described in Goeddel; Gene Expression Technology: Methods in Enzymology, Academic Press, San Diego, Calif. (1990). It should be understood that the design of the expression vector may depend on such factors as the choice of the host cell (e.g., Chinese Hamster Ovary cells) to be transformed and/or the type of protein desired to be expressed. Moreover, the vector's copy number, the ability to control that copy number and the expression of any other protein encoded by the vector, such as antibiotic markers, should also be considered.

In some embodiments, a nucleic acid construct, comprising a nucleotide sequence that encodes an alpha-amylase polypeptide or a bioactive fragment thereof, is operably linked to a nucleotide sequence that encodes an internalizing moiety, wherein the nucleic acid construct encodes a chimeric polypeptide having alpha-amylase biological activity. In certain embodiments, the nucleic acid constructs may further comprise a nucleotide sequence that encodes a linker.

This disclosure also pertains to a host cell transfected with a recombinant gene which encodes an alpha-amylase polypeptide or a chimeric polypeptide of the disclosure. The host cell may be any prokaryotic or eukaryotic cell. For example, an alpha-amylase polypeptide or a chimeric polypeptide may be expressed in bacterial cells such as E. coli, insect cells (e.g., using a baculovirus expression system), yeast, or mammalian cells. Other suitable host cells are known to those skilled in the art.

The present disclosure further pertains to methods of producing an alpha-amylase polypeptide or a chimeric polypeptide of the disclosure. For example, a host cell transfected with an expression vector encoding a alpha-amylase polypeptide or a chimeric polypeptide can be cultured under appropriate conditions to allow expression of the polypeptide to occur. The polypeptide may be secreted and isolated from a mixture of cells and medium containing the polypeptides. Alternatively, the polypeptides may be retained cytoplasmically or in a membrane fraction and the cells harvested, lysed and the protein isolated. A cell culture includes host cells, media and other byproducts. Suitable media for cell culture are well known in the art. The polypeptides can be isolated from cell culture medium, host cells, or both using techniques known in the art for purifying proteins, including ion-exchange chromatography, gel filtration chromatography, ultrafiltration, electrophoresis, and immunoaffinity purification with antibodies specific for particular epitopes of the polypeptides (e.g., an alpha-amylase polypeptide). In a preferred embodiment, the polypeptide is a fusion protein containing a domain which facilitates its purification.

A recombinant alpha-amylase nucleic acid can be produced by ligating the cloned gene, or a portion thereof, into a vector suitable for expression in either prokaryotic cells, eukaryotic cells (yeast, avian, insect or mammalian), or both. Expression vehicles for production of a recombinant polypeptide include plasmids and other vectors. For instance, suitable vectors include plasmids of the types: pBR322-derived plasmids, pEMBL-derived plasmids, pEX-derived plasmids, pBTac-derived plasmids and pUC-derived plasmids for expression in prokaryotic cells, such as E. coli. The preferred mammalian expression vectors contain both prokaryotic sequences to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pHEBo, pREP-derived and p205) can be used for transient expression of proteins in eukaryotic cells. The various methods employed in the preparation of the plasmids and transformation of host organisms are well known in the art. For other suitable expression systems for both prokaryotic and eukaryotic cells, as well as general recombinant procedures, see Molecular Cloning A Laboratory Manual, 2nd Ed., ed. by Sambrook, Fritsch and Maniatis (Cold Spring Harbor Laboratory Press, 1989) Chapters 16 and 17. In some instances, it may be desirable to express the recombinant polypeptide by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the 13-gal containing pBlueBac III).

Techniques for making fusion genes are well known. Essentially, the joining of various DNA fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed to generate a chimeric gene sequence (see, for example, Current Protocols in Molecular Biology, eds. Ausubel et al., John Wiley & Sons: 1992).

The disclosure contemplates methods of producing chimeric proteins recombinantly, such as described above. Suitable vectors and host cells may be readily selected for expression of proteins in, for example, yeast or mammalian cells. Host cells may express a vector encoding a chimeric polypeptide stably or transiently. Such host cells may be cultured under suitable conditions to express chimeric polypeptide which can be readily isolated from the cell culture medium.

Chimeric polypeptides of the disclosure (e.g., polypeptides comprising a mature alpha-amylase portion and an internalizing moiety portion) may be expressed as a single polypeptide chain or as more than one polypeptide chains. An example of a single polypeptide chain is when an alpha-amylase portion is fused inframe to an internalizing moiety, which internalizing moiety is an scFv. In certain embodiments, this single polypeptide chain is expressed from a single vector as a fusion protein.

An example of more than one polypeptide chains is when the internalizing moiety is an antibody or Fab. In certain embodiments, the heavy and light chains of the antibody or Fab may be expressed in a host cell expressing a single vector or two vectors (one expressing the heavy chain and one expressing the light chain). In either case, the alpha-amylase polypeptide may be expressed as an inframe fusion to, for example, the C-terminus of the heavy chain such that the alpha-amylase polypeptide is appended to the internalizing moiety but at a distance to the antigen binding region of the internalizing moiety.

As noted above, methods for recombinantly expressing polypeptides, including chimeric polypeptides, are well known in the art. Nucleotide sequences expressing a mature alpha-amylase polypeptide, such as a human mature alpha-amylase polypeptide, having a particular amino acid sequence are available and can be used. Moreover, nucleotide sequences expressing an internalizing moiety portion, such as expressing a 3E10 antibody, scFv, or Fab comprising the VH and VL set forth in SEQ ID NO: 2 and 3) are publicly available and can be combined with nucleotide sequence encoding suitable heavy and light chain constant regions. The disclosure contemplates nucleotide sequences encoding any of the chimeric polypeptides of the disclosure, vectors (single vector or set of vectors) comprising such nucleotide sequences, host cells comprising such vectors, and methods of culturing such host cells to express chimeric polypeptides of the disclosure.

### V. Methods of Treatment and Other Methods of Use

For any of the methods described herein, the disclosure contemplates the use of any of the chimeric polypeptides and/or compositions described throughout the application. In addition, for any of the methods described herein, the disclosure contemplates the combination of any step or steps of one method with any step or steps from another method.

For example, a chimeric polypeptide of the disclosure comprising a mature alpha-amylase polypeptide (e.g., a mature alpha-amylase polypeptide) portion and an internalizing moiety portion can be used in any of the methods of the disclosure. In other examples, a chimeric polypeptide of the disclosure comprising a mature acid alpha-glucosidase (GAA) portion and an internalizing moiety portion can be used in any of the methods of the disclosure.

In certain embodiments, a chimeric polypeptide of the disclosure (e.g., a polypeptide comprising a mature alpha-amylase polypeptide portion and an internalizing moiety portion or a polypeptide comprising a mature acid alpha-glucosidase portion and an internalizing moiety portion) is delivered to the cytoplasm of cells, such as muscle (e.g., diaphragm muscle, skeletal muscle, and/or cardiac muscle), neuronal cells (e.g., neuronal cells of the brain) and/or liver cells to decrease cytoplasmic glycogen accumulation (e.g., deleterious accumulation of normal of abnormal glycogen, such as polyglucosan). Such cells may be present in vitro or in a subject (e.g., a patient, such as a human) In some embodiments, the subject is a subject having, or suspected of having, a polyglucosan accumulation disease (e.g., a non-central nervous system polyglucosan accumulation disease). In certain embodiments, the subject is a subject having, or suspected of having, a glycogen storage disorder, particularly Danon Disease, Pompe Disease, Adult Polyglucosan Body Disease (APBD), GSD III, GSD IV, GSD V, or GSD XV, and/or a glycogen metabolism disorder, such as GSD VII, Lafora Disease, PRKAG2 associated cardiomyopathy (PAC), or RBCK1 deficiency. In some embodiments, a chimeric polypeptide of the disclosure is suitable for use in delivering alpha-amylase or acid alpha-glucosidase to cells in a subject in need thereof, such as a subject Danon Disease, Pompe Disease, APBD, GSD III, GSD IV, GSD V, GSD XV, GSD VII, Lafora Disease, PRKAG2 associated cardiomyopathy (PAC), or RBCK1 deficiency. In certain embodiments, a chimeric polypeptide of the disclosure is suitable for use in delivering alpha-amylase to cytoplasm in a subject in need thereof, such as a subject having Pompe Disease, GSD III, or GSD IV, and/or a glycogen metabolism disorder, such as Lafora Disease.

In certain embodiments, the subject in need thereof has or is suspected of having GSD III. In certain embodiments, the subject in need thereof has or is suspected of having GSD IV. In certain embodiments, the subject in need thereof has or is suspected of having GSD V. In certain embodiments, the subject in need thereof has or is suspected of having GSDVII. In certain embodiments, the subject in need thereof has or is suspected of having GSD XV. In certain embodiments, the subject in need thereof has or is suspected of having PAC. In certain embodiments, the subject in need thereof has or is suspected of having Alzheimer's Disease and/or dementia. In certain embodiments, the subject in need thereof has or is suspected of having Lafora Disease. In certain embodiments, the subject in need thereof has or is suspected of having Danon Disease.

In certain embodiments, the disclosure provides a method of treating (e.g., improving one or more symptoms of; decreasing glycogen accumulation, such as cytoplasmic glycogen accumulation) GSD III. In certain embodiments, the disclosure provides a method of treating (e.g., improving one or more symptoms of; decreasing glycogen accumulation, such as cytoplasmic glycogen accumulation) GSD IV. In certain embodiments, the disclosure provides a method of treating (e.g., improving one or more symptoms of; decreasing glycogen accumulation) Lafora Disease. In certain embodiments, the disclosure provides a method of treating a disease or disorder associated with hypoxia-induced glycogen accumulation. In some embodiments, the disease or disorder associated with hypoxia-induced glycogen accumulation is cancer. Further methods are described herein.

In some embodiments, any of the chimeric polypeptides disclosed herein may be used to decrease glycogen accumulation in an acidic cellular compartment (e.g., a lysosome or an autophagosome). In some embodiments, the chimeric polypeptides may be used to decrease glycogen accumulation in one or more cells of a patient having a disease associated with glycogen accumulation in acidic cellular compartments (e.g., lysosomes or autophagosomes). In some embodiments, the chimeric polypeptides may be used to decrease glycogen accumulation in a Pompe Disease (GSD II) cell. In some embodiments, the chimeric polypeptides may be used to decrease glycogen accumulation in a Danon Disease (GSD IIb) cell. In some embodiments, the chimeric polypeptides may be used to treat a patient having Pompe Disease (GSD II). In some embodiments, the chimeric polypeptides may be used to treat a patient having Danon Disease (GSD IIb).

In some embodiments, any of the chimeric polypeptides disclosed herein may be used to decrease glycogen accumulation in neuronal cells. In some embodiments, the chimeric polypeptides may be used to decrease glycogen accumulation in one or more cells of a patient having a disease associated with glycogen accumulation in neuronal cells. In some embodiments, the chimeric polypeptides may be used to decrease glycogen accumulation in an Alzheimer's Disease or dementia cell. In some embodiments, the chimeric polypeptides may be used to treat a patient having Alzheimer's Disease or dementia.

In some embodiments, the chimeric polypeptides of the disclosure may be used to increase glycogen clearance in a cell. In some embodiments, the cell is a muscle (e.g., cardiac or diaphragm muscle), liver or neuronal (e.g., of the brain) cell. In some embodiments, the cell is in a subject having Danon Disease and/or Alzheimer's Disease.

In certain embodiments, chimeric polypeptides comprising any of the alpha-amylase polypeptides or acid alpha-glucosidase polypeptides disclosed herein can be used to treat Danon Disease. In certain embodiments, chimeric polypeptides comprising any of the alpha-amylase polypeptides disclosed herein can be used to treat Alzheimer's Disease and/or dementia. In certain embodiments, the present disclosure provides methods of delivering chimeric polypeptides to cells, including cells in culture (in vitro or ex vivo) and cells in a subject. Delivery to cells in culture, such as healthy cells or cells from a model of disease, have numerous uses. These uses include to identify alpha-amylase substrates or binding partners, to evaluate localization and/or trafficking (e.g., to cytoplasm, lysosome, and/or autophagic vesicles), to evaluate enzymatic activity under a variety of conditions (e.g., pH), to assess glycogen accumulation, and the like. In certain embodiments, chimeric polypeptides of the disclosure can be used as reagents to understand alpha-amylase activity, localization, and trafficking in healthy or disease contexts.

Delivery to subjects, such as to cells in a subject, has numerous uses. Exemplary therapeutic uses are described below. Moreover, the chimeric polypeptides may be used for diagnostic or research purposes. For example, a chimeric polypeptide of the disclosure may be detectably labeled and administered to a subject, such as an animal model of disease or a patient, and used to image the chimeric polypeptide in the subject's tissues (e.g., localization to muscle, brain and/or liver). Additionally exemplary uses include delivery to cells in a subject, such as to an animal model of disease (e.g., Forbes-Cori and/or Andersen Disease and/or Pompe Disease and/or von Gierke Disease and/or Lafora Disease and/or Danon Disease and/or Alzheimer's Disease). By way of example, chimeric polypeptides of the disclosure may be used as reagents and delivered to animals to understand alpha-amylase bioactivity, localization and trafficking, protein-protein interactions, enzymatic activity, and impacts on animal physiology in healthy or diseased animals.

In certain embodiments, the present disclosure provides methods of treating conditions associated with, dysfunction of laforin, alpha-amylase, and/or malin, with aberrant glycogen accumulation and/or with Forbes-Cori, Pompe Disease, von Gierke Disease, Lafora Disease, Andersen Disease, Danon Disease, and/or Alzheimer's Disease. In certain embodiments, the glycogen accumulation is in the cytoplasm, and delivery of alpha-amylase reduces cytoplasmic glycogen accumulation, such as in cardiac muscle or neuronal cells. In certain embodiments, the subject does not have dysfunction in endogenous laforin, alpha-amylase, and/or malin (e.g., the methods do not comprise replacement of the protein that is mutated or for which there is dysfunction).

In certain embodiments, these methods involve administering to the individual a therapeutically effective amount of a chimeric polypeptide as described above (e.g., a chimeric polypeptide comprising (i) an alpha-amylase polypeptide and (ii) an internalizing moiety portion). These methods are particularly aimed at therapeutic and prophylactic treatments of animals, and more particularly, humans. With respect to methods for treating Forbes-Cori and/or Andersen Disease and/or Pompe Disease and/or von Gierke Disease and/or Lafora Disease and/or Danon Disease and/or Alzheimer's Disease, the disclosure contemplates all combinations of any of the foregoing aspects and embodiments, as well as combinations with any of the embodiments set forth in the detailed description and examples. Accordingly, chimeric polypeptides of the disclosure are, in certain embodiments, suitable for treating diseases such as Forbes-Cori and/or Andersen Disease and/or Pompe Disease and/or von Gierke Disease and/or Lafora Disease and/or Danon Disease and/or Alzheimer's Disease. In certain embodiments, the chimeric polypeptide decreases glycogen accumulation in cells, such as muscle cells (e.g., diaphragm muscle or cardiac muscle cells), liver cells, and/or neuronal cells, to treat Forbes-Cori and/or Andersen Disease and/or Pompe Disease and/or von Gierke Disease and/or Lafora Disease and/or Danon Disease and/or Alzheimer's Disease in a patient in need thereof.

The present disclosure provides a method of delivering a chimeric polypeptide or nucleic acid construct into a cell via an equilibrative nucleoside transporter (ENT2) pathway, comprising contacting a cell with a chimeric polypeptide or nucleic acid construct. In certain embodiments, the method comprises contacting a cell with a chimeric polypeptide, which chimeric polypeptide comprises an alpha-amylase polypeptide or bioactive fragment thereof, or an acid alpha-glucosidase polypeptide or bioactive fragment thereof, and an internalizing moiety which can mediate transport across a cellular membrane via an ENT2 pathway (and optionally via another ENT transporter, such as ENT3), thereby delivering the chimeric polypeptide into the cell. In certain embodiments, the cell is a muscle cell. The muscle cells targeted using any of the methods disclosed herein may include skeletal (e.g., diaphragm), cardiac or smooth muscle cells. In other embodiments, the chimeric polypeptides are delivered to liver or neuronal (e.g., brain) cells.

The present disclosure also provides a method of delivering a chimeric polypeptide or nucleic acid construct into a cell via a pathway that allows access to cells other than muscle cells. Other cell types that could be targeted using any of the methods disclosed herein include, for example, liver cells, neurons (e.g., of the brain), epithelial cells, uterine cells, and kidney cells.

In certain embodiments, the internalizing moiety is an antibody or antigen binding fragment, such as an antibody or antigen binding fragment that binds DNA. In certain embodiments, the internalizing moiety is an antibody, such as a full length antibody or a Fab. In certain embodiments, the full length antibody or Fab comprises one or more substitutions, relative to a native immunoglobulin constant region, such as to decrease effector function.

Forbes-Cori Disease, also known as Glycogen Storage Disease Type III, GSD III, or limit dextrinosis, is an autosomal recessive neuromuscular/hepatic disease with an estimated incidence of 1 in 83,000-100,000 live births. Forbes-Cori Disease represents approximately 24% of all Glycogen Storage Disorders. The clinical picture in Forbes-Cori Disease is reasonably well established but variable. Forbes-Cori Disease patients may suffer from skeletal myopathy, cardiomyopathy, cirrhosis of the liver, hepatomegaly, hypoglycemia, short stature, dyslipidemia, slight mental retardation, facial abnormalities, and/or increased risk of osteoporosis (Ozen et al., 2007, World J Gastroenterol, 13(18): 2545-46). Forms of Forbes-Cori Disease with muscle involvement may present muscle weakness, fatigue and muscle atrophy. Progressive muscle weakness and distal muscle wasting frequently become disabling as the patients enter the third or fourth decade of life, although this condition has been reported to begin in childhood in many Japanese patients.

Andersen Disease, also known as Glycogen Storage Disease Type IV or GSD IV, is also an autosomal recessive neuromuscular/hepatic disease with an estimated incidence of 1 in 600,000 to 800,000 individuals worldwide. The age of onset ranges from fetus to adulthood and is divided into four groups: (i) perinatal, presenting as fetal akinesia deformation sequence and perinatal death; (ii) congenital, with hydrops fetalis, neuronal involvement and death in early infancy; (iii) childhood, with myopathy or cardiomyopathy; and (iv) adult, with isolated myopathy or adult polyglucosan body disease (Lee, et al., 2010). Absence of enzyme activity is usually lethal in utero or in infancy, affecting primarily muscle and liver. However, residual enzyme activity (5-20%) leads to a juvenile or adult-onset disorder that affects primarily muscle and both central and peripheral nervous systems. Symptoms observed in Andersen Disease patients include liver dysfunction, arthrogryposis, neuronal dysfunction, failure to thrive, cirrhosis, portal vein hypertension, esophageal varices, ascites, hepatosplenomegaly, portal hypertension, hypotonia, myopathy, dilated cardiomyopathy, and shortened life expectancy. These symptoms may vary in severity depending on the type of Andersen Disease affecting the subject.

Glycogen storage disease type I (GSD I) or von Gierke Disease, is the most common of the glycogen storage diseases with an incidence of approximately 1 in 50,000 to 100,000 births. The deficiency impairs the ability of the liver to produce free glucose from glycogen and from gluconeogenesis, causes severe hypoglycemia and results in increased glycogen storage in liver and kidneys. This can lead to enlargement of both organs.

The most common forms of GSD I are designated GSD Ia and GSD Ib, the former accounting for over 80% of diagnosed cases and the latter for less than 20%. A few rarer forms have been described. GSD Ia results from mutations of G6PC, the gene for glucose-6-phosphatase. GSD Ib results from mutations of the SLC37A4, the glucose-6-phosphatase transporter. In certain embodiments, patients in need of treatment with the subject methods are patient having GSD Ia. In other embodiments, patients in need of treatment are patients having GSD Ib.

Clinical manifestations in von Gierke Disease result, directly or indirectly, from: the inability to maintain an adequate blood glucose level during the post-absorptive hours of each day; organ changes due to glycogen accumulation; excessive lactic acid generation; and damage to tissue from hyperuricemia. Glycogen accumulation includes accumulation in the liver and in the kidneys and small intestines. Hepatomegaly, usually without splenomegaly, begins to develop in fetal life and is usually noticeable in the first few months of life. By the time the child is standing and walking, the hepatomegaly may be severe enough to cause the abdomen to protrude.

The kidneys of von Gierke Disease patients are usually 10 to 20% enlarged with stored glycogen. This does not usually cause clinical problems in childhood, with the occasional exception of a Fanconi syndrome with multiple derangements of renal tubular reabsorption, including proximal renal tubular acidosis with bicarbonate and phosphate wasting. However, prolonged hyperuricemia can cause uric acid nephropathy. In adults with GSD I, chronic glomerular damage similar to diabetic nephropathy may lead to renal failure.

Hepatic complications have been serious in some von Gierke Disease patients. Adenomas of the liver can develop in the second decade or later, with a small chance of later malignant transformation to hepatoma or hepatic carcinomas. Additional problems reported in adolescents and adults with GSD I have included hyperuricemic gout, pancreatitis, and chronic renal failure.

Glycogen storage disease type VII (GSD VII) results from mutations in PFKM (the muscle isoform of phosphofructokinase). GSD VII is an autosomal recessive disorder with broad, age-related phenotypic variability, ranging from a severe, fatal infantile type with myopathy and cardiomyopathy; a classic childhood type with muscle pain and cramping and rhabdomyolysis; a late onset myopathy with exercise intolerance and a hemolytic anemia without muscle involvement.

Glycogen storage disease type XV (GSD XV) results from mutations in GYG1 (the gene for glycogenin). GSD XV is an autosomal dominant disorder that includes a spectrum of phenotypes spanning pure skeletal myopathy to pure cardiomyopathy with cardiac failure. Onset is typically in the fifth decade of life or later, but can occur earlier.

RBCK1 deficiency is an autosomal recessive disorder with moderate phenotypic variability. Mutations in the N-terminal portion of the protein result primarily in immunological defects, while those in the mid-portion and C-terminal portion of the protein result in myopathy, generally starting in childhood or early adolescence with a later onset of cardiomyopathy. Missense mutations are generally limited to myopathy, whereas truncating mutations are associated with both myopathy and a progressive dilated cardiomyopathy frequently requiring transplantation.

PRKAG2 associated cardiomyopathy (PAC) is one of the most common polyglucosan accumulation diseases, occurring in approximately 1% of patients with hypertrophic cardiomyopathy, and is among the least variable, phenotypically. PAC is an autosomal dominant, largely heart-specific, non-lysosomal glycogenosis generally presenting in adolescence or later, but occasionally presenting in infancy. PAC is characterized by accumulation of polyglucosan bodies in the heart in association with cardiac hypertrophy, atrioventricular accessory pathways, and conduction system abnormalities. These features frequently lead to cardiac failure and ventricular pre-excitation with a high incidence of arrhythmias and sudden dead, necessitating the placement of a pacemaker or defibrillator. Skeletal muscle involvement is uncommon. PRKAG2 encodes the γ2 regulatory subunit of adenosine monophosphate-activated protein kinase (AMPK) which regulates glucose and fatty acid metabolic pathways. Studies in transgenic mouse models of PAC suggest that depletion of accumulated polyglucosan can restore normal electrophysiologic function and possible reduce cardiomegaly.

Lafora Disease, also called Lafora progressive myoclonic epilepsy or MELF, is a rare, fatal neurodegenerative disorder characterized by the accumulation of cytoplasmic polyglucosan inclusion bodies (known as Lafora bodies) in cells from most tissues of affected individuals, including the brain, heart, liver, muscle and skin. Lafora Disease patients typically first develop symptoms in adolescence. Symptoms include temporary blindness, depression, seizures, drop attacks, myoclonus, visual hallucinations, absences, ataxia and quickly developing and severe dementia. Death usually occurs 2-10 years (5 years mean) after onset.

The prevalence of Lafora Disease is unknown. While this disease occurs worldwide, it is most common in Mediterranean countries, parts of Central Asia, India, Pakistan, North Africa and the Middle East. In Western countries, the prevalence is estimated to be below 1/1,000,000.

There is currently no cure or effective treatment for patients having Lafora Disease. However, the seizures and myoclonus can be managed, at least in early stages of the disease, with antiepileptic medications.

Danon Disease or Glycogen Storage Disease IIb is a rare metabolic disorder associated with hypertrophic cardiomyopathy, skeletal muscle weakness, and intellectual disability. Cardiomyopathy may be severe and eventually lead to heart failure. In addition, the cardiomyopathy may be associated with atrial fibrillation and embolic strokes. Danon Disease involves a genetic defect in LAMP2, which results in a change to the normal protein structure. The symptoms of Danon Disease are generally more severe in men than in women.

Neuronal disorders or diseases, including Alzheimer's Disease and/or dementia, may be characterized by the accumulation of glycogen in cells (e.g., neuronal cells) from the brain tissue of affected individuals Alzheimer's Disease is a chronic neurodegenerative disease that usually starts slowly and worsens over time. It is the cause of 60% to 70% of dementia cases, with the most common early symptom being short-term memory loss. Alzheimer's Disease patients may suffer from language problems, disorientation, mood swings, loss of motivation, lack of self-care, and behavioral issues. The cause of Alzheimer's is unclear with multiple hypotheses existing to explain the cause, including a genetic hypothesis, a cholinergic hypothesis, an amyloid hypothesis, and a tau hypothesis, among others Alzheimer's Disease may be characterized by the build-up of beta-amyloid peptides causing neuron degeneration. Beta-amyloids that build up in the mitochondria in the cells may inhibit certain enzyme functions, as well as the utilization of glucose by neurons.

Dementia can refer to a broad category of brain diseases that may be associated with Alzheimer's, as well as with vascular dementia, Lewy body dementia, frontotemporal dementia, Parkinson's Disease, syphilis, Creutzfeldt-Jakob disease, and normal pressure hydrocephalus, among others. Dementia patients may experience a long-term and generally gradual decrease in the ability to think clearly and remembering daily details. Dementia affects about 46 million people, and about 10% of people will develop the disorder at some point during their lives. The disease becomes more common as an individual ages, with about 3% of people between the ages of 65-74 having dementia, while about 19% of people between the ages of 75 and 84 have dementia.

The terms “treatment”, “treating”, and the like are used herein to generally mean obtaining a desired pharmacologic and/or physiologic effect. “Treating” a condition or disease refers to curing as well as ameliorating at least one symptom of the condition or disease, and includes administration of a composition which reduces the frequency of, or delays the onset of, symptoms of a medical condition in a subject in need relative to a subject which does not receive the composition. “Treatment” as used herein covers any treatment of a disease or condition of a mammal, particularly a human, and includes: (a) preventing symptoms of the disease or condition from occurring in a subject which may be predisposed to the disease or condition but has not yet begun experiencing symptoms; (b) inhibiting the disease or condition (e.g., arresting its development); or (c) relieving the disease or condition (e.g., causing regression of the disease or condition, providing improvement in one or more symptoms). For example, “treatment” of Forbes-Cori, Pompe Disease, Andersen Disease, Danon Disease, and/or Alzheimer's Disease is contemplated and encompasses a complete reversal or cure of the disease, or any range of improvement in symptoms and/or adverse effects attributable to the disease.

Merely to illustrate, “treatment” of Forbes-Cori Disease includes an improvement in any of the following effects associated with Forbes-Cori Disease or combination thereof: skeletal myopathy, cardiomyopathy, cirrhosis of the liver, hepatomegaly, hypoglycemia, short stature, dyslipidemia, failure to thrive, mental retardation, facial abnormalities, osteoporosis, muscle weakness, fatigue and muscle atrophy. Treatment may also include one or more of reduction of abnormal levels of cytoplasmic glycogen, decrease in elevated levels of one or more of alanine transaminase, aspartate transaminase, alkaline phosphatase, or creatine phosphokinase, such as decrease in such levels in serum. Improvements in any of these conditions can be readily assessed according to standard methods and techniques known in the art. Other symptoms not listed above may also be monitored in order to determine the effectiveness of treating Forbes-Cori Disease. The population of subjects treated by the method of the disclosure includes subjects suffering from the undesirable condition or disease, as well as subjects at risk for development of the condition or disease.

Merely to illustrate, “treatment” of Andersen Disease includes an improvement in any of the following effects associated with Andersen Disease or combination thereof: liver dysfunction, arthrogryposis, neuronal dysfunction, failure to thrive, cirrhosis, portal vein hypertension, esophageal varices, ascites, hepatosplenomegaly, portal hypertension, hypotonia, myopathy, dilated cardiomyopathy, and shortened life expectancy. Treatment may also include one or more of reduction of abnormal levels of cytoplasmic glycogen. Other symptoms not listed above may also be monitored in order to determine the effectiveness of treating Andersen Disease. The population of subjects treated by the method of the disclosure includes subjects suffering from the undesirable condition or disease, as well as subjects at risk for development of the condition or disease.

In certain embodiments, the subjects in need of treatment are subjects having the perinatal form of Andersen Disease (e.g., perinatal form of GSD IV). In other embodiments, the subjects in need of treatment are subjects having the congenital (infantile) form of Andersen Disease. In other embodiments, the subjects in need of treatment are subjects having the childhood (juvenile) form of Andersen Disease. In some embodiments, the subjects in need thereof are subjects having the adult form of Andersen Disease. Thus, in certain embodiments, the disclosure provides methods of treating any of the foregoing patients by administering a chimeric polypeptide of the disclosure. In certain embodiments, the disclosure provides methods of decreasing cytoplasmic glycogen accumulation, such as in skeletal muscle, cardiac muscle, and/or liver, in any of the foregoing subjects in need by administering a chimeric polypeptide of the disclosure.

Merely to illustrate, “treatment” of Pompe Disease includes an improvement in any of the following effects associated with dysfunction of alpha-amylase (or combination thereof): decreased alpha amylase activity (e.g., treatment increases alpha amylase activity), glycogen accumulation in cells (e.g., treatment decreases glycogen accumulation), increased creatine kinase levels, elevation of urinary glucose tetrasaccharide, heart size, hypertrophic cardiomyopathy, respiratory complications, dependence on a ventilator, muscle dysfunction and/or weakening, loss of motor function, dependence on a wheelchair or other form of mobility assistance, dependence on neck or abdominal support for sitting upright, ultrastructural damage of muscle fibers, loss of muscle tone and function. Improvements in any of these symptoms can be readily assessed according to standard methods and techniques known in the art. Other symptoms not listed above may also be monitored in order to determine the effectiveness of treating Pompe Disease.

In certain embodiments, the subjects in need of treatment are subjects having infantile form of Pompe Disease. In other embodiments, the subjects in need of treatment are subjects having juvenile onset or adult onset Pompe Disease. Thus, in certain embodiments, the disclosure provides methods of treating any of the foregoing patients by administering a chimeric polypeptide of the disclosure. In certain embodiments, the disclosure provides methods of decreasing cytoplasmic glycogen accumulation, such as in skeletal muscle, cardiac muscle, and/or liver, in any of the foregoing subjects in need by administering a chimeric polypeptide of the disclosure.

Merely to illustrate, “treatment” of von Gierke Disease includes an improvement in any of the following effects associated with von Gierke Disease or combination thereof: constant hunger, easy bruising and nosebleeds, fatigue, irritability, puffy cheeks, thin chest and limbs, swollen belly, delayed puberty, enlarged liver, gout, inflammatory bowel disease, kidney disease, kidney failure, osteoporosis, seizures, lethargy, short height, ulcers of mouth, ulcers of the bowel, liver tumors, hypoglycemia, arthritis, stunted growth, pulmonary hypertension, and/or failure to grow. Other symptoms not listed above may also be monitored in order to determine the effectiveness of treating von Gierke Disease. The population of subjects treated by the method of the disclosure includes subjects suffering from the undesirable condition or disease, as well as subjects at risk for development of the condition or disease. In certain embodiments, the subject being treated is an adolescent and is treated before the onset of puberty.

Merely to illustrate, “treatment” of Lafora Disease includes an improvement in any of the following effects associated with Lafora Disease or combination thereof: blindness, depression, seizures, drop attacks, hepatic disease, muscle atrophy, myoclonus, visual hallucinations, absences, ataxia, dementia, and/or shortened lifespan. Treatment may also include a reduction of Lafora bodies and or aberrant accumulation of polyglucosan in, for example, muscle (e.g., cardiac or diaphragm), liver and/or brain. Other symptoms not listed above may also be monitored in order to determine the effectiveness of treating Lafora Disease. The population of subjects treated by the method of the disclosure includes subjects suffering from the undesirable condition or disease, as well as subjects at risk for development of the condition or disease. In certain embodiments, the subject being treated is treated before onset of dementia or before onset of measureable, appreciable dementia.

Merely to illustrate, “treatment” of Danon Disease includes an improvement in any of the following effects associated with dysfunction of alpha-amylase (or combination thereof): decreased alpha amylase activity (e.g., treatment increases alpha amylase activity), glycogen accumulation in cells (e.g., treatment decreases glycogen accumulation), increased creatine kinase levels, heart size, hypertrophic cardiomyopathy, respiratory complications, dependence on a ventilator, muscle dysfunction and/or weakening, loss of motor function, dependence on a wheelchair or other form of mobility assistance, dependence on neck or abdominal support for sitting upright, ultrastructural damage of muscle fibers, loss of muscle tone and function. Improvements in any of these symptoms can be readily assessed according to standard methods and techniques known in the art. Other symptoms not listed above may also be monitored in order to determine the effectiveness of treating Danon Disease.

Merely to illustrate, “treatment” of a neuronal disease (e g, Alzheimer's Disease or dementia) includes an improvement in any of the following effects associated with dysfunction of alpha-amylase (or combination thereof): decreased alpha amylase activity (e.g., treatment increases alpha amylase activity), decreased glycogen accumulation in cells (e.g., treatment decreases glycogen accumulation), decreased glycogen uptake by neuronal cells. Improvements in any of these symptoms can be readily assessed according to standard methods and techniques known in the art. Other symptoms not listed above may also be monitored in order to determine the effectiveness of treating Alzheimer's Disease and/or dementia.

In certain embodiments, the disclosure provides methods of delivering alpha-amylase activity to cells, such as muscle and/or liver and/or kidney and/or neuronal cells of a subject having Forbes Cori Disease, Andersen Disease, Pompe Disease, von Gierke Disease, Lafora Disease, Danon Disease, or Alzheimer's Disease comprising administering a chimeric polypeptide of the disclosure or a composition comprising a chimeric polypeptide of the disclosure formulated with one or more pharmaceutically acceptable carriers and/or excipients.

By the term “therapeutically effective dose” is meant a dose that produces the desired effect for which it is administered. The exact dose will depend on the purpose of the treatment, and will be ascertainable by one skilled in the art using known techniques (see, e.g., Lloyd (1999) The Art, Science and Technology of Pharmaceutical Compounding).

In certain embodiments, administration of a chimeric polypeptide of the disclosure is via any one of the routes of administration described herein, such as subcutaneous, intravenous, or via the hepatic portal vein. In other words, the disclosure contemplates methods of delivery by administering via any such route of administration.

In certain embodiments, the method results in delivery of greater alpha-amylase activity to the cytoplasm, in comparison, to that following deliver of an alpha-amylase polypeptide that is not conjugated to an internalizing moiety and/or in comparison to that of an alpha-amylase polypeptide conjugated to a different internalizing moiety.

In certain embodiments, one or more chimeric polypeptides of the present disclosure can be administered, together (simultaneously) or at different times (sequentially). In addition, chimeric polypeptides of the present disclosure can be administered alone or in combination with one or more additional compounds or therapies for treating Pompe Disease and/or Forbes-Cori Disease and/or von Gierke Disease and/or Lafora Disease and/or Andersen Disease and/or Danon Disease and/or Alzheimer's Disease. For example, one or more chimeric polypeptides can be co-administered in conjunction with one or more other therapeutic compounds. When co-administration is indicated, the combination therapy may encompass simultaneous or alternating administration. In addition, the combination may encompass acute or chronic administration. Optionally, the chimeric polypeptide of the present disclosure and additional compounds act in an additive or synergistic manner for treating Lafora Disease. Additional compounds to be used in combination therapies include, but are not limited to, small molecules, polypeptides, antibodies, antisense oligonucleotides, and siRNA molecules. Depending on the nature of the combinatory therapy, administration of the chimeric polypeptides of the disclosure may be continued while the other therapy is being administered and/or thereafter. Administration of the chimeric polypeptides may be made in a single dose, or in multiple doses. In some instances, administration of the chimeric polypeptides is commenced at least several days prior to the other therapy, while in other instances, administration is begun either immediately before or at the time of the administration of the other therapy.

In some embodiments, any of the chimeric polypeptides described herein are administered to a subject in combination with an anti-epileptic drug. In some embodiments, any of the chimeric polypeptides described herein are administered to a subject in combination with any of the chimeric polypeptides disclosed in WO 2015/192092, which is incorporated by reference in its entirety. In particular embodiments, any of the chimeric polypeptides described herein are administered to a subject in combination with any of the malin and/or laforin chimeric polypeptides disclosed in WO 2015/192092.

One type of combination therapy makes use of molecules that promote muscle synthesis and/or fat reduction. Molecules such as IGF-1, growth hormones, steroids, β-2 agonists (for example Clenbuterol), and myostatin inhibitors may be administered to patients in order to build muscle tissue and reduce fat infiltration. These molecules may also increase ENT2 levels. Accordingly, the molecules may be administered before treatment with a chimeric polypeptide of the disclosure begins, in between treatments, or after treatment with a chimeric polypeptide of the disclosure.

In another example of combination therapy, one or more chimeric polypeptides of the disclosure can be used as part of a therapeutic regimen combined with one or more additional treatment modalities. By way of example, such other treatment modalities include, but are not limited to, dietary therapy, occupational therapy, physical therapy, ventilator supportive therapy, massage, acupuncture, acupressure, mobility aids, assistance animals, and the like.

In certain embodiments, one or more chimeric polypeptides of the present disclosure can be administered prior to or following a liver transplant.

Note that although the chimeric polypeptides described herein can be used in combination with other therapies, in certain embodiments, a chimeric polypeptide is provided as the sole form of therapy. Regardless of whether administrated alone or in combination with other medications or therapeutic regiments, the dosage, frequency, route of administration, and timing of administration of the chimeric polypeptides is determined by a physician based on the condition and needs of the patient. The disclosure contemplates that a method may comprise administration at a dose and on a dosing schedule, such as administration at specified intervals over a period of time. In such cases, each dose contributes to efficacy, and is thus effective, although improvement in symptoms may only be observed after administration of multiple doses.

Chimeric polypeptides of the disclosure have numerous uses, including in vitro and in vivo uses. In vivo uses include not only therapeutic uses but also diagnostic and research uses in, for example, any of the foregoing animal models. By way of example, chimeric polypeptides of the disclosure may be used as research reagents and delivered to animals to understand alpha-amylase bioactivity, localization and trafficking, protein-protein interactions, enzymatic activity, and impacts on animal physiology in healthy or diseases animals.

Chimeric polypeptides may also be used in vitro to evaluate, for example, alpha-amylase bioactivity, localization and trafficking, protein-protein interactions, and enzymatic activity in cells in culture, including healthy and alpha-amylase deficient cells in culture. The disclosure contemplates that chimeric polypeptides of the disclosure may be used to deliver alpha-amylase to cytoplasm, lysosome, and/or autophagic vesicles of cells, including cells in culture.

The disclosure contemplates that any of the methods described herein may be carried out by administering or contacting cells with a chimeric polypeptide of the disclosure and/or a composition of the disclosure (e.g., a composition comprising a chimeric polypeptide of the disclosure formulated with one or more pharmaceutically acceptable carriers and/or excipients).

### VI. Gene Therapy

Conventional viral and non-viral based gene transfer methods can be used to introduce nucleic acids encoding polypeptides of alpha-amylase or acid alpha-glucosidase (e.g., a mature alpha-amylase or a mature acid alpha-glucosidase) and or chimeric polypeptides comprising alpha-amylase or acid alpha-glucosidase in mammalian cells or target tissues. Such methods can be used to administer nucleic acids encoding polypeptides of the disclosure (e.g., alpha-amylase including variants thereof, and include chimeric polypeptides) to cells in vitro. The disclosure contemplates that gene transfer methods may be used to deliver nucleic acid encoding any of the chimeric polypeptides of the disclosure or alpha-amylase polypeptides. In some embodiments, the nucleic acids encoding alpha-amylase are administered for in vivo or ex vivo gene therapy uses. In other embodiments, gene delivery techniques are used to study the activity of chimeric polypeptides or alpha-amylase polypeptide or to study Lafora Disease in cell based or animal models, such as to evaluate cell trafficking, enzyme activity, and protein-protein interactions following delivery to healthy or diseased cells and tissues. Non-viral vector delivery systems include DNA plasmids, naked nucleic acid, and nucleic acid complexed with a delivery vehicle such as a liposome. Viral vector delivery systems include DNA and RNA viruses, which have either episomal or integrated genomes after delivery to the cell. Such methods are well known in the art.

Methods of non-viral delivery of nucleic acids encoding engineered polypeptides of the disclosure include lipofection, microinjection, biolistics, virosomes, liposomes, immunoliposomes, polycation or lipid:nucleic acid conjugates, naked DNA, artificial virions, and agent-enhanced uptake of DNA. Lipofection methods and lipofection reagents are well known in the art (e.g., Transfectam™ and Lipofectin™). Cationic and neutral lipids that are suitable for efficient receptor-recognition lipofection of polynucleotides include those of Feigner, WO 91/17424, WO 91/16024. Delivery can be to cells (ex vivo administration) or target tissues (in vivo administration). The preparation of lipid:nucleic acid complexes, including targeted liposomes such as immunolipid complexes, is well known to one of skill in the art.

The use of RNA or DNA viral based systems for the delivery of nucleic acids encoding alpha-amylase or its variants take advantage of highly evolved processes for targeting a virus to specific cells in the body and trafficking the viral payload to the nucleus. Viral vectors can be administered directly to patients (in vivo) or they can be used to treat cells in vitro and the modified cells are administered to patients (ex vivo). Conventional viral based systems for the delivery of polypeptides of the disclosure could include retroviral, lentivirus, adenoviral, adeno-associated and herpes simplex virus vectors for gene transfer. Viral vectors are currently the most efficient and versatile method of gene transfer in target cells and tissues. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immuno deficiency virus (SW), human immuno deficiency virus (HIV), and combinations thereof, all of which are well known in the art.

In applications where transient expression of the polypeptides of the disclosure is preferred, adenoviral based systems are typically used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors are also used to transduce cells with target nucleic acids, e.g., in the in vitro production of nucleic acids and peptides, and for in vivo and ex vivo gene therapy procedures. Construction of recombinant AAV vectors are described in a number of publications, including U.S. Pat. No. 5,173,414; Tratschin et al., Mol. Cell. Biol. 5:3251-3260 (1985); Tratschin, et al.; Mol. Cell. Biol. 4:2072-2081 (1984); Hermonat & Muzyczka, PNAS 81:6466-6470 (1984); and Samulski et al., J. Virol. 63:03822-3828 (1989).

Recombinant adeno-associated virus vectors (rAAV) are a promising alternative gene delivery systems based on the defective and nonpathogenic parvovirus adeno-associated type 2 virus. All vectors are derived from a plasmid that retains only the AAV 145 bp inverted terminal repeats flanking the transgene expression cassette. Efficient gene transfer and stable transgene delivery due to integration into the genomes of the transduced cell are key features for this vector system.

Replication-deficient recombinant adenoviral vectors (Ad) can be engineered such that a transgene replaces the Ad E1a, E1b, and E3 genes; subsequently the replication defector vector is propagated in human 293 cells that supply deleted gene function in trans. Ad vectors can transduce multiple types of tissues in vivo, including nondividing, differentiated cells such as those found in the liver, kidney and muscle system tissues. Conventional Ad vectors have a large carrying capacity.

Packaging cells are used to form virus particles that are capable of infecting a host cell. Such cells include 293 cells, which package adenovirus, and 42 cells or PA317 cells, which package retrovirus. Viral vectors used in gene therapy are usually generated by producer cell line that packages a nucleic acid vector into a viral particle. The vectors typically contain the minimal viral sequences required for packaging and subsequent integration into a host, other viral sequences being replaced by an expression cassette for the protein to be expressed. The missing viral functions are supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV.

In many gene therapy applications, it is desirable that the gene therapy vector be delivered with a high degree of specificity to a particular tissue type. A viral vector is typically modified to have specificity for a given cell type by expressing a ligand as a fusion protein with a viral coat protein on the viruses outer surface. The ligand is chosen to have affinity for a receptor known to be present on the cell type of interest. This principle can be extended to other pairs of virus expressing a ligand fusion protein and target cell expressing a receptor. For example, filamentous phage can be engineered to display antibody fragments (e.g., FAB or Fv) having specific binding affinity for virtually any chosen cellular receptor. Although the above description applies primarily to viral vectors, the same principles can be applied to nonviral vectors. Such vectors can be engineered to contain specific uptake sequences thought to favor uptake by specific target cells, such as muscle cells.

Gene therapy vectors can be delivered in vivo by administration to an individual patient, by systemic administration (e.g., intravenous, intraperitoneal, intramuscular, subdermal, or intracranial infusion) or topical application. Alternatively, vectors can be delivered to cells ex vivo, such as cells explanted from an individual patient (e.g., lymphocytes, bone marrow aspirates, tissue biopsy) or universal donor hematopoietic stem cells, followed by reimplantation of the cells into a patient, usually after selection for cells which have incorporated the vector.

Ex vivo cell transfection for diagnostics, research, or for gene therapy (e.g., via re-infusion of the transfected cells into the host organism) is well known to those of skill in the art. For example, cells are isolated from the subject organism, transfected with a nucleic acid (gene or cDNA) encoding, e.g., alpha-amylase or its variants, and re-infused back into the subject organism (e.g., patient). Various cell types suitable for ex vivo transfection are well known to those of skill in the art.

In certain embodiments, stem cells are used in ex vivo procedures for cell transfection and gene therapy. The advantage to using stem cells is that they can be differentiated into other cell types in vitro, or can be introduced into a mammal (such as the donor of the cells) where they will engraft in the bone marrow. Stem cells are isolated for transduction and differentiation using known methods.

Vectors (e.g., retroviruses, adenoviruses, liposomes, etc.) containing therapeutic nucleic acids can be also administered directly to the organism for transduction of cells in vivo. Alternatively, naked DNA can be administered. Administration is by any of the routes normally used for introducing a molecule into ultimate contact with blood or tissue cells. Suitable methods of administering such nucleic acids are available and well known to those of skill in the art, and, although more than one route can be used to administer a particular composition, a particular route can often provide a more immediate and more effective reaction than another route.

Pharmaceutically acceptable carriers are determined in part by the particular composition being administered, as well as by the particular method used to administer the composition. Accordingly, there is a wide variety of suitable formulations of pharmaceutical compositions of the present disclosure, as described herein.

### VII. Methods of Administration

Various delivery systems are known and can be used to administer the chimeric polypeptides of the disclosure. Any such methods may be used to administer any of the chimeric polypeptides described herein. The disclosure contemplates than any of the methods of administration disclosed herein may be used to deliver any of the chimeric polypeptides of the disclosure in the context of any of the methods described herein (e.g., methods of treatment; methods of reducing cytoplasmic glycogen accumulation).

Methods of introduction can be enteral or parenteral, including but not limited to, intradermal, intramuscular, intraperitoneal, intramyocardial, intravenous, subcutaneous, pulmonary, intranasal, intraocular, epidural, intrathecal, intracranial, intraventricular (e.g., intracerebroventricular) and oral routes. The chimeric polypeptides may be administered by any convenient route, for example, by infusion or bolus injection, by absorption through epithelial or mucocutaneous linings (e.g., oral mucosa, rectal and intestinal mucosa, etc.) and may be administered together with other biologically active agents. Administration can be systemic or local.

In certain embodiments, the chimeric polypeptide is administered intravenously.

In certain embodiments, it may be desirable to administer the chimeric polypeptides of the disclosure locally to the area in need of treatment (e.g., muscle); this may be achieved, for example, and not by way of limitation, by local infusion during surgery, by means of a catheter, or by means of an implant, the implant being of a porous, non-porous, or gelatinous material, including membranes, such as sialastic membranes, fibers, or commercial skin substitutes.

In another embodiment, such local administration can be to all or a portion of the heart. For example, administration can be by intrapericardial or intramyocardial administration. Similarly, administration to cardiac tissue can be achieved using a catheter, wire, and the like intended for delivery of agents to various regions of the heart.

In another embodiment, local administration is directed to the liver. Glycogen storage and glycogenolysis in the liver affect the availability of glycogen for many other tissues in the body. For example, a venous catheter may be placed in the hepatic portal vein to deliver chimeric polypeptides directly to the liver. In addition, in some embodiments where the internalizing moieties of the chimeric polypeptides show a lower affinity for liver cells than for other cell types, delivery through the hepatic portal vein ensures that adequate concentrations of alpha-amylase reach the liver cells.

Note that the disclosure contemplates methods in which chimeric polypeptides are administered, at the same or different times, via one than one route of administration. For example, the disclosure contemplates a regimen in which chimeric polypeptides are administered systemically, such as by intravenous infusion, in combination with local administration via the hepatic portal vein.

In other embodiments, the chimeric polypeptides of the disclosure can be delivered in a vesicle, in particular, a liposome (see Langer, 1990, Science 249:1527-1533). In yet another embodiment, the chimeric polypeptides of the disclosure can be delivered in a controlled release system. In another embodiment, a pump may be used (see Langer, 1990, supra). In another embodiment, polymeric materials can be used (see Howard et al., 1989, J. Neurosurg. 71:105). In certain specific embodiments, the chimeric polypeptides of the disclosure can be delivered intravenously.

In certain embodiments, the chimeric polypeptides are administered by intravenous infusion. In certain embodiments, the chimeric polypeptides are infused over a period of at least 10, at least 15, at least 20, or at least 30 minutes. In other embodiments, the chimeric polypeptides are infused over a period of at least 60, 90, or 120 minutes. Regardless of the infusion period, the disclosure contemplates that each infusion is part of an overall treatment plan where chimeric polypeptide is administered according to a regular schedule (e.g., weekly, monthly, etc.).

The foregoing applies to any of the chimeric polypeptides, compositions, and methods described herein. The disclosure specifically contemplates any combination of the features of such chimeric polypeptides, compositions, and methods (alone or in combination) with the features described for the various pharmaceutical compositions and route of administration described in this section.

### VIII. Pharmaceutical Compositions

In certain embodiments, the subject chimeric polypeptides for use in any of the methods disclosed herein are formulated with a pharmaceutically acceptable carrier (e.g., formulated with one or more pharmaceutically acceptable carriers and/or excipients). One or more chimeric polypeptides can be administered alone or as a component of a pharmaceutical formulation (composition). Any of the chimeric polypeptides described herein may be formulated, as described herein, and any such compositions (e.g., pharmaceutical compositions, or preparations, or formulations) may be used in any of the methods described herein. In other embodiments, the composition comprises a chimeric polypeptide comprising an alpha-amylase polypeptide. The chimeric polypeptides may be formulated for administration in any convenient way for use in human or veterinary medicine. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Formulations of the subject chimeric polypeptides include, for example, those suitable for oral, nasal, topical, parenteral, rectal, and/or intravaginal administration. The formulations may conveniently be presented in unit dosage form and may be prepared by any methods well known in the art of pharmacy. The amount of active ingredient which can be combined with a carrier material to produce a single dosage form will vary depending upon the host being treated and the particular mode of administration. The amount of active ingredient which can be combined with a carrier material to produce a single dosage form will generally be that amount of the compound which produces a therapeutic effect.

In certain embodiments, methods of preparing these formulations or compositions include combining another type of therapeutic agents and a carrier and, optionally, one or more accessory ingredients. In general, the formulations can be prepared with a liquid carrier, or a finely divided solid carrier, or both, and then, if necessary, shaping the product.

In certain embodiments, any of the pharmaceutical compositions described herein comprise concentrated amounts of any of the chimeric polypeptides described herein. In some embodiments, the compositions have 50%, 100%, 150%, 200%, 250%, 300%, 350% or 400% more concentrated levels of the chimeric polypeptide as compared to the levels of chimeric polypeptide originally purified from the cells producing the chimeric polypeptide. In some embodiments, the concentration of the chimeric polypeptide is at least 2, 3, 4, 5, 6, 7, 8, 9, 10, 11, 12, 13, 14, 15, 16, 17, 18, 19, or 20 mg/ml. In some embodiments, the concentration of the chimeric polypeptide is at least 10 mg/ml or greater. In some embodiments, the concentration of the chimeric polypeptide is at least 15 mg/ml or greater. In some embodiments, the concentration of the chimeric polypeptide is at least 20 mg/ml or greater. In some embodiments, the concentration of the chimeric polypeptide is at least 30 mg/ml or greater. In some embodiments, the concentration of the chimeric polypeptide is at least 50 mg/ml or greater. In some embodiments, the concentration of the chimeric polypeptide is at least 70 mg/ml or greater. In some embodiments, the concentration of the chimeric polypeptide is at least 90 mg/ml or greater. In some embodiments, the concentration of the chimeric polypeptide is at least 110 mg/ml or greater. In some embodiments, the concentration of the chimeric polypeptide is 10-50 mg/ml, 10-40 mg/ml, 10-30 mg/ml, 10-25 mg/ml, 10-20 mg/ml. 20-50 mg/ml, 50-70 mg/ml, 70-90 mg/ml or 90-110 mg/ml. In some embodiments, any of the compositions described herein preserve at least 80%, 90%, 95% or 100% biological activity (as defined herein) for at least 24 hours, 2 days, 4 days, 1 week, 2 weeks or 1 month when stored in a pharmaceutically acceptable formulation at 4° C. In some embodiments of any of the foregoing, the chimeric polypeptide portion of the composition is substantially pure, as described herein (e.g., greater than 85% of the alpha-amylase present is in association or interconnected with an internalizing moiety).

Formulations for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a subject chimeric polypeptide therapeutic agent as an active ingredient. Suspensions, in addition to the active compounds, may contain suspending agents such as ethoxylated isostearyl alcohols, polyoxyethylene sorbitol, and sorbitan esters, microcrystalline cellulose, aluminum metahydroxide, bentonite, agar-agar and tragacanth, and mixtures thereof.

In solid dosage forms for oral administration (capsules, tablets, pills, dragees, powders, granules, and the like), one or more chimeric polypeptide therapeutic agents of the present disclosure may be mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose, and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like. Liquid dosage forms for oral administration include pharmaceutically acceptable emulsions, microemulsions, solutions, suspensions, syrups, and elixirs. In addition to the active ingredient, the liquid dosage forms may contain inert diluents commonly used in the art, such as water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring, perfuming, and preservative agents.

In certain embodiments, methods of the disclosure include topical administration, either to skin or to mucosal membranes such as those on the cervix and vagina. The topical formulations may further include one or more of the wide variety of agents known to be effective as skin or stratum corneum penetration enhancers. Examples of these are 2-pyrrolidone, N-methyl-2-pyrrolidone, dimethylacetamide, dimethylformamide, propylene glycol, methyl or isopropyl alcohol, dimethyl sulfoxide, and azone. Additional agents may further be included to make the formulation cosmetically acceptable. Examples of these are fats, waxes, oils, dyes, fragrances, preservatives, stabilizers, and surface active agents. Keratolytic agents such as those known in the art may also be included. Examples are salicylic acid and sulfur. Dosage forms for the topical or transdermal administration include powders, sprays, ointments, pastes, creams, lotions, gels, solutions, patches, and inhalants. The subject polypeptide therapeutic agents may be mixed under sterile conditions with a pharmaceutically acceptable carrier, and with any preservatives, buffers (e.g., HEPES buffer), or propellants which may be required. The ointments, pastes, creams and gels may contain, in addition to a subject chimeric polypeptide agent, excipients, such as animal and vegetable fats, oils, waxes, paraffins, starch, tragacanth, cellulose derivatives, polyethylene glycols, silicones, bentonites, silicic acid, talc and zinc oxide, or mixtures thereof. Powders and sprays can contain, in addition to a subject chimeric polypeptides, excipients such as lactose, talc, silicic acid, aluminum hydroxide, calcium silicates, and polyamide powder, or mixtures of these substances. Sprays can additionally contain customary propellants, such as chlorofluorohydrocarbons and volatile unsubstituted hydrocarbons, such as butane and propane.

Pharmaceutical compositions suitable for parenteral administration may comprise one or more chimeric polypeptides in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers (e.g., HEPES buffer), bacteriostats, solutes which render the formulation isotonic with the blood of the intended recipient or suspending or thickening agents. Examples of suitable aqueous and nonaqueous carriers which may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants, such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption, such as aluminum monostearate and gelatin.

Injectable depot forms are made by forming microencapsule matrices of one or more polypeptide therapeutic agents in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

In a preferred embodiment, the chimeric polypeptides of the present disclosure are formulated in accordance with routine procedures as a pharmaceutical composition adapted for intravenous administration to human beings. Where necessary, the composition may also include a solubilizing agent and a local anesthetic such as lidocaine to ease pain at the site of the injection. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

In another embodiment, the chimeric polypeptides of the present disclosure are formulated for subcutaneous administration to human beings.

In certain embodiments, the chimeric polypeptides of the present disclosure are formulated for intrathecal, intracranial and/or intraventricular delivery. In certain embodiments, a chimeric polypeptide of the disclosure for use in treating Alzheimer's Disease and/or dementia or for use in decreasing glycogen accumulation in neurons, such as in a subject having Alzheimer's Disease and/or dementia, is formulated for intrathecal, intracranial and/or intraventricular delivery. In certain embodiments, a method of the disclosure, such as a method of treating Alzheimer's Disease and/or dementia or for decreasing glycogen accumulation in neurons comprising delivering a chimeric polypeptide of the disclosure intrathecally, intracranially and/or intraventricularly (e.g., intracerebroventricularly).

In certain embodiments, the chimeric polypeptides of the present disclosure are formulated for deliver to the heart, such as for intramyocardial or intrapericaridal delivery.

In certain embodiments, the composition is intended for local administration to the liver via the hepatic portal vein, and the chimeric polypeptides are formulated accordingly.

Note that, in certain embodiments, a particular formulation is suitable for use in the context of deliver via more than one route. Thus, for example, a formulation suitable for intravenous infusion may also be suitable for delivery via the hepatic portal vein. However, in other embodiments, a formulation is suitable for use in the context of one route of delivery, but is not suitable for use in the context of a second route of delivery.

The amount of the chimeric polypeptides of the disclosure which will be effective in the treatment of a tissue-related condition or disease (e.g., Pompe Disease and/or Forbes-Cori and/or Andersen Disease and/or von Gierke Disease and/or Lafora Disease and/or

Danon Disease) can be determined by standard clinical techniques. In addition, in vitro assays may optionally be employed to help identify optimal dosage ranges. The precise dose to be employed in the formulation will also depend on the route of administration, and the seriousness of the condition, and should be decided according to the judgment of the practitioner and each subject's circumstances. However, suitable dosage ranges for intravenous administration are generally about 20-5000 micrograms of the active chimeric polypeptide per kilogram body weight. Suitable dosage ranges for intranasal administration are generally about 0.01 pg/kg body weight to 1 mg/kg body weight. Effective doses may be extrapolated from dose-response curves derived from in vitro or animal model test systems.

In certain embodiments, compositions of the disclosure, including pharmaceutical preparations, are non-pyrogenic. In other words, in certain embodiments, the compositions are substantially pyrogen free. In one embodiment the formulations of the disclosure are pyrogen-free formulations which are substantially free of endotoxins and/or related pyrogenic substances. Endotoxins include toxins that are confined inside a microorganism and are released only when the microorganisms are broken down or die. Pyrogenic substances also include fever-inducing, thermostable substances (glycoproteins) from the outer membrane of bacteria and other microorganisms. Both of these substances can cause fever, hypotension and shock if administered to humans. Due to the potential harmful effects, even low amounts of endotoxins must be removed from intravenously administered pharmaceutical drug solutions. The Food & Drug Administration (“FDA”) has set an upper limit of 5 endotoxin units (EU) per dose per kilogram body weight in a single one hour period for intravenous drug applications (The United States Pharmacopeial Convention, Pharmacopeial Forum 26 (1):223 (2000)). When therapeutic proteins are administered in relatively large dosages and/or over an extended period of time (e.g., such as for the patient's entire life), even small amounts of harmful and dangerous endotoxin could be dangerous. In certain specific embodiments, the endotoxin and pyrogen levels in the composition are less then 10 EU/mg, or less then 5 EU/mg, or less then 1 EU/mg, or less then 0.1 EU/mg, or less then 0.01 EU/mg, or less then 0.001 EU/mg.

In some embodiments, the disclosure provides a composition, such as a pharmaceutical composition comprising a chimeric polypeptide of the disclosure formulated with one or more pharmaceutically acceptable carriers and/or excipients. Such compositions include compositions comprising any of the internalizing moiety portions, described herein, and an alpha-amylase portion comprising, as described herein. For example, the disclosure provides compositions comprising an alpha-amylase-containing chimeric polypeptide. In certain embodiments, any of the compositions described herein may be based on any of the alpha-amylase portions and/or internalizing moiety portions described herein. Moreover, any such compositions may be described based on any of the structural and/or functional features described herein. Any such compositions may be used in any of the methods described herein, such as administered to cells and/or to subjects in need of treatment, such as administered to cells and/or to subjects having Pompe Disease, von Gierke Disease, Forbes Cori Disease, Lafora Disease, Andersen Disease, Danon Disease, or Alzheimer's Disease. Any such compositions may be used to deliver alpha-amylase activity into cells, such as into muscle, liver, and/or neuronal cells in a patient in need thereof (e.g., a patient having Pompe Disease, von Gierke Disease, Forbes Cori Disease, Lafora Disease, Andersen Disease, Danon Disease, or Alzheimer's Disease).

Such compositions, including any of the compositions described herein, may be provided, for example, in a bottle or syringe and stored prior to administration.

The foregoing applies to any of the chimeric polypeptides, compositions, and methods described herein. The disclosure specifically contemplates any combination of the features of such chimeric polypeptides, compositions, and methods (alone or in combination) with the features described for the various pharmaceutical compositions and route of administration described in this section.

### IX. Animal Models

Mice engineered to be deficient in malin display a phenotype similar to that observed in human cases of Lafora Disease. Specifically, malin^{-/-} mice presented in an age-dependent manner neurodegeneration, increased synaptic excitability, and propensity to suffer myoclonic seizures. Valles-Ortega et al., 2011, EMBO Mol Med, 3(11):667-681. In addition, these mice accumulated glycogen-filled inclusion bodies that were most abundant in the hippocampus and cerebellum, but that were also found in skeletal and cardiac muscle cells. Valles-Ortega et al. Glycogen was also found to be less branched in the cells of malin^{-/-} mice as compared to glycogen observed in the cells of healthy control mice. Valles-Ortega et al. An increased level of glycogen hyperphosphorylation has also been described in this mouse model. Turnbull et al., 2010, Ann Neurol, 68(6):925-33. Mice engineered to be deficient in laforin also display some phenotypic similarities to human cases of Lafora Disease. Specifically, laforin^{-/-} mice are born developmentally normal, but develop an age-dependent ataxia and myoclonus epilepsy. Ganesh et al., 2002, Hum Mol Genet, 11(11):1251-62. In addition, laforin^{-/-} mice display widespread degeneration of neurons by two months of age and the development of inclusion bodies by 4-12 months of age. Ganesh et al., 2002. Mice deficient for laforin also display hyperphosphorylation and aggregation of tau protein in the brain. Puri et al., 2009, J Biol Chem, 284(34):22657-63.

Accordingly, in certain embodiments, the present disclosure contemplates methods of surveying improvements in disease phenotypes using any of the alpha-amylase (e.g., a mature alpha-amylase) constructs of the disclosure disclosed herein in any one or more animal models, such as the mouse models described herein. By way of example, various parameters can be examined in experimental animals treated with a subject chimeric polypeptide, and such animals can be compared to controls. Exemplary parameters that can be assessed to evaluate potential efficacy include, but are not limited to: increase in lifespan; increase in glycogen clearance, decrease in glycogen accumulation, and improved muscle strength, for example in open field and open wire hang paradigms, improved heart function, improved liver function or decrease in liver size. Increase in glycogen clearance and decrease in glycogen accumulation may be assessed, for example, by periodic acid Schiff staining in a biopsy (e.g., muscle (e.g., cardiac or diaphragm), liver or neuronal) from a treated or untreated animal model. Further parameters that may be observed include a reduction in: neurodegeneration, number/duration/intensity of seizures, number or size of inclusion bodies, amount of glycogen hyperphosphorylation, ataxia, tau hyperphosphorylation and/or tau aggregation. In certain embodiments, the disclosure provides a method of decreasing cytoplasmic glycogen accumulation in a subject having any of the foregoing conditions. In particular embodiments, any of the parameters disclosed herein may be monitored in the skeletal muscle (e.g., diaphragm), liver, cardiac muscle, and or brain neurons from a Lafora Disease animal model.

Moreover, a complete pharmacokinetic study to determine the effective dose, clearance rate, volume of distribution, and half-life of any of the chimeric polypeptides described herein is determined. The PK/PD/TK of the final product can be examined in larger animals such as rats, dogs, and primates.

The above models are exemplary of suitable animal model systems for assessing the activity and effectiveness of the subject chimeric polypeptides and/or formulations. These models have correlations with symptoms of Lafora Disease, and thus provide appropriate models for studying Lafora Disease. Activity of the subject chimeric polypeptides and/or formulations is assessed in any one or more of these models, and the results compared to that observed in wildtype control animals and animals not treated with the chimeric polypeptides (or treated with alpha-amylase alone). Similarly, the subject chimeric polypeptides can be evaluated using cells in culture, for example, cells prepared from any of the foregoing mutant mice or other animals, as well as wild type cells, such as fibroblasts, myoblasts or hepatocytes. Cells from subjects having the disease may also be used. An example of an in vitro assay for testing activity of the chimeric polypeptides disclosed herein would be to treat Lafora Disease cells with or without the chimeric polypeptides and then, after a period of incubation, stain the cells for the presence of glycogen, e.g., by using a periodic acid Schiff (PAS) stain. The amount of inclusion bodies and glycogen hyperphosphorylation may also be monitored. Cell proliferation, morphology and cell death may also be monitored in treated or untreated cells.

Chimeric polypeptides of the disclosure have numerous uses, including in vitro and in vivo uses. In vivo uses include not only therapeutic uses but also diagnostic and research uses in, for example, any of the foregoing animal models. By way of example, chimeric polypeptides of the disclosure may be used as research reagents and delivered to animals to understand alpha-amylase bioactivity, localization and trafficking, protein-protein interactions, enzymatic activity, and impacts on animal physiology in healthy or diseased animals.

Chimeric polypeptides may also be used in vitro to evaluate, for example, alpha-amylase bioactivity, localization and trafficking, protein-protein interactions, and enzymatic activity in cells in culture, including healthy, diseased (but not alpha-amylase deficient) and laforin, alpha-amylase and/or malin deficient cells in culture. The disclosure contemplates that chimeric polypeptides of the disclosure may be used to deliver alpha-amylase to cytoplasm, lysosome, and/or autophagic vesicles of cells, including cells in culture. In some embodiments, the cultured cells are obtained from a Lafora Disease subject, such as from a Lafora Disease human patient or from a Lafora Disease animal model. In some embodiments, the chimeric polypeptides may be used in a hypoxic cell model, similar to that described in Pelletier et al., Frontiers in Oncology, 2(18):1-9.

Additionally, cell free systems may be used to assess, for example, enzymatic activity of the subject chimeric polypeptides. For example, glycogen may be obtained from a sample from a healthy and/or a diseased subject (e.g. from a Lafora Disease subject), and the ability of any of the chimeric polypeptides disclosed herein to hydrolyze the glycogen may be assessed, e.g., in a manner similar to that described in the Example section provided herein. In some embodiments, the glycogen for used in such cell-free systems may be obtained from a muscle (e.g., diaphragm or cardiac muscle), liver, or neuronal (e.g., brain) cells from a subject (e.g., from a Lafora Disease subject). In some embodiments, the subject is a human Lafora Disease patient or an animal model of Lafora Disease.

Chimeric polypeptide, such as alpha-amylase chimeric polypeptides, may further be used to identify protein-protein interactions in systems where a protein such as alpha-amylase is not deficient, such as in Forbes-Cori Disease. Chimeric polypeptides may further be used to understand the relative benefit of decreasing accumulation of glycogen in certain cell types but potentially not all cell types in which symptoms are present. Chimeric polypeptides may be used to identify substrates for alpha-amylase particularly in settings where endogenous alpha-amylase is not mutated. Chimeric polypeptides are useful for evaluating trafficking of alpha-amylase and the chimeric polypeptides in healthy, as well as diseased cells where glycogen accumulation is due to different underlying causes.

### X. Kits

In certain embodiments, the disclosure also provides a pharmaceutical package or kit comprising one or more containers filled with at least one chimeric polypeptide of the disclosure. Optionally associated with such container(s) can be a notice in the form prescribed by a governmental agency regulating the manufacture, use or sale of pharmaceuticals or biological products, which notice reflects (a) approval by the agency of manufacture, use or sale for human administration, (b) directions for use, or both.

In certain embodiments, the kit includes additional materials to facilitate delivery of the subject chimeric polypeptides. For example, the kit may include one or more of a catheter, tubing, infusion bag, syringe, and the like. In certain embodiments, the chimeric polypeptide is packaged in a lyophilized form, and the kit includes at least two containers: a container comprising the lyophilized chimeric polypeptide and a container comprising a suitable amount of water, buffer (e.g., HEPES buffer), or other liquid suitable for reconstituting the lyophilized material.

The foregoing applies to any of the chimeric polypeptides, compositions, and methods described herein. The disclosure specifically contemplates any combination of the features of such chimeric polypeptides, compositions, and methods (alone or in combination) with the features described for the various kits described in this section.

### Exemplification

The disclosure now being generally described, it will be more readily understood by reference to the following examples, which are included merely for purposes of illustration of certain aspects and embodiments of the present disclosure, and are not intended to limit the disclosure. For example, the particular constructs and experimental design disclosed herein represent exemplary tools and methods for validating proper function. As such, it will be readily apparent that any of the disclosed specific constructs and experimental plan can be substituted within the scope of the present disclosure.

### Example 1: Generation and Characterization of a Fab-Alpha-Amylase Protein

A. Synthesis of a Fab-Alpha-Amylase Protein

Chimeric polypeptides comprising a mature alpha-amylase polypeptide portion and an internalizing moiety portion were made recombinantly in two different mammalian cell lines, CHO-3E7 and HEK-293 6E cells. An alpha-amylase polypeptide comprising a mature alpha-amylase polypeptide (e.g., a polypeptide having the amino acid sequence of SEQ ID NO: 1) was fused to a Fab of a humanized 3E10 antibody comprising the heavy chain variable domain set forth in SEQ ID NO: 7. Specifically, an alpha-amylase polypeptide having the amino acid sequence of SEQ ID NO: 1 was fused to the C-terminus of the heavy chain constant region of a humanized 3E10 Fab fragment (which included the signal sequence of SEQ ID NO: 4) by means of a linker having the amino acid sequence of SEQ ID NO: 6 to generate a fusion polypeptide having the amino acid sequence of SEQ ID NO: 9. The light chain comprises the amino acid sequence of SEQ ID NO: 8 and the signal sequence of SEQ ID NO: 5 to provide the sequence of SEQ ID NO: 10. The resulting “Fab-alpha-amylase” comprising both the heavy chain and light chains is referred to in the experimental designs described below.

This Fab was made by expressing a vector encoding the light chain and a vector encoding the heavy chain-amylase fusion in either of two cell lines. Although two separate vectors were used, a single vector encoding both the heavy and light chain could also have been employed.

A nucleotide sequence encoding the recombinant heavy chain (SEQ ID NO: 9) and a nucleotide sequence encoding the light chain (SEQ ID NO: 10) was codon optimized for mammalian cell expression and cloned into the pTT5 vector using standard methods. Low endotoxin, giga-prep scale production of the expression plasmid encoding the sequence of SEQ ID NO: 9 and the expression plasmid encoding the sequence of SEQ ID NO: 10 resulted in 7.0 mg of each plasmid DNA (each, a vector). CHO-3E7 and HEK-293-6E cells were then each transfected with these two plasmids in a manner summarized below.

i. CHO-3E7

Four, 1 L cultures of CHO-3E7 cells (initial density of 1.9×10⁶ cells/mL) in 2 L shake flasks were transfected with 1 mg total (1:1 ratio HC:LC) of plasmid DNA/L culture using PolyPlus linear Q-PEI at a 1:4 (w/v) DNA:PEI ratio. Culture parameters were monitored using a CedexXS (days 0-1) or a Vi-Cell XR (days 2-8) for density and viability. The culture media was F17 supplemented with 0.1% Pluronic F-68, 4 mM GlutaMAX. Cells were maintained at a density between 0.5-5×10⁶ cells/mL in shake flasks. The flasks were incubated at 37° C. in a humidified 5% CO₂ environment with shaking at 135 rpm. Cultures were harvested 8 days post-transfection via centrifugation for 5 minutes at 1000×g. The conditioned culture supernatant was clarified by centrifugation for 30 minutes at 9300×g.

Fab-alpha-amylase was purified from the CHO-3E7 cells using a CaptureSelect IgG-CH1 affinity matrix (Life Technologies, #194320001). The CaptureSelect IgG-CH1 affinity resin (bed volume of 5 mL) was equilibrated in Buffer A (1×PBS (2.7 mM KCl, 1.7 mM KH₂PO₄, 136 mM NaCl, 10.1 mM Na₂HPO₄), pH 7.2 (23° C.)). Fab-alpha-amylase from 4L of exhausted supernatant was batch bound with the CaptureSelect IgG-CH1 affinity resin at 4° C. overnight with stirring. The resin was collected in a 2.5 cm diameter Econo-column and washed with approximately 15 column volumes (CV) of Buffer A, 15 CV of Buffer B (1×PBS, 500 mM NaCl, pH 7.2 (23° C.)) and 15 CV Buffer A. The resin-bound Fab-alpha-amylase was eluted with ˜4 CV of Buffer C (30 mM NaOAc, pH 3.5-3.6 (23° C.)) followed by ˜4 CV of Buffer D (100 mM Glycine, pH 2.7 (23° C.)) collecting the protein in 2 mL fractions diluted in 1/10th volume Buffer E (3 M NaAcetate, ˜pH 9.0 (23° C.)) to neutralize. To minimize the elution volume, elution was paused for several minutes between each fraction collected. Fractions were analyzed by A280 prior to pooling fractions 6-12 and 1-11 from the Buffer C and Buffer D elutions, respectively. The combined CaptureSelect IgG-CH1 affinity pool (50 mL) was dialyzed against 3×1 L of dialysis buffer (20 mM Histidine, 150 mM NaCl, pH 6.5 (23° C.)) at 4° C. The dialyzed pool was concentrated to −10 mg/mL using a VivaSpin 20 (10K MWCO, PES membrane) centrifugal device prior to final analysis and storage at −80° C. Select fractions were analyzed by SDS-PAGE and by size exclusion chromotography, where it was confirmed that the Fab-alpha-amylase was being produced and successfully purified (data not shown).

ii. HEK-293-6E Cells

Twenty, 1 L cultures of 293-6E cells (initial density of 2.6×10⁶ cells/mL) in 2 L shake flasks were transfected with 1 mg total (1:1 ratio HC:LC) of plasmid DNA/L culture using PolyPlus linear Q-PEI at a 1:1.5 (w/v) DNA:PEI ratio. Culture parameters were monitored using a ViCell XR for density and viability. The culture media was F17 supplemented with 0.1% Pluronic F-68, 4 mM GlutaMAX, 25 μg/mL G418. Cells were maintained at a density between 0.5-5×10⁶ cells/mL in shake flasks. The flasks were incubated at 37° C. in a humidified 5% CO₂ environment with shaking at 135 rpm. Cultures were harvested 6 days post-transfection via centrifugation for 5 minutes at 1000×g. The conditioned culture supernatant was clarified by centrifugation for 30 minutes at 9300×g.

Fab-alpha-amylase was purified from the HEK-293-6E cells using a CaptureSelect IgG-CH1 affinity matrix (Life Technologies, #194320001). The CaptureSelect IgG-CH1 affinity resin was equilibrated in buffer A (1×PBS (2.7 mM KCl, 1.7 mM KH₂PO₄, 136 mM NaCl, 10.1 mM Na₂HPO₄), pH 7.2 (23° C.)). Fab-alpha-amylase from 20 L of exhausted supernatant was batch bound with the CaptureSelect IgG-CH1 affinity resin (40 mL bed volume) at 4° C. overnight with stirring. The resin was collected in a 5 cm diameter Econo-column and washed with approximately 15 column volumes (CV) of Buffer A, 15 CV of Buffer B (1×PBS, 500 mM NaCl, pH 7.2 (23° C.)) and 15 CV buffer A. The resin-bound fusion protein was eluted with ˜4 CV of Buffer C (30 mM NaOAc, pH 3.5-3.6 (23° C.)) followed by ˜4 CV of Buffer D (100 mM Glycine, pH 2.7 (23° C.)) collecting the protein in 10 mL fractions diluted in 1/10th volume Buffer E (3M NaAcetate, ˜pH 9.0 (23° C.)) to neutralize. To minimize the elution volume, elution was paused for several minutes between each fraction collected. Fractions were analyzed by A₂₈₀ prior to pooling fractions 7-25. Select fractions were analyzed by SDS PAGE. Fab-alpha-amylase remained in the non-bound pool from the first affinity chromatography pass. The above procedure was repeated to capture remaining Fab-alpha-amylase. The affinity pools were combined prior to dialysis.

The combined CaptureSelect IgG-CH1 affinity pool (250 mL) was dialyzed against 3×4 L of dialysis buffer (20 mM Histidine, 150 mM NaCl, pH 6.5 (23° C.)) at 4° C. The dialyzed pool was concentrated to −10 mg/mL using a VivaCell 100 (10K MWCO, PES membrane) centrifugal device prior to final analysis and storage at −80° C. Select fractions were analyzed by SDS-PAGE and by size exclusion chromotography, where it was confirmed that the Fab-alpha-amylase was being produced and successfully purified (data not shown).

In alternative embodiments, a protein comprising a full-length humanized 3E10 antibody and the alpha-amylase protein may be generated. Other chimeric proteins of the disclosure may be, for example, similarly made, and any such proteins may be used in any of the methods described herein.

B. Fab-Alpha-Amylase in a Cell-Free Activity Assay

The ability of Fab-alpha-amylase to digest glycogen was assessed in a cell-free assay. Glucose standards were prepared by dilution in water from 1 mg/mL glucose from the Glucose Oxidase kit (Sigma GAGO20-1KT): 0.08 mg/mL, 0.06 mg/mL, 0.04 mg/mL, 0.02 mg/mL, 0.01 mg/mL, 0.005 mg/mL (0.1 mg/mL=555.1 μM). Twenty mL of citrate/phosphate buffers from pH 3.5-7.0 were prepared by adding 0.1 M citric acid and 0.2 M sodium phosphate dibasic in the amounts indicated in Table 1. The buffers were spiked in 10% Tween-80 to 0.02% final, and pH was verified with a pH meter. The 0.1M sodium acetate pH 4.3+0.02% tween-80 was also prepared.

TABLE 1

| | mL 0.1M | mL 0.2M |
|---|---|---|
| pH | Citric Acid | NaPO4 Dibasic |
| 3.5 | 6.04 | 13.96 |
| 4 | 7.72 | 12.28 |
| 4.3 | 8.49 | 11.51 |
| 4.5 | 9 | 11 |
| 5 | 10.28 | 9.72 |
| 5.5 | 11.36 | 8.64 |
| 6 | 12.84 | 7.16 |
| 6.5 | 14.2 | 5.8 |
| 7 | 17.44 | 2.56 |

Ten mg/mL glycogen was then prepared in each buffer solution to be tested. The Fab-alpha-amylase was diluted to 1 mg/mL in reaction buffer, and 1.8 μL of 1 mg/mL Fab-alpha-amylase was then added to 178.2 μL glycogen solution in a 500 μL vial (10 μg/mL final Fab-alpha-amylase concentration). The samples were mixed well and incubated at ambient temperature for 1 hour. Glycogen solution was also retained as a negative control. The digestion was terminated by heating the samples at 95° C. for 10 minutes. The Fab-alpha-amylase negative glycogen samples were heated as a negative control/blank sample. The glucose standards and digested glycogen test samples (40 μL/well) were then pipetted into 96 well plate in triplicate, and 80 μL Glucose Oxidase kit Reagent Mix (Sigma GAGO20-1KT; prepared as described in kit) was added to each well at room temperature with a multi-channel pipette, mixed well and incubated at 37° C. for 30 minutes. The reaction was terminated by adding 80 μL 12 N sulfuric acid with multi-channel pipette and mixing well. The plate was then read at 540 nm. No meaningful glycogen digestion was observed in the negative control samples. By comparison, glycogen digestion was observed in samples having the Fab-alpha-amylase protein, with the most robust activity observed at slightly acidic pHs. Representative results from test samples are shown below in Table 2.

TABLE 2

| pH | Specific Activity (μM/min/mg) |
|---|---|
| 5.5 | 753.5 |
| 6.0 | 689.4 |
| 6.5 | 562.3 |
| 7.0 | 435.1 |
| 7.5 | 390.2 |

The Fab-alpha-amylase protein was also found to be inactive at a pH of 4.3 (Data Not Shown).

In an additional or alternative experiment, polyglucosan bodies are isolated from a Lafora Disease animal model (e.g., the mouse model of Ganesh et al., 2002, Hum Mol Genet, 11:1251-1262) in a manner similar to that described in Zeng et al., 2012, FEBS J, 279(14):2467-78. Briefly, forebrain cortical neurons are microdissected from the brains of postnatal day 2 Epm2a wildtype or knockout mice into Neurobasal medium in a manner similar to that described in Wang et al., 2013, Mol Neurobiol, 48(1):49-61. Polyglucosan is then isolated in a manner similar to that described in Wang et al. Purified Fab-Alpha-Amylase fusion proteins are incubated with the isolated polyglucosan at various doses and for various timepoints, and the ability of the Fab-alpha-amylase to digest the polyglucosan is monitored.

C. Fab-Alpha-Amylase in Cell Culture

The efficacy of the Fab-alpha-amylase proteins on reducing polyglucosan levels in a primary neuron cell culture is tested in a manner similar to that described in Wang et al. (also see cell isolation protocol described above). Alternatively, N2A cells may be used in which a Lafora Disease phenotype is mimicked by treating these cells with the ER stressor thapsigargin in a manner similar to that described in Wang et al. The primary neuron cells or ER-stressed N2A cells (or control unstressed N2A cells) are then administered (or not) the Fab-alpha-amylase proteins, and the effect of the proteins on polyglucosan levels is monitored by PAS staining. A reduction in PAS staining in the protein treated cells is consistent with the polyglucosan being cleared from the cells by the chimeric polypeptides. In some embodiments, the effect of Fab-alpha-amylase on glycogen levels is tested on primary cells from a GSDIII and/or GSDIV human patient or animal model, or in an animal model.

In some embodiments, the effect of the Fab-alpha-amylase on glycogen levels is tested in a hypoxia cell model. In particular embodiments, the hypoxia tumor cell model is the same or similar to the one described in Pelletier et al., Frontiers in Oncology, 2(18):1-9, where it was shown that hyopoxia induces glycogen accumulation in certain cell types. Briefly, non-cancerous cells (e.g., Chinese hamser lung fibroblasts (CCL39) or mouse embryonic fibroblasts (MEF)) and/or cancerous cells (e.g., LS174 or BE colon carcinoma cells) are cultured at normoxic or hypoxic (1% O₂) conditions for 96 hours in the presence or absence of the Fab-alpha-amylase. Glycogen levels are assessed by electron microscopy and/or Periodic Acid Schiff staining. A reduction in glycogen levels in the Fab-alpha-amylase treated hypoxic cells as compared to the untreated hypoxic cells is assessed.

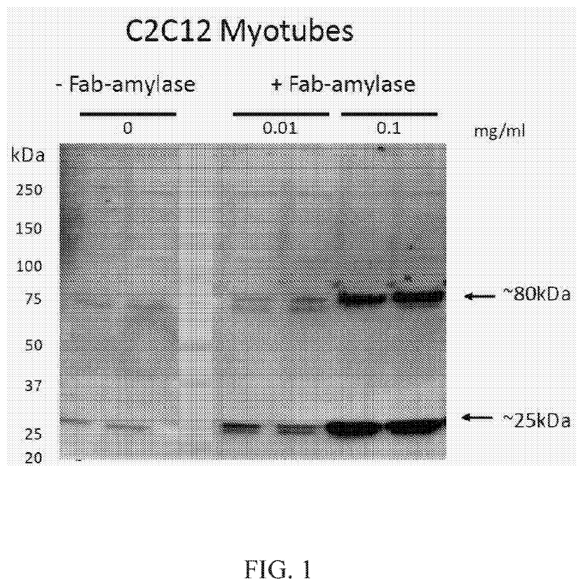

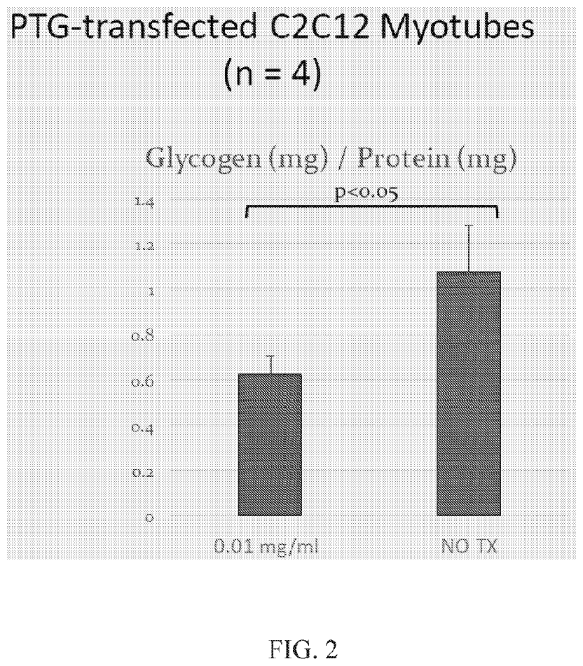

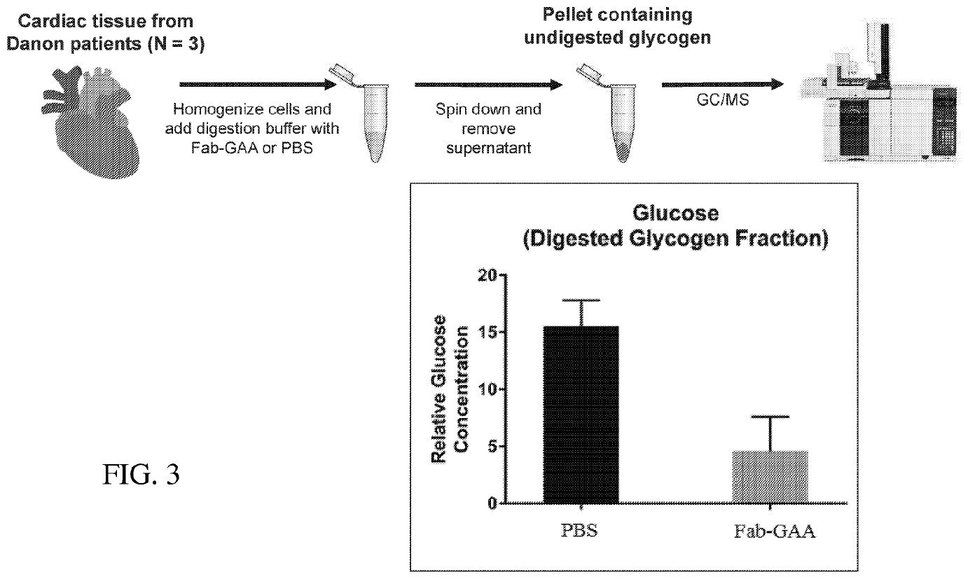

The efficacy of Fab-amylase on reducing polyglucosan levels in ENT2+C2C12 myotubes is tested. The dose dependent uptake of Fab-amylase in ENT2+C2C12 myotubes is shown in FIG. 1. A comparison of −Fab-amylase and +Fab-amylase at 0.01 mg/ml and 0.1 mg/ml is provided. The reduction of glycogen in ENT2+C2C12 myotubes by Fab-amylase is demonstrated by comparing glycogen (mg)/protein (mg) levels for non-transfected C2C12 myotubes to treated C2C12 myotubes (FIG. 2). Treated C2C12 myotubes are prepared by transfecting C2C12 myotubes with PTG and then treating the transfected myotubes with 0.01 mg/ml Fab-Amylase in the media after 24 hours.

D. Effect of Fab-Amylase on Lafora Bodies

Lafora Disease may be characterized by the accumulation of glycogen-filled inclusion bodies (also referred to herein as Lafora bodies or polyglucosan bodies) within the cytoplasm of the cells in the brain, heart, liver, muscle and skin.

i. Assessment of Purified Inclusion Bodies

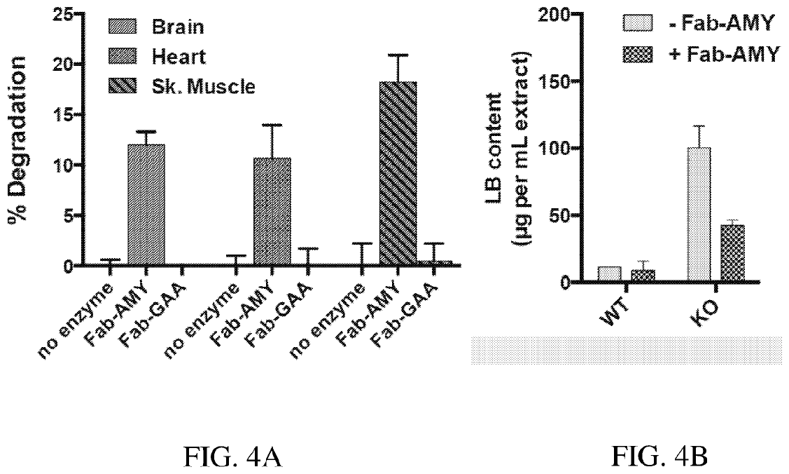

The efficacy of Fab-fusions can be assessed using purified inclusion bodies. A degradation assay is performed applying Fab-amylase and Fab-glucosidase to purified inclusion bodies isolated from tissue of the brain, heart, and skeletal muscle of Lafora knock out mice. The results show that Fab-amylase degrades the purified inclusion bodies (FIG. 4A). The effect of Fab-amylase on inclusion bodies is further assessed by measuring the inclusion body content (μg per mL extract) of samples obtained from wild type mice and knock out mice treated with −Fab-amylase and +Fab-amylase ex vivo (FIG. 4B).

E. Fab-Alpha-Amylase Activity

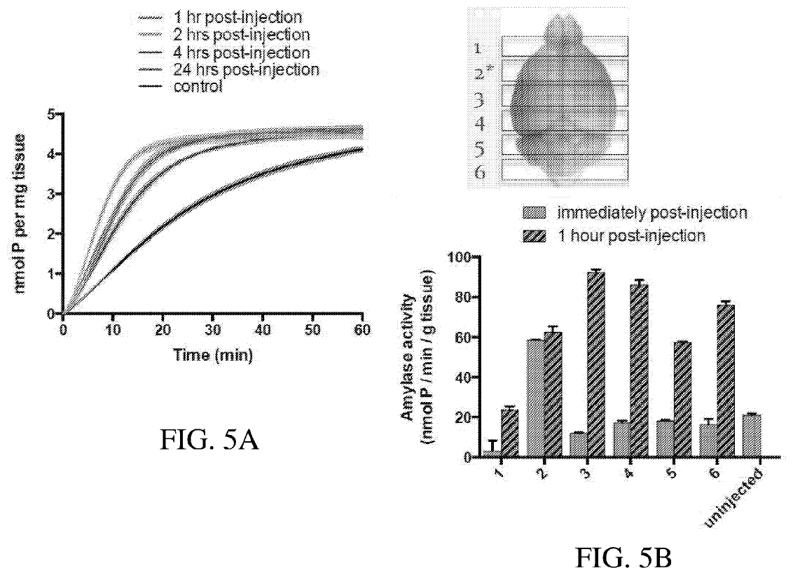

The activity of Fab-amylase can be measured using an amylase activity colorimetric assay kit (BioVision). The methods for using the assay kit are optimized by identifying a choice of time points to measure the sample at OD 405 nm and selecting the optimum time point. Fab-amylase activity (nmol P per mg tissue) is measured in the muscle at various time points post injection, including at 1 hour post-injection, 2 hours post-injection, 4 hours post-injection, and 24 hours post-injection (FIG. 5A). Amylase activity (nmol P/min/g tissue) is also measured for various sections of the brain (as identified in upper panel of FIG. 5B) immediately post-injection and 1 hour post-injection (FIG. 5B, lower panel).

F. Fab-Alpha-Amylase In Vivo

Mice engineered to be deficient in malin display a phenotype similar to that observed in human cases of Lafora Disease. Specifically, malin^{-/-} mice presented in an age-dependent manner neurodegeneration, increased synaptic excitability, and propensity to suffer myoclonic seizures. Valles-Ortega et al., 2011, EMBO Mol Med, 3(11):667-681. In addition, these mice accumulated glycogen-filled inclusion bodies that were most abundant in the hippocampus and cerebellum, but that were also found in skeletal and cardiac muscle cells. Valles-Ortega et al. Glycogen was also found to be less branched in the cells of malin^{-/-} mice as compared to glycogen observed in the cells of healthy control mice. Valles-Ortega et al. An increased level of glycogen hyperphosphorylation has also been described in this mouse model. Turnbull et al., 2010, Ann Neurol, 68(6):925-33. Alternative mouse models that could be used in the in vivo experiments described herein include the laforin mouse model described in Ganesh et al., 2002, Hum Mol Genet, 11(11):1251-62.

i. Selection of Dose of Fab-Alpha-Amylase

The evaluation dose of the Fab-alpha-amylase delivered to the Lafora Disease mice is determined empirically. To minimize the confounding effect of a neutralizing immune response to Fab-alpha-amylase and to maximize the ability to demonstrate a therapeutic effect, two high doses of 5 mg/kg of Fab-alpha-amylase are delivered in one week, followed by assessment of changes in disease endpoints. The development of anti-Fab-alpha-amylase antibodies is also monitored. Following establishment that intravenous Fab-alpha-amylase results in an improvement in aberrant glycogen storage in mice brain, heart, diaphragm or liver, subsequent in vivo assessments in other models (e.g., primates) are initiated, followed by assessment of changes in glycogen clearance, as determined by immunohistochemistry (e.g., PAS staining)

ii. Materials and Methods
a) Injection of Chemically and Genetically Conjugated Fab-Alpha-Amylase

Fab-alpha-amylase is formulated and diluted in a buffer that is consistent with intravenous injection (e.g. sterile saline solution or a buffered solution of 50 mM Tris-HCl, pH 7.4, 0.15 M NaCl). The amount of Fab-alpha-amylase given to each mouse is calculated as follows: dose (mg/kg)×mouse weight (kg)×stock concentration (mg/ml)=volume (ml) of stock per mouse, q.s. to 100 ul with vehicle.

b) Blood Collection

Blood is collected by cardiac puncture at the time that animals are sacrificed for tissue dissection. Serum is removed and frozen at −80° C. To minimize the effects of thawing and handling all analysis of Fab-alpha-amylase circulating in the blood is performed on the same day.

c) Tissue Collection and Preparation

Sampled tissues are divided for immunoblot, glycogen analysis, formalin-fixed paraffin-embedded tissue blocks and frozen sections in OCT. Heart, liver, lung, spleen, kidneys, quadriceps, EDL, soleus, diaphragm, brain, and biceps tissue (50-100 mg) are subdivided and frozen in plastic tubes for further processing for immunoblot and glycogen analysis. Additional samples of heart, liver, lung, spleen, kidneys, quadriceps, EDL, soleus, diaphragm, brain and biceps are subdivided, frozen in OCT tissue sectioning medium, or fixed in 3% glutaraldehyde formaldehyde fixation for 24 to 48 hours at 4° C. and embedded in Epon resin, or fixed in 10% NBF and processed into paraffin blocks. Some samples are homogenized in 30% KOH for 15 mM, and glycogen levels are determined using an amyloglucosidase-based assay described in Valles-Ortega et al. In addition, glycogen branching are assessed in the homogenized samples using the methods described in Valles-Ortega et al. A reduction in glycogen accumulation and an increase in glycogen branching in samples from mice treated with Fab-alpha-amylase as compared to untreated control mice is indicative that the chimeric polypeptides are capable of clearing glycogen and improving glycogen branching in the cells of the mice.

d) Histological Evaluation

Epon-resin embedded samples are cut at 1 μm and stained with PAS-Richardson's stain for glycogen staining. Reduced levels of glycogen accumulation in tissues (e.g., muscle or liver) of Lafora Disease mice treated with Fab-alpha-amylase as compared to control-treated Lafora Disease mice is indicative that the Fab-alpha-amylase is capable of reducing glycogen levels in vivo.

e) Immunofluorescence

Exogenously delivered Fab-alpha-amylase are detected using a polyclonal or monoclonal anti-alpha-amylase antibody. Ten micrometer frozen sections are cut and placed on Superfrost Plus microscope slides.

f) Immunoblot

Immunoblotting is used to detect 3E10 and alpha-amylase immune reactive material in Fab-alpha-amylase treated muscles (e.g., diaphragm), heart and brain tissues. Protein isolation and immunoblot detection of 3E10 and alpha-amylase are performed according to routine immunoblot methods. Alpha-amylase is detected with an antibody specific for this protein. Antibody detection of blotted proteins use NBT/BCIP as a substrate. Controls include vehicle and treated Lafora Disease mice and vehicle and treated homozygous wildtype mice.

g) Analysis of Circulating Fab-Alpha-Amylase

An ELISA specific to human Fab-alpha-amylase is developed and validated using available anti-human amylase antibodies (or anti-CH1 antibodies to detect the constant heavy chain of the Fab portion of the Fab-alpha-amylase) and horseradish peroxidase conjugated anti-mouse secondary antibody (Jackson Immunoresearch). Recombinant Fab-alpha-amylase is diluted and used to generate a standard curve. Levels of Fab-alpha-amylase are determined from dilutions of serum (normalized to ng/ml of serum) or tissue extracts (normalized to ng/mg of tissue). Controls include vehicle and treated wildtype and Lafora Disease mice.

h) Monitoring of anti-Fab-Alpha-Amylase antibody responses

Purified Fab-alpha-amylase used to inject Lafora Disease mice are plated onto high-binding 96 well ELISA plates at 1 ug/ml in coating buffer (Pierce Biotech), allowed to coat overnight, blocked for 30 minutes in 1% nonfat drymilk (Biorad) in TBS, and rinsed three times in TBS. Two-fold dilutions of sera from vehicle and Fab-alpha-amylase injected animals are loaded into wells, allowed to incubate for 30 minutes at 37° C., washed three times, incubated with horseradish peroxidase (HRP)-conjugated rabbit anti-mouse IgA, IgG, and IgM, allowed to incubate for 30 minutes at 37° C., and washed three times. Mouse anti-Fab-alpha-amylase antibodies are detected with TMB liquid substrate and read at 405 nm in ELISA plate reader. A polyclonal anti-alph-amylase antibody, followed by HRP-conjugated goat anti-rabbit serve as the positive control antibody reaction. Any absorbance at 405 nm greater than that of vehicle treated Lafora mice constitutes a positive anti-Fab-alpha-amylase antibody response. Controls include vehicle and treated wildtype mice and Lafora mice.

i) Tissue Glycogen Analysis

Tissue glycogen content is assayed using the protocol described in Akman (2011). Samples of frozen muscle (e.g., diaphragm or cardiac muscle), brain and liver tissue (˜30-60 mg) are boiled in 200 μl of 30% (wt/vol) KOH for 30 min with occasional shaking. After cooling, 67 μl of 0.25 m Na2SO4 and 535 μl of ethanol is added. Next, samples are centrifuged at 14500g for 20 min at 4° C. to collect glycogen. The glycogen pellet is suspended in water (100 μl), 200 μl of ethanol are added and centrifugation as described above is used to harvest glycogen. This ethanol precipitation step is repeated, and the glycogen pellet is dried in a Speed-Vac. Dried glycogen pellets are suspended in 100 μl of amyloglucosidase [0.3 mg/ml in 0.2 m sodium acetate (pH 4.8)] and incubated at 37° C. for 3 h to digest glycogen. To determine the glucose concentration in the samples, an aliquot (5 μl) of digested glycogen is added to 95 μl of a solution containing 0.3 m triethanolamine (pH 7.6), 0.4 mm MgCl2, 0.9 mm NADP, 1 mm ATP and 0.1 μg of glucose-6-phosphate dehydrogenase/ml. The absorbance at 340 nm is read before and after the addition of 0.1 μg of hexokinase.

j) Seizure Assessment

The malin^{-/-} mice described by Valles-Ortega et al. were generated in the C57BL6 strain of mice, which are normally resistant to seizures. However, while administration of kainate did not induce any seizures in wildtype C57BL6 mice, malin^{-/-} mice treated with kainate displayed clonic hippocampal seizures. Valles-Ortega et al. Malin^{-/-} mice are treated with kainate and with or without Fab-alpha-amylase. If the mice treated with kainate and Fab-alpha-amylase display reduced seizures as compared to malin^{-/-} mice treated with kainate but not with any chimeric polypeptides, this is indicative that the chimeric polypeptides are effective in treating some of the neurological defects observed in the malin^{-/-} mice.

k) Neurodegeneration Analysis

The total number of parvalbumin positive interneurons is assessed in the hippocampus of malin^{-/-} mice treated with or without Fab-alpha-amylase. Valles-Ortega et al. If the hippocampi from mice treated with Fab-alpha-amylase display less parvalbumin-positive neurodegeneration than in the hippocampi from untreated mice, than this is indicative that the chimeric polypeptides are effective in reducing neurodegeneration in the malin^{-/-} mice.

1) Statistical Analysis

Pairwise comparisons employs Student's t-test. Comparisons among multiple groups employ ANOVA. In both cases a p-value <0.05 is considered statistically significant.

iii. Assessment of Fab-Alpha-Amylase Upon Intramuscular Injection

The effect of intramuscular injections of Fab-amylase is assessed by comparing Fab-amylase treated mice with control mice. In the Fab-amylase treated mice, four 20 ul (10 mg/ml) intramuscular injections are administered into the Tibialis anterior (TA) muscle of the right leg over the course of two weeks, while PBS is injected into the left leg. In the control mice, PBS is injected into both the right and left legs of the mice. At the end of the two weeks, the mice were sacrificed and the Tibialis anterior muscles were embedded with OCT mounting media, flash frozen in liquid nitrogen cooled isobutane, and then later sectioned for Periodic acid-Schiff (PAS) staining.

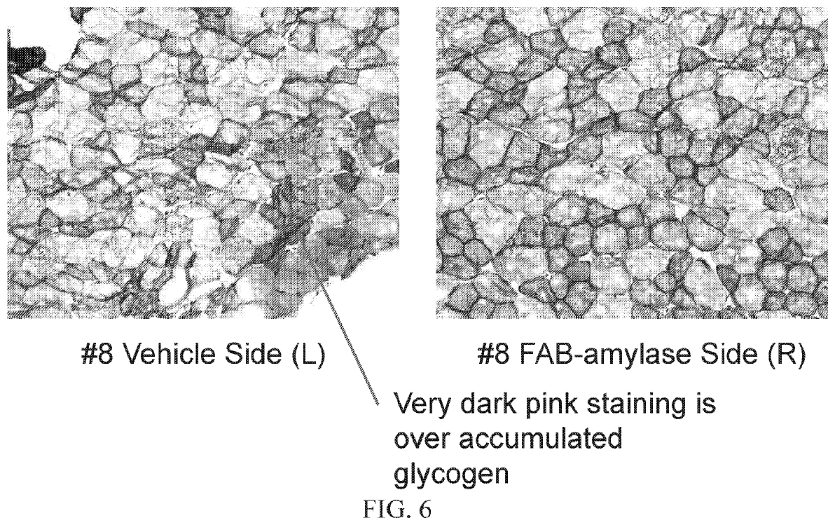

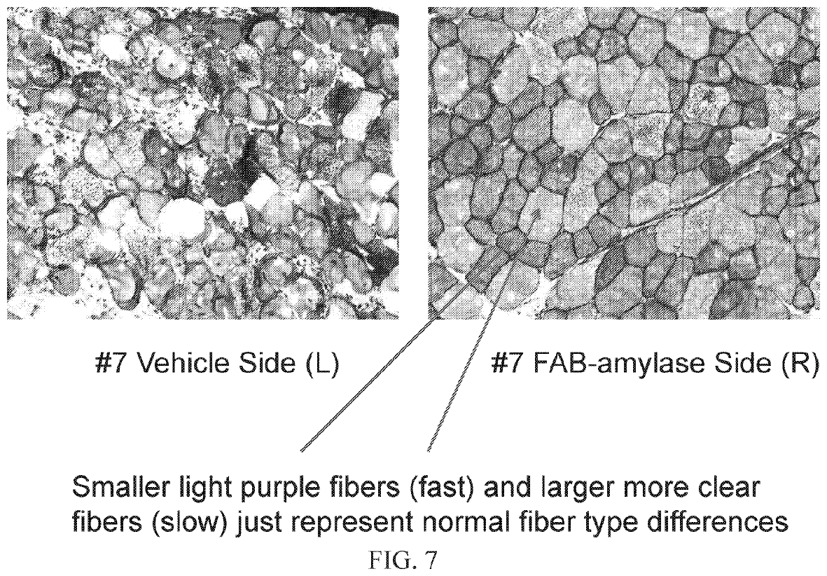

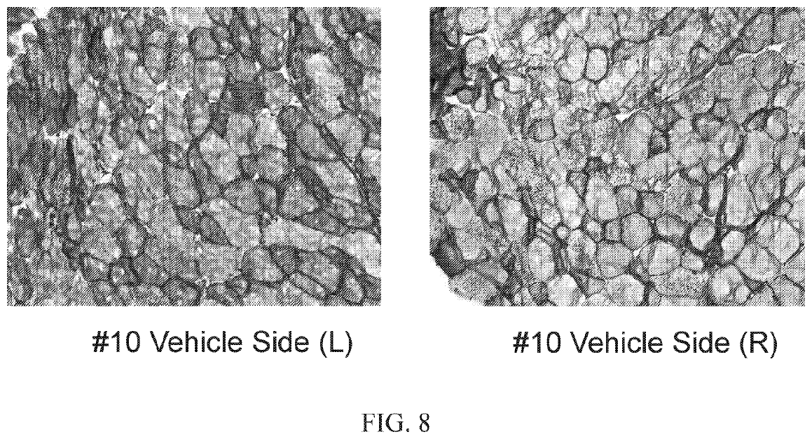

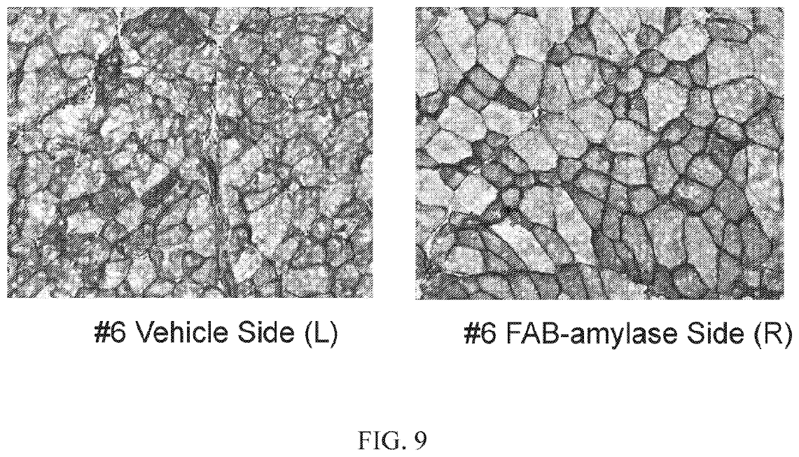

The mice that were treated with Fab-amylase showed a reduction in very strong instances of dark pink glycogen detection with PAS staining, as well as an improvement in muscle architecture (e.g., clear distinction between fast and slow muscle fibers). For example, as seen in FIG. 6, a treated 8.5 month old female mouse (specimen #8) demonstrates very dark pink staining in the left leg (PBS treated) (left panel) signifying over accumulated glycogen. In comparison, the Fab-amylase treated muscle does not show the same staining (right panel). A second treated 8.5 month old female mouse (specimen #7) exhibits similar results as seen in FIG. 7. The Fab-amylase treated muscle (right panel) also shows normal fiber differences between fast fibers (small, light purple) and slow fibers (larger, more clear), as compared to the PBS treated muscle (left panel). FIG. 9, which provides a comparison of PBS treated muscle (left panel) to Fab-amylase treated muscle (right panel) of a 4 month old female mouse (specimen #6), further supports these findings. An 8.5 month old female mouse (specimen #10) acts as a control (FIG. 8) with PBS treated muscles for both the left and right legs (left panel and right panel, respectively).

iii. Assessment of Fab-Amylase in the Brain Upon ICV Administration

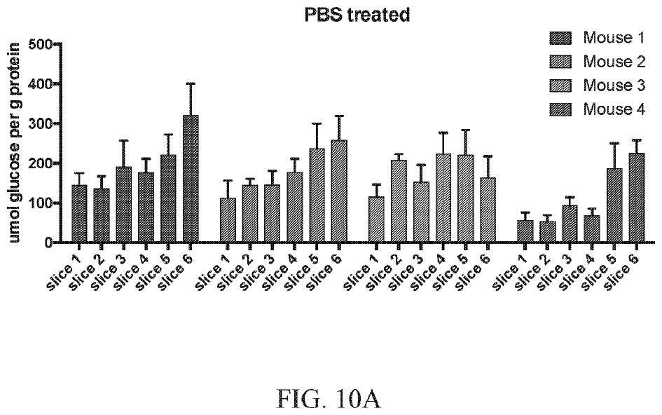

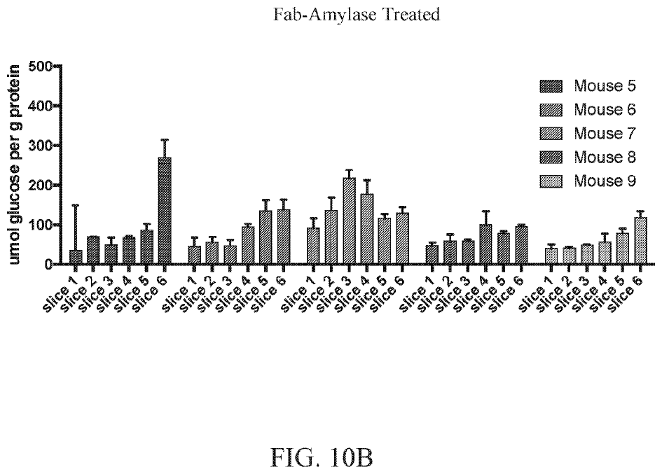

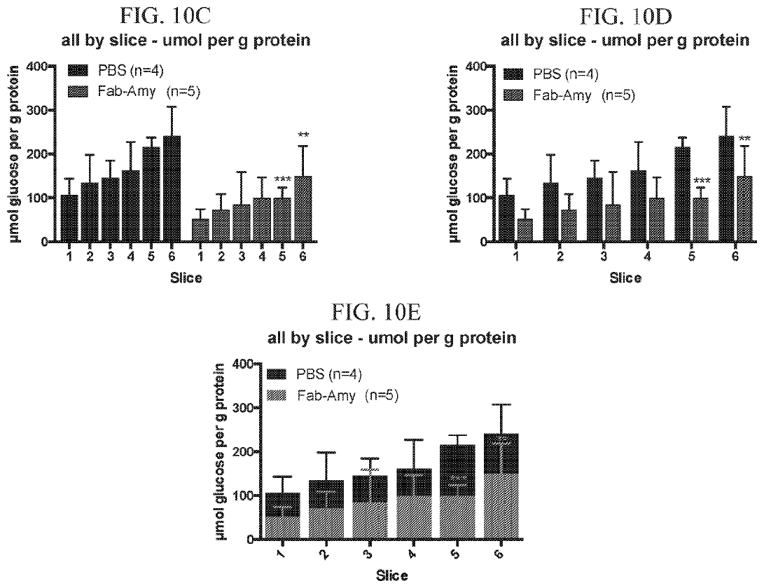

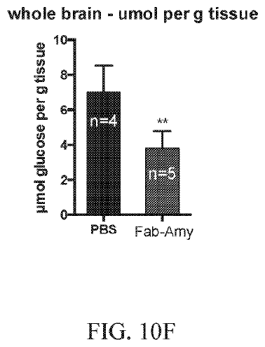

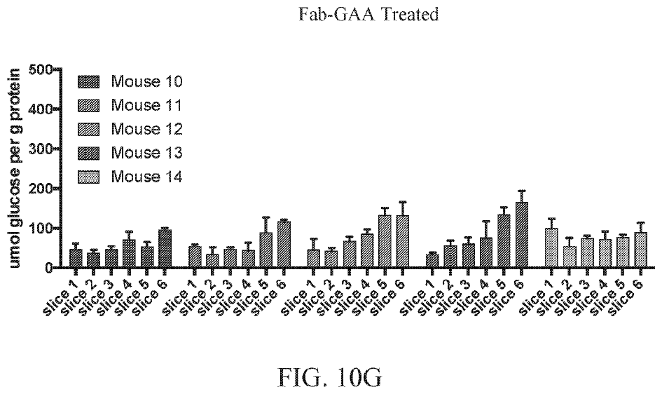

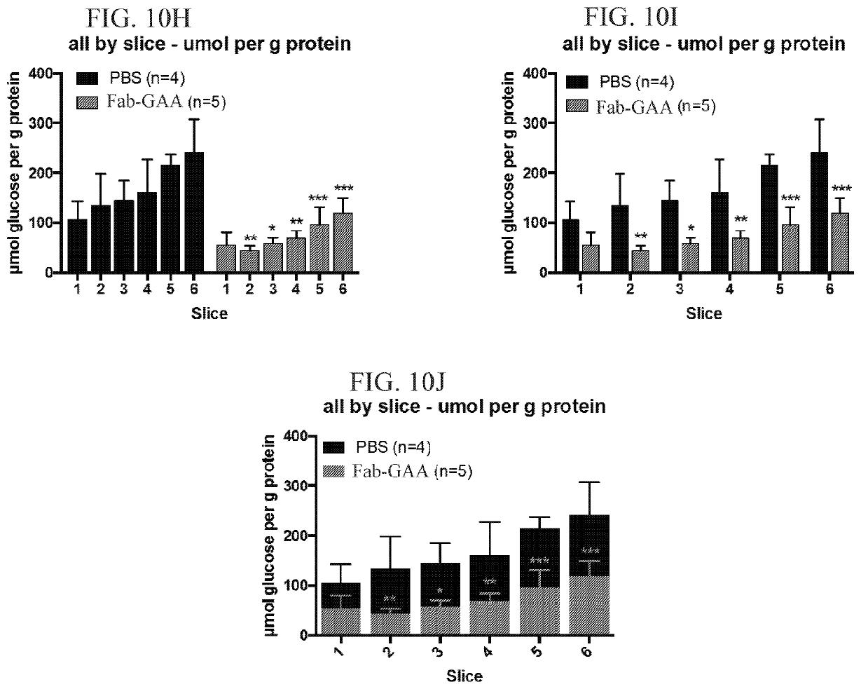

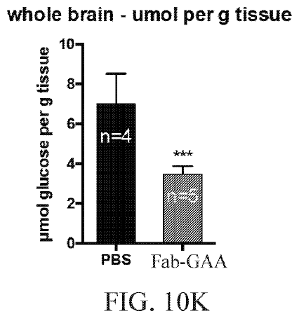

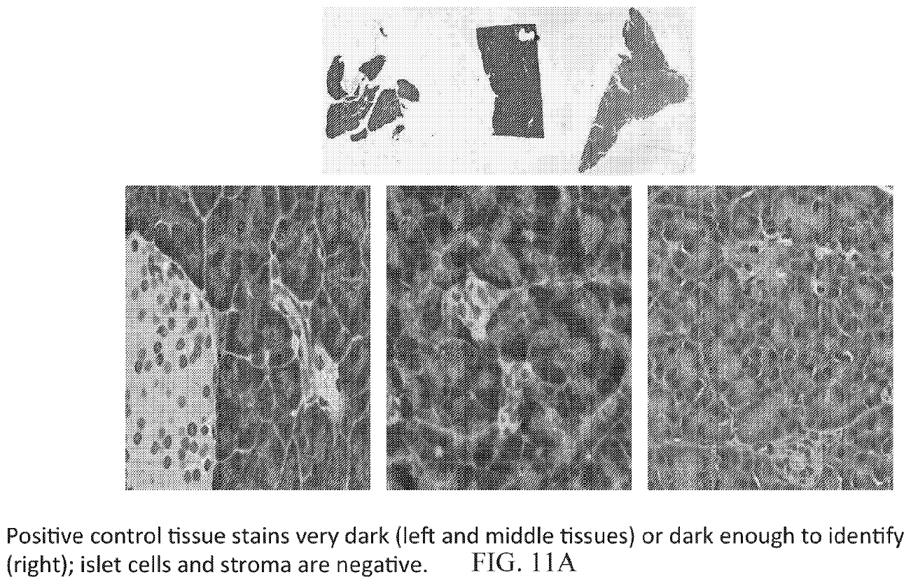

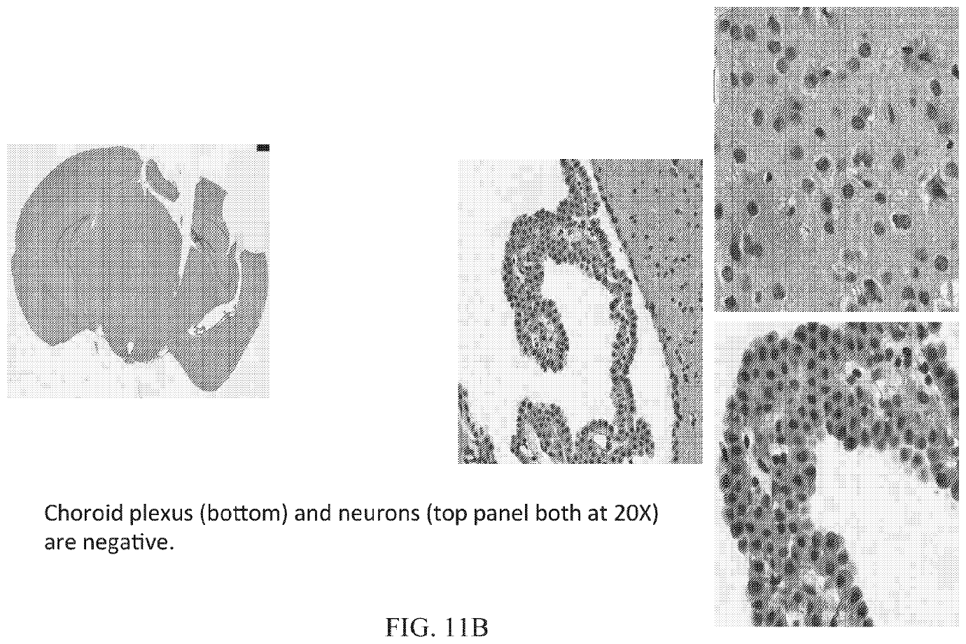

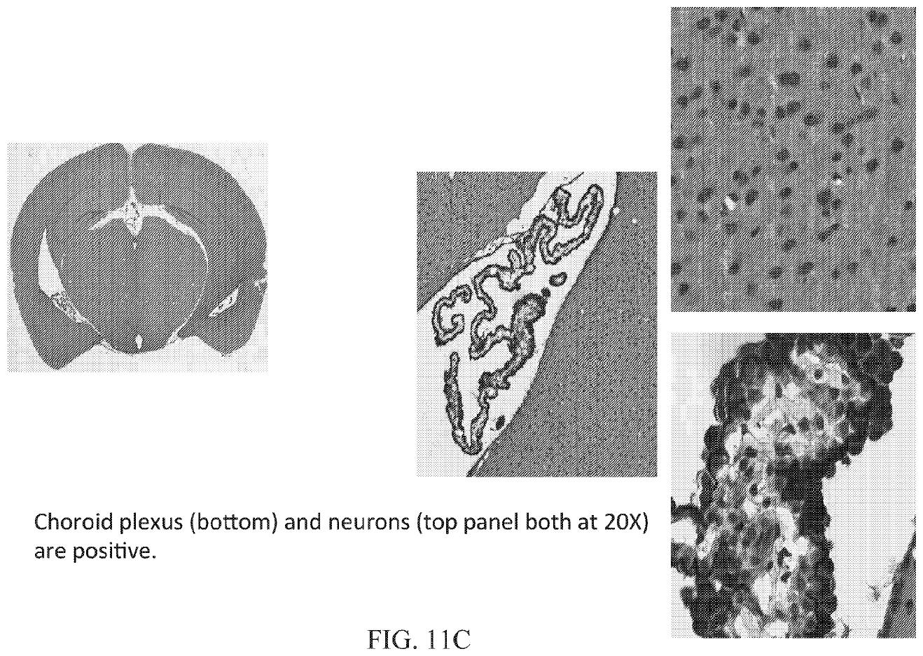

The effect of ICV pump administration of Fab-amylase is assessed by comparing Fab-amylase treated mice with PBS-treated mice. In the PBS treated mice, four mice were administered PBS via ICV pump for 28 days and in the Fab-amylase mice, five mice were administered Fab-Amylase via ICV pump over the course of 28 days. At the end of the 28 days the mice were sacrificed and brains were sectioned into six slices. Glucose levels were measured in each brain section of each mouse (PBS treated and Fab-Amylase treated) (FIGS. 10A-10F). The mice that were treated with Fab-Amylase showed a clearance of glycogen in the brain. This was further demonstrated by IHC (anti-amylase staining) showing wide distribution through the brain and uptake into neurons of Fab-Amylase. (FIGS. 11A-11D).

iv. Assessment of Fab-Amylase on Gastrocnemius Muscle

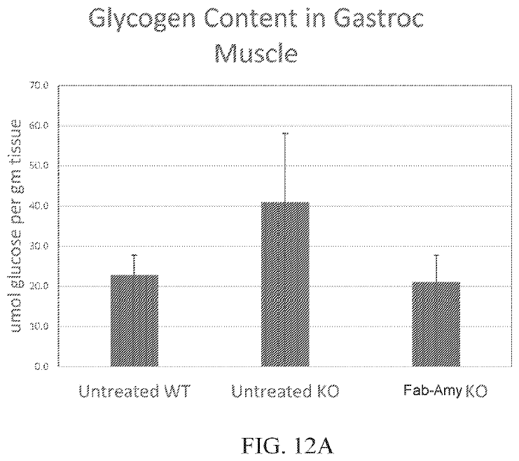

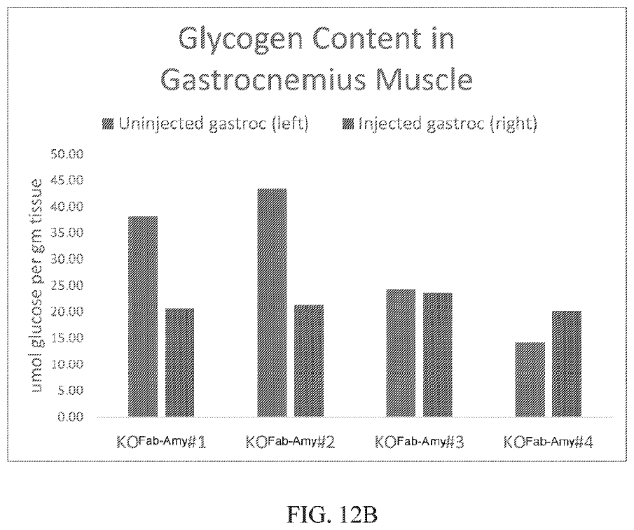

The effect of intramuscular gastroc muscle injections of Fab-Amylase is assessed using wild type mice and laforin knock-out mice. In four 10 month old laforin knock-out mice, the right gastroc was injected with 30 mg/ml Fab-Amylase three times over 7 days. The mice were sacrificed 24 hours after the last injection and glycogen was measured in the right and left gastrocs. Glycogen content samples were additionally taken from age-matched wild-type animals (N=4), as well as from the untreated muscle in the laforin knock-out mice (N=4) (FIGS. 12A-12B).

### Example 2: Generation and Characterization of a Fab-Acid Alpha-Glucosidase

A. Design of Acid Alpha-Glucosidase Constructs

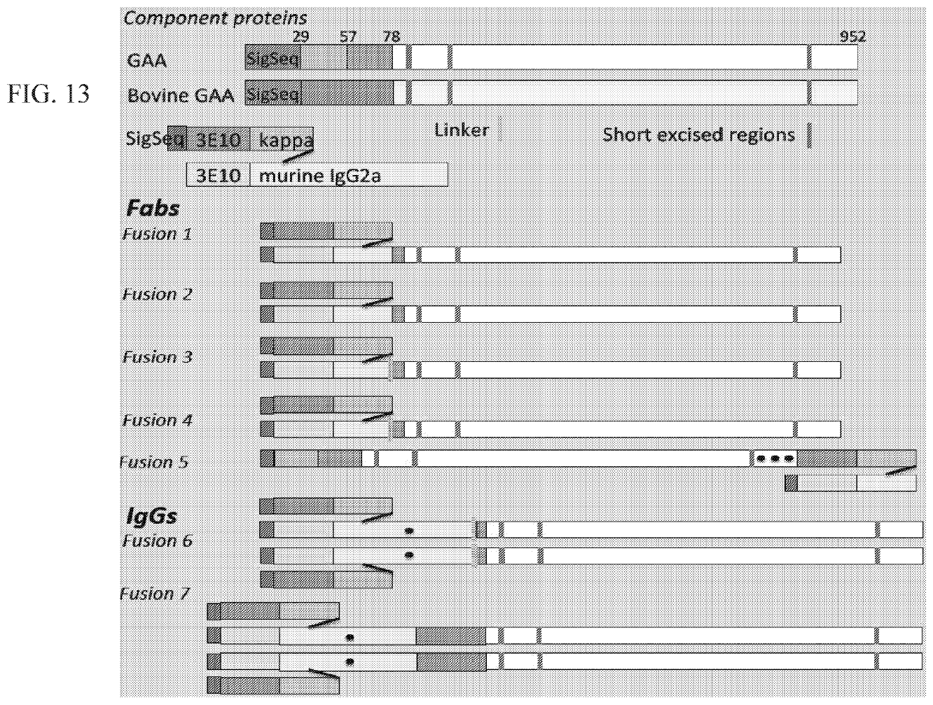

Examples of GAA constructs are designed to include 3E10 Fab and whole-antibody fusions to the GAA enzyme. The Fab-GAA constructs included 1) 3E10 Fab with GAA 70-952 fused to the C-terminus of the heavy chain Fab segment; 2) 3E10 Fab with GAA 61-952 fused to the C-terminus of the heavy chain Fab segment; 3) 3E10 Fab with a 5-amino acid linker and GAA 57-952 fused to the C-terminus of the heavy chain Fab segment; 4) 3E10 Fab with a 13-amino acid linker and GAA 67-952 fused to the C-terminus of the heavy chain Fab segment; and 5) GAA with point mutations designed to enhance C-terminal fusion, a 13-amino acid linker, and a 3E10 Fab fused at the N-terminus of the light chain. The Mabs constructs included 6) a 3E10 whole antibody fused to GAA at the C-terminus of the heavy chain, with a junction similar to that of construct 4 above; and 7) a 3E10 whole antibody fused to GAA at the C-terminus of the heavy chain, with a bovine GAA pro-sequence upstream of the mature GAA sequence. Schematics of the various construct designs are provided in FIG. 13. Fusion 4 is identified as a fusion of interest and is selected for further examination. One rationale for starting at GAA residue 67 in Fusion 4, rather than including the entire 57-77 “pro” segment involved in GAA processing, was to avoid any potential Arg-C protease cleavage after Arg66.

B. Fab-Acid Alpha-Glucosidase in a Cell-Free Activity Assay

A cell-free activity assay is performed to compare activity of mAB-GAA and Fab-GAA samples. The samples are thawed on ice and 10-fold serial dilutions (10×, 100×, and 1000×, and 10000×) are made with water. Acid and neutral GAA activity is measured at pH4.3 and pH6.7, respectively, for each sample of different dilutions using 4-methylumbelliferyl a-D-glucoside as fluorescent substrate. (J. L. Van Hove, et al., “High-level production of recombinant human lysosomal acid alpha-glucosidase in Chinese hamster ovary cells which targets to heart muscle and corrects glycogen accumulation in fibroblasts from patients with Pompe disease” PNAS 93 (1996) 65-70; J.Y. Wu, et al., “Expression of catalytically active human multifunctional glycogen-debranching enzyme and lysosomal acid alpha-glucosidase in insect cells” Biochem Mol Biol Int 39 (1996) 755-764.) The assay demonstrates that the murine Fab-GAA samples are more active than a mAB-fusion (Table 3). Additionally, it was shown that GAA activity was much less (i.e., 3%) at high pH (ph 6.7) vs low pH (ph 4.3).

TABLE 3

| | | | GAA activity | |
|---|---|---|---|---|
| | GAA activity | GAA activity | (% pH 6.7/ | |
| Sample | (pH = 4.3) | (pH = 6.7) | pH 4.3) | |
| name | nmol/hr/mL | nmol/hr/mL | Raw | Average |
| mAb-GAA | 979.39 | 29.18 | 2.98 | 2.98 |
| mAb-GAA | 966.70 | 32.57 | 3.37 | |
| mAb-GAA | 994.39 | 31.68 | 3.19 | |
| mAb-GAA | 999.45 | 29.32 | 2.93 | |
| Fab-GAA | 1514.10 | 46.47 | 3.07 | 3.07 |
| Fab-GAA | 1528.10 | 46.76 | 3.06 | |
| Fab-GAA | 1497.60 | 44.25 | 2.95 | |
| Fab-GAA | 1471.40 | 45.14 | 3.07 | |

C. pH Dependent Specific Activity of Fab-Acid Alpha-Glucosidase

A Fab-GAA glycogen assay is performed to assess pH dependent specific activity of Fab-GAA. The materials for performing the assay include Fab-GAA, Glucose Oxidase Kit (Sigma GAGO20-1KT), and Glycogen from a rabbit liver (Sigma G8876-1G). Glucose standards are prepared by serial dilution in water from 1 mg/mL glucose in G.O. kit: 0.1 mg/mL, 0.05 mg/mL, 0.025 mg/mL, 0.0125 mg/mL, 0.00625 mg/mL, 0.003125 mg/mL (0.1 mg/mL=555.1 μM). 20 mL of citrate and/or phosphate buffers are prepared from pH 3.5-7.0 by adding 0.1M citric acid and 0.2M sodium phosphate dibasic in the amounts recited in the table below. Spike in 10% Tween-80 to 0.02% final and verify pH with pH meter. In addition, 0.1M sodium acetate pH 4.3+0.02% tween-80 is prepared.

TABLE 4

| | mL 0.1M | mL 0.2M |
|---|---|---|
| pH | Citric Acid | NaPO₄ Dibasic |
| 3.5 | 6.04 | 13.96 |
| 4 | 7.72 | 12.28 |
| 4.3 | 8.49 | 11.51 |
| 4.5 | 9 | 11 |
| 5 | 10.28 | 9.72 |
| 5.5 | 11.36 | 8.64 |
| 6 | 12.84 | 7.16 |
| 6.5 | 14.2 | 5.8 |
| 7 | 17.44 | 2.56 |

10 mg/mL of glycogen is prepared in each buffer solution to be tested. For example, a 10 mg/mL glycogen solution is prepared in 0.1M acetate 0.02% Tween 80 pH 4.3. Fab-GAA is diluted to 1 mg/mL in reaction buffer, and then 1.8 μL 1 mg/mL Fab-GAA is added to 178.2 μL glycogen solution in 500 μL vial providing a final Fab-GAA concentration of 10 μg/mL. The solution is mixed well and incubated at ambient temperature for 1 hour. A portion of the glycogen solution is retained as a negative control. Digestion may be terminated by heating the sample at 95° C. for 10 minutes. A Fab-GAA negative glycogen sample is also heated as a negative control/blank sample. Standards and digested glycogen (40 μL/well) are pipetted into a 96 well plate in triplicate. 80 μL room temperature G.O. Reagent Mix (prepared as described in kit) is added to each well with a multi-channel pipette, mixed well and incubated 37° C. for 30 min. A pale brown color should begin forming. The reaction is then terminated and the plate developed by adding 80 μL 12N sulfuric acid with a multi-channel pipette and mixing well. The color should turn pink. The plate is then read at 540 nm.

In the present assay, five samples were prepared and assayed, including: 1 standard digest of glycogen by Fab-GAA; 1 sample with 0.05 mg/mL (278 μM) glucose spiked in prior to digestion; 1 sample with 0.025 mg/mL (139 μM) glucose spiked in prior to digestion; 1 sample with 0.05 mg/mL (278 μM) glucose spiked in after digestion; and 1 sample with 0.025 mg/mL (139 μM) glucose spiked in after digestion. Assuming no glucose inhibition, it is expected that pre-digestion and post-digestion samples will be similar. Additionally, it is expected that the wells will read 55.5 μM and 27.8 μM higher than the no-spike sample (samples are diluted 5× between digestion and assay at 540 nm).

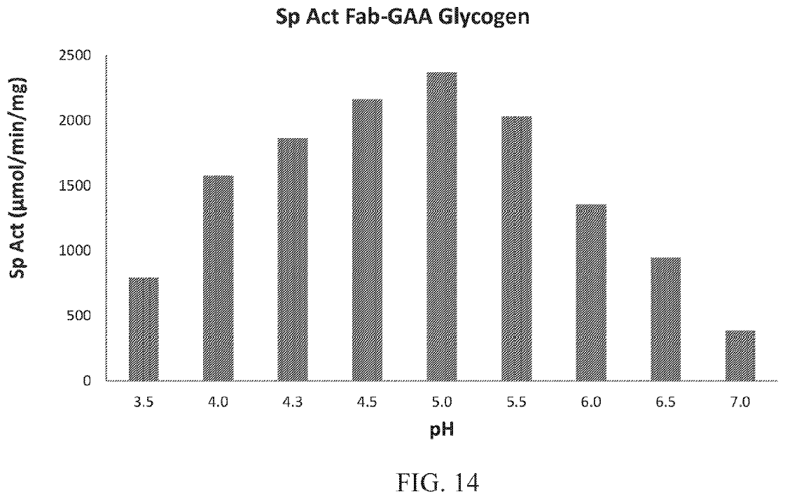

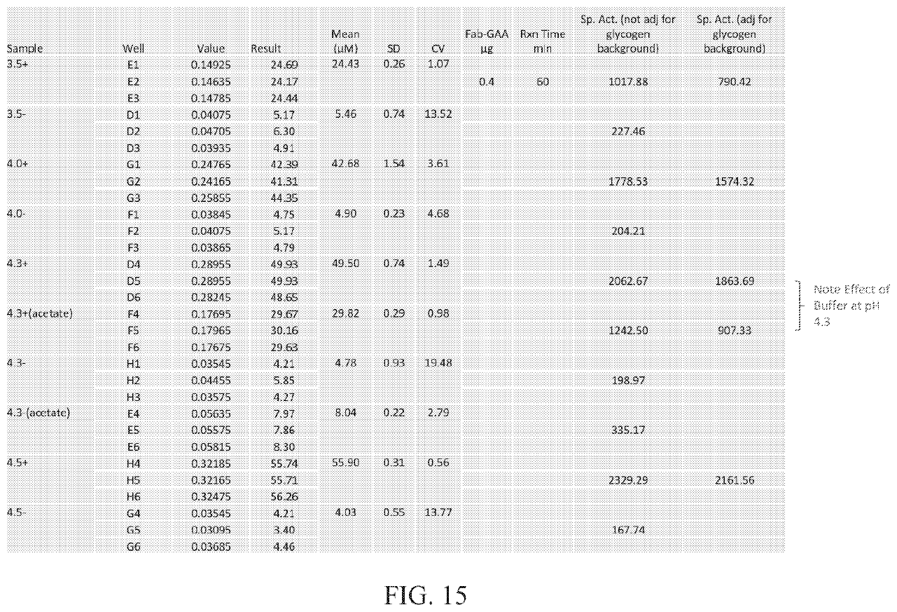

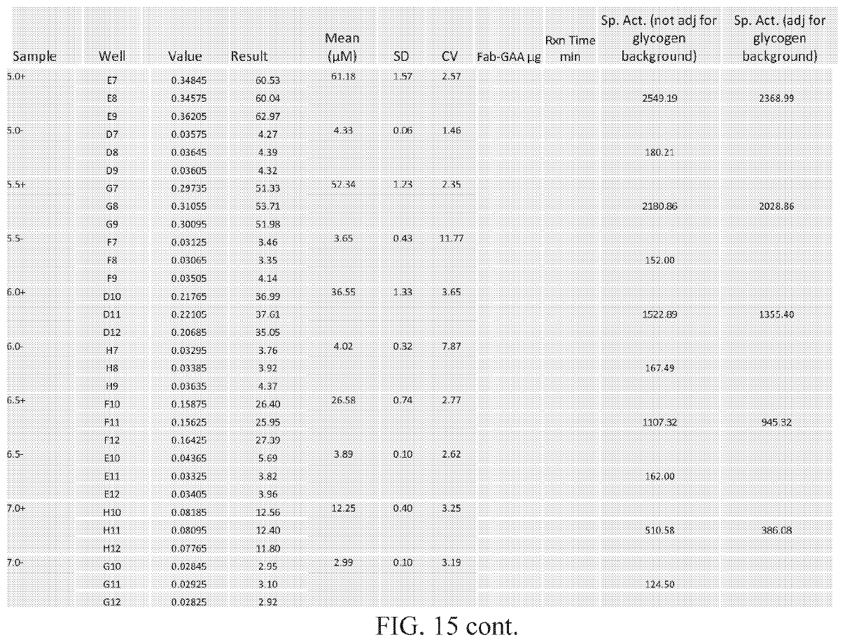

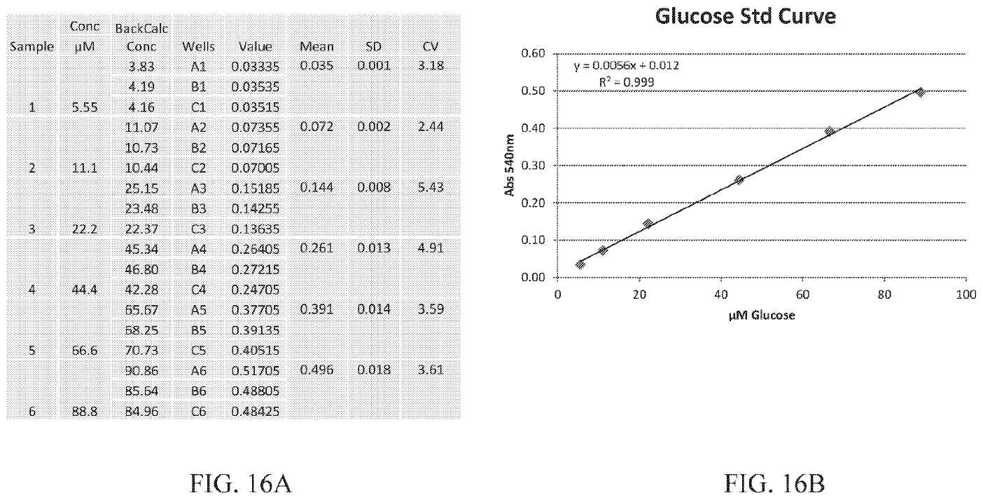

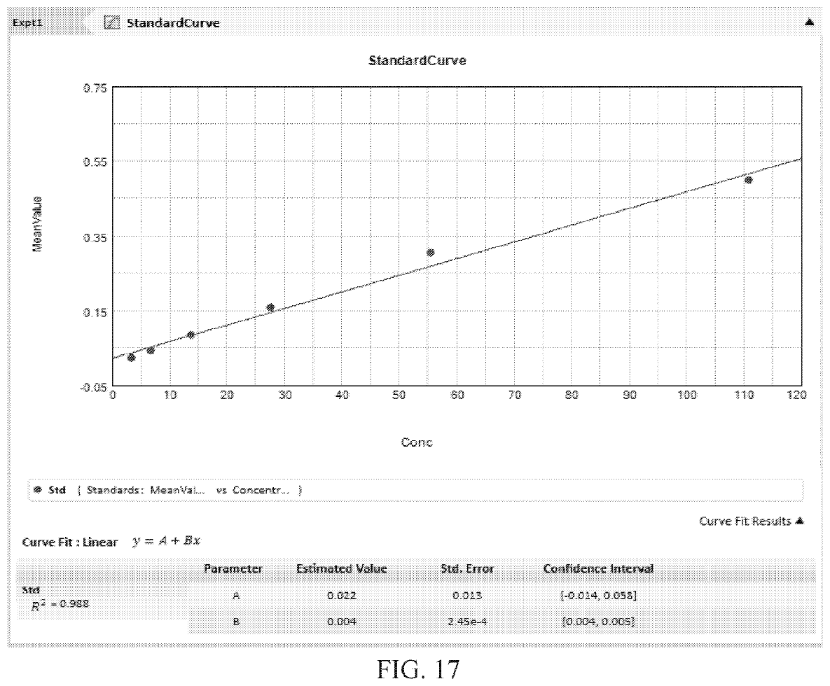

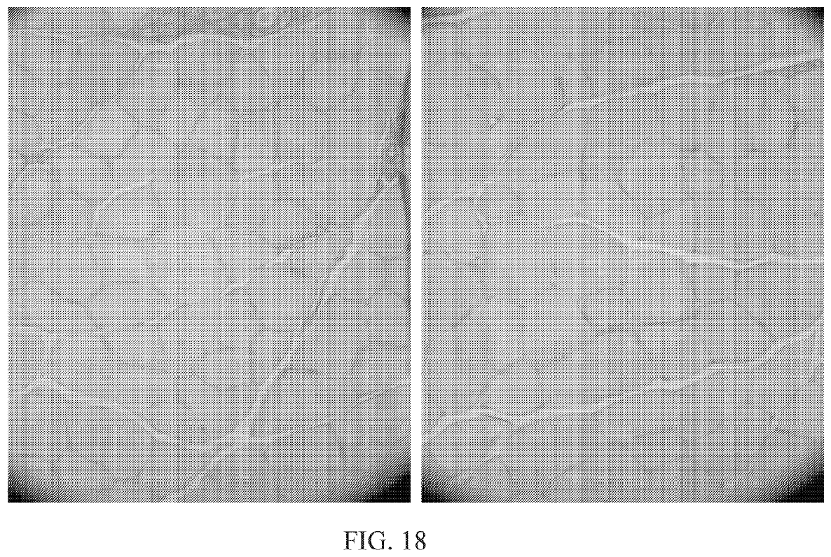

The Fab-GAA glycogen assay results are shown in Tables 5 and 6 (shown below) and in FIGS. 14-16. There is minimal difference between samples spiked with glucose before or after glycogen digest, which suggests low glucose inhibition at these concentrations. In addition, all spikes are within 10% of expected values. Finally, the Fab-GAA glycogen specific activity measured at 1140.17 uM/min/mg. A Fab-GAA glycogen standard curve and relevant data is provided in FIG. 16, as well as in Table 7 and corresponding FIG. 17. The standard curve shown in FIG. 17 has a R² value that is slightly below target with some non-linearity of the standard curve noted at max range (the signal begins to plateau). A slight downward adjustment of the upper limit is required for evaluating 75-100 μM (currently evaluating 111 μM).

TABLE 5
Fab-GAA Glycogen Assay Results

| | | | | | | | | | Digest | Specific |
|---|---|---|---|---|---|---|---|---|---|---|
| | | | | | MeanResult | | | | Time | Activity |
| Sample | Wells | Value | R | Result | (uM) | SD | CV | ug/assay | (min) | (uM/min/mg) |
| Fab-GAA, | C2 | 0.1458 | | 27.821 | 27.364 | 0.696 | 2.5 | | | |
| no spike | D2 | 0.1453 | | 27.708 | | | | | | |
| | E2 | 0.1402 | | 26.562 | | | | 0.4 | 60 | 1140.17 |

TABLE 6
Fab-GAA Glycogen Assay Results

| | | | | | | | | | | Percentage of |
|---|---|---|---|---|---|---|---|---|---|---|
| | | | | | MeanResult | | | Spike | Corrected | non-spiked |
| Sample | Wells | Value | R | Result | (uM) | SD | CV | Adjustment | Result | sample |
| 55.5 uM spike | C6 | 0.3979 | | 84.475 | 81.426 | 2.641 | 3.2 | −55.5 uM | 25.926 | 94.7% |
| (Post) | D6 | 0.3778 | | 79.958 | | | | | | |
| | E6 | 0.3773 | | 79.846 | | | | | | |
| 55.5 uM spike | C4 | 0.3835 | | 81.239 | 83.786 | 2.306 | 2.8 | −55.5 uM | 28.286 | 103.4% |
| (Pre) | D4 | 0.4035 | | 85.734 | | | | | | |
| | E4 | 0.3975 | | 84.385 | | | | | | |
| 27.8 uM spike | C5 | 0.2805 | | 58.092 | 57.35 | 2.219 | 3.9 | −27.8 uM | 29.55 | 108.0% |
| (Post) | D5 | 0.2661 | | 54.856 | | | | | | |
| | E5 | 0.285 | | 59.103 | | | | | | |
| 27.8 uM spike | C3 | 0.2716 | | 56.092 | 57.852 | 2.298 | 4 | −27.8 uM | 30.052 | 109.8% |
| (Pre) | D3 | 0.291 | | 60.451 | | | | | | |
| | E3 | 0.2757 | | 57.013 | | | | | | |

TABLE 7
Fab-GAA Glycogen Standard Curve Raw Data

| | Concentration | | | | | | |
|---|---|---|---|---|---|---|---|
| Sample | μM | BackCalcConc | Wells | Value | MeanValue | SD | CV |
| 1 | 3.5 | −0.08 | A1 | 0.02165 | 0.021 | 0.001 | 4.4 |
| | | −0.372 | B1 | 0.02035 | | | |
| 2 | 6.9 | 4.617 | A2 | 0.04255 | 0.042 | 0.001 | 2.5 |
| | | 4.28 | B2 | 0.04105 | | | |
| 3 | 13.9 | 15.067 | A3 | 0.08905 | 0.085 | 0.006 | 7 |
| | | 13.179 | B3 | 0.08065 | | | |
| 4 | 27.8 | 30.259 | A4 | 0.15665 | 0.157 | 0 | 0.3 |
| | | 30.394 | B4 | 0.15725 | | | |
| 5 | 55.5 | 62.732 | A5 | 0.30115 | 0.303 | 0.003 | 0.8 |
| | | 63.541 | B5 | 0.30475 | | | |
| 6 | 111 | 101.476 | A6 | 0.47355 | 0.497 | 0.033 | 6.7 |
| | | 112.106 | B6 | 0.52085 | | | |

The data obtained from the Fab-GAA glycogen assay demonstrates that the humanized Fab-GAA construct retains better activity (i.e., 43%) at high pH (pH 6.5) vs. low pH (pH 4.5). It is hypothesized that this is due to better stability and purity of the humanized Fab-GAA fragment.

D. Fab-GAA In Vivo

i. Assessment of Fab-GAA in the Brain Upon ICV Administration

The effect of ICV pump administration of Fab-GAA is assessed by comparing Fab-GAA treated mice with PBS-treated mice. In the PBS treated mice, four mice were administered PBS via ICV pump for 28 days and in the Fab-GAA treated mice, five mice were administered Fab-GAA via ICV pump over the course of 28 days. At the end of the 28 days the mice were sacrificed and brains were sectioned into six slices. Glucose levels were measured in each brain section of each mouse (PBS treated and Fab-GAA treated) (FIGS. 10A and 10G-10K). The mice that were treated with Fab-GAA showed a clearance of glycogen in the brain. This was unexpected because FAb-GAA failed to efficiently degrade isolated lafora bodies in vitro.

ii. Assessment of Fab-GAA on Skeletal Muscle

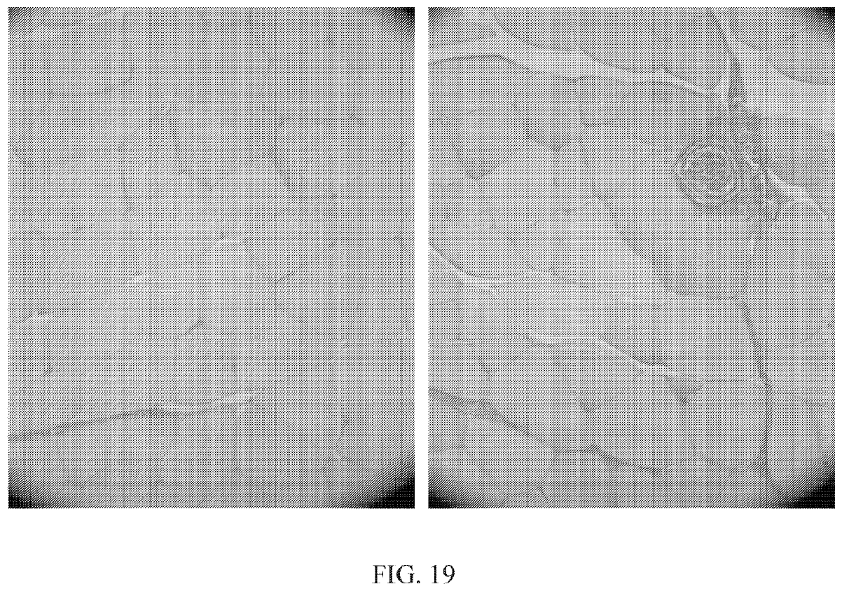

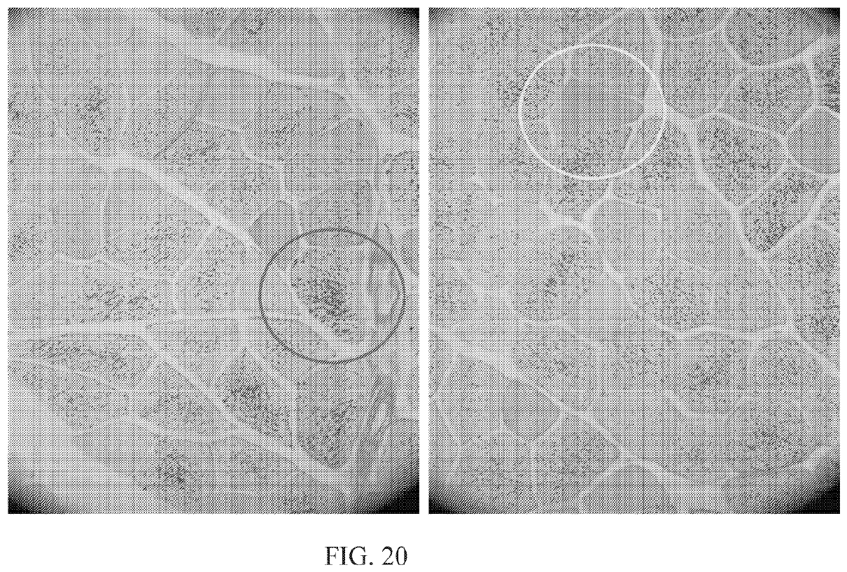

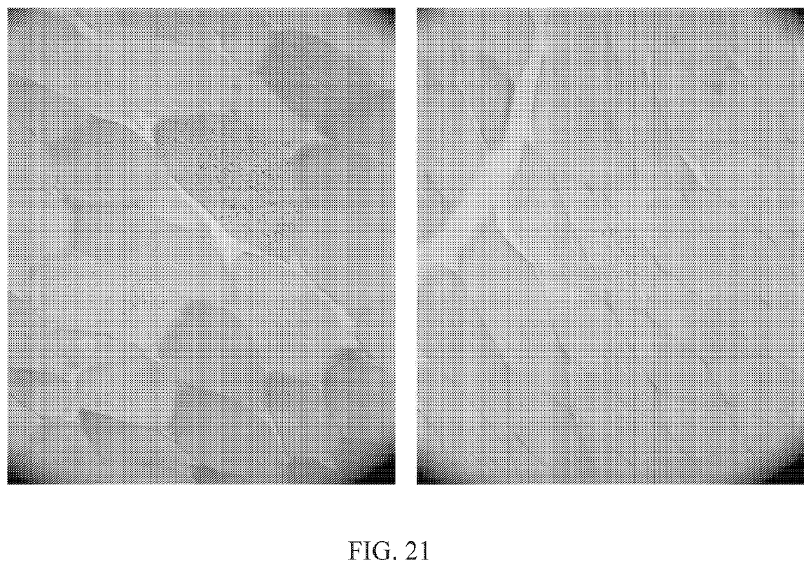

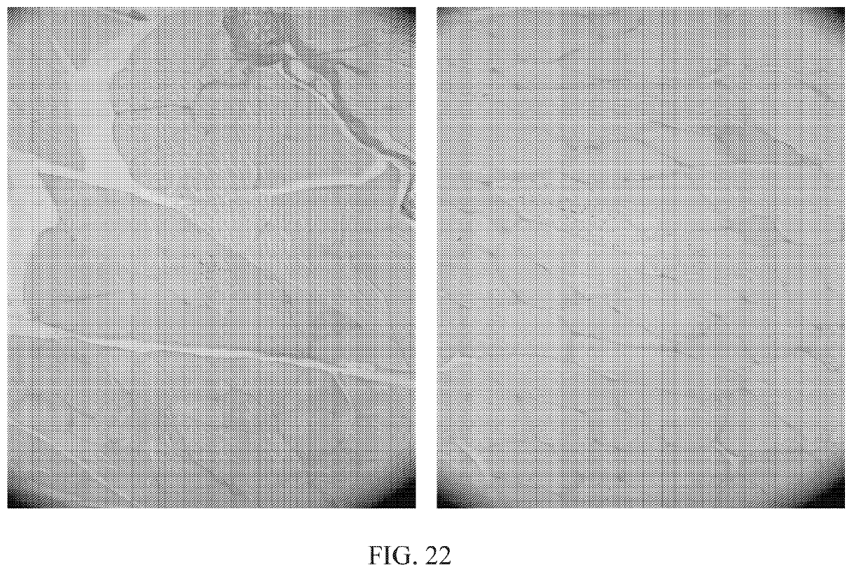

The effect of Fab-GAA on glycogen clearance in skeletal muscle is assessed using wild type mice and Lafora knock-out mice. Wild type and Lafora knock-out mice are pretreated with an IP injection of diphenhydramine (15 mg/kg) 10-15 minutes prior to each enzyme administration to prevent anaphylactic reactions. The mice are given two tail vein injections every week for two weeks for a total of four injections of Fab-GAA (90 uL at 10 mg/mL), Myozyme (120 uL at 5 mg/mL), or PBS. Injections are given on days 1, 5, 9, and 13 for a total dose of Fab-GAA of 3600 ug (180 mg/kg for a 20 g mouse) or Myozyme of 2400 ug (120 mg/kg for a 20 g mouse). The mice were then sacrificed and muscle sections of the treated mice were PAS stained (FIGS. 18-22).

iii. Assessment of Fab-GAA on Cardiac Muscle

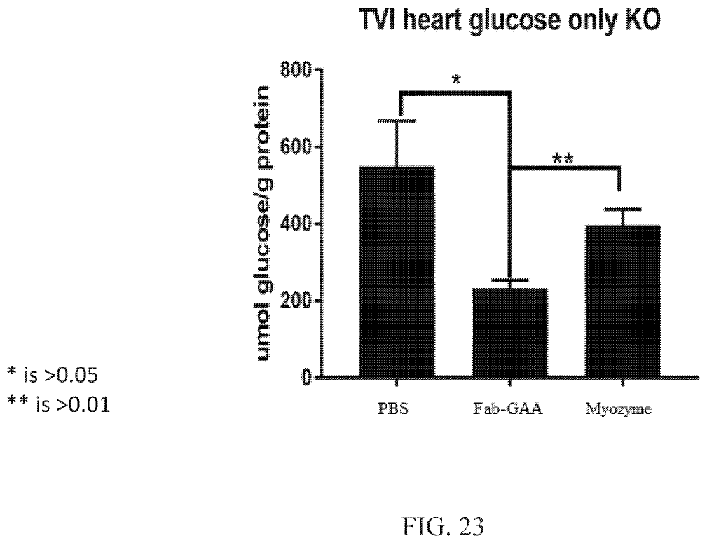

A quantitative biochemical comparison of cardiac glycogen load in Myozyme versus Fab-GAA treated Lafora knock-out mice is conducted. Lafora knock-out mice are pretreated with an IP injection of diphenhydramine (15 mg/kg) 10-15 minutes prior to each enzyme administration to prevent anaphylactic reactions. The mice are given two tail vein injections every week for two weeks for a total of four injections of Fab-GAA (90 uL at 10 mg/mL), Myozyme (120 uL at 5 mg/mL), or PBS. Injections are given on days 1, 5, 9, and 13 for a total dose of Fab-GAA of 3600 ug (180 mg/kg for a 20 g mouse) or Myozyme of 2400 ug (120 mg/kg for a 20 g mouse) (i.e., an equimolar dose to Fab-GAA). The mice were then sacrificed and the mouse heart tissue was homogenized in HEPES buffer. Tissue lysate was then used for BCA analysis of protein concentration and analysis of glucose concentration. For the glucose analysis, soluble and insoluble glycogen was collected, digested with amyloglucosidase and analyzed via glucose assay kit do determine the glucose equivalents released from the amyloglucosidase digestion (FIG. 23). Significance was measured via t test. A first biochemical analysis of glycogen load in the hearts of treated mice demonstrates a clear differentiation of Fab-GAA from Myozyme. The stored glycogen (i.e., the released glucose after the hearts are harvested and broken down ex vivo) is significantly less in Fab-GAA treated mice.

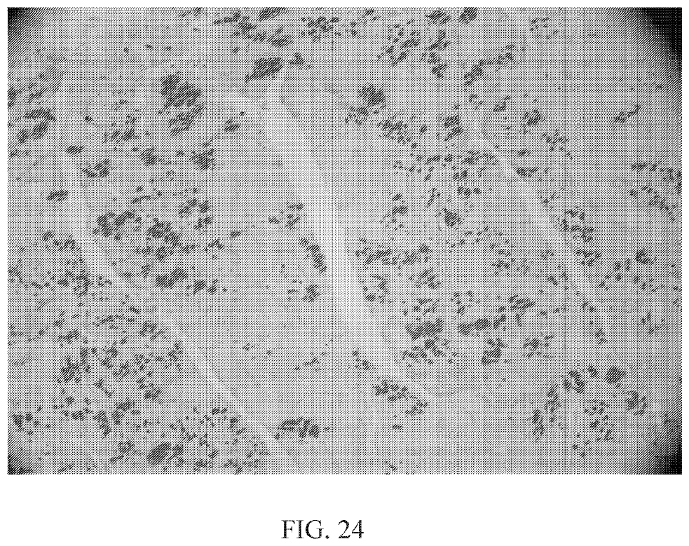

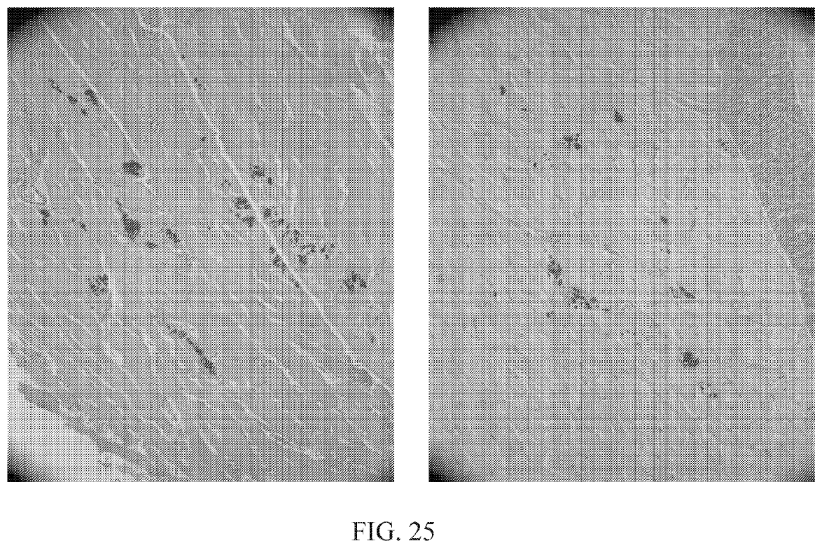

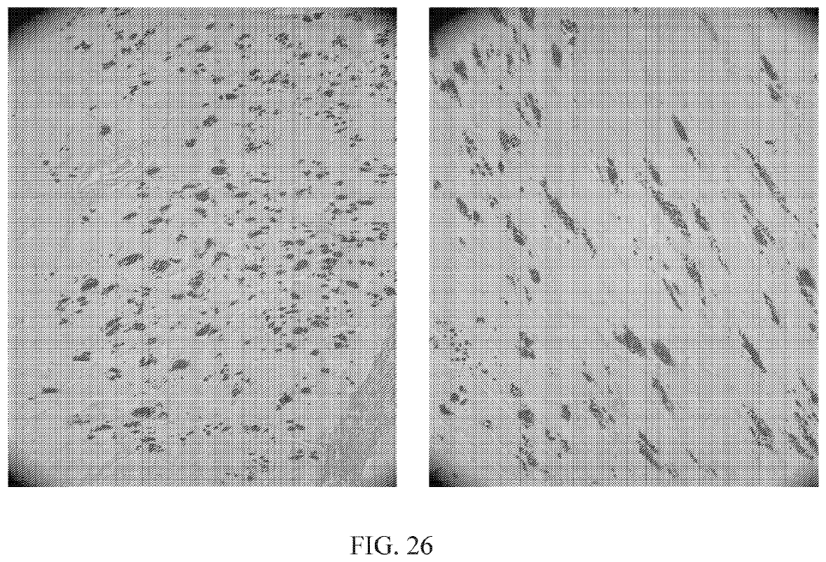

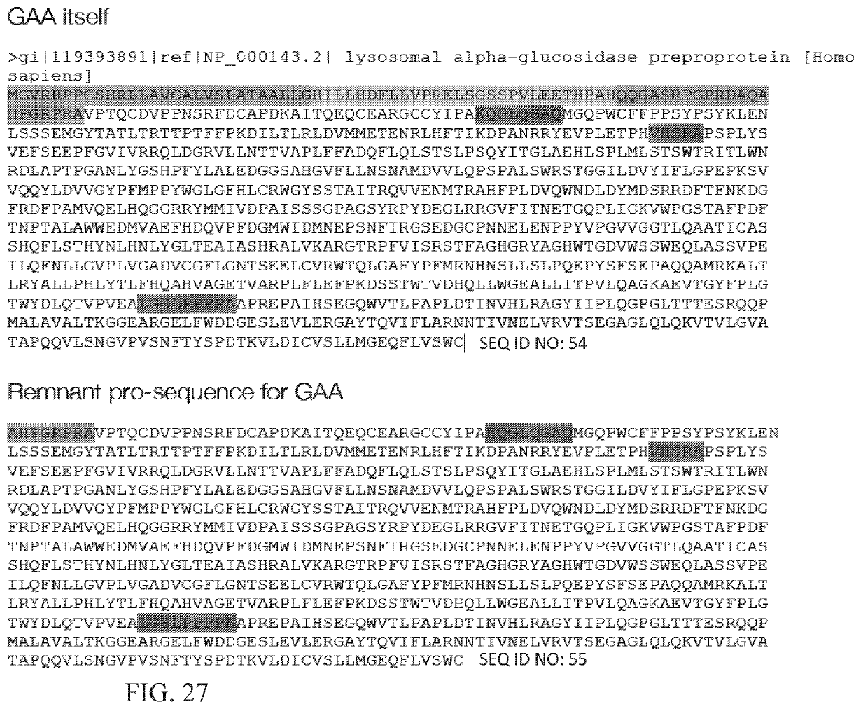

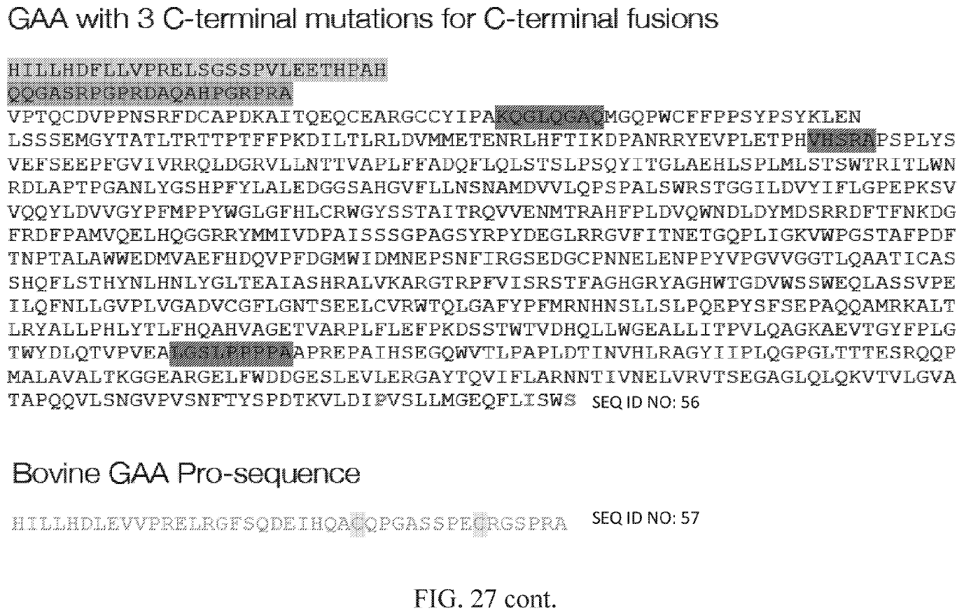

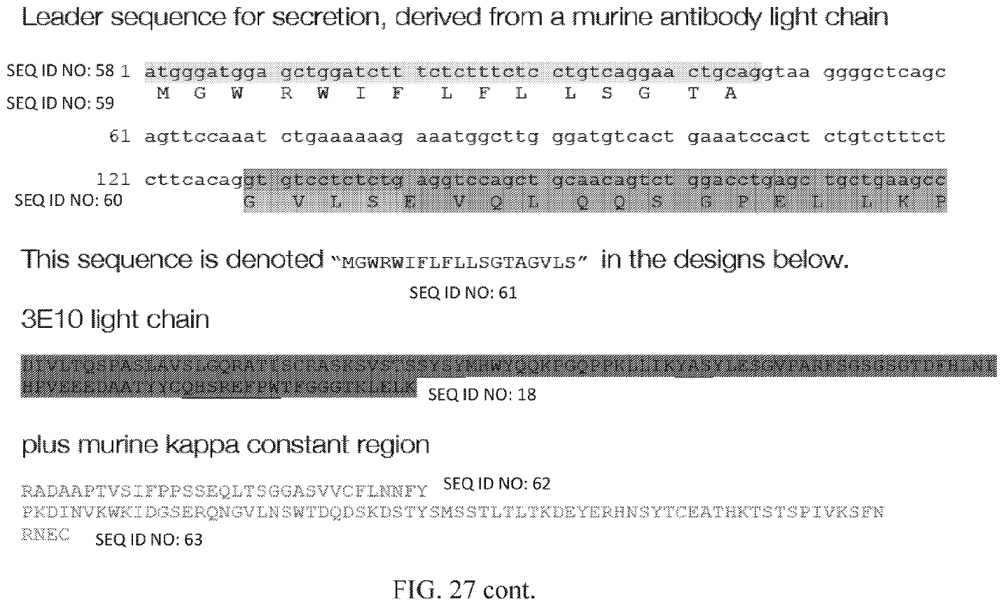

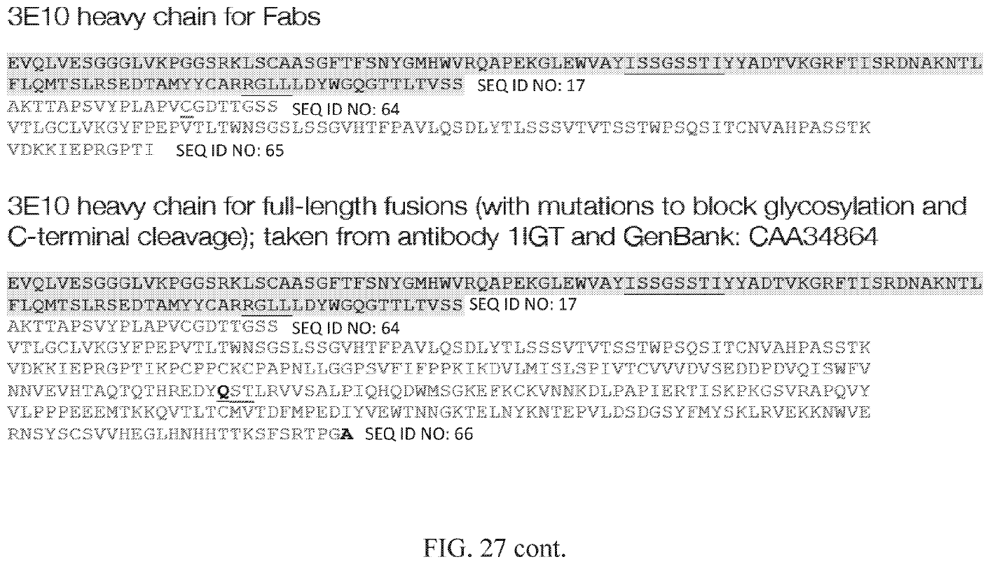

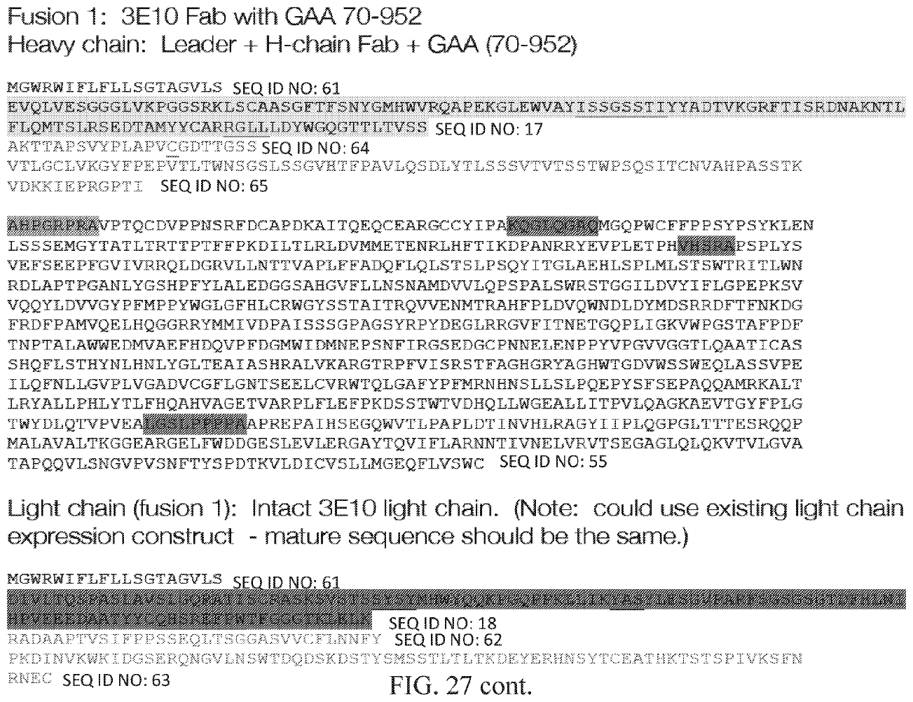

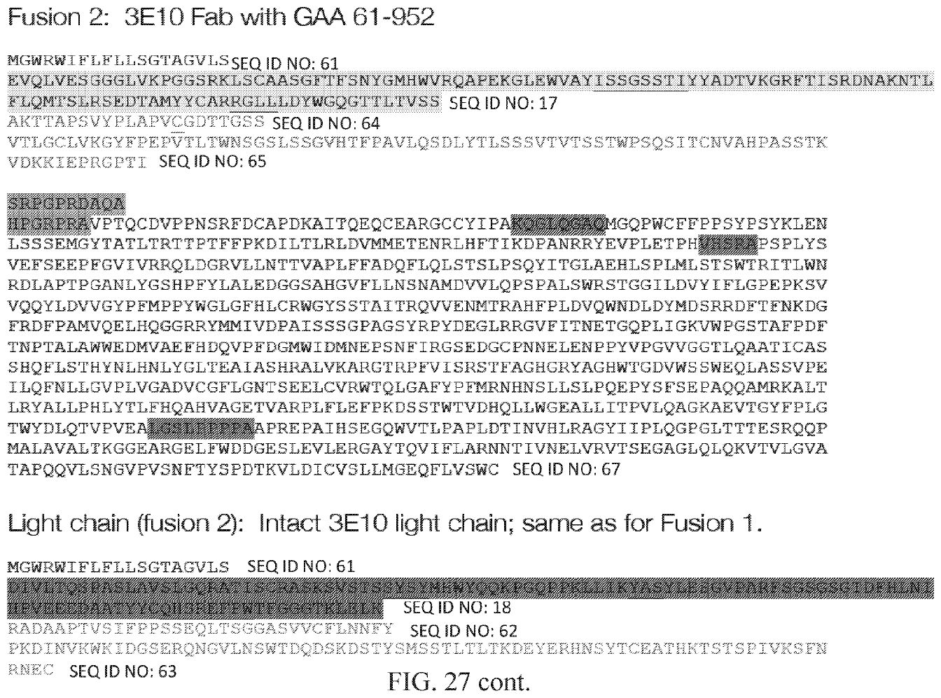

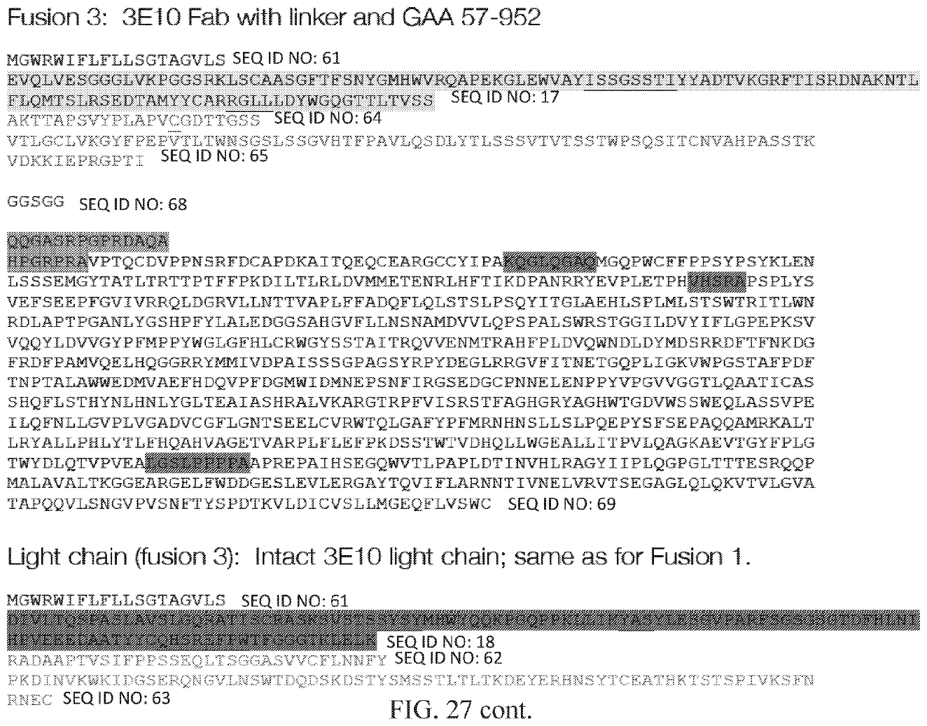

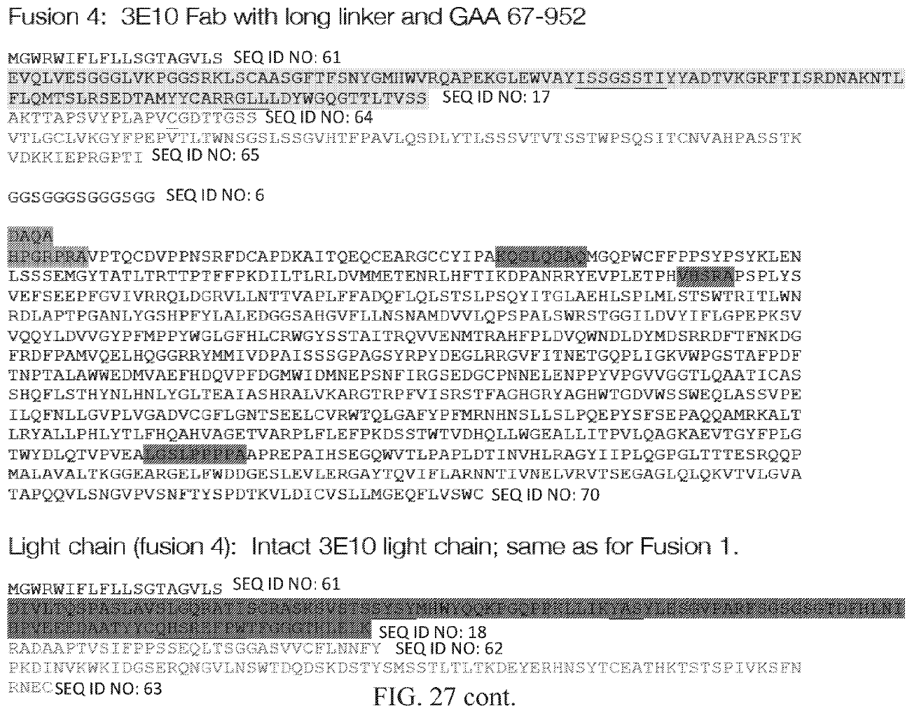

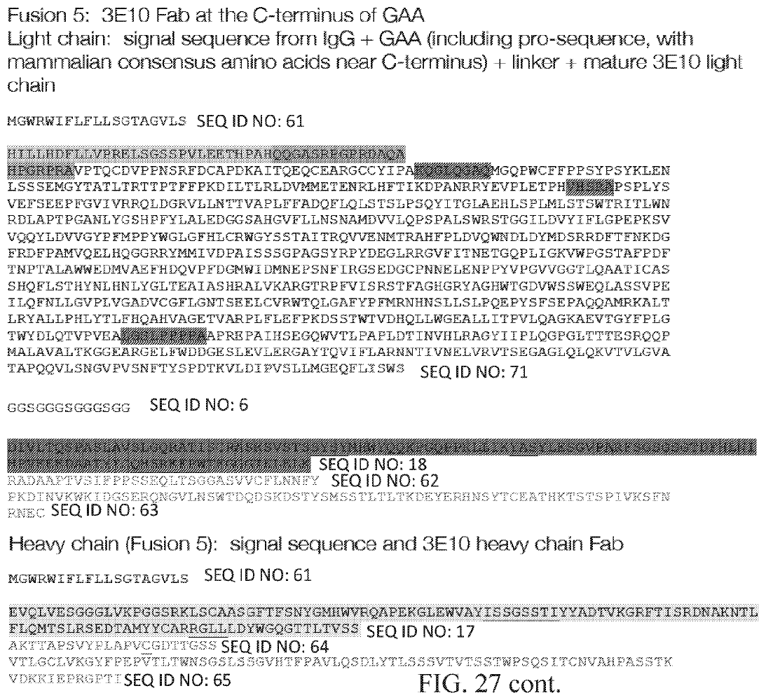

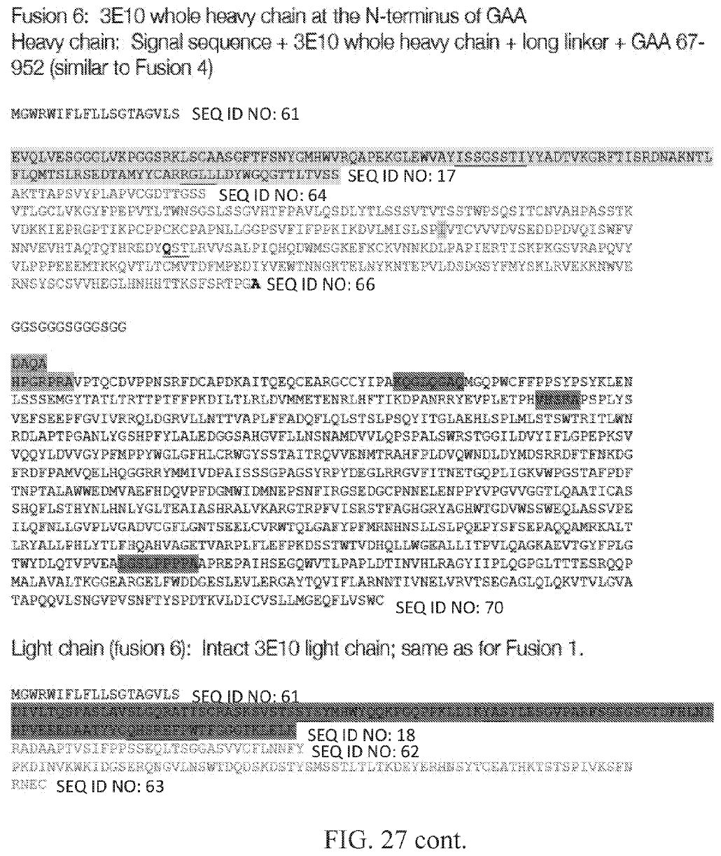

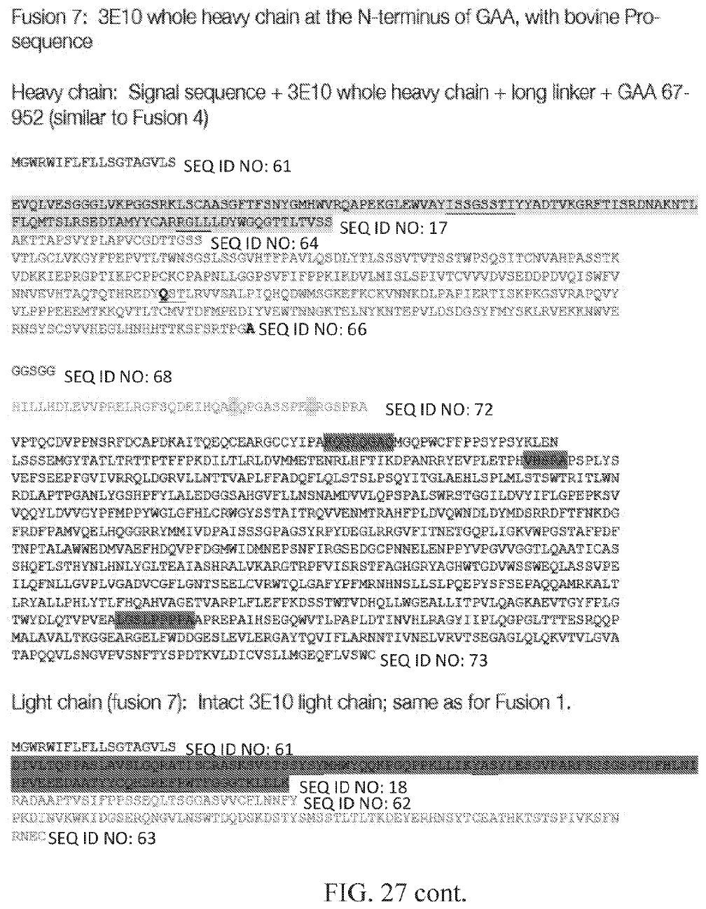

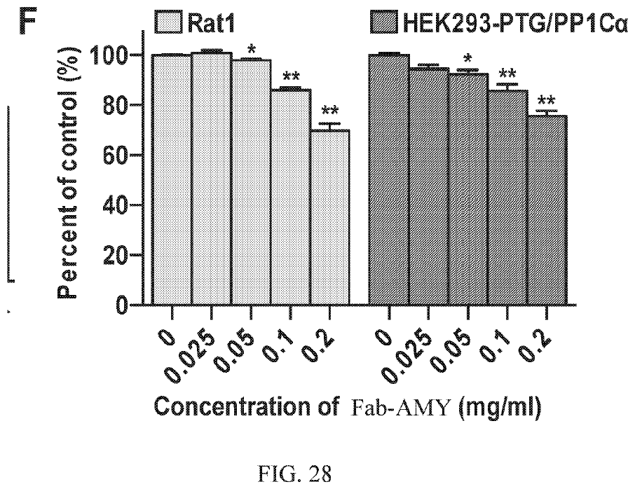

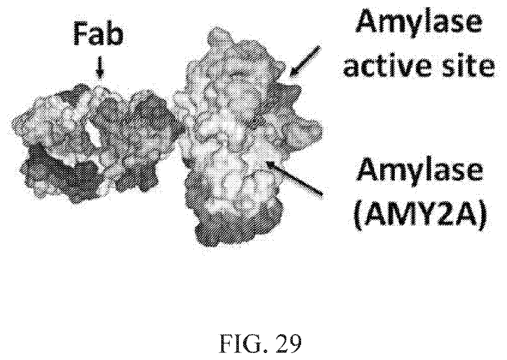

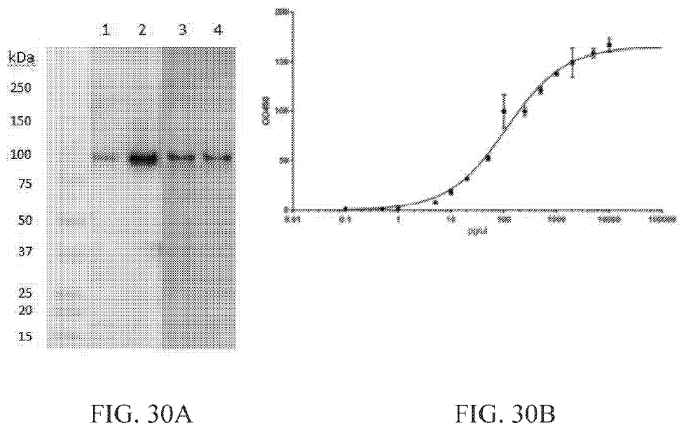

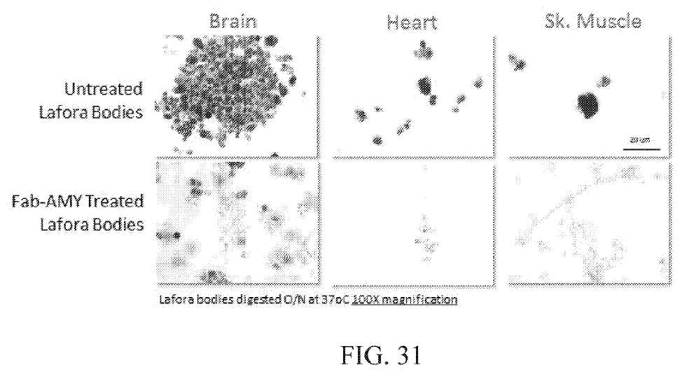

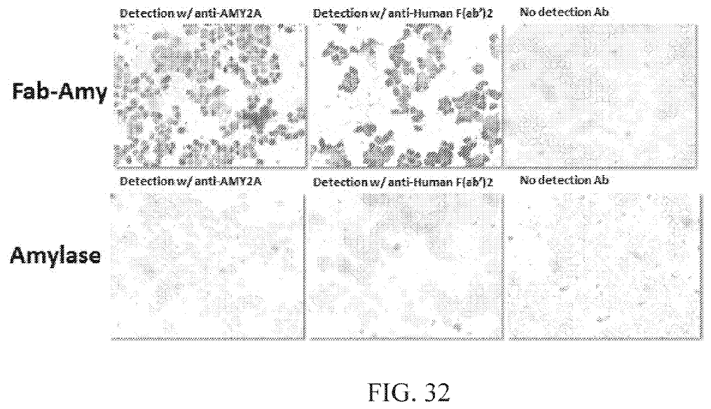

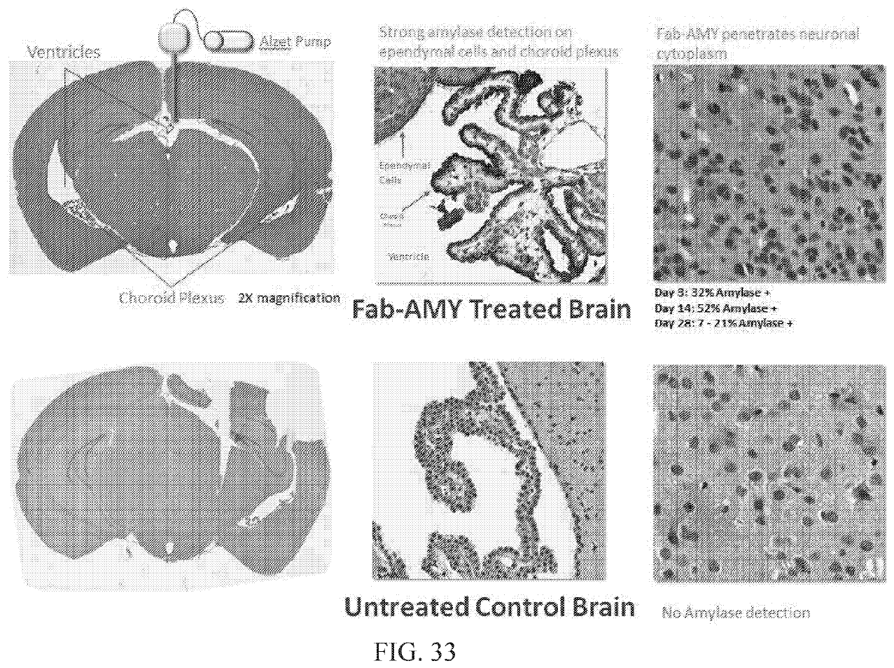

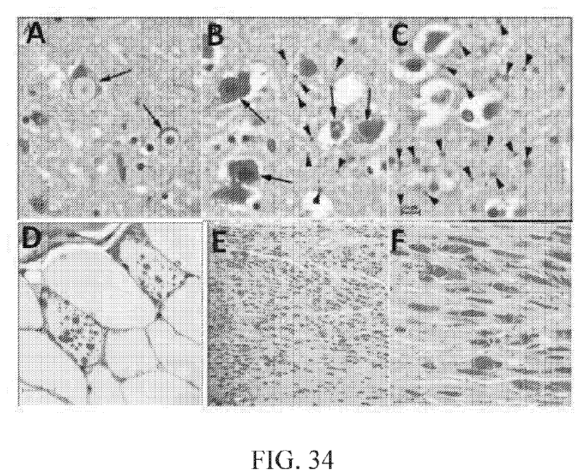

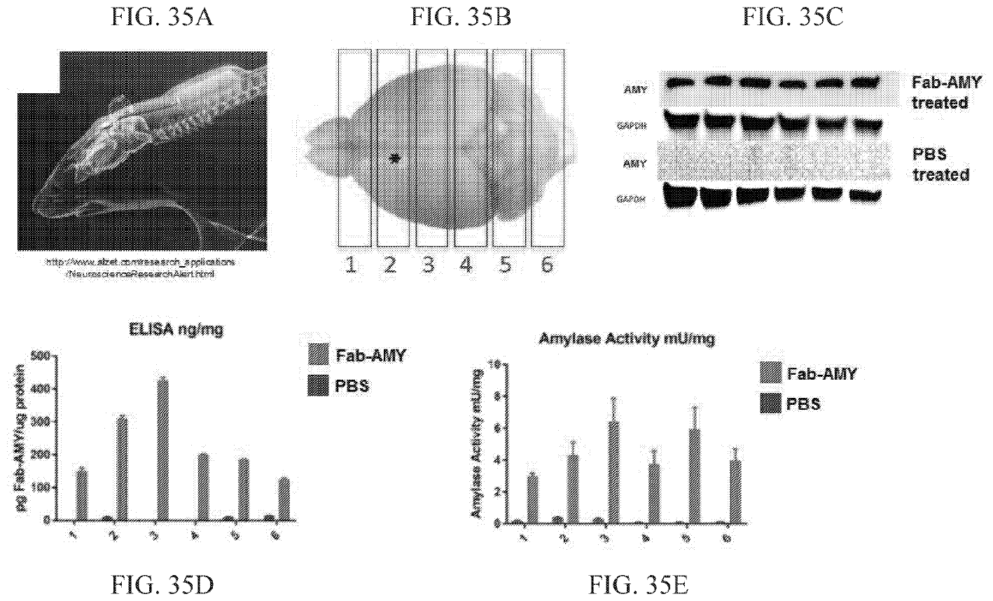

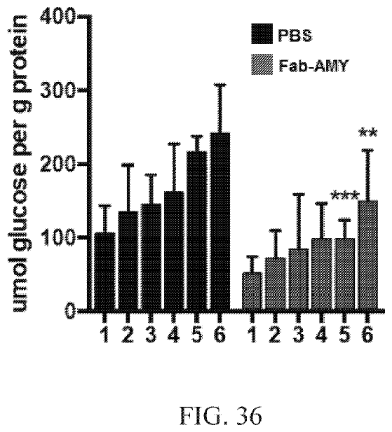

The effect of Fab-GAA on glycogen clearance in cardiac muscle is assessed using Lafora knock-out mice. Lafora knock-out mice are pretreated with an IP injection of diphenhydramine (15 mg/kg) 10-15 minutes prior to each enzyme administration to prevent anaphylactic reactions. The mice are given two tail vein injections every week for two weeks for a total of four injections of Fab-GAA (90 uL at 10 mg/mL), Myozyme (120 uL at 5 mg/mL), or PBS. Injections are given on days 1, 5, 9, and 13 for a total dose of Fab-GAA of 3600 ug (180 mg/kg for a 20 g mouse) or Myozyme of 2400 ug (120 mg/kg for a 20 g mouse) (i.e., an equimolar dose to Fab-GAA). The mice were then sacrificed and muscle sections of the treated mice were PAS stained (FIGS. 24-26). The results of the PAS staining of cardiac muscle that Lafora glycogen is lysosomal and perhaps cytoplasmic. In addition, 90% of cardiac myofibers are PAS+ in PBS treated knock-out mice. It was further shown that Fab-GAA clears glycogen better than Myozyme, with Myozyme clearing about 50% of Lafora glycogen, while Fab-GAA clears about 90% of Lafora glycogen.

E. Fab-GAA Treatment of Lafora Disease and Other Polyglusan Disease

Fab-GAA is currently being tested in a clinical trial as a therapy for Pompe Disease, a glycogen storage disease that primarily effects skeletal muscle and heart. The current therapy for Pompe disease is rhGAA (Myozyme), which utilizes the mannose-6-phosphate receptor (M6PR) to enter the lysosome and degrade glycogen. The advantage of Fab-GAA over Myozyme is that, in addition to M6PR-mediated transport into the lysosome, it can also enter the cell cytoplasm via the ENT2 receptor and clear glycogen that has accumulated from ruptured lysosomes and autophagic vacuoles.

It has recently been demonstrated that Fab-GAA delivered by intracerebroventricular (ICV) infusion can reduce the Lafora body/glycogen load in laforin KO mouse brain by 50%. In fact, the glycogen levels in Fab-GAA-treated brain approached those of wild type mice. These results begged the question as to whether Fab-GAA might be an effective therapy for Lafora disease as well as other polyglusan diseases that affect other organs such as skeletal muscle and heart.

An unexpected finding from those studies was that Fab-GAA appeared to be slightly more effective than Fab-amylase, another agent that was developed to treat Lafora disease. However, the animal numbers were small (N=5 each group) and the study was only performed in the laforin knock-out mouse model of Lafora disease. In vitro studies have shown that the activity of Fab-Amylase is augmented in the presence of laforin. Repeating the study in the malin knock-out mouse model of Lafora disease may yield the opposite results. It has been estimated that 58% of patients worldwide harbor the EPM2B (malin gene) knock-out mutation making it crucial to test potential therapies in both models of the disease (Turnbull 2016).

Using Lafora bodies as models for polyglusan found in other diseases, the studies outlined below are designed to answer key questions regarding the efficacy and dosing requirements for using Fab-GAA to clear polyglusan in the brain, heart, and skeletal muscle. When possible, additional organs will be collected to test for systemic dissemination, enzyme activity, and glycogen degradation in the peripheral organs. Additional experiments testing Fab-Amylase in malin KO models are also proposed.

i. Comparison of Fab-GAA and Myozyme for Lafora Body Clearance

The ability of Fab-GAA to reduce Lafora bodies and glycogen load to wild type levels is assessed. Additionally, the activity of Fab-GAA against normal glycogen in wild type mice is assessed. Finally, when administered via IV, the ability of Fab-GAA to get into the brain is assessed, as well as its ability to degrade Lafora bodies and glycogen in the brain once it gets there.

Animals are pretreated with an IP injection of diphenhydramine (15 mg/kg) 10-15 minutes prior to each enzyme administration to prevent anaphylactic reactions. Two IV injections are given of vehicle, Fab-GAA (90 uL at 10 mg/mL), Myozyme (120 uL at 5 mg/ml), or PBS every week for two weeks for a total of 4 injections. Injections occur on days 1, 5, 9, and 13. A total dose of Fab-GAA is 3600 ug (180 mg/kg for a 20 g mouse) and a total dose of Myozyme is 2400 ug (120 mg/kg ug for a 20 g mouse) (i.e., equimolar doses to Fab-GAA). The animals are sacrificed 24 hours after the last injection and heart, muscle, brain, foot pad, spleen, kidney, and liver are harvested and assayed for glycogen content and GAA activity. If possible, heart and muscle are fixed in 4% paraformaldehyde for PAS staining.

Using the results of the IM experiment, where 600 ug Fab-GAA was injected into gastroc in 10 month mice (˜35 g which equaled 17 mg/kg whole body equivalent dose) resulted in heart glucose levels of 13+/−10 μmol/g tissue compared with 50 μmol/g tissue for untreated aged-matched heart. The effect size was so large that a sample size of 3 for each group gives 100% power (one-tailed test as glycogen cannot decrease with treatment). The number of mice used in the study is N=5 laforin knock out mice for each group to account for animals that are lost during the experiment, and N=4 age-matched WT mice in the PBS and Fab-GAA groups only for a total of 15 laforin knock out mice and 8 wild-type mice.

ii. Fab-Amylase and Amylase Efficacy in Laforin Knock Out Model of Lafora Disease

Fab-Amylase and amylase only are compared for clearing Lafora bodies and glycogen from laforin knock out mouse brain. Laforin knock out mice were administered PBS (N=3), Fab-Amylase (N=3), or amylase in the Fab-amylase formulation (N=5) for 28 days via ICV infusion. At the end of the 28 days the mice are sacrificed and the brains are sectioned. Glucose levels are measured in each brain section of each mouse.

iii. Fab-GAA and Fab-Amylase Efficacy in Malin Knock Out Model of Lafora Disease

Malin knock out mice are implanted with Alzet pump 1003D and infused at 0.11 uL/hr for 28 days with vehicle (N=3), Fab-Amylase (N=4), or Fab-GAA (N=4). After 28 days the mice are sacrificed (i.e., implant Day 1 and sac on Day 29) and the brain is harvested, sectioned into six thick slices, and flash frozen for biochemical assessment of glycogen content. Blood at necropsy is obtained to check for GAA and amylase activity. In addition, samples from the heart, foot pads, quadriceps, diaphragm, triceps, gastroc, liver, kidney, and spleen are collected and assayed for glycogen content, GAA activity, and amylase activity.

iv. Assessment of Fab-GAA in a Large Animal Model

A cynomologus monkey is infused with 1 mL Fab-GAA (10 mg protein) over 10 min and monitored for clinical signs of adverse effects. If no effects are observed after one week, the animal receives weekly doses for 4 weeks and an additional 3 monkeys will be included in the study. If adverse effects are observed in the initial animal during the first week after dosing, the dose is reduced in the second week. A second animal will receive a dose of Fab-GAA formulation vehicle (no enzyme) to determine whether the enzyme or the formulation is the cause of the adverse effect. Both animals will receive lower doses of their respective infusates until a tolerated dose is achieved. Serum and CSF samples will be collected for PK (GAA activity) analysis.

v. Multiple Time Point Dose-Response Study

For continuous ICV infusion of Fab-GAA at a constant rate, the relationship between the duration of infusion and the degree of glycogen degradation is assessed. Laforin knock out mice and wild type mice are implanted for continuous ICV infusion at 0.11 uL/hr with vehicle or Fab-GAA on Day 1. Animals are sacrificed according to Table 8, the brains are harvested and flash frozen, and then assayed for glycogen content and GAA activity. Samples are also collected for heart, foot pads, quadriceps, diaphragm, triceps, gastroc, liver, kidney, and spleen and then assayed for glycogen content and GAA activity. Using the results of the ICV experiment in laforin knock out mice, where 700 ug Fab-GAA was infused over 28 days in 6.5 month mice resulted in brain glucose levels of approximately 3.8+/−0.2 μmol glucose/g tissue compared with 7 μmol glucose/g tissue for PBS-treated aged-matched mice. The effect size is so large that a sample size of 3 for each group gives 100% power (one-tailed test as glycogen cannot decrease with treatment). The sample size includes a total of 53 laforin knock out mice (46 study+7 replacement) and 12 WT mice (10 study+2 replacement). N=5 for each group to account for a smaller effect size for the animals infused shorter than 28 days.

TABLE 8

| | | Infusion end day | | | | |
|---|---|---|---|---|---|---|
| Model | Therapy | DAY 4 | DAY 8 | DAY 15 | DAY 22 | DAY 29 |
| laforin KO | PBS (N = 23) | 5 | 5 | 5 | 5 | 3 |
| laforin KO | VAL-1221 (N = 23) | 5 | 5 | 5 | 5 | 3 |
| WT | PBS (N = 5) | 0 | 0 | 0 | 0 | 5 |
| WT | VAL-1221 (N = 5) | 0 | 0 | 0 | 0 | 5 |

### Example 3: Fab-Amylase Penetrates Cells and Degrades Cytoplasmic Glycogen in a Mouse Model of Lafora Disease

A. Cell Penetration Platform

A proprietary antibody-based platform (Fab) is developed uniquely capable of penetrating cells and delivering therapeutic cargo to the cytoplasm. Fab penetrates cells via the ENT2 receptor, a nucleoside transporter highly expressed in skeletal and cardiac muscles, and the brain. A Fab-GAA fusion is currently being tested in the clinic as a potential therapy for Pompe disease.

Fab-Amylase (Fab-AMY) is a novel protein composed of a cell penetrating Fab fragment genetically fused to human pancreatic amylase (AMY2A). Fab-AMY is optimized to penetrate the cytoplasm of cells and degrade glycogen at neutral pH. Here, Fab-AMY is demonstrated to degrade pathogenic glycogen found in Lafora disease, a rare and inextricably fatal epileptic disease.

B. Lafora Disease

Lafora Disease (LD) is a rare neurodegenerative disorder and typically fatal within 10 years of onset. LD is characterized by the transformation of glycogen into malformed, aggregated inclusions called Lafora bodies (LBs). Insoluble Lafora bodies overtake the cytoplasm of neurons—eliciting a severe and lethal form of epilepsy (Raththagala M, et al. “Structural mechanism of laforin function in glycogen dephosphorylation and lafora disease” Mol Cell. 2015 Jan. 22; 57(2):261-72; Turnbull J, et al. “Lafora disease” Epileptic Disord. 2016 Sep. 1; 18(52):38-62. Review).

C. Results

It is determined whether the Fab-AMY fusion is efficacious in Lafora Disease. In vitro, Fab-AMY degrades isolated Lafora bodies from various Lafora tissues and penetration of cells requires the Fab fragment. In vivo, Fab-AMY administration by continuous intracerebroventricular (ICV) distributed to all brain regions, entered the cytoplasm of neuronal cells, and degraded Lafora glycogen by ˜50%.

D. Conclusions

These results show a clear promise for pursuing an antibody-enzyme treatment for Lafora disease. Amylase activity against typically refractory LBs strongly indicates these fusions will be active in other glycogen-driven diseases. Furthermore, the efficient distribution, uptake, and activity mediated by this platform is broadly applicable to other neuromuscular diseases; especially those requiring enzymatic clearance of toxic oligomers or aggregated intracellular deposits.

E. Fab-AMY Summary

In vitro: penetrates cells and delivers active amylase to the cytoplasm and degrades Lafora bodies in vitro.

In vivo, ICV administration: uptake throughout the brain; cytoplasmic penetration and amylase activity in neuronal tissue; and degradation of Lafora bodies in the brain.

### Example 4: Use of Fab-GAA to Treat Non-CNS Polyglucosan Accumulation Diseases

A. Polyglucosan Accumulation Diseases

Polyglucosan accumulation diseases are extraordinarily rare genetic disorders. Each disease has a prevalence of 1:50,000 general population or less. They are characterized by an accumulation of long, unbranched, poorly soluble polymers of glycogen that tend to aggregate and ultimately form distinct cellular inclusions within the cell that are partially resistant to digestion by amylases. These inclusions are generally referred to as “polyglucosan bodies”, although in Lafora disease they have been called “Lafora bodies”. The phenotypes of polyglucosan accumulation diseases depend, in part, on which tissues accumulate glycogen, most commonly cardiac muscle, but also skeletal muscle, liver, neurons and astroglia. Other than supportive therapy and transplantation of failing tissues, there are no definitive treatments for patients with polyglucosan accumulation diseases.

Polyglucosan accumulation appears to result from an imbalance between the rate of glycogen synthesis and branching enzyme activity. Thus, it can result from defective synthesis of glycogen, as in brancher enzyme (GBE1) deficiency (GSD IV or adult polyglucosan disease) or glycogenin-1 deficiency (GSD XV), or from defective degradation of glycogen, as in phosphofructokinase deficiency (GSD VII), Epm2a/laforin or Epm2b/malin deficiency (Lafora disease) or in RBCK1 deficiency (a ubiquitin ligase). Mutations in PRKAG2 may also cause polyglucosan accumulation due to defects in glucose metabolism, possibly related to constitutive activation of glycogen synthase.

There is enormous phenotypic variability both within polyglucosan accumulation diseases resulting from mutations in the same gene and between polyglucosan accumulation diseases resulting from different genes. GSD IV, for example, has 5 distinct phenotypes that vary with age at symptom onset and tissue involvement (developmental, progressive hepatic, non-progressive hepatic, skeletal muscle, cardiac muscle, and neuronal) whereas Lafora and PRKAG2 associated cardiomyopathy (PAC) are relatively specific to neuronal tissue and heart, respectively.

B. In Vitro and In Vivo Studies

Fab-GAA has been studied in several animal models of excessive glycogen storage including polyglucosan disease models.

In a glycogen branching enzyme deficient (GBE1 neo/neo) mouse model of Adult Polyglucosan Body Disease (APBD), a variant of GSD IV in which polyglucosan bodies (PB) develop throughout the bodies of the mice within 6 weeks of birth, the effect of Fab-GAA on tissue glycogen clearance was assessed in vitro.

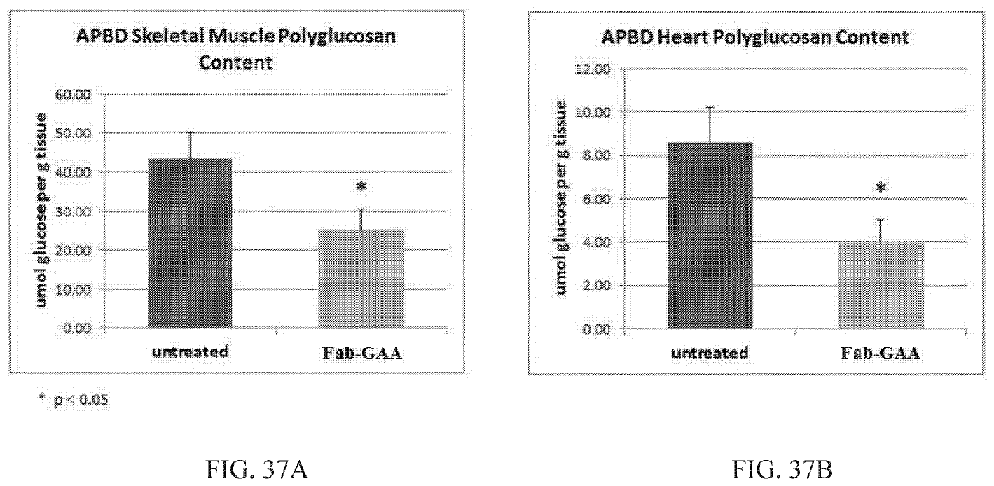

Heart and skeletal muscle were harvested from GBE1 neo/neo mice and homogenized and then treated with Fab-GAA. The ability of Fab-GAA to breakdown polyglucosan was determined by measuring the residual glucose in the homogenate. In response to Fab-GAA, there was a 50% reduction in the glucose derived from both heart (FIG. 37B) and muscle (FIG. 37A), indicating effective degradation of polyglucosan by Fab-GAA.

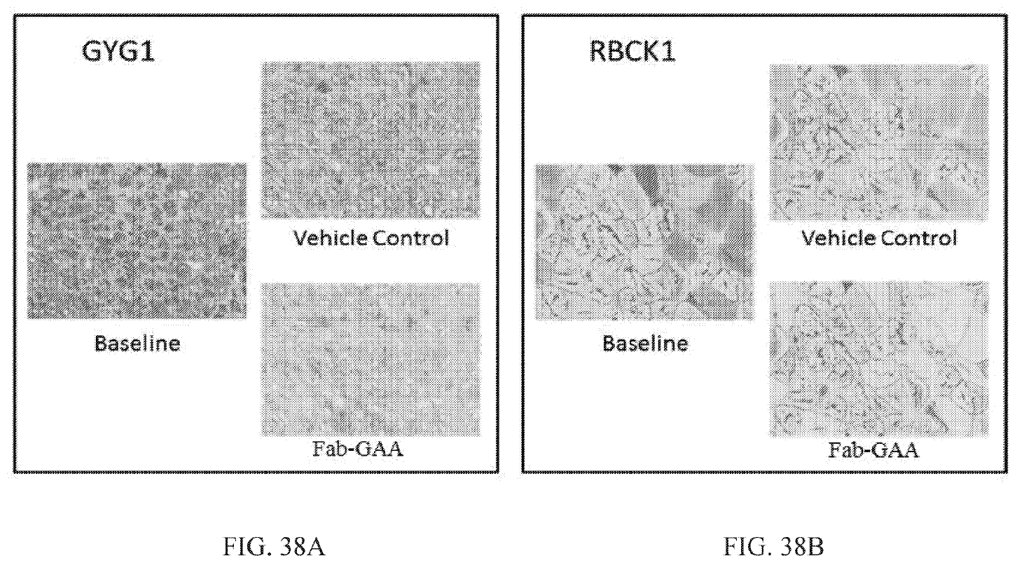

Building on this proof-of-concept study in a mouse model, experiments were performed treating human tissue specimens from patients with a variety of polyglucosan accumulation diseases with Fab-GAA. Frozen sections were incubated in either 10 mg/ml Fab-GAA or vehicle at 37° C. for 12 hours. Specimens were then PAS stained to compare the glycogen content in the two specimen groups. FIG. 38 shows early promising results from these studies. FIG. 38A shows a heart specimen from a patient with a GYG1 missense mutation (c.304G>C, p.(Asp102His) that had severe glycogenin-1 deficiency resulting in dilated cardiomyopathy that required a cardiac transplant. FIG. 38B shows a skeletal muscle specimen from a patient with multiple RBCK1 mutations (c.817dupC, p.(Leu273Profs*27)) and c.1465delA, p.(Thr489Profs*9) resulting in severe RBCK1 deficiency. The patient was wheelchair bound and exhibited dilated cardiomyopathy requiring a cardiac heart transplant. Fab-GAA clearly reduced polyglucosan in both tissue types despite the difference in the etiologies of the two glycogen storage abnormalities.

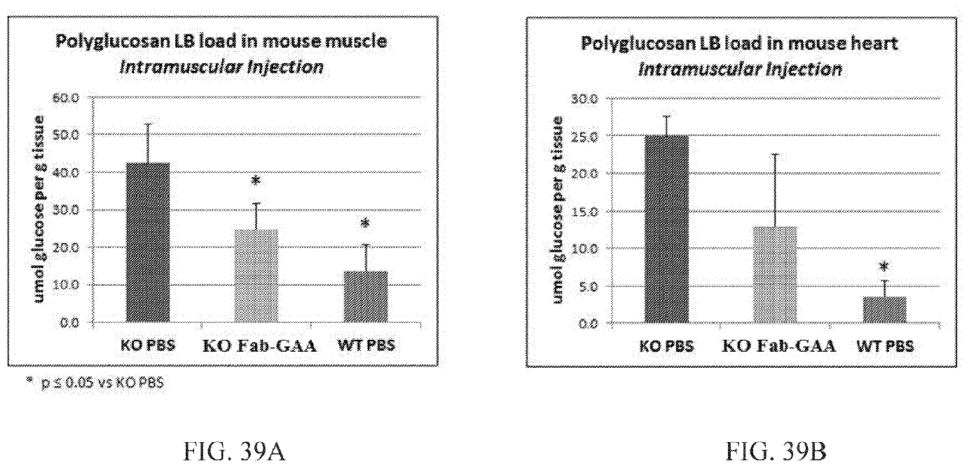

Like patients with Lafora disease, Epm2a−/− mice present with Lafora bodies (LB) in multiple tissues, including brain, muscle, heart and skin, although pathology is primarily neurological. Three serial injections of 20 μL of 10 mg/mL Fab-GAA (N=3) or PBS (N=4) were administered into the right gastrocnemius of 10-month-old Epm2a−/− mice over the course of one week, on days 1, 4, and 7 to determine the effect on polyglucosan accumulation. Age-matched wild type C57BL/6 mice were treated with PBS (N=3) using the same regimen. On day 8 the mice were euthanized and muscles were collected for polyglucosan determination. Previous studies have shown that intramuscularly administered Fab-GAA can move to other organs in mice, therefore hearts were also collected from the mice and the polyglucosan content was determined. FIG. 39A shows that Fab-GAA treatment reduced polyglucosan levels by 42% relative to the PBS treated muscle. Since polyglucosan is only found inside cells, these data indicate that intramuscularly-injected Fab-GAA indeed penetrates cells in vivo, remains active, and degrades polyglucosans. The trend toward lower polyglucosan content in the heart of the Epm2a−/− animals treated with Fab-GAA shown in FIG. 39B suggests that even the limited amount of Fab-GAA that entered the systemic circulation and traveled to the heart was efficacious. Indeed, in two of the three animals treated with Fab-GAA, the polyglucosan levels in the heart were reduced to near WT levels (9.9 and 5.2 μmol glucose per g tissue), while levels in the third animal were similar to the PBS treated animals (23.7 μmol glucose per g tissue). Thus, the lack of an effect in the latter animal was more likely due to limited or no systemic movement of drug in that animal rather than limited activity of Fab-GAA.

As a more direct measure of Fab-GAA treatment efficacy, four serial tail vein injections of 0.90 mg VAL-1221 or PBS were administered to 6 month old Epm2a−/− mice (N=5 each treatment). The mice weighed 30 g on average resulting in a 120 mg/kg dose of Fab-GAA being administered to the treatment group. Age-matched wild type C57BL/6 mice (N=4) were injected with PBS as a control cohort. Hearts and quadriceps muscle were collected and quantified for total polyglucosan content.

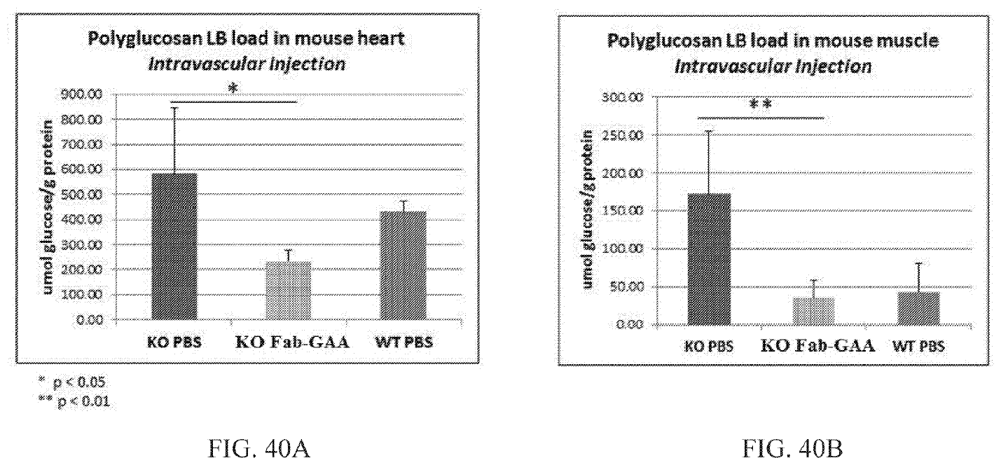

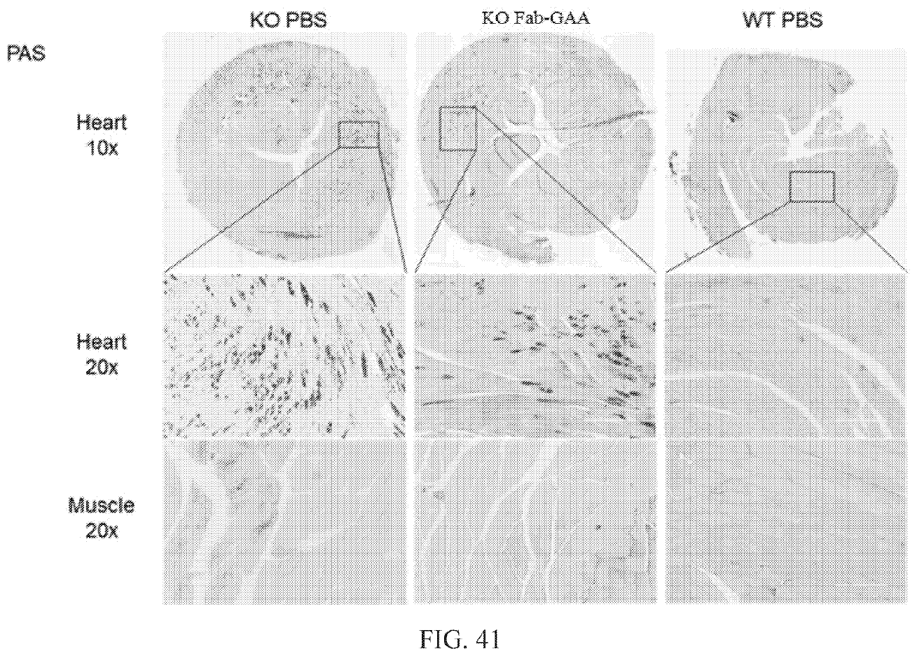

Fab-GAA treatment reduced polyglucosan LB loads in Epm2a−/− (KO) mice to wild type (WT) levels (FIG. 40). Periodic acid Schiff staining (FIG. 41) showed a reduction in the number of polyglucosan bodies in both tissues after treatment with Fab-GAA.

In comparison, Epm2a−/− mice treated intravenously with equimolar doses of Myozyme showed minimal reductions in polyglucosan inclusions in the heart compared to the 80% reduction observed in the Fab-GAA treated animals (FIGS. 23-26).

Thus, Fab-GAA is able to enter the cytoplasm and clear accumulated polyglucosan in mouse models of polyglucosan diseases.

Preventing the formation of glycogen, and hence polyglucosan precipitates, prevents disease in a model of Lafora Disease. Therefore, clearing aggregates would be expected to improve or reverse symptoms in the other polyglucosan disorders.

C. Toxicology Summary

Toxicology studies have been conducted using juvenile and adult rats, as well as adolescent and adult monkeys.

Repeat-dose (once per week for 4 weeks, 5 total injections) non-GLP toxicology studies were performed in juvenile and adult rats (50 mg/kg); a GLP study was done in juvenile rats at 3, 10 and 30 mg/kg once per week for 4 weeks. Single and repeat-dose studies were performed in adult and adolescent non-human primates at 50 mg/kg (single dose and repeat-dose, non-GLP) and a GLP study at 3, 10 or 30 mg/kg (weekly for up to 6 months) was also performed. Under the conditions of these toxicity studies in rats and monkeys, the no-observed-effect-level (NOEL) and no-observed-adverse-effect-level (NOAEL) for 5 or more IV injections of Fab-GAA were above 30 mg/kg in GLP studies. Toxicology studies suggest that Fab-GAA is safe at repeat doses up to 30 mg/kg in rats and non-human primates.

D. Treatment of PAC

Based on nonclinical and clinical data, Fab-GAA in its current formulation appears to be well suited for the treatment of non-CNS polyglucosan accumulation diseases. Because PRKAG2 associated cardiomyopathy (PAC) is relatively more common than other polyglucosan accumulation diseases and has somewhat less phenotypic variability, Fab-GAA will be assessed for safety and efficacy in treating PAC patients. A 2-part, double-blind, saline-controlled study will be performed in approximately 36 patients with PAC to assess the safety, PK, PD and efficacy of Fab-GAA in the treatment of this disorder. Part 1, in 12 patients, will be used adaptively to confirm the dose, number of patients and key efficacy assessments to be brought forward in a larger group of patients in Part 2.

In summary, Fab-GAA represents a novel antibody-enzyme fusion replacement candidate for non-CNS polyglucosan diseases which demonstrates improved tissue targeting and offers the potential to clear polyglucosan bodies from affected tissues.

### Exemplary Sequences

Alpha Amylase Polypeptide Amino Acid Sequence (Genbank accession
number NP_000690)
SEQ ID NO: 1
QYSPNTQQGRTSIVHLFEWRWVDIALECERYLAPKGFGGVQVSPPNENVAIYNPFR
PWWERYQPVSYKLCTRSGNEDEFRNMVTRCNNVGVRIYVDAVINHMCGNAVSAG
TSSTCGSYFNPGSRDFPAVPYSGWDFNDGKCKTGSGDIENYNDATQVRDCRLTGL
LDLALEKDYVRSKIAEYMNHLIDIGVAGFRLDASKHMWPGDIKAILDKLHNLNSN
WFPAGSKPFIYQEVIDLGGEPIKSSDYFGNGRVTEFKYGAKLGTVIRKWNGEKMSY
LKNWGEGWGFVPSDRALVFVDNHDNQRGHGAGGASILTFWDARLYKMAVGFML
AHPYGFTRVMSSYRWPRQFQNGNDVNDWVGPPNNNGVIKEVTINPDTTCGNDWV
CEHRWRQIRNMVIFRNVVDGQPFTNWYDNGSNQVAFGRGNRGFIVFNNDDWSFS
LTLQTGLPAGTYCDVISGDKINGNCTGIKIYVSDDGKAHFSISNSAEDPFIAIHAESK
L
Humanized Variable Heavy Chain Amino Acid Sequence (VH3)
SEQ ID NO: 2
EVQLQESGGGVVQPGGSLRLSCAASGFTFSNYGMHWIRQAPGKGLEWVSYISSGSS
TIYYADSVKGRFTISRDNSKNTLYLQMNSLRSEDTAVYYCARRGLLLDYWGQGTL
VTVSS
Humanized Variable Light Chain Amino Acid Sequence (VL2)
SEQ ID NO: 3
DIQMTQSPSSLSASVGDRVTISCRASKSVSTSSYSYMHWYQQKPEKAPKLLIKYAS
YLQSGVPSRFSGSGSGTDFTLTISSLQPEDVATYYCQHSREFPWTFGAGTKLELK
Heavy Chain Leader Amino Acid Sequence
SEQ ID NO: 4
MEFGLSWLFLVAILKGVQC
Light Chain Leader Amino Acid Sequence
SEQ ID NO: 5
MDMRVPAQLLGLLLLWLRGARC
Glycine-Serine Linker Amino Acid Sequence
SEQ ID NO: 6
GGSGGGSGGGSGG
Humanized Heavy Chain Amino Acid Sequence (including human IgG1
CH1 and truncated hinge constant regions)
SEQ ID NO: 7
EVQLQESGGGVVQPGGSLRLSCAASGFTFSNYGMHWIRQAPGKGLEWVSYISSGSS
TIYYADSVKGRFTISRDNSKNTLYLQMNSLRSEDTAVYYCARRGLLLDYWGQGTL
VTVSSASTKGPSVFPLAPSSKSTSGGTAALGCLVKDYFPEPVTVSWNSGALTSGVH
TFPAVLQSSGLYSLSSVVTVPSSSLGTQTYICNVNHKPSNTKVDKKVEPKSCDKTHT
Humanized Light Chain Amino Acid Sequence (including human kappa
light chain region)
SEQ ID NO: 8
DIQMTQSPSSLSASVGDRVTISCRASKSVSTSSYSYMHWYQQKPEKAPKLLIKYAS
YLQSGVPSRFSGSGSGTDFTLTISSLQPEDVATYYCQHSREFPWTFGAGTKLELKRT
VAAPSVFIFPPSDEQLKSGTASVVCLLNNFYPREAKVQWKVDNALQSGNSQESVTE
QDSKDSTYSLSSTLTLSKADYEKHKVYACEVTHQGLSSPVTKSFNRGEC
Heavy Chain + Alpha-Amylase Fusion Protein Amino Acid Sequence
(Including Leader and Linker Sequences)
SEQ ID NO: 9
MEFGLSWLFLVAILKGVQCEVQLQESGGGVVQPGGSLRLSCAASGFTFSNYGMHW
IRQAPGKGLEWVSYISSGSSTIYYADSVKGRFTISRDNSKNTLYLQMNSLRSEDTAV
YYCARRGLLLDYWGQGTLVTVSSASTKGPSVFPLAPSSKSTSGGTAALGCLVKDY
FPEPVTVSWNSGALTSGVHTFPAVLQSSGLYSLSSVVTVPSSSLGTQTYICNVNHKP
SNTKVDKKVEPKSCDKTHTGGSGGGSGGGSGGQYSPNTQQGRTSIVHLFEWRWV
DIALECERYLAPKGFGGVQVSPPNENVAIYNPFRPWWERYQPVSYKLCTRSGNEDE
FRNMVTRCNNVGVRIYVDAVINHMCGNAVSAGTSSTCGSYFNPGSRDFPAVPYSG
WDFNDGKCKTGSGDIENYNDATQVRDCRLTGLLDLALEKDYVRSKIAEYMNHLID
IGVAGFRLDASKHMWPGDIKAILDKLHNLNSNWFPAGSKPFIYQEVIDLGGEPIKSS
DYFGNGRVTEFKYGAKLGTVIRKWNGEKMSYLKNWGEGWGFVPSDRALVFVDN
HDNQRGHGAGGASILTFWDARLYKMAVGFMLAHPYGFTRVMSSYRWPRQFQNG
NDVNDWVGPPNNNGVIKEVTINPDTTCGNDWVCEHRWRQIRNMVIFRNVVDGQP
FTNWYDNGSNQVAFGRGNRGFIVFNNDDWSFSLTLQTGLPAGTYCDVISGDKING
NCTGIKIYVSDDGKAHFSISNSAEDPFIAIHAESKL
Light Chain + Leader Amino Acid Sequence
SEQ ID NO: 10
MDMRVPAQLLGLLLLWLRGARCDIQMTQSPSSLSASVGDRVTISCRASKSVSTSSY
SYMHWYQQKPEKAPKLLIKYASYLQSGVPSRFSGSGSGTDFTLTISSLQPEDVATY
YCQHSREFPWTFGAGTKLELKRTVAAPSVFIFPPSDEQLKSGTASVVCLLNNFYPRE
AKVQWKVDNALQSGNSQESVTEQDSKDSTYSLSSTLTLSKADYEKHKVYACEVT
HQGLSSPVTKSFNRGEC
Heavy Chain IgG1 CH1 and Truncated Hinge Constant Domain
SEQ ID NO: 11
ASTKGPSVFPLAPSSKSTSGGTAALGCLVKDYFPEPVTVSWNSGALTSGVHTFPAV
LQSSGLYSLSSVVTVPSSSLGTQTYICNVNHKPSNTKVDKKVEPKSCDKTHT
Human Kappa Constant Domain of Km3 Allotype
SEQ ID NO: 12
RTVAAPSVFIFPPSDEQLKSGTASVVCLLNNFYPREAKVQWKVDNALQSGNSQESV
TEQDSKDSTYSLSSTLTLSKADYEKHKVYACEVTHQGLSSPVTKSFNRGEC
GS3 linker
SEQ ID NO: 13
GGGGSGGGGSGGGGS
Linker
SEQ ID NO: 14
GSTSGSGKSSEGKG
His tag
SEQ ID NO: 15
HHHHHHH
c-Myc tag
SEQ ID NO: 16
EQKLISEEDL
exemplary 3E10 Variable Heavy Chain (V_{H} having D31N substitution;
see examples)
SEQ ID NO: 17
EVQLVESGGGLVKPGGSRKLSCAASGFTFSNYGMHWVRQAPEKGLEWVAYISSGS
STIYYADTVKGRFTISRDNAKNTLFLQMTSLRSEDTAMYYCARRGLLLDYWGQGT
TLTVSS
3E10 Variable Light Chain (V_{L})
SEQ ID NO: 18
DIVLTQSPASLAVSLGQRATISCRASKSVSTSSYSYMHWYQQKPGQPPKLLIKYASY
LESGVPARFSGSGSGTDFHLNIHPVEEEDAATYYCQHSREFPWTFGGGTKLELK
heavy chain variable domain CDR1 of 3E10 VH (as that VH is defined
with reference to SEQ ID NO: 17), in accordance with Kabat system
SEQ ID NO: 19
NYGMH
heavy chain variable domain CDR2 of 3E10 VH (as that VH is defined
with reference to SEQ ID NO: 17), in accordance with Kabat system
SEQ ID NO: 20
YISSGSSTIYYADTVKG
heavy chain variable domain CDR3 of 3E10 VH (as that VH is defined
with reference to SEQ ID NO: 17), in accordance with Kabat system
SEQ ID NO: 21
RGLLLDY
light chain variable domain CDR1 of 3E10 VL (as that VL is defined
with reference to SEQ ID NO: 18), in accordance with Kabat system
SEQ ID NO: 22
RASKSVSTSSYSYMH
light chain variable domain CDR2 of 3E10 VL (as that VL is defined
with reference to SEQ ID NO: 18), in accordance with Kabat system
SEQ ID NO: 23
YASYLES
light chain variable domain CDR3 of 3E10 VL (as that VL is defined
with reference to SEQ ID NO: 18), in accordance with Kabat system
SEQ ID NO: 24
QHSREFPWT
“AGIH”
SEQ ID NO: 25
AGIH
“SAGIH”
SEQ ID NO: 26
SAGIH
heavy chain variable (V_{H}) domain CDR1 of exemplary 3E10 molecule,
in accordance with CDRs as defined by the IMGT system
SEQ ID NO: 27
GFTFSNYG
heavy chain variable (V_{H}) domain CDR2 of exemplary 3E10 molecule,
in accordance with CDRs as defined by the IMGT system
SEQ ID NO: 28
ISSGSSTI
heavy chain variable (V_{H}) domain CDR3 of exemplary 3E10 molecule,
in accordance with CDRs as defined by the IMGT system
SEQ ID NO: 29
ARRGLLLDY
light chain variable (V_{L}) domain CDR1 of exemplary 3E10 molecule, in
accordance with CDRs as defined by the IMGT system
SEQ ID NO: 30
KSVSTSSYSY
light chain variable (V_{L}) domain CDR2 of exemplary 3E10 molecule, in
accordance with CDRs as defined by the IMGT system
SEQ ID NO: 31
YAS
light chain variable (V_{L}) domain CDR3 of exemplary 3E10 molecule, in
accordance with CDRs as defined by the IMGT system
SEQ ID NO: 32
QHSREFPWT
amino acid sequence of humanized 3E10 heavy chain (hVH1)
SEQ ID NO: 33
EVQLVQSGGGLIQPGGSLRLSCAASGFTFSNYGMHWVRQAPGKGLEWVSYISSGSS
TIYYADSVKGRFTISRDNSKNTLYLQMNSLRAEDTAVYYCARRG
LLLDYWGQGTTVTVSS
amino acid sequence of humanized 3E10 heavy chain (hVH2)
SEQ ID NO: 34
EVQLVESGGGLIQPGGSLRLSCAASGFTFSNYGMHWVRQAPGKGLEWVSYISSGSS
TIYYADSVKGRFTISRDNSKNTLYLQMTSLRAEDTAVYYCARRG
LLLDYWGQGTTLTVSS
amino acid sequence of humanized 3E10 light chain (hVL1)
SEQ ID NO: 35
DIQMTQSPSSLSASVGDRVTITCRASKSVSTSSYSYLAWYQQKPEKAPKLLIKYASY
LQSGVPSRFSGSGSGTDFTLTISSLQPEDFATYYCQHSREFPWTFGAGTKLELK
Human Pancreatic Alpha Amylase Amino Acid Sequence (GenBank
Accession No.: NP_000690.1)
SEQ ID NO: 36
MKFFLLLFTIGFCWAQYSPNTQQGRTSIVHLFEWRWVDIALECERYLAPKGFGGVQ
VSPPNENVAIYNPFRPWWERYQPVSYKLCTRSGNEDEFRNMVTRCNNVGVRIYVD
AVINHMCGNAVSAGTSSTCGSYFNPGSRDFPAVPYSGWDFNDGKCKTGSGDIENY
NDATQVRDCRLTGLLDLALEKDYVRSKIAEYMNHLIDIGVAGFRLDASKHMWPG
DIKAILDKLHNLNSNWFPAGSKPFIYQEVIDLGGEPIKSSDYFGNGRVTEFKYGAKL
GTVIRKWNGEKMSYLKNWGEGWGFVPSDRALVFVDNHDNQRGHGAGGASILTF
WDARLYKMAVGFMLAHPYGFTRVMSSYRWPRQFQNGNDVNDWVGPPNNNGVI
KEVTINPDTTCGNDWVCEHRWRQIRNMVIFRNVVDGQPFTNWYDNGSNQVAFGR
GNRGFIVFNNDDWSFSLTLQTGLPAGTYCDVISGDKINGNCTGIKIYVSDDGKAHFS
ISNSAEDPFIAIHAESKL
heavy chain variable domain CDR2 of certain antibodies of the
disclosure, in accordance with CDRs as defined by Kabat
SEQ ID NO: 37
YISSGSSTIYYADSVKG
light chain variable domain CDR1 of certain antibodies of the disclosure,
in accordance with CDRs as defined by Kabat
SEQ ID NO: 38
RASKSVSTSSYSYLA
light chain variable domain CDR2 of certain antibodies of the disclosure,
in accordance with CDRs as defined by Kabat
SEQ ID NO: 39
YASYLQS
amino acid sequence of a reference humanized 3E10 light chain
SEQ ID NO: 40
DIVLTQSPASLAVSPGQRATITCRASKSVSTSSYSYMHWYQQKPGQPPKLLIYYASY
LESGVPARFSGSGSGTDFTLTINPVEANDTANYYCQHSREFPWTFGQGTKVEIK
amino acid sequence of a reference humanized 3E10 heavy chain
SEQ ID NO: 41
EVQLVESGGGLVQPGGSLRLSCSASGFTFSNYGMHWVRQAPGKGLEYVSYISSGSS
TIYYADTVKGRFTISRDNSKNTLYLQMSSLRAEDTAVYYCVKRGLLLDYWGQGTL
VTVSS
Reference Humanized Fv3E10
SEQ ID NO: 42
DIVLTQSPASLAVSPGQRATITCRASKSVSTSSYSYMHWYQQKPGQPPKLLIYYASY
LESGVPARFSGSGSGTDFTLTINPVEANDTANYYCQHSREFPWTFGQGTKVEIKGG
GGSGGGGSGGGGSEVQLVESGGGLVQPGGSLRLSCSASGFTFSNYGMHWVRQAP
GKGLEYVSYISSGSSTIYYADTVKGRFTISRDNSKNTLYLQMSSLRAEDTAVYYCV
KRGLLLDYWGQGTLVTVSS
Heavy Chain + Alpha-Amylase Fusion Protein Amino Acid Sequence
(excluding Linker Sequence)
SEQ ID NO: 43
EVQLQESGGGVVQPGGSLRLSCAASGFTFSNYGMHWIRQAPGKGLEWVSYISSGSS
TIYYADSVKGRFTISRDNSKNTLYLQMNSLRSEDTAVYYCARRGLLLDYWGQGTL
VTVSSASTKGPSVFPLAPSSKSTSGGTAALGCLVKDYFPEPVTVSWNSGALTSGVH
TFPAVLQSSGLYSLSSVVTVPSSSLGTQTYICNVNHKPSNTKVDKKVEPKSCDKTHT
GGSGGGSGGGSGGQYSPNTQQGRTSIVHLFEWRWVDIALECERYLAPKGFGGVQV
SPPNENVAIYNPFRPWWERYQPVSYKLCTRSGNEDEFRNMVTRCNNVGVRIYVDA
VINHMCGNAVSAGTSSTCGSYFNPGSRDFPAVPYSGWDFNDGKCKTGSGDIENYN
DATQVRDCRLTGLLDLALEKDYVRSKIAEYMNHLIDIGVAGFRLDASKHMWPGDI
KAILDKLHNLNSNWFPAGSKPFIYQEVIDLGGEPIKSSDYFGNGRVTEFKYGAKLGT
VIRKWNGEKMSYLKNWGEGWGFVPSDRALVFVDNHDNQRGHGAGGASILTFWD
ARLYKMAVGFMLAHPYGFTRVMSSYRWPRQFQNGNDVNDWVGPPNNNGVIKEV
TINPDTTCGNDWVCEHRWRQIRNMVIFRNVVDGQPFTNWYDNGSNQVAFGRGNR
GFIVFNNDDWSFSLTLQTGLPAGTYCDVISGDKINGNCTGIKIYVSDDGKAHFSISNS
AEDPFIAIHAESKL
Human Salivary Alpha Amylase Amino Acid Sequence (GenBank
Accession No.: AAI44453.1)
SEQ ID NO: 44
MKLFWLLFTIGFCWAQYSSNTQQGRTSIVHLFEWRWVDIALECERYLAPKGFGGV
QVSPPNENVAIHNPFRPWWERYQPVSYKLCTRSGNEDEFRNMVTRCNNVGVRIYV
DAVINHMCGNAVSAGTSSTCGSYFNPGSRDFPAVPYSGWDFNDGKCKTGSGDIEN
YNDATQVRDCRLSGLLDLALGKDYVRSKIAEYMNHLIDIGVAGFRIDASKHMWPG
DIKAILDKLHNLNSNWFPEGSKPFIYQEVIDLGGEPIKSSDYFGNGRVTEFKYGAKL
GTVIRKWNGEKMSYLKNWGEGWGFMPSDRALVFVDNHDNQRGHGAGGASILTF
WDARLYKMAVGFMLAHPYGFTRVMSSYRWPRYFENGKDVNDWVGPPNDNGVT
KEVTINPDTTCGNDWVCEHRWRQIRNMVNFRNVVDGQPFTNWYDNGSNQVAFG
RGNRGFIVFNNDDWTFSLTLQTGLPAGTYCDVISGDKINGNCTGIKIYVSDDGKAH
FSISNSAEDPFIAIHAESKL
full-length, immature GAA amino acid sequence (952 amino acids;
signal sequence indicated in bold/underline)
SEQ ID NO: 45
MGVRHPPCSHRLLAVCALVSLATAALLGHILLHDFLLVPRELSGSSPVLEETHPAH
QQGASRPGPRDAQAHPGRPRAVPTQCDVPPNSRFDCAPDKAITQEQCEARGCCYIP
AKQGLQGAQMGQPWCFFPPSYPSYKLENLSSSEMGYTATLTRTTPTFFPKDILTLRL
DVMMETENRLHFTIKDPANRRYEVPLETPHVHSRAPSPLYSVEFSEEPFGVIVRRQL
DGRVLLNTTVAPLFFADQFLQLSTSLPSQYITGLAEHLSPLMLSTSWTRITLWNRDL
APTPGANLYGSHPFYLALEDGGSAHGVFLLNSNAMDVVLQPSPALSWRSTGGILD
VYIFLGPEPKSVVQQYLDVVGYPFMPPYWGLGFHLCRWGYSSTAITRQVVENMTR
AHFPLDVQWNDLDYMDSRRDFTFNKDGFRDFPAMVQELHQGGRRYMMIVDPAIS
SSGPAGSYRPYDEGLRRGVFITNETGQPLIGKVWPGSTAFPDFTNPTALAWWEDM
VAEFHDQVPFDGMWIDMNEPSNFIRGSEDGCPNNELENPPYVPGVVGGTLQAATIC
ASSHQFLSTHYNLHNLYGLTEAIASHRALVKARGTRPFVISRSTFAGHGRYAGHWT
GDVWSSWEQLASSVPEILQFNLLGVPLVGADVCGFLGNTSEELCVRWTQLGAFYP
FMRNHNSLLSLPQEPYSFSEPAQQAMRKALTLRYALLPHLYTLFHQAHVAGETVA
RPLFLEFPKDSSTWTVDHQLLWGEALLITPVLQAGKAEVTGYFPLGTWYDLQTVP
VEALGSLPPPPAAPREPAIHSEGQWVTLPAPLDTINVHLRAGYIIPLQGPGLTTTESR
QQPMALAVALTKGGEARGELFWDDGESLEVLERGAYTQVIFLARNNTIVNELVRV
TSEGAGLQLQKVTVLGVATAPQQVLSNGVPVSNFTYSPDTKVLDICVSLLMGEQFL
VSWC
full-length, immature GAA amino acid sequence (957 amino acids;
signal sequence indicated in bold/underline) (GenBank Accession No. EAW89583.1)
SEQ ID NO: 46
MGVRHPPCSHRLLAVCALVSLATAALLGHILLHDFLLVPRELSGSSPVLEETHPAH
QQGASRPGPRDAQAHPGRPRAVPTQCDVPPNSRFDCAPDKAITQEQCEARGCCYIP
AKQGLQGAQMGQPWCFFPPSYPSYKLENLSSSEMGYTATLTRTTPTFFPKDILTLRL
DVMMETENRLHFTIKDPANRRYEVPLETPHVHSRAPSPLYSVEFSEEPFGVIVRRQL
DGRVLLNTTVAPLFFADQFLQLSTSLPSQYITGLAEHLSPLMLSTSWTRITLWNRDL
APTPGANLYGSHPFYLALEDGGSAHGVFLLNSNAMDVVLQPSPALSWRSTGGILD
VYIFLGPEPKSVVQQYLDVVGYPFMPPYWGLGFHLCRWGYSSTAITRQVVENMTR
AHFPLDVQWNDLDYMDSRRDFTFNKDGFRDFPAMVQELHQGGRRYMMIVDPAIS
SSGPAGSYRPYDEGLRRGVFITNETGQPLIGKVWPGSTAFPDFTNPTALAWWEDM
VAEFHDQVPFDGMWIDMNEPSNFIRGSEDGCPNNELENPPYVPGVVGGTLQAATIC
ASSHQFLSTHYNLHNLYGLTEAIASHRALVKARGTRPFVISRSTFAGHGRYAGHWT
GDVWSSWEQLASSVPEILQFNLLGVPLVGADVCGFLGNTSEELCVRWTQLGAFYP
FMRNHNSLLSLPQEPYSFSEPAQQAMRKALTLRYALLPHLYTLFHQAHVAGETVA
RPLFLEFPKDSSTWTVDHQLLWGEALLITPVLQAGKAEVTGYFPLGTWYDLQTVPI
EALGSLPPPPAAPREPAIHSEGQWVTLPAPLDTINVHLRAGYIIPLQGPGLTTTESRQ
QPMALAVALTKGGEARGELFWDDGESLEVLERGAYTQVIFLARNNTIVNELVRVT
SEGAGLQLQKVTVLGVATAPQQVLSNGVPVSNFTYSPDTKARGPRVLDICVSLLM
GEQFLVSWC
exemplary mature GAA amino acid sequence (corresponding to residues
123-782 of SEQ ID NO: 45; one embodiment of a mature GAA polypeptide)
SEQ ID NO: 47
GQPWCFFPPSYPSYKLENLSSSEMGYTATLTRTTPTFFPKDILTLRLDVMMETENRL
HFTIKDPANRRYEVPLETPHVHSRAPSPLYSVEFSEEPFGVIVRRQLDGRVLLNTTV
APLFFADQFLQLSTSLPSQYITGLAEHLSPLMLSTSWTRITLWNRDLAPTPGANLYG
SHPFYLALEDGGSAHGVFLLNSNAMDVVLQPSPALSWRSTGGILDVYIFLGPEPKS
VVQQYLDVVGYPFMPPYWGLGFHLCRWGYSSTAITRQVVENMTRAHFPLDVQW
NDLDYMDSRRDFTFNKDGFRDFPAMVQELHQGGRRYMMIVDPAISSSGPAGSYRP
YDEGLRRGVFITNETGQPLIGKVWPGSTAFPDFTNPTALAWWEDMVAEFHDQVPF
DGMWIDMNEPSNFIRGSEDGCPNNELENPPYVPGVVGGTLQAATICASSHQFLSTH
YNLHNLYGLTEAIASHRALVKARGTRPFVISRSTFAGHGRYAGHWTGDVWSSWEQ
LASSVPEILQFNLLGVPLVGADVCGFLGNTSEELCVRWTQLGAFYPFMRNHNSLLS
LPQEPYSFSEPAQQAMRKALTLRYALLPHLYTLFHQAHVAGETVARPLFLEFPKDS
STWTVDHQLLWGEALLITPVLQAGKAEVTGYFPLGTWYDLQTVPVEA
exemplary mature GAA amino acid sequence (corresponding to residues
288-782 of SEQ ID NO: 45; one embodiment of a mature GAA polypeptide)
SEQ ID NO: 48
GANLYGSHPFYLALEDGGSAHGVFLLNSNAMDVVLQPSPALSWRSTGGILDVYIFL
GPEPKSVVQQYLDVVGYPFMPPYWGLGFHLCRWGYSSTAITRQVVENMTRAHFPL
DVQWNDLDYMDSRRDFTFNKDGFRDFPAMVQELHQGGRRYMMIVDPAISSSGPA
GSYRPYDEGLRRGVFITNETGQPLIGKVWPGSTAFPDFTNPTALAWWEDMVAEFH
DQVPFDGMWIDMNEPSNFIRGSEDGCPNNELENPPYVPGVVGGTLQAATICASSHQ
FLSTHYNLHNLYGLTEAIASHRALVKARGTRPFVISRSTFAGHGRYAGHWTGDVW
SSWEQLASSVPEILQFNLLGVPLVGADVCGFLGNTSEELCVRWTQLGAFYPFMRNH
NSLLSLPQEPYSFSEPAQQAMRKALTLRYALLPHLYTLFHQAHVAGETVARPLFLE
FPKDSSTWTVDHQLLWGEALLITPVLQAGKAEVTGYFPLGTWYDLQTVPVEA
exemplary GAA polypeptide comprising mature GAA (residues 61-952;
one embodiment of a GAA polypeptide)
SEQ ID NO: 49
SRPGPRDAQAHPGRPRAVPTQCDVPPNSRFDCAPDKAITQEQCEARGCCYIPAKQG
LQGAQMGQPWCFFPPSYPSYKLENLSSSEMGYTATLTRTTPTFFPKDILTLRLDVM
METENRLHFTIKDPANRRYEVPLETPHVHSRAPSPLYSVEFSEEPFGVIVRRQLDGR
VLLNTTVAPLFFADQFLQLSTSLPSQYITGLAEHLSPLMLSTSWTRITLWNRDLAPT
PGANLYGSHPFYLALEDGGSAHGVFLLNSNAMDVVLQPSPALSWRSTGGILDVYIF
LGPEPKSVVQQYLDVVGYPFMPPYWGLGFHLCRWGYSSTAITRQVVENMTRAHFP
LDVQWNDLDYMDSRRDFTFNKDGFRDFPAMVQELHQGGRRYMMIVDPAISSSGP
AGSYRPYDEGLRRGVFITNETGQPLIGKVWPGSTAFPDFTNPTALAWWEDMVAEF
HDQVPFDGMWIDMNEPSNFIRGSEDGCPNNELENPPYVPGVVGGTLQAATICASSH
QFLSTHYNLHNLYGLTEAIASHRALVKARGTRPFVISRSTFAGHGRYAGHWTGDV
WSSWEQLASSVPEILQFNLLGVPLVGADVCGFLGNTSEELCVRWTQLGAFYPFMR
NHNSLLSLPQEPYSFSEPAQQAMRKALTLRYALLPHLYTLFHQAHVAGETVARPLF
LEFPKDSSTWTVDHQLLWGEALLITPVLQAGKAEVTGYFPLGTWYDLQTVPVEAL
GSLPPPPAAPREPAIHSEGQWVTLPAPLDTINVHLRAGYIIPLQGPGLTTTESRQQPM
ALAVALTKGGEARGELFWDDGESLEVLERGAYTQVIFLARNNTIVNELVRVTSEG
AGLQLQKVTVLGVATAPQQVLSNGVPVSNFTYSPDTKVLDICVSLLMGEQFLVSW
C
exemplary GAA polypeptide comprising mature GAA (residues 67-952;
one embodiment of a GAA polypeptide)
SEQ ID NO: 50
DAQAHPGRPRAVPTQCDVPPNSRFDCAPDKAITQEQCEARGCCYIPAKQGLQGAQ
MGQPWCFFPPSYPSYKLENLSSSEMGYTATLTRTTPTFFPKDILTLRLDVMMETEN
RLHFTIKDPANRRYEVPLETPHVHSRAPSPLYSVEFSEEPFGVIVRRQLDGRVLLNTT
VAPLFFADQFLQLSTSLPSQYITGLAEHLSPLMLSTSWTRITLWNRDLAPTPGANLY
GSHPFYLALEDGGSAHGVFLLNSNAMDVVLQPSPALSWRSTGGILDVYIFLGPEPK
SVVQQYLDVVGYPFMPPYWGLGFHLCRWGYSSTAITRQVVENMTRAHFPLDVQW
NDLDYMDSRRDFTFNKDGFRDFPAMVQELHQGGRRYMMIVDPAISSSGPAGSYRP
YDEGLRRGVFITNETGQPLIGKVWPGSTAFPDFTNPTALAWWEDMVAEFHDQVPF
DGMWIDMNEPSNFIRGSEDGCPNNELENPPYVPGVVGGTLQAATICASSHQFLSTH
YNLHNLYGLTEAIASHRALVKARGTRPFVISRSTFAGHGRYAGHWTGDVWSSWEQ
LASSVPEILQFNLLGVPLVGADVCGFLGNTSEELCVRWTQLGAFYPFMRNHNSLLS
LPQEPYSFSEPAQQAMRKALTLRYALLPHLYTLFHQAHVAGETVARPLFLEFPKDS
STWTVDHQLLWGEALLITPVLQAGKAEVTGYFPLGTWYDLQTVPVEALGSLPPPP
AAPREPAIHSEGQWVTLPAPLDTINVHLRAGYIIPLQGPGLTTTESRQQPMALAVAL
TKGGEARGELFWDDGESLEVLERGAYTQVIFLARNNTIVNELVRVTSEGAGLQLQ
KVTVLGVATAPQQVLSNGVPVSNFTYSPDTKVLDICVSLLMGEQFLVSWC
exemplary GAA polypeptide comprising mature GAA (residues 70-952;
one embodiment of a GAA polypeptide)
SEQ ID NO: 51
AHPGRPRAVPTQCDVPPNSRFDCAPDKAITQEQCEARGCCYIPAKQGLQGAQMGQ
PWCFFPPSYPSYKLENLSSSEMGYTATLTRTTPTFFPKDILTLRLDVMMETENRLHF
TIKDPANRRYEVPLETPHVHSRAPSPLYSVEFSEEPFGVIVRRQLDGRVLLNTTVAP
LFFADQFLQLSTSLPSQYITGLAEHLSPLMLSTSWTRITLWNRDLAPTPGANLYGSH
PFYLALEDGGSAHGVFLLNSNAMDVVLQPSPALSWRSTGGILDVYIFLGPEPKSVV
QQYLDVVGYPFMPPYWGLGFHLCRWGYSSTAITRQVVENMTRAHFPLDVQWNDL
DYMDSRRDFTFNKDGFRDFPAMVQELHQGGRRYMMIVDPAISSSGPAGSYRPYDE
GLRRGVFITNETGQPLIGKVWPGSTAFPDFTNPTALAWWEDMVAEFHDQVPFDGM
WIDMNEPSNFIRGSEDGCPNNELENPPYVPGVVGGTLQAATICASSHQFLSTHYNL
HNLYGLTEAIASHRALVKARGTRPFVISRSTFAGHGRYAGHWTGDVWSSWEQLAS
SVPEILQFNLLGVPLVGADVCGFLGNTSEELCVRWTQLGAFYPFMRNHNSLLSLPQ
EPYSFSEPAQQAMRKALTLRYALLPHLYTLFHQAHVAGETVARPLFLEFPKDSSTW
TVDHQLLWGEALLITPVLQAGKAEVTGYFPLGTWYDLQTVPVEALGSLPPPPAAPR
EPAIHSEGQWVTLPAPLDTINVHLRAGYIIPLQGPGLTTTESRQQPMALAVALTKGG
EARGELFWDDGESLEVLERGAYTQVIFLARNNTIVNELVRVTSEGAGLQLQKVTVL
GVATAPQQVLSNGVPVSNFTYSPDTKVLDICVSLLMGEQFLVSWC
bovine GAA precursor protein (GenBank Accession No. NP_776338.1)
SEQ ID NO: 52
MMRWPPCSRPLLGVCTLLSLALLGHILLHDLEVVPRELRGFSQDEIHQACQPGASSP
ECRGSPRAAPTQCDLPPNSRFDCAPDKGITPQQCEARGCCYMPAEWPPDAQMGQP
WCFFPPSYPSYRLENLTTTETGYTATLTRAVPTFFPKDIMTLRLDMLMETESRLHFT
IKDPANRRYEVPLETPRVYSQAPFTLYSVEFSEEPFGVVVRRKLDGRVLLNTTVAPL
FFADQFLQLSTSLPSQHITGLAEHLGSLMLSTNWTKITLWNRDIAPEPNVNLYGSHP
FYLVLEDGGLAHGVFLLNSNAMDVVLQPSPALSWRSTGGILDVYIFLGPEPKSVVQ
QYLDVVGYPFMPPYWGLGFHLCRWGYSTSAITRQVVENMTRAYFPLDVQWNDLD
YMDARRDFTFNKDHFGDFPAMVQELHQGGRRYIMIVDPAISSSGPAGTYRPYDEG
LRRGVFITNETGQPLIGQVWPGLTAFPDFTNPETLDWWQDMVTEFHAQVPFDGM
WIDMNEPSNFVRGSVDGCPDNSLENPPYLPGVVGGTLRAATICASSHQFLSTHYDL
HNLYGLTEALASHRALVKARGMRPFVISRSTFAGHGRYSGHWTGDVWSNWEQLS
YSVPEILLFNLLGVPLVGADICGFLGNTSEELCVRWTQLGAFYPFMRNHNALNSQP
QEPYRFSETAQQAMRKAFTLRYVLLPYLYTLFHRAHVRGETVARPLFLEFPEDPST
WTVDRQLLWGEALLITPVLEAEKVEVTGYFPQGTWYDLQTVPMEAFGSLPPPAPL
TSVIHSKGQWVTLSAPLDTINVHLRAGHIIPMQGPALTTTESRKQHMALAVALTAS
GEAQGELFWDDGESLGVLDGGDYTQLIFLAKNNTFVNKLVHVSSEGASLQLRNVT
VLGVATAPQQVLCNSVPVSNFTFSPDTETLAIPVSLTMGEQFVISWS
full linker region (residues 57-78 of GAA)
SEQ ID NO: 53
HILLHDFLLVPRELSGSSPVLEETHPAH

### INCORPORATION BY REFERENCE

All publications and patents mentioned herein, including WO 2018/049237 and WO 2015/0192092, are hereby incorporated by reference in their entirety as if each individual publication or patent was specifically and individually indicated to be incorporated by reference.

While specific embodiments of the subject disclosure have been discussed, the above specification is illustrative and not restrictive. Many variations of the disclosure will become apparent to those skilled in the art upon review of this specification and the claims below. The full scope of the disclosure should be determined by reference to the claims, along with their full scope of equivalents, and the specification, along with such variations.

We claim:

1. A method for treating a subject having a polyglucosan accumulation disease, comprising administering to the subject a therapeutically effective amount of a chimeric polypeptide comprising: (i) a mature acid alpha-glucosidase (GAA) polypeptide, and (ii) an internalizing moiety.

2. A method for delivering acid alpha-glucosidase activity into a cell from or of a subject having a polyglucosan accumulation disease, comprising contacting the cell with a chimeric polypeptide comprising: (i) a mature acid alpha-glucosidase polypeptide, and (ii) an internalizing moiety.

3. The method of claim 2, wherein the cell is in vitro.

4. The method of claim 2, wherein the cell is a muscle cell.

5. The method of claim 2, wherein the cell is a diaphragm muscle cell.

6. The method of claim 2, wherein the cell is a brain cell.

7. The method of claim 2, wherein the cell is a neuron.

8. The method of any one of claims 1-7, wherein the chimeric polypeptide has acid alpha-glucosidase activity.

9. The method of any one of claims 1-8, wherein the chimeric polypeptide or mature GAA polypeptide comprises the amino acid sequence of SEQ ID NO: 49.

10. The method of any one of claims 1-9, wherein the chimeric polypeptide or mature GAA polypeptide comprises the amino acid sequence of SEQ ID NO: 50.

11. The method of any one of claims 1-10, wherein the chimeric polypeptide or mature GAA polypeptide comprises the amino acid sequence of SEQ ID NO: 51.

12. The method of any one of claims 1-11, wherein the mature GAA polypeptide has a molecular weight of approximately 70-76 kilodaltons.

13. The method of any one of claims 1-12, herein the mature GAA polypeptide has a molecular weight of approximately 70 kilodaltons.

14. The method of any one of claims 1-13, wherein the mature GAA polypeptide has a molecular weight of approximately 76 kilodaltons.

15. The method of any one of claims 1-14, wherein the subject is a non-human animal.

16. The method of claim 15, wherein the non-human animal is a mouse.

17. The method of any one of claims 1-16, wherein the subject is a human.

18. The method of any one of claims 1-17, wherein the method results in clearance of glycogen.

19. The method of any one of claims 1-18, wherein the polyglucosan accumulation disease is glycogen storage disorder IV (GSD IV).

20. The method of any one of claims 1-18, wherein the polyglucosan accumulation disease is glycogen storage disorder VII (GSD VII).

21. The method of any one of claims 1-18, wherein the polyglucosan accumulation disease is glycogen storage disorder XV (GSD XV).

22. The method of any one of claims 1-18, wherein the polyglucosan accumulation disease is a RBCK1 deficiency.

23. The method of any one of claims 1-18, wherein the polyglucosan accumulation disease is PRKAG2 associated cardiomyopathy (PAC).

24. The method of any one of claims 1-18, wherein the polyglucosan accumulation disease is glycogen storage disorder V (GSD V).

25. The method of any one of claims 1-24, wherein the internalizing moiety is an antibody or antigen binding fragment, wherein the antibody or antigen binding fragment comprises a heavy chain variable domain and a light chain variable domain; wherein the heavy chain variable domain comprises the amino acid sequence of SEQ ID NO: 2; and wherein the light chain variable domain comprises the amino acid sequence of SEQ ID NO: 3.

26. A method for treating a subject having Lafora Disease, comprising administering to the subject a therapeutically effective amount of a chimeric polypeptide comprising: (i) a mature acid alpha-glucosidase (GAA) polypeptide, and (ii) an internalizing moiety.

27. A method for delivering acid alpha-glucosidase activity into a cell from or of a subject having Lafora Disease, comprising contacting the cell with a chimeric polypeptide comprising: (i) a mature acid alpha-glucosidase polypeptide, and (ii) an internalizing moiety.

28. The method of claim 27, wherein the cell is in vitro.

29. The method of claim 27, wherein the cell is a muscle cell.

30. The method of claim 27, wherein the cell is a diaphragm muscle cell.

31. The method of claim 27, wherein the cell is a brain cell.

32. The method of claim 27, wherein the cell is a neuron.

33. The method of any one of claims 26-32, wherein the chimeric polypeptide has acid alpha-glucosidase activity.

34. The method of any one of claims 26-33, wherein the internalizing moiety is an antibody or antigen binding fragment, wherein the antibody or antigen binding fragment comprises a heavy chain variable domain and a light chain variable domain; wherein the heavy chain variable domain comprises the amino acid sequence of SEQ ID NO: 2; and wherein the light chain variable domain comprises the amino acid sequence of SEQ ID NO: 3.

35. The method of any one of claims 26-34, wherein the chimeric polypeptide or mature GAA polypeptide comprises the amino acid sequence of SEQ ID NO: 49.

36. The method of any one of claims 26-35, wherein the chimeric polypeptide or mature GAA polypeptide comprises the amino acid sequence of SEQ ID NO: 50.

37. The method of any one of claims 26-36, wherein the chimeric polypeptide or mature GAA polypeptide comprises the amino acid sequence of SEQ ID NO: 51.

38. The method of any one of claims 26-37, wherein the mature GAA polypeptide has a molecular weight of approximately 70-76 kilodaltons.

39. The method of any one of claims 26-38, herein the mature GAA polypeptide has a molecular weight of approximately 70 kilodaltons.

40. The method of any one of claims 26-39, wherein the mature GAA polypeptide has a molecular weight of approximately 76 kilodaltons.

41. The method of any one of claims 26-40, wherein the subject is a non-human animal.

42. The method of claim 41, wherein the non-human animal is a mouse.

43. The method of any one of claims 26-40, wherein the subject is a human.

44. The method of any one of claims 26-43, wherein the method results in clearance of glycogen.

45. The method of any one of claims 26-44, wherein the method results in degradation of Lafora bodies.

46. A method for treating a subject having Danon Disease, comprising administering to the subject a therapeutically effective amount of a chimeric polypeptide comprising: (i) a mature acid alpha-glucosidase (GAA) polypeptide, and (ii) an internalizing moiety.

47. A method for delivering acid alpha-glucosidase activity into a cell from or of a subject having Danon Disease, comprising contacting the cell with a chimeric polypeptide comprising: (i) a mature acid alpha-glucosidase polypeptide, and (ii) an internalizing moiety.

48. The method of claim 47, wherein the cell is in vitro.

49. The method of claim 47, wherein the cell is a muscle cell.

50. The method of claim 47, wherein the cell is a diaphragm muscle cell.

51. The method of claim 47, wherein the cell is a brain cell.

52. The method of claim 47, wherein the cell is a neuron.

53. The method of any one of claims 46-52, wherein the chimeric polypeptide has acid alpha-glucosidase activity.

54. The method of any one of claims 46-53, wherein the internalizing moiety is an antibody or antigen binding fragment, wherein the antibody or antigen binding fragment comprises a heavy chain variable domain and a light chain variable domain; wherein the heavy chain variable domain comprises the amino acid sequence of SEQ ID NO: 2; and wherein the light chain variable domain comprises the amino acid sequence of SEQ ID NO: 3.

55. The method of any one of claims 46-54, wherein the chimeric polypeptide or mature GAA polypeptide comprises the amino acid sequence of SEQ ID NO: 49.

56. The method of any one of claims 46-55, wherein the chimeric polypeptide or mature GAA polypeptide comprises the amino acid sequence of SEQ ID NO: 50.

57. The method of any one of claims 46-56, wherein the chimeric polypeptide or mature GAA polypeptide comprises the amino acid sequence of SEQ ID NO: 51.

58. The method of any one of claims 46-57, wherein the mature GAA polypeptide has a molecular weight of approximately 70-76 kilodaltons.

59. The method of any one of claims 46-58, herein the mature GAA polypeptide has a molecular weight of approximately 70 kilodaltons.

60. The method of any one of claims 46-59, wherein the mature GAA polypeptide has a molecular weight of approximately 76 kilodaltons.

61. The method of any one of claims 46-60, wherein the subject is a non-human animal.

62. The method of claim 61, wherein the non-human animal is a mouse.

63. The method of any one of claims 46-60, wherein the subject is a human.

64. The method of any one of claims 46-63, wherein the method results in clearance of glycogen.

65. A method for treating a subject having Danon Disease, comprising administering to the subject a therapeutically effective amount of a chimeric polypeptide comprising: (i) an alpha-amylase polypeptide, and (ii) an internalizing moiety; wherein the alpha-amylase polypeptide comprises the amino acid sequence of SEQ ID NO: 1; and wherein the internalizing moiety is an antibody or antigen binding fragment, wherein the antibody or antigen binding fragment comprises a heavy chain variable domain and a light chain variable domain; wherein the heavy chain variable domain comprises the amino acid sequence of SEQ ID NO: 2; and wherein the light chain variable domain comprises the amino acid sequence of SEQ ID NO: 3.

66. A method for treating a subject having Alzheimer's Disease, comprising administering to the subject a therapeutically effective amount of a chimeric polypeptide comprising: (i) an alpha-amylase polypeptide, and (ii) an internalizing moiety; wherein the alpha-amylase polypeptide comprises the amino acid sequence of SEQ ID NO: 1; and wherein the internalizing moiety is an antibody or antigen binding fragment, wherein the antibody or antigen binding fragment comprises a heavy chain variable domain and a light chain variable domain; wherein the heavy chain variable domain comprises the amino acid sequence of SEQ ID NO: 2; and wherein the light chain variable domain comprises the amino acid sequence of SEQ ID NO: 3.

67. The method of claim 65 or 66, wherein the alpha-amylase polypeptide consists of the amino acid sequence of SEQ ID NO: 1.

68. The method of any one of claims 65-67, wherein the heavy chain comprises the leader sequence of SEQ ID NO: 4.

69. The method of any one of claims 65-68, wherein the light chain comprises the leader sequence of SEQ ID NO: 5.

70. The method of any one of claims 65-69, wherein the chimeric polypeptide has alpha-1,4-glucosidic bonds hydrolytic activity.

71. The method of any one of claims 65-70, wherein the chimeric polypeptide is capable of hydrolyzing alpha-1,4-glucosidic bonds in a cell-free system.

72. The method of any one of claims 65-71, wherein the chimeric polypeptide is capable of hydrolyzing alpha-1,4-glucosidic bonds in a cell from a subject having the disease.

73. The method of claim 72, wherein the subject is a non-human animal.

74. The method of claim 73, wherein the non-human animal is a mouse.

75. The method of claim 72, wherein the subject is a human.

76. The method of any one of claims 72-75, wherein the cell is in vitro.

77. The method of any one of claims 72-75, wherein the cell is a muscle cell.

78. The method of any one of claims 72-75, wherein the cell is a diaphragm muscle cell.

79. The method of any one of claims 72-75, wherein the cell is a brain cell.

80. The method of any one of claims 72-75, wherein the cell is a neuron.

81. The method of any one of claims 65-80, wherein the alpha-amylase polypeptide is chemically conjugated to the internalizing moiety.

82. The method of any one of claims 65-81, wherein the chimeric polypeptide comprises a fusion protein comprising the alpha-amylase polypeptide and all or a portion of the internalizing moiety.

83. The method of claim 82, wherein the chimeric polypeptide does not include a linker interconnecting the alpha-amylase polypeptide to the internalizing moiety.

84. The method of claim 82, wherein the fusion protein comprises a linker.

85. The method of claim 84, wherein the linker conjugates or joins the alpha-amylase polypeptide to the internalizing moiety.

86. The method of claim 84 or 85, wherein the linker is a cleavable linker.

87. The method of any one of claims 84-86, wherein the linker comprises the amino acid sequence of SEQ ID NO: 6.

88. The method of any one of claims 65-87, wherein all or a portion of the internalizing moiety is conjugated or joined, directly or via a linker, to the N-terminal amino acid of the alpha-amylase polypeptide.

89. The method of any one of claims 65-87, wherein all or a portion of the internalizing moiety is conjugated or joined, directly or via a linker, to the C-terminal amino acid of the alpha-amylase polypeptide.

90. The method of any one of claims 65-87, wherein all or a portion of the internalizing moiety is conjugated or joined, directly or indirectly to an internal amino acid of the alpha-amylase polypeptide.

91. The method of any one of claims 65-90, wherein the internalizing moiety promotes delivery of the chimeric polypeptide into cells via an equilibrative nucleoside transporter (ENT) transporter.

92. The method of any one of claims 65-91, wherein the internalizing moiety promotes delivery of the chimeric polypeptide into cells via ENT2.

93. The method of any one of claims 65-92, wherein the internalizing moiety promotes delivery of the chimeric polypeptide into a muscle cell.

94. The method of claim 93, wherein the muscle cell is a cardiac muscle cell.

95. The method of any one of claims 65-94, wherein the internalizing moiety promotes delivery of the chimeric polypeptide into a neuronal cell.

96. The method of claim 95, wherein the neuronal cell is a brain neuronal cell.

97. The method of any one of claims 65-96, wherein the internalizing moiety comprises an antibody.

98. The method of claim 97, wherein the antibody is a monoclonal antibody.

99. The method of any one of claims 65-96, wherein the internalizing moiety comprises an antigen-binding fragment.

100. The method of claim 99, wherein the antigen-binding fragment is a Fab.

101. The method of claim 99, wherein the antigen-binding fragment is a Fab′.

102. The method of claim 99, wherein the antigen-binding fragment is an scFv.

103. The method of any one of claims 65-102, wherein the chimeric polypeptide is produced recombinantly.

104. The method of claim 103, wherein the chimeric polypeptide is produced in a prokaryotic or eukaryotic cell.

105. The method of claim 104, wherein the eukaryotic cell is selected from a yeast cell, an avian cell, an insect cell, or a mammalian cell.

106. The method of any one of claims 65-105, wherein one or more glycosylation groups are conjugated to the chimeric polypeptide.

107. The method of any one of claims 65-106, wherein the chimeric polypeptide comprises the amino acid sequence of SEQ ID NO: 7.

108. The method of any one of claims 65-107, wherein the chimeric polypeptide comprises the amino acid sequence of SEQ ID NO: 8.

109. The method of any one of claims 65-108, wherein the chimeric polypeptide comprises the amino acid sequence of SEQ ID NOs: 7 and 8.

110. The method of any one of claims 65-106, wherein the chimeric polypeptide comprises the amino acid sequence of SEQ ID NO: 9.

111. The method of any one of claims 65-106, wherein the chimeric polypeptide comprises the amino acid sequence of SEQ ID NO: 10.

112. The method of any one of claims 65-106, wherein the chimeric polypeptide comprises the amino acid sequence of SEQ ID NOs: 9 and 10.

113. The method of any one of claims 65-106, wherein the chimeric polypeptide comprises the amino acid sequence of SEQ ID NO: 43.

114. The method of any one of claims 65-106, wherein the chimeric polypeptide comprises the amino acid sequence of SEQ ID NO: 8.

115. The method of any one of claims 65-106, wherein the chimeric polypeptide comprises the amino acid sequences of SEQ ID NOs: 8 and 43.
